(19) United States
(12) Patent Application Publication
(10) Pub. No.: US 2019/0336757 A1
(43) Pub. Date: Nov. 7, 2019

(54) ELECTROPORATION SYSTEMS, METHODS, AND APPARATUS

(71) Applicant: OncoSec Medical Incorporated, San Diego, CA (US)

(72) Inventors: John F. Rodriguez, Vista, CA (US); Brandon Dang Phung, San Diego, CA (US); Christopher G. Twitty, San Diego, CA (US); Jason Jin, Irvine, CA (US)

(21) Appl. No.: 16/401,811

(22) Filed: May 2, 2019

Related U.S. Application Data
(60) Provisional application No. 62/665,553, filed on May 2, 2018.
(60) Provisional application No. 62/742,684, filed on Oct. 8, 2018.
(60) Provisional application No. 62/745,699, filed on Oct. 15, 2018.
(60) Provisional application No. 62/755,001, filed on Nov. 2, 2018.
(60) Provisional application No. 62/824,011, filed on Mar. 26, 2019.

(51) Int. Cl. A61N 1/32; A61N 1/04; A61B 18/14

(52) U.S. Cl. CPC A61N 1/327; A61B 2018/00613; A61B 18/1477; A61N 1/0412

(57) ABSTRACT

Provided herein are systems, methods, and apparatus for electroporation, which may include an applicator; an endoscope, trocar or the like; a generator; and a drug delivery device. The applicator may include a control portion, an insertion tube connected to the control portion, an actuator engaged with the control portion, and a plurality of electrodes comprising a first electrode having a first tip and a second electrode having a second tip. The plurality electrodes may be configured to move between a retracted position and a deployed position in response to actuation by the actuator. A distance between the first tip of the first electrode and the second tip of the second electrode may be greater in the deployed position than in the retracted position. Various treatment methods are also provided.

### CROSS-REFERENCE TO RELATED APPLICATIONS

This application claims the benefit of the following U.S. Provisional Patent Application Nos. 62/665,553, filed May 2, 2018; 62/742,684 filed Oct. 8, 2018; 62/745,699 filed Oct. 15, 2018; 62/755,001 filed Nov. 2, 2018; and 62/824,011 filed Mar. 26, 2019, each of which is hereby incorporated by reference herein in its entirety as if fully set forth herein.

### BACKGROUND

Electrical fields may be used to create pores in cells through a process known as electroporation to increase the permeability of target cells and administer various localized treatments to a patient. There is a need for electroporation therapy in difficult to reach areas of the body, such as to treat tumors within the lungs, and there is a need to provide a large treatment area while still being able to fit the electroporation devices into these difficult to reach areas. There is also a need to administer a variety of treatment agents and therapies with a high degree of precision and minimal invasiveness.

Through applied effort, ingenuity, and innovation, many of these identified problems have been solved by developing solutions that are included in embodiments of the present invention, many examples of which are described in detail herein.

### BRIEF SUMMARY

Disclosed herein are electroporation systems, applicators, associated methods of treatment and use, and associated apparatus. In some embodiments, an applicator for electroporation may be provided. The applicator may include a control portion, an insertion tube connected to the control portion, an actuator engaged with the control portion, and a plurality of electrodes comprising a first electrode having a first tip and a second electrode having a second tip. In some embodiments, at least a portion of the actuator may be movable relative to the control portion and the insertion tube. The plurality of electrodes may be configured to move between a retracted position and a deployed position in response to actuation by the actuator. In some embodiments, a distance between the first tip of the first electrode and the second tip of the second electrode may be greater in the deployed position than in the retracted position.

In some embodiments, the plurality of electrodes may be recessed entirely within the insertion tube in the retracted position. At least a portion of the first electrode and the second electrode may be configured to extend from the insertion tube into adjacent tissue in the deployed position.

In the deployed position, the distance between the first tip of the first electrode and the second tip of the second electrode may be greater than an external diameter of the insertion tube.

In some embodiments, the insertion tube may include a first angled channel and a second angled channel defined at a distal end of the insertion tube. The first angled channel and the second angled channel may each be oriented at acute angles to a longitudinal axis of the insertion tube. The first electrode may be configured to extend at least partially through the first angled channel in the deployed position. In some embodiments, the second electrode may be configured to extend at least partially through the second angled channel in the deployed position. In the retracted position, the first electrode and the second electrode may be disposed parallel to each other within the insertion tube. In the deployed position, at least a portion of the first electrode and at least a portion of the second electrode may be disposed at the respective acute angles of the first angled channel and the second angled channel.

In some embodiments, the applicator may include a bladder engaged with the first electrode and the second electrode. The bladder may be disposed entirely within the insertion tube in the retracted position, and the bladder may be disposed at least partially outside the insertion tube in the deployed position.

In some embodiments, at least a portion of the first electrode and the second electrode may comprise nitinol. The nitinol may be configured to change shape in an instance in which the plurality of electrodes are in the deployed position, and the nitinol may be configured to change shape above human body temperature.

In some embodiments, the applicator may include a nitinol sleeve attached to each of the first electrode and a second electrode, wherein the nitinol is configured to change shape in an instance in which the plurality of electrodes are in the deployed position, and wherein the nitinol is configured to change shape above human body temperature.

In some embodiments, the first electrode and the second electrode may be non-linear.

The applicator may include a carrier movably disposed at least partially within the insertion tube. The first electrode and the second electrode may each be disposed at least partially within the carrier. The carrier may define a first portion associated with the first electrode and a second portion associated with the second electrode, and the first portion and the second portion may be configured to expand radially away from each other when moving from the retracted position to the expanded position. The applicator may include an inner member configured to receive a force from the actuator to expand the first portion and the second portion of the carrier radially outwardly. The applicator may include a spring disposed between the first portion and the second portion. The spring may be configured to expand the first portion and the second portion of the carrier radially outwardly. In some embodiments, the applicator may include a drug delivery channel configured to fluidly connect a drug delivery device with a target site via the insertion tube of the applicator.

In some embodiments, the actuator may be configured to displace the drug delivery channel towards the target site. The drug delivery channel may be configured to move between a retracted position of the drug delivery channel and the deployed position of the drug delivery channel simultaneously with the plurality of electrodes in response to actuation by the actuator. In some embodiments, the insertion tube defines a piercing tip at a distal end.

In another embodiment, a system for electroporation is provided. The system may include an applicator that may include a control portion, an insertion tube connected to the control portion, an actuator engaged with the control portion, and a plurality of electrodes comprising a first electrode having a first tip and a second electrode having a second tip. The system may further include an endoscope, trocar, or the like defining a working channel, a generator electrically connected to the plurality of electrodes, and a drug delivery device configured to deliver one or more treatment agents through the working channel of the endoscope, (e.g., a flexible endoscope, a rigid endoscope, trocar, or the like).

As used herein, the term “control portion” may refer to a user-operable portion of the applicator having one or more electrical and/or hydraulic connections for receiving electrical pulses and/or one or more treatment agents, respectively. As used herein, the term “insertion tube” may refer to any elongate, hollow portion of the applicator having any cross-sectional shape, at least a portion of which is configured to be inserted into a patient and through which electrical pulses and/or the one or more treatment agents are configured to be directed to the target treatment site.

In some embodiments, at least a portion of the actuator is movable relative to the control portion and the insertion tube. The plurality of electrodes may be configured to move between a retracted position and a deployed position in response to actuation by the actuator. A distance between the first tip of the first electrode and the second tip of the second electrode may be greater in the deployed position than in the retracted position. At least a portion of the insertion tube of the applicator may be configured to pass through the working channel. The generator may be configured to deliver electrical signals to the plurality of electrodes.

In some embodiments, in the deployed position, the distance between the first tip of the first electrode and the second tip of the second electrode may be greater than an internal diameter of the working channel.

In some embodiments, in the retracted position, the insertion tube and plurality of electrodes may be configured to pass through the working channel of the endoscope or the like.

The system may include a processor configured to cause the generator to transmit electrical signals to the first electrode and the second electrode and receive electrical signals indicative of an impedance of a tissue disposed between the first electrode and the second electrode.

In some embodiments, the endoscope may be a bronchoscope.

In yet another embodiment, a method of endoscopically or laparoscopically treating a tumor may be provided. The method may include inserting an endoscope or the like into a patient until a distal end of the endoscope is disposed adjacent to a target site, inserting a portion of a drug delivery device into a working channel of the endoscope, such that the portion of the drug delivery device is positioned adjacent to the target site, administering a treatment agent to the target site from the drug delivery device, removing the portion of the drug delivery device from the endoscope, inserting an insertion tube of an applicator into the working channel of the endoscope, such that a distal end of the insertion tube, including a plurality of electrodes, is positioned adjacent to the target site, delivering one or more electrical pulses from a generator to the electrodes to electroporate the tissue at the target site, and removing the applicator and endoscope from the patient.

In another embodiment, a system for electroporation may be provided. The system may include an applicator that may include a control portion, an insertion tube connected to the control portion, an actuator engaged with the control portion, and a plurality of electrodes comprising a first electrode having a first tip and a second electrode having a second tip. The system may further include a trocar defining a working channel, a generator electrically connected to the plurality of electrodes, and a drug delivery device configured to deliver one or more treatment agents through the working channel of the trocar. In some embodiments, the trocar may be configured to puncture or otherwise access a body cavity of a subject under guided imagery to administer one or more therapies.

In some embodiments, at least a portion of the actuator may be movable relative to the control portion and the insertion tube. The plurality of electrodes may be configured to move between a retracted position and a deployed position in response to actuation by the actuator. In some embodiments, a distance between the first tip of the first electrode and the second tip of the second electrode is greater in the deployed position than in the retracted position. At least a portion of the insertion tube of the applicator may be configured to pass through the working channel to access a visceral lesion. The generator may be configured to deliver electrical signals to the plurality of electrodes.

In some embodiments, methods of treating a visceral lesion are provided. The methods may include inserting a trocar into a patient until a distal end of the trocar is disposed adjacent to a target site comprising the visceral lesion; inserting a portion of a drug delivery device into a working channel of the trocar, such that the portion of the drug delivery device is positioned adjacent to the target site; administering a treatment agent to the target site from the drug delivery device; removing the portion of the drug delivery device from the trocar; inserting an insertion tube of an applicator into the working channel of the trocar, such that a distal end of the insertion tube, including a plurality of electrodes, is positioned adjacent to the target site; delivering one or more electrical pulses from a generator to the electrodes to electroporate the tissue at the target site; and removing the applicator and trocar from the patient.

In some embodiments, methods of treating a subject having a tumor are provided. The methods include administering to the subject an effective dose of a therapeutic molecule, and administering electroporation therapy to the tumor. The electroporation therapy may include administering an electric pulse to the tumor using any of the electroporation systems described herein. The tumor can be cancerous or non-cancerous. The tumor can be, but is not limited to, a solid tumor, a surface lesion, a non-surface lesion, visceral a lesion within 15 cm of body surface, or a visceral lesion. In some embodiments, the described methods can be used to treat primary tumors as well as distant tumors and metastases. In some embodiments, the described methods provide for reducing the size of, debulking, or inhibiting the growth of a tumor, inhibiting the growth of cancer cells, inhibiting or reducing metastasis, reducing or inhibiting the development of metastatic cancer, and/or reducing recurrence of cancer in a subject suffering from cancer. The tumor is not limited to a specific type of tumor or cancer.

In some embodiments, the therapeutic molecule is administered a drug delivery device of the applicator. The therapeutic molecule may include an expression vector encoding a therapeutic polypeptide. In some embodiments, the expression vector encodes one or more of: co-stimulatory polypeptide, immunomodulatory polypeptide, immunostimulatory cytokine, checkpoint inhibitor, adjuvant, antigen, or genetic adjuvant-antigen fusion polypeptide. The co-stimulatory molecule may be selected from the group consisting of: GITR, CD137, CD134, CD40L, and CD27 agonists. In some embodiments, the expression vector encodes a polypeptide comprising CXCL9, anti-CD3 scFv, or anti-CTLA-4 scFv. The immunostimulatory cytokine may be selected from the group consisting of: TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ, and a combination of any two of TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ. In some embodiments, the expression vector encodes an anti-CD3 scFv, CXCL9, or anti-CTLA-4 scFv. In some embodiments, the expression vector encodes and anti-CD3 scFv and IL-12. In some embodiments, the expression vector encodes IL-12 and CXCL9.

The methods may further include administering an effective dose of a checkpoint inhibitor to the subject. In some embodiments, the checkpoint inhibitor is administered systemically. The checkpoint inhibitor may be encoded on the expression vector encoding an immunostimulatory cytokine or on a second expression vector and delivered to the cancerous tumor by the electroporation therapy. The checkpoint inhibitor may be administered prior to, concurrent with, or subsequent to electroporation of the immunostimulatory cytokine.

In some embodiments, the expression vector comprises:
a) P-A-T-C,
b) P-A-T-B-T-C, or
c) P-C-T-A-T-B
   wherein P is a promoter, T is a translation modification element, A encodes an immunomodulatory molecule, a chain of an immunomodulatory molecule or a co-stimulatory molecule, B encodes an immunomodulatory molecule, a chain of an immunomodulatory molecule or a co-stimulatory molecule, and C encodes a immunomodulatory molecule, chain of an immunomodulatory molecule a costimulatory molecule, genetic adjuvant, antigen, genetic adjuvant-antigen fusion polypeptide, chemokine, or antigen binding polypeptide.

The methods may also include piercing a tissue with a distal end of the applicator to access the tumor. The methods may further comprise optimizing the electroporation parameters using EIS.

In some embodiments, methods of reducing recurrence of tumor cell growth in a mammalian tissue are provided. The methods may include administering a therapeutic molecule to the tumor and/or a tumor margin tissue, and administering electroporation therapy to the tumor and/or the tumor margin tissue using any of the electroporation systems disclosed herein.

In some embodiments, administering a therapeutic molecule includes injecting an expression vector encoding the therapeutic molecule into the tumor and/or a tumor margin tissue. The electroporation therapy may be administered prior to or after surgical resection or ablation of the tumor cell growth.

In some embodiments, methods of treating a subject having a tumor are provided. The methods may include administering to the subject an effective dose of at least one DNA-based treatment agent, and transfecting the at least one DNA-based treatment agent into a plurality of cells of the tumor using an electroporation applicator and generator. In some embodiments, the generator may apply low voltage electroporation pulses to the tumor via the electroporation applicator. In some embodiments, at least 4%, at least 5%, at least 6%, at least 7%, at least 8%, at least 9%, or at least 10% of the tumor cells in a treatment area are transfected.

In some embodiments, the low voltage electroporation pulses include a field of 700V/cm or less. In some embodiments, the low voltage electroporation pulses include a field of 600V/cm or less. In some embodiments, the low voltage electroporation pulses include a field of 500V/cm or less. In some embodiments, the low voltage electroporation pulses include a field of 400V/cm or less.

In some embodiments, each low voltage electroporation pulse defines a duration of 1 ms or greater. In some embodiments, each low voltage electroporation pulse defines a duration from 1 ms to 1 s.

In some embodiments, the low voltage electroporation pulses define a voltage of 600V or less. In some embodiments, the low voltage electroporation pulses comprise a voltage from 600V to 5V.

In some embodiments, the applicator may include a control portion; an insertion tube connected to the control portion; an actuator engaged with the control portion; and a plurality of electrodes comprising a first electrode having a first tip and a second electrode having a second tip. At least a portion of the actuator may be movable relative to the control portion and the insertion tube. In some embodiments, the plurality of electrodes may be configured to move between a retracted position and a deployed position in response to actuation by the actuator. A distance between the first tip of the first electrode and the second tip of the second electrode may be greater in the deployed position than in the retracted position. In some embodiments, the generator may be electrically connected to the plurality of electrodes, and the generator may deliver electrical signals to the plurality of electrodes.

In some embodiments, a method of treating a subject having a tumor is provided. The method may include administering to the subject an effective dose of at least one DNA-based treatment agent, transfecting the at least one DNA-based treatment agent into a plurality of cells of the tumor using an electroporation applicator and generator, wherein the generator is configured to apply high voltage electroporation pulses to the tumor via the electroporation applicator; and wherein 8-10% of the at least one DNA-based treatment agent is transfected into cells of the tumor.

In some embodiments, a method of modulating checkpoint inhibitor non-responsiveness in a non-responsive subject may be provided. The method may include administering to the non-responsive subject at least one checkpoint inhibitor; injecting a tumor in the non-responsive subject with an effective dose of at least one plasmid coding for a cytokine; and administering electroporation therapy to the tumor.

In some embodiments of the method, the tumor may be in the liver. In some embodiments, the tumor may be hepatocellular carcinoma. In some embodiments, the cytokine may be selected from the group consisting of: TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ, and a combination of any two of TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ. In some embodiments, the cytokine may be IL-12. In some embodiments, a plasmid encoding CXCL9, anti-CD3 scFv, or anti-CTLA-4 scFv may be administered to a liver tumor.

In some embodiments, a trocar-based system for electroporation may be provided. In some embodiments, the trocar-based system may include an applicator comprising a control portion; an insertion tube connected to the control portion; an actuator engaged with the control portion, wherein at least a portion of the actuator is movable relative to the control portion and the insertion tube; and a plurality of electrodes comprising a first electrode having a first tip and a second electrode having a second tip, wherein the plurality of electrodes are configured to move between a retracted position and a deployed position in response to actuation by the actuator. In some embodiments, a distance between the first tip of the first electrode and the second tip of the second electrode is greater in the deployed position than in the retracted position. The system may further include a trocar defining a working channel, wherein at least a portion of the insertion tube of the applicator is configured to pass through the working channel. In some embodiments, the system may include a generator electrically connected to the plurality of electrodes, wherein the generator is configured to deliver electrical signals to the plurality of electrodes. The system may further include a drug delivery device configured to deliver one or more treatment agents through the working channel of the trocar.

In one aspect, the present disclosure relates to an applicator for electroporation of tissue. In some embodiments, an applicator includes a control portion, an insertion tube connected to the control portion, an actuator engaged with the control portion and a plurality of electrodes. The plurality of electrodes includes a first electrode having a first tip and a second electrode having a second tip. The plurality of electrodes are configured to move between a retracted position and a deployed position in response to actuation of the actuator.

In some embodiments, a distance between the first tip of the first electrode and the second tip of the second electrode is greater in the deployed position than in the retracted position. In some embodiments, the insertion tube includes a drug delivery channel disposed therein, the drug delivery channel configured to receive at least one treatment agent. In some examples, the drug delivery channel is configured to retract and deploy with the plurality of electrodes. In some embodiments, a system includes the applicator and a separate drug delivery applicator. In some embodiments, a system includes the applicator and a low-voltage generator operatively connected to the applicator.

In one aspect, the present disclosure relates to a system for electroporation of tissue. In some embodiments, an applicator of a system includes a body with an insertion tube, an actuator engaged with the body and at least one electrode. The at least one electrode includes a first electrode having a first tip. The at least one electrode is configured to move between a retracted position and a deployed position in response to actuation of the actuator. The generator is low-voltage and is electrically connected to the at least one electrode.

In some embodiments, the system includes an endoscope configured for the disposal of the insertion tube therein. In some embodiments, the applicator includes a drug delivery channel disposed therein, the drug delivery channel configured to deliver at least one treatment agent.

In one aspect, the present disclosure relates to a method of treating a diseased tissue, such as a visceral lesion. In some embodiments, a method includes inserting an endoscope into a patient until a distal end of the endoscope is disposed adjacent to a target site comprising the diseased tissue; inserting a portion of an applicator into a working channel of the endoscope, such that the portion of the applicator is positioned adjacent to the target site with the endoscope disposed adjacent to the target site; administering at least one treatment agent to the target site through the applicator; actuating the applicator to deploy a plurality of electrodes of the applicator; and delivering one or more electrical pulses from a generator to the electrodes to electroporate the tissue at the target site.

In some embodiments, a method of treating diseased tissue includes inserting a endoscope into a patient until a distal end of the endoscope is disposed adjacent to a target site comprising the diseased tissue; inserting a portion of a drug delivery device into a working channel of the endoscope, such that the portion of the drug delivery device is positioned adjacent to the target site with the endoscope disposed adjacent to the target site; administering at least one treatment agent to the target site from the drug delivery device; removing the portion of the drug delivery device from the endoscope; inserting an insertion tube of an applicator into the working channel of the endoscope, such that a distal end of the insertion tube, including a plurality of electrodes, is positioned adjacent to the target site with the endoscope disposed adjacent to the target site; delivering one or more electrical pulses from a generator to the electrodes to electroporate the tissue at the target site; and removing the applicator and endoscope from the patient.

In some embodiments, a method of treating diseased tissue includes inserting a drug delivery device into a patient until a portion of the drug delivery device is positioned adjacent to a target site comprising the diseased tissue; administering a treatment agent to the target site from the drug delivery device; removing the drug delivery device from the patient; inserting an endoscope into a patient until a distal end of the endoscope is disposed adjacent to a target site comprising the diseased tissue; inserting an insertion tube of an applicator into the working channel of the endoscope, such that a distal end of the insertion tube, including a plurality of electrodes, is positioned adjacent to the target site with the endoscope disposed adjacent to the target site; delivering one or more electrical pulses from a generator to the electrodes to electroporate the tissue at the target site; and removing the applicator and endoscope from the patient.

In an example embodiment, a method of treating a lesion at a lung of a subject who is non-responsive or predicted to be non-responsive to anti-PD-1 or anti-PD-L1 therapy may include administering to the lesion an effective dose of at least one plasmid coding for IL-12; administering electroporation therapy to the lesion; and administering to the subject an effective dose of at least one checkpoint inhibitor; wherein administering the electroporation therapy comprises administering an electric pulse to the lesion using an electroporation system comprising: an applicator comprising: a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position. The system may further include a generator electrically connected to the plurality of electrodes, wherein administering the electric pulse to the lesion comprises disposing the first electrode and the second electrode into or adjacent to the lesion, and delivering the electric pulse from the generator to the first electrode and the second electrode.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the electroporation system further comprises an insertion device comprising one of a rigid trocar or flexible endoscope defining at least one working channel, wherein at least a portion of the applicator is configured to pass through the at least one working channel to access the lesion.

In some embodiments, the electroporation system further comprises a drug delivery device configured to deliver at least one of the at least one plasmid or the at least one checkpoint inhibitor through the at least one working channel of the insertion device.

In some embodiments, the applicator further defines a drug delivery channel configured to deliver at least one of the at least one plasmid or the at least one checkpoint inhibitor to the lesion.

In some embodiments, the electroporation system further comprises at least one robotic arm engaged with the applicator to control a position of the applicator during administration of at least one of the at least one plasmid, the at least one checkpoint inhibitor, or the electroporation therapy.

In some embodiments, the electroporation system further comprises at least one visualization device configured to generate imagery of the lesion before or during administration of at least one of the at least one plasmid, the at least one checkpoint inhibitor, or the electroporation therapy. In some embodiments, the at least one visualization device comprises a computed tomography scanner.

In some embodiments, the generator is configured to output low-voltage electric pulses. The electric pulses may have a field strength of 700V/cm or less.

In some embodiments, the generator is configured to output high-voltage electric pulses.

In some embodiments, the at least one plasmid comprises tavokinogene telseplasmid.

In some embodiments, the checkpoint inhibitor is administered systemically.

In some embodiments, the checkpoint inhibitor is an anti-PD-1 antibody or an anti-PD-L1 antibody.

In some embodiments, the checkpoint inhibitor comprises: nivolumab, pembrolizumab, pidilizumab, or MPDL3280A.

In another example embodiment, a system for treating a lesion at a lung of a subject who is non-responsive or predicted to be non-responsive to anti-PD-1 or anti-PDL1 therapy may include an applicator comprising a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position; a generator electrically connected to the plurality of electrodes, wherein the generator is configured to deliver an electric pulse to the first electrode and second electrode to administer the electric pulse to the lesion; and at least one drug delivery device configured to deliver to the subject an effective dose of at least one plasmid coding for IL-12 and an effective dose of at least one checkpoint inhibitor.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the system may include an insertion device comprising one of a rigid trocar or flexible endoscope defining at least one working channel, wherein at least a portion of the applicator is configured to pass through the at least one working channel to access the lesion.

In some embodiments, the system may include a drug delivery device configured to deliver the at least one plasmid through the at least one working channel of the insertion device.

In some embodiments, the applicator further defines a drug delivery channel configured to deliver the at least one plasmid to the lesion.

In some embodiments, the system may include at least one robotic arm engaged with the applicator to control a position of the applicator during administration of at least one of the at least one plasmid or the electroporation therapy.

In some embodiments, the system may include at least one visualization device configured to generate imagery of the lesion before or during administration of at least one of the at least one plasmid or the electroporation therapy. The at least one visualization device may include a computed tomography scanner.

In some embodiments, the generator is configured to output low-voltage electric pulses. In some embodiments, the electric pulses have a field strength of 700V/cm or less.

In some embodiments, the generator is configured to output high-voltage electric pulses.

In some embodiments, the at least one plasmid comprises tavokinogene telseplasmid.

In yet another example embodiment, a method of treating a lesion at a lung of a subject may include administering to the lesion an effective dose of at least one treatment agent; administering electroporation therapy to the lesion, the electroporation therapy comprising administering an electric pulse to the lesion using an electroporation system comprising: an applicator comprising: a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position. The system may further include a generator electrically connected to the plurality of electrodes, wherein administering the electric pulse to the lesion comprises disposing the first electrode and the second electrode into or adjacent to the lesion, and delivering the electric pulse from the generator to the first electrode and the second electrode.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the electroporation system may further include an insertion device defining at least one working channel, wherein at least a portion of the applicator is configured to pass through the at least one working channel to access the lesion.

In some embodiments, the electroporation system may further include a drug delivery device configured to deliver the at least one treatment agent through the at least one working channel of the insertion device. In some embodiments, the insertion device may include a bronchoscope, and wherein the applicator is at least partially flexible.

In some embodiments, the applicator further defines a drug delivery channel configured to deliver the at least one treatment agent to the lesion.

In some embodiments, the electroporation system further comprises at least one robotic arm engaged with the applicator to control a position of the applicator during administration of at least one of the at least one treatment agent or the electroporation therapy.

In some embodiments, the electroporation system further comprises at least one visualization device configured to generate imagery of the lesion before or during administration of at least one of the at least one treatment agent or the electroporation therapy. The at least one visualization device may include a computed tomography scanner.

In some embodiments, the generator is configured to output low-voltage electric pulses. The electric pulses may have a field strength of 700V/cm or less.

In some embodiments, the generator is configured to output high-voltage electric pulses.

In some embodiments, administering to the subject the effective dose of the at least one treatment agent comprises administering an effective dose of at least one plasmid coding for a cytokine. The at least one plasmid may include tavokinogene telseplasmid. In some embodiments, administering to the subject the effective dose of the at least one treatment agent may further include administering to the subject an effective dose of at least one checkpoint inhibitor.

In some embodiment, the method may include inserting a portion of the applicator into the lung of the subject via an esophagus of the subject.

In another example embodiment, a system for treating a lesion at a lung of a subject may include an applicator comprising a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position; a generator electrically connected to the plurality of electrodes, wherein the generator is configured to deliver an electric pulse to the first electrode and second electrode to administer the electric pulse to the lesion; and at least one drug delivery channel configured to deliver to the subject an effective dose of at least one treatment agent.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the system may include an insertion device defining at least one working channel, wherein at least a portion of the applicator is configured to pass through the at least one working channel to access the lesion.

In some embodiments, the system may include a drug delivery device configured to deliver the at least one treatment agent through the at least one working channel of the insertion device. The insertion device may include a bronchoscope, and wherein the applicator is at least partially flexible.

In some embodiments, the applicator further defines a drug delivery channel configured to deliver the at least one treatment agent to the lesion.

In some embodiments, the system may include at least one robotic arm engaged with the applicator to control a position of the applicator during delivery of at least one of the at least one treatment agent or the electroporation therapy.

In some embodiments, the system may include at least one visualization device configured to generate imagery of the lesion before or during delivery of at least one of the at least one treatment agent or the electroporation therapy. The at least one visualization device may include a computed tomography scanner.

In some embodiments, the generator is configured to output low-voltage electric pulses. The electric pulses may have a field strength of 700V/cm or less.

In some embodiments, the generator is configured to output high-voltage electric pulses.

In an example embodiment, a method of treating a visceral lesion at a pancreas of a subject may include administering to the subject an effective dose of at least one treatment agent; administering electroporation therapy to the visceral lesion, the electroporation therapy comprising administering an electric pulse to the visceral lesion using an electroporation system comprising: an applicator comprising: a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position. The system may further include a generator electrically connected to the plurality of electrodes, wherein administering the electric pulse to the visceral lesion comprises disposing the first electrode and the second electrode into or adjacent to the visceral lesion, and delivering the electric pulse from the generator to the first electrode and the second electrode.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the system may include an insertion device defining at least one working channel, wherein at least a portion of the applicator is configured to pass through the at least one working channel to access the visceral lesion. In some embodiments, the electroporation system further comprises a drug delivery device configured to deliver the at least one treatment agent through the at least one working channel of the insertion device. In some embodiments, the insertion device comprises an endoscope, and wherein the applicator is at least partially flexible.

In some embodiments, the applicator further defines a drug delivery channel configured to deliver the at least one treatment agent to the visceral lesion.

In some embodiments, the electroporation system further comprises at least one robotic arm engaged with the applicator to control a position of the applicator during administration of at least one of the at least one treatment agent or the electroporation therapy.

In some embodiments, the electroporation system further comprises at least one visualization device configured to generate imagery of the visceral lesion before or during administration of at least one of the at least one treatment agent or the electroporation therapy. The at least one visualization device may include a computed tomography scanner.

In some embodiments, the generator is configured to output low-voltage electric pulses. The electric pulses may have a field strength of 700V/cm or less.

In some embodiments, the generator is configured to output high-voltage electric pulses.

In some embodiments, administering to the subject the effective dose of the at least one treatment agent comprises administering an effective dose of at least one plasmid coding for a cytokine. The at least one plasmid may include tavokinogene telseplasmid.

In some embodiments, administering to the subject the effective dose of the at least one treatment agent further comprises administering to the subject an effective dose of at least one checkpoint inhibitor.

In some embodiments, the applicator further comprises a piercing tip. The method may further include inserting a portion of the applicator into a stomach of the subject; piercing a stomach wall with the piercing tip; and moving the plurality of electrodes from the retracted position to the deployed position.

In an example embodiment, a system for treating a visceral lesion at a pancreas of a subject may include an applicator comprising a plurality of electrodes comprising a first electrode having a first tip and a second electrode having a second tip, wherein the plurality electrodes are configured to move between a retracted position and a deployed position in response to actuation by the actuator; wherein a distance between the first tip of the first electrode and the second tip of the second electrode is greater in the deployed position than in the retracted position; a generator electrically connected to the plurality of electrodes, wherein the generator is configured to deliver an electric pulse to the first electrode and second electrode to administer the electric pulse to the visceral lesion; and at least one drug delivery channel configured to deliver to the subject an effective dose of at least one treatment agent.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the system may include an insertion device defining at least one working channel, wherein at least a portion of the applicator is configured to pass through the at least one working channel to access the visceral lesion. In some embodiments, the system may include a drug delivery device configured to deliver the at least one treatment agent through the at least one working channel of the insertion device. In some embodiments, the insertion device comprises a bronchoscope, and wherein the applicator is at least partially flexible.

In some embodiments, the applicator further defines a drug delivery channel configured to deliver the at least one treatment agent to the visceral lesion.

In some embodiments, the system may include at least one robotic arm engaged with the applicator to control a position of the applicator during delivery of at least one of the at least one treatment agent or the electroporation therapy.

In some embodiments, the system may include at least one visualization device configured to generate imagery of the visceral lesion before or during delivery of at least one of the at least one treatment agent or the electroporation therapy. The at least one visualization device may include a computed tomography scanner.

In some embodiments, the generator is configured to output low-voltage electric pulses. The electric pulses may have a field strength of 700V/cm or less.

In some embodiments, the generator is configured to output high-voltage electric pulses.

In some embodiments, the applicator further comprises a piercing tip configured to pierce a stomach wall of the subject to administer at least one of the at least one treatment agent or the electric pulse to or proximate the visceral lesion on the pancreas.

In an example embodiment, a method of treating a lesion of a subject may include administering to the subject an effective dose of at least one treatment agent; administering electroporation therapy to the lesion, the electroporation therapy comprising administering an electric pulse to the lesion using an electroporation system comprising: an applicator comprising a plurality of electrodes comprising a first electrode having a first tip and a second electrode having a second tip. The electroporation system may further include a generator electrically connected to the plurality of electrodes, wherein administering the electric pulse to the lesion comprises disposing the first electrode and the second electrode into or adjacent to the lesion, and delivering the electric pulse from the generator to the first electrode and the second electrode.

In some embodiments, the plurality electrodes are configured to move between a retracted position and a deployed position, and wherein a distance between the first tip of the first electrode and the second tip of the second electrode is greater in the deployed position than in the retracted position.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the electroporation system further comprises an insertion device defining at least one working channel, wherein at least a portion of the applicator is configured to pass through the at least one working channel to access the lesion.

In some embodiments, the electroporation system further comprises a drug delivery device configured to deliver the at least one treatment agent through the at least one working channel of the insertion device.

In some embodiments, the insertion device comprises an endoscope, and wherein the applicator is at least partially flexible.

In some embodiments, the insertion device comprises a trocar, and wherein the applicator is substantially rigid.

In some embodiments, the applicator further defines a drug delivery channel configured to deliver the at least one treatment agent to the lesion.

In some embodiments, the electroporation system further comprises at least one robotic arm engaged with the applicator to control a position of the applicator during administration of at least one of the at least one treatment agent or the electroporation therapy.

In some embodiments, the electroporation system further comprises at least one visualization device configured to generate imagery of the lesion before or during administration of at least one of the at least one treatment agent or the electroporation therapy. The at least one visualization device may include a computed tomography scanner.

In some embodiments, the generator is configured to output low-voltage electric pulses. The electric pulses may have a field strength of 700V/cm or less.

In some embodiments, the generator is configured to output high-voltage electric pulses.

In some embodiments, treating the lesion comprises administering an effective dose of at least one plasmid coding for a cytokine. In some embodiments, the cytokine comprises IL-12. In some embodiments, the at least one plasmid comprises tavokinogene telseplasmid. In some embodiments, treating the lesion further comprises administering to the subject an effective dose of at least one checkpoint inhibitor.

In some embodiments, the treatment agent comprises at least one plasmid encoding an immunomodulatory polypeptide. In some embodiments, the immunomodulatory polypeptide comprises: a cytokine, a costimulatory molecule, a genetic adjuvant, an antigen, a genetic adjuvant-antigen fusion polypeptide, a chemokine, or an antigen binding polypeptide.

In an example embodiment, a system for treating a lesion of a subject may include an applicator comprising a plurality of electrodes comprising a first electrode having a first tip and a second electrode having a second tip; a generator electrically connected to the plurality of electrodes, wherein the generator is configured to deliver an electric pulse to the first electrode and second electrode to administer the electric pulse to the lesion; and at least one drug delivery channel configured to deliver to the subject an effective dose of at least one treatment agent.

In some embodiments, the plurality electrodes are configured to move between a retracted position and a deployed position, and wherein a distance between the first tip of the first electrode and the second tip of the second electrode is greater in the deployed position than in the retracted position.

In some embodiments, the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

In some embodiments, the system may include an insertion device defining at least one working channel, wherein at least a portion of the applicator is configured to pass through the at least one working channel to access the lesion.

In some embodiments, the system may include a drug delivery device configured to deliver the at least one treatment agent through the at least one working channel of the insertion device.

In some embodiments, the insertion device comprises an endoscope, and wherein the applicator is at least partially flexible.

In some embodiments, the insertion device comprises a trocar, and wherein the applicator is substantially rigid.

In some embodiments, the applicator further defines a drug delivery channel configured to deliver the at least one treatment agent to the lesion.

In some embodiments, the system may include at least one robotic arm engaged with the applicator to control a position of the applicator during delivery of at least one of the at least one treatment agent or the electric pulse.

In some embodiments, the system may include at least one visualization device configured to generate imagery of the lesion before or during delivery of at least one of the at least one treatment agent or the electric pulse. The at least one visualization device may include a computed tomography scanner.

In some embodiments, the generator is configured to output low-voltage electric pulses. The electric pulses may have a field strength of 700V/cm or less.

In some embodiments, the generator is configured to output high-voltage electric pulses.

In some embodiments, treating the lesion comprises delivering an effective dose of at least one plasmid coding for a cytokine. In some embodiments, the at least one plasmid comprises tavokinogene telseplasmid. In some embodiments, delivering to the lesion the effective dose of the at least one treatment agent further comprises delivering to the subject an effective dose of at least one checkpoint inhibitor.

In some embodiments, the treatment agent comprises at least one plasmid encoding an immunomodulatory polypeptide.

In some embodiments, the immunomodulatory polypeptide comprises: a cytokine, a costimulatory molecule, a genetic adjuvant, an antigen, a genetic adjuvant-antigen fusion polypeptide, a chemokine, or an antigen binding polypeptide.

In some embodiments, the immunomodulatory molecule comprises: CXCL9, anti-CD3 scFv, or anti-CTLA-4 scFv

### BRIEF DESCRIPTION OF THE SEVERAL VIEWS OF THE DRAWINGS

Having thus described embodiments of the invention in general terms, reference will now be made to the accompanying drawings, which are not necessarily drawn to scale, and wherein:

FIG. 1 shows a block diagram of an electroporation system in accordance with some embodiments;

FIG. 2 shows a cross sectional view of a portion of an applicator in accordance with some embodiments;

FIG. 3 shows a generator and simplified applicator in accordance with some embodiments;

FIG. 4 shows an endoscope in accordance with some embodiments;

FIG. 5 shows a portion of an insertion tube and electrodes of an applicator in a retracted position in accordance with some embodiments;

FIG. 6 shows the portion of the insertion tube and electrodes of FIG. 5 in a deployed position;

FIG. 7 shows a portion of an insertion tube, electrodes, and bladder of an applicator in a retracted position in accordance with some embodiments;

FIG. 8 shows the portion of the insertion tube, electrodes, and bladder of FIG. 7 in a deployed position;

FIG. 9 shows a portion of an insertion tube and electrodes of an applicator in a retracted position in accordance with some embodiments;

FIG. 10 shows the portion of the insertion tube and electrodes of FIG. 9 in a deployed position;

FIG. 11 shows an electrode having a nitinol sleeve in accordance with some embodiments;

FIG. 12 shows a portion of an insertion tube and electrodes of an applicator in a retracted position in accordance with some embodiments;

FIG. 13 shows the portion of the insertion tube and electrodes of FIG. 12 in a deployed position;

FIG. 14 shows a portion of an insertion tube, carrier, and electrodes of an applicator in a retracted position in accordance with some embodiments;

FIG. 15 shows the portion of the insertion tube, carrier, and electrodes of FIG. 14 in a deployed position;

FIG. 16 shows a portion of an insertion tube, carrier, and electrodes of an applicator in a retracted position in accordance with some embodiments;

FIG. 17 shows the portion of the insertion tube, carrier, and electrodes of FIG. 16 in a deployed position;

FIG. 18 shows a flow chart of an example method of treatment in accordance with some embodiments;

FIG. 19 shows a side view of an applicator in accordance with some embodiments;

FIG. 20 shows a perspective view of an applicator with electrodes in a deployed position in accordance with some embodiments;

FIG. 21 shows a portion of an insertion tube and electrodes of an applicator in a retracted position in accordance with some embodiments;

FIG. 22 shows a side view of an applicator with electrodes in a deployed position in accordance with some embodiments;

FIG. 23 shows a partial view of a control portion and actuator of an applicator in accordance with some embodiments;

FIG. 24 shows a portion of an insertion tube and electrodes in a deployed position in accordance with some embodiments;

FIG. 25 shows a perspective view of an applicator with electrodes in a retracted position in accordance with some embodiments;

FIG. 26 shows a portion of an insertion tube and electrodes in a deployed position in accordance with some embodiments;

FIG. 27 shows a cross sectional, top view of an applicator in accordance with some embodiments;

FIG. 28 shows a side view of an applicator with electrodes in a deployed position in accordance with some embodiments;

FIG. 29 shows a perspective view of an insertion tube, carrier, and electrodes in accordance with some embodiments;

FIG. 30 shows a partial, cross-sectional view of an insertion tube, carrier, and electrodes in a deployed position in accordance with some embodiments;

FIG. 31 shows a perspective view of an applicator with electrodes in a deployed position in accordance with some embodiments;

FIG. 32 shows a perspective view of an applicator with electrodes in a retracted position in accordance with some embodiments;

FIG. 33 shows a partial, cross-sectional view of an insertion tube, a carrier, a pushing element, a wire, and an inner member in accordance with some embodiments;

FIG. 34 shows a side, cross-sectional view of an applicator in accordance with some embodiments;

FIG. 35 shows a side view of an applicator with electrodes in a deployed position in accordance with some embodiments;

FIG. 36 shows a cross-sectional view of a wire, a pushing element, an insertion tube, and a hollow mandrel in accordance with some embodiments;

FIG. 37 shows a second actuator according to some embodiments;

FIG. 38 shows a cross-sectional view of a portion of an insertion tube, a carrier, an inner member, an electrode, a pushing element, and a wire in accordance with some embodiments;

FIG. 39 shows a partial perspective view of a control portion and actuator in accordance with some embodiments;

FIG. 40 shows a perspective view of an applicator with electrodes in a retracted position in accordance with some embodiments;

FIG. 41 shows a portion of an insertion tube and electrodes in a deployed position in accordance with some embodiments;

FIG. 42 shows a portion of an insertion tube and electrodes in a deployed position in accordance with some embodiments;

FIG. 43 shows a perspective view of an applicator with electrodes in a retracted position in accordance with some embodiments;

FIG. 44 shows a cable and connector in accordance with some embodiments;

FIG. 45 shows the cable and connector of FIG. 44;

FIG. 46 shows a cross-sectional view of the connector of FIG. 44 taken along line A-A;

FIG. 47 shows a perspective view of an applicator having electrodes in a retracted position in accordance with some embodiments;

FIG. 48 shows a zoomed perspective view of the applicator of FIG. 47;

FIG. 49 shows another zoomed perspective view of the applicator of FIG. 47;

FIG. 50 shows a perspective view of the distal end of the applicator of FIG. 47;

FIG. 51 shows a cross-sectional view of the applicator of FIG. 47;

FIG. 52 shows another cross-sectional view of the applicator of FIG. 47;

FIG. 53 shows a cross-sectional view of a portion of the insertion tube, electrodes, and pushing element of the applicator of FIG. 47;

FIG. 54 shows the perspective view of the applicator of FIG. 47 having electrodes in a deployed position in accordance with some embodiments;

FIG. 55 shows a zoomed side view of the applicator of FIG. 54;

FIG. 56 shows a perspective view of the distal end of the applicator of FIG. 54;

FIG. 57 shows a cross-sectional view of the applicator of FIG. 54;

FIG. 58 shows a cross-sectional view of the distal end of the applicator of FIG. 54;

FIG. 59 shows a pushing element capable of carrying electrical pulses in accordance with some embodiments;

FIG. 60 shows a portion of an insertion tube, electrodes, and drug delivery tube of an applicator in a deployed position in accordance with some embodiments;

FIG. 61 shows a cross-sectional view of the insertion tube, electrodes, and drug delivery tube of the applicator of FIG. 60 in a deployed position in accordance with some embodiments;

FIG. 62 shows a portion of an insertion tube, electrodes, and drug delivery tube of an applicator in a deployed position in accordance with some embodiments;

FIG. 63 shows a cross-sectional view of the insertion tube, electrodes, and drug delivery tube of the applicator of FIG. 62 in a deployed position in accordance with some embodiments;

FIG. 64 shows a portion of an insertion tube, electrodes, and drug delivery tube of an applicator in a deployed position in accordance with some embodiments;

FIG. 65 shows a cross-sectional view of the insertion tube, electrodes, and drug delivery tube of the applicator of FIG. 64 in a deployed position in accordance with some embodiments;

FIG. 66 shows a portion of an insertion tube, carrier, inner member, electrodes, and drug delivery tube of an applicator in a deployed position in accordance with some embodiments;

FIG. 67 shows another flow chart of an example method of treatment in accordance with some embodiments;

FIG. 68 shows a yet another flow chart of an example method of treatment in accordance with some embodiments;

FIG. 69 shows an example applicator and endoscope extending into a stomach to access the pancreas in accordance with some embodiments;

FIG. 70 shows a cutaway view of the applicator, endoscope, stomach, and pancreas of FIG. 69;

FIG. 71 shows a zoomed perspective view of the distal ends of the endoscope and applicator of FIG. 69;

FIG. 72 shows a zoomed perspective view of the distal ends of the endoscope and applicator of FIG. 69 piercing a stomach wall;

FIG. 73 shows another zoomed perspective view of the distal ends of the endoscope and applicator of FIG. 69 piercing a stomach wall;

FIG. 74 shows a zoomed perspective view of the distal ends of the endoscope and applicator of FIG. 69 having electrodes and a drug delivery channel in the deployed position piercing the pancreas;

FIG. 75 shows an example applicator and bronchoscope extending into the lungs to access a lesion in accordance with some embodiments;

FIG. 76 shows cutaway view of the applicator, bronchoscope, and lungs of FIG. 75;

FIG. 77 shows a zoomed perspective view of the distal ends of the applicator and bronchoscope of FIG. 75;

FIG. 78 shows a zoomed perspective view of the distal ends of the bronchoscope and applicator of FIG. 75 having electrodes and a drug delivery channel in the deployed position piercing the lesion;

FIG. 79 shows experimental results of tumor volume vs time for five different trials;

FIG. 80 shows a plot of transfection rates for high and low voltage RFP-Luc;

FIG. 81 shows expression of mIL-12p70 by electroporation into established B16-F10 tumors;

FIG. 82 shows LacZ staining after electroporation of a Lax Z expressing plasmid in B16-F10 tumors;

FIG. 83 shows expression of trimeric CD40L by electroporation in B16-F10 tumors;

FIG. 84 shows expression of trimeric CD80 by electroporation in B16-F10 tumors;

FIG. 85 shows IT expression of sdAbs by electroporation in B16-F10 tumors;

FIG. 86 shows a perspective view of an applicator in accordance with some embodiments;

FIG. 87 shows a flexible applicator in accordance with some embodiments;

FIG. 88 shows a flexible applicator in use in accordance with some embodiments;

FIG. 89 shows a partial view of an applicator having the electrodes retracted in accordance with some embodiments;

FIG. 90 shows a partial view of an applicator having the electrodes deployed in accordance with some embodiments; and

FIG. 91 shows a rigid, trocar-based applicator in accordance with some embodiments.

FIGS. 92A-102D show schematics of a digital board for a low-voltage generator, according to some embodiments of the disclosure.

FIG. 103 shows a block diagram of a power generation board for a low-voltage generator according to some embodiments of the disclosure.

FIGS. 104A-109C show schematics of a power generation board for a low-voltage generator, according to some embodiments of the disclosure.

### DETAILED DESCRIPTION

Some embodiments of the present invention will now be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

### System Overview

Disclosed herein are various electroporation systems, apparatus, and methods. In some embodiments, the electroporation systems, apparatus, and methods disclosed herein may be used in connection with minimally-invasive procedures involving inserting portions of an applicator into a patient via a narrow opening and, in some embodiments, administering various therapies and treatment agents therethrough. The systems, apparatus, and method used herein may be used to deliver any treatment agent (e.g., nucleic acid-based therapies) and apply any electroporation therapy viscerally. In some embodiments, the electroporation systems, apparatus, and methods disclosed herein may be used in connection with an insertion device.

As used herein, the term “insertion device” means any apparatus or structure capable of allowing a portion of an applicator to be inserted into a patient, for example via a cannula or other working channel. In some embodiments, the electroporation systems, apparatus, and methods disclosed herein may be used in connection with endoscopic devices and procedures to reach and treat remote tissues (e.g., visceral lesions, such as tumors) within a patient. In some embodiments, various types of endoscopic devices may be used along with the electroporation systems, apparatus, and methods disclosed herein depending on the particular location of the remote tissue, such as bronchoscopic devices, laparoscopic devices or other cannulated devices suitable for providing access to such remote tissues. Such endoscopic devices may be of any type, including for example either a flexible endoscopic instrument or a rigid endoscopic instrument (e.g., a trocar, such as for use in laparoscopic procedures), which may be selected based on the anticipated procedure and/or location of the remote tissue. In some embodiments, the electroporation systems, apparatus, and methods disclosed herein may be used to access lesions anywhere in or adjacent to the alimentary canal. In some embodiments, the electroporation systems, apparatus, and methods disclosed herein may be used to access lesions in the lungs. In some embodiments, the electroporation systems, apparatus, and methods disclosed herein may be used in connection with minimally invasive electroporation, one example being in connection with any such aforementioned endoscopic instrument.

In a variety of medical treatments, electroporation may be used to increase the permeability of cells by using electrical fields to create pores in biological cells without causing permanent damage (e.g., reversible electroporation). In some instances, the increased permeability of reversible electroporation may enable a contemporaneous treatment, such as drug administration or gene therapy, to be more effective because the treatment is better able to permeate the cells. During electroporation, a voltage may be applied across two or more electrodes to create an electric field therebetween. In some examples, the electrodes may be disposed on either side of, embedded within, or otherwise be positioned relative to, cell tissue that is then subjected to the electric field. The electric field creates the pores within the cell tissue which then allow the cell to be permeated by one or more treatment agents. Performance of electroporation with a low voltage generator as described herein is particularly advantageous in satisfying the conditions necessary to achieve reversible electroporation. Although tissue around the target site may have varying electric field thresholds, the application of low voltage is intended to, even among the extant range of threshold values, apply a voltage amount that is below such a threshold in order to minimize or avoid damage to the tissue during the electroporation procedure.

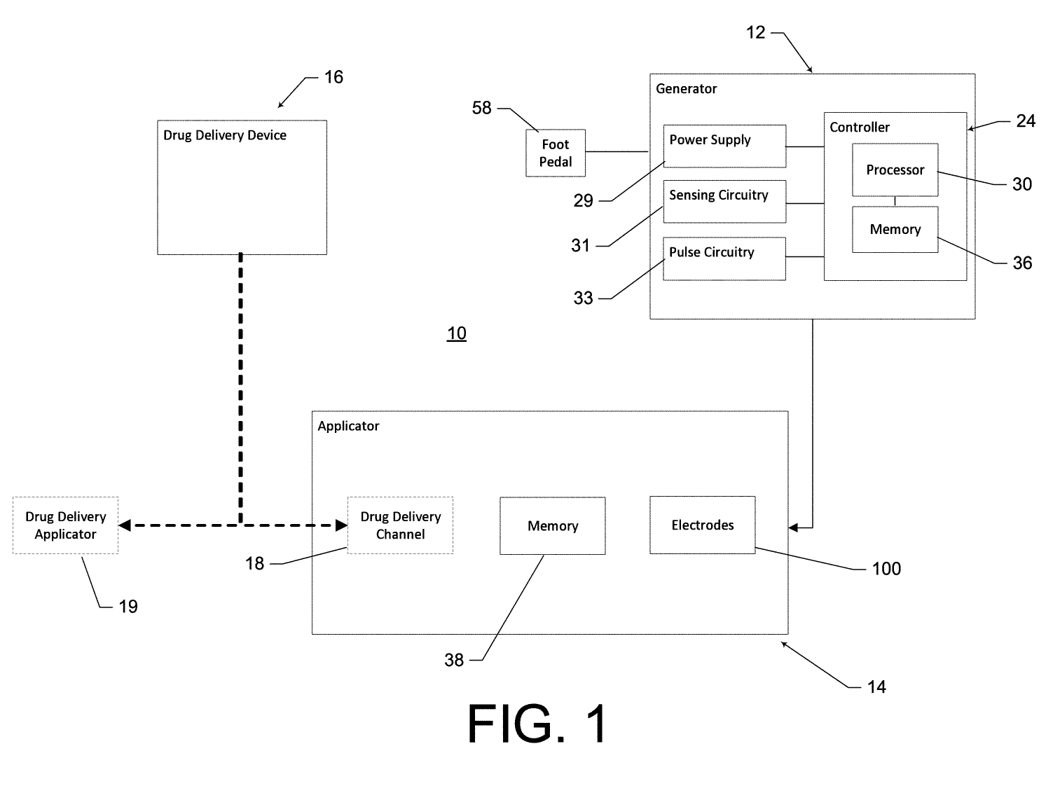

With reference to FIGS. 1-3, an example electroporation system 10 is shown. In the embodiment as illustrated, the system 10 includes a generator 12 for generating and delivering electrical signals to at least two electrodes 100 and an applicator 14 including the at least two electrodes. The applicators 14 described herein using reference numeral 14 may be generally representative of each of the embodiments of specific applicators 14, 60, 70, 110, 1000 described herein as if each applicator were discussed individually. The electrodes 100 described herein using reference numeral 100 may be generally representative of each of the embodiments of electrodes 100, 200, 300, 400, 500, 600, 700, 800 described herein as if each electrode were discussed individually. To the extent there are differences among the various embodiments of the applicators and electrodes in the various embodiments of the present disclosure, such differences are described as applicable. In some embodiments, the electrodes 100 may include two or more electrodes, which may each define a pointed tip at a distal end for piercing the tissue at the target site. In some embodiments, the tips of the electrodes are exposed while adjacent surfaces of the electrodes are insulated so that current passes through the tips only. In some embodiments, a region on the respective electrode away from the tip is exposed while surrounding surfaces are insulated and current is directed only through these exposed surfaces between the electrodes. The location of exposure may be close enough to the tip, and/or at the tip, so that the exposed portion of the electrode is outside of the insertion tube 15 of the applicator, described below, when the electrodes are in the deployed position. In some embodiments, as discussed in further detail below, the tips of the electrodes 100 may be closer together in a retracted position for insertion into the patient (e.g., via the working channel), and once in position, the electrodes may be deployed to a deployed position in which the tips of the electrodes are spread farther apart for administering electroporation to a larger treatment area. In some embodiments, the electrodes are included as part of an applicator with a predetermined spacing such that whether the electrodes are in the retracted or deployed position, the spacing remains constant. In one example, the spacing of the electrodes in such embodiments is about 4 mm. The electrodes may still be housed in a tube or other delivery structure in these embodiments. In yet another example, the applicator may only include a single electrode 100, while a second electrode can be constituted by, for instance, a distal-most portion of a housing tube or other portion of an applicator body or like structure. In such an example, the applicator would only have a single needle (which could thus be fixed or deployable) which need only be spaced a sufficient distance from the structure constituting the second electrode to be effective in providing a voltage to the desired tissue and to prevent arcing.

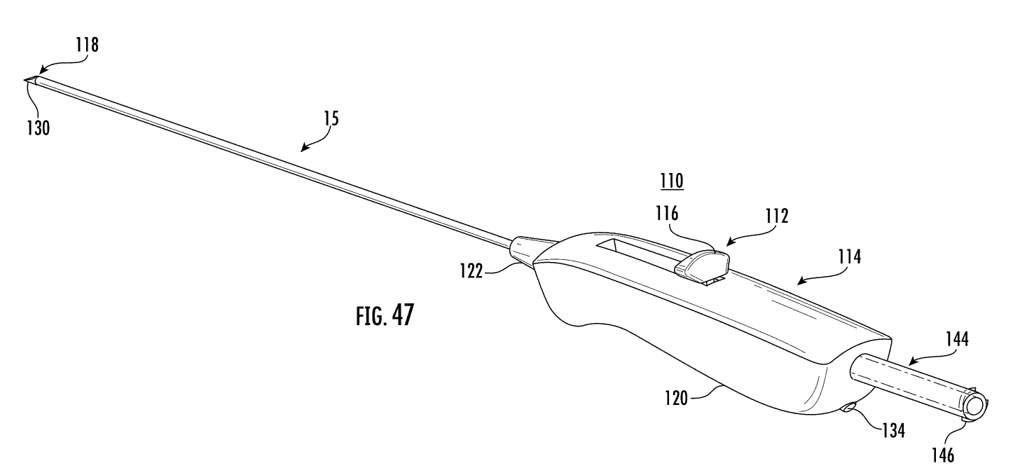

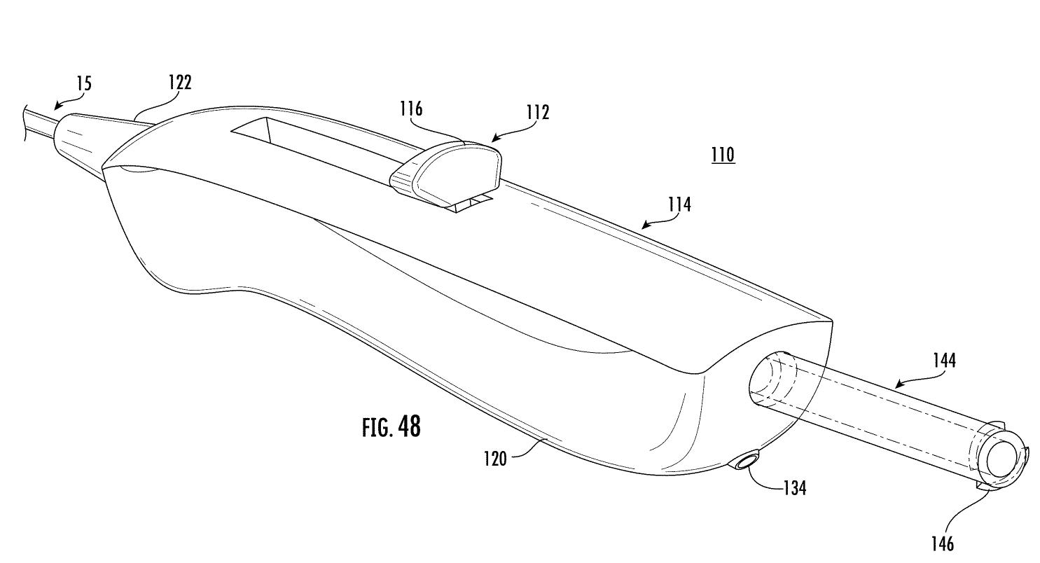

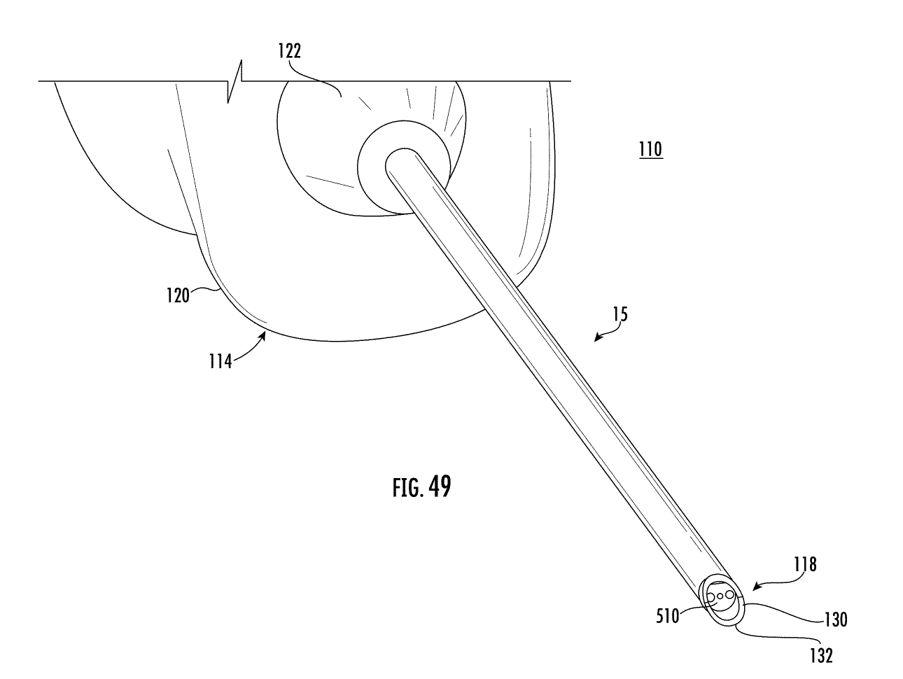

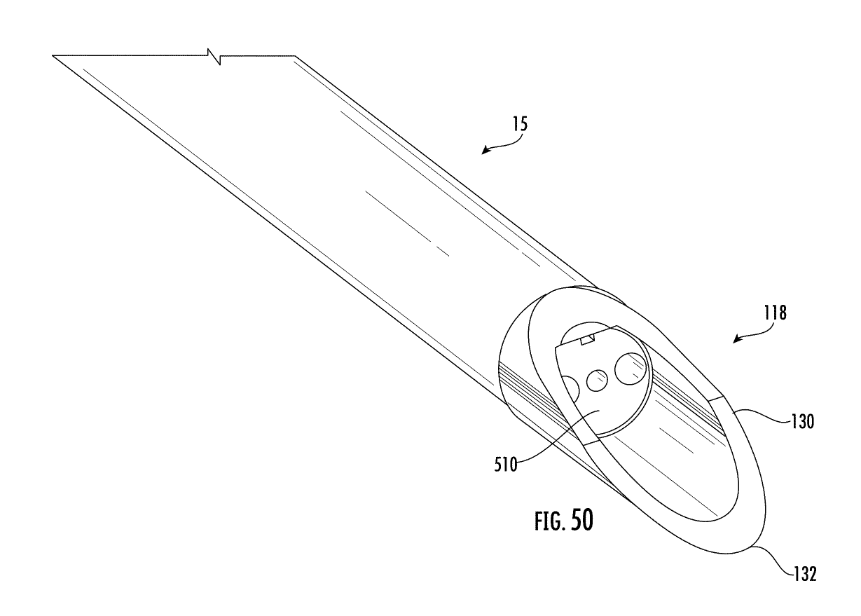

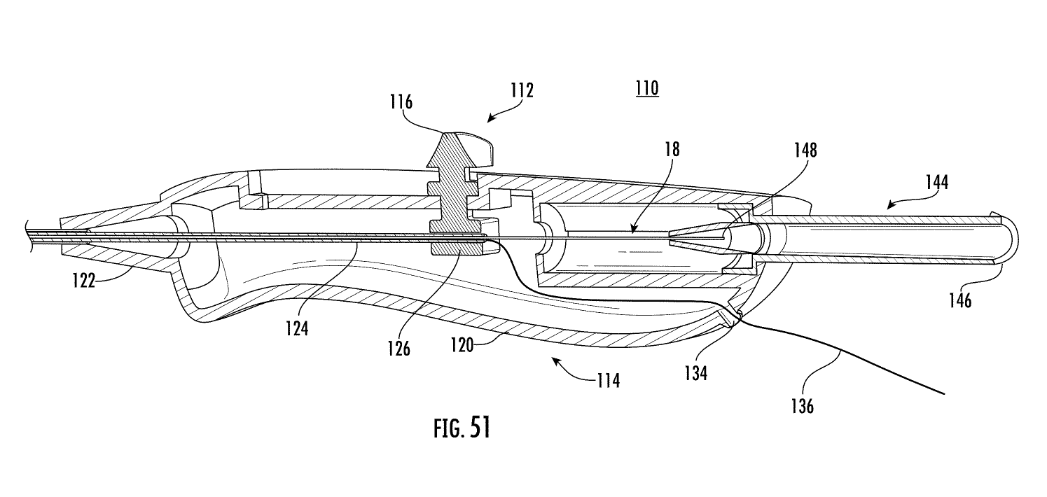

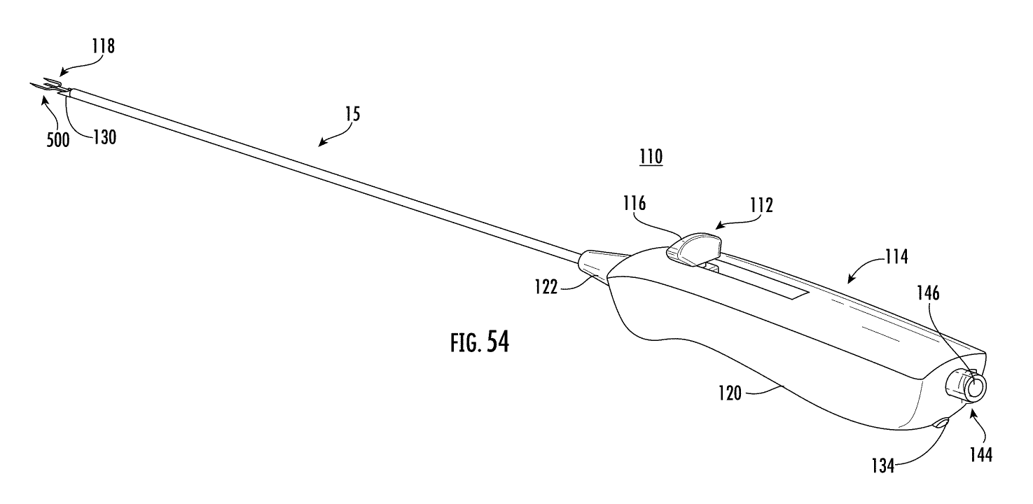

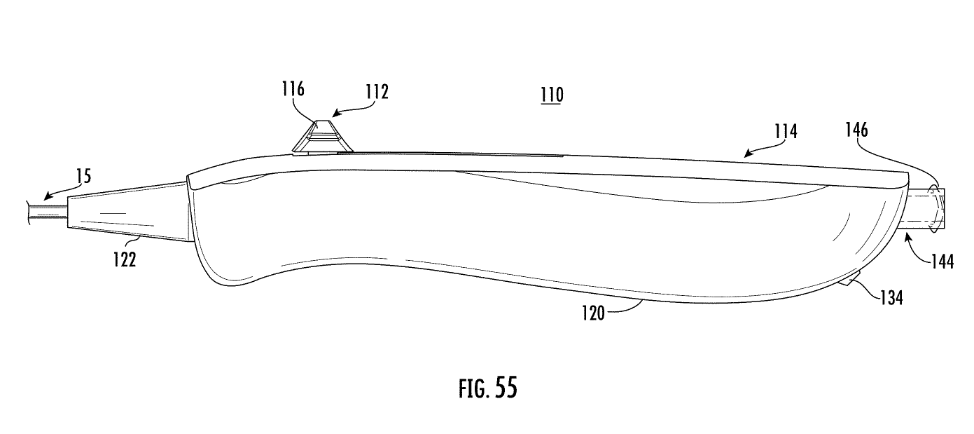

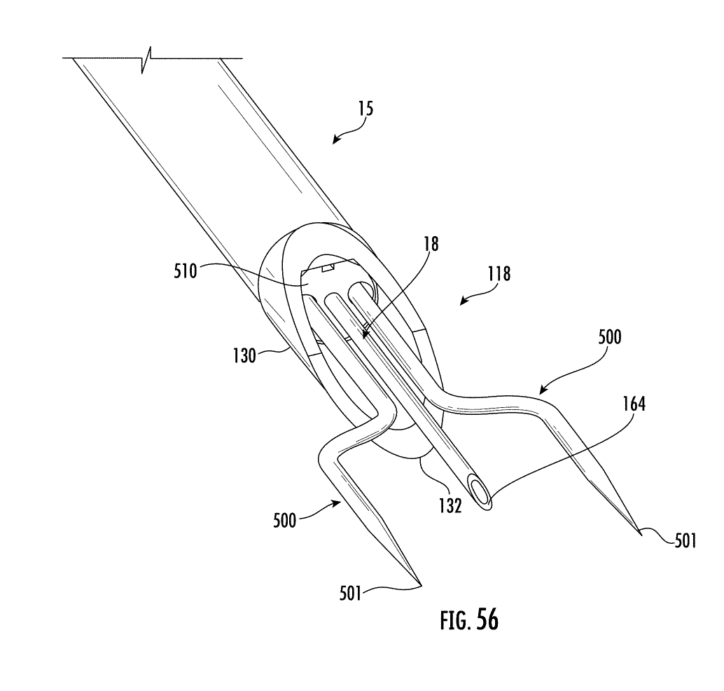

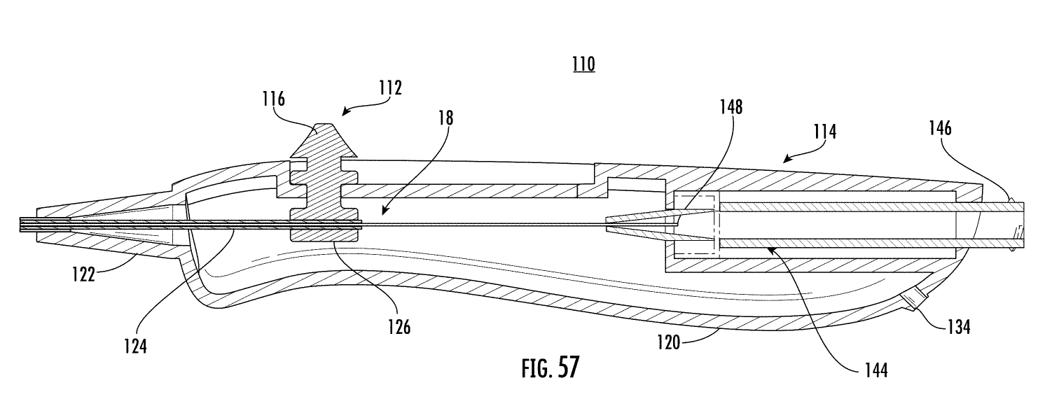

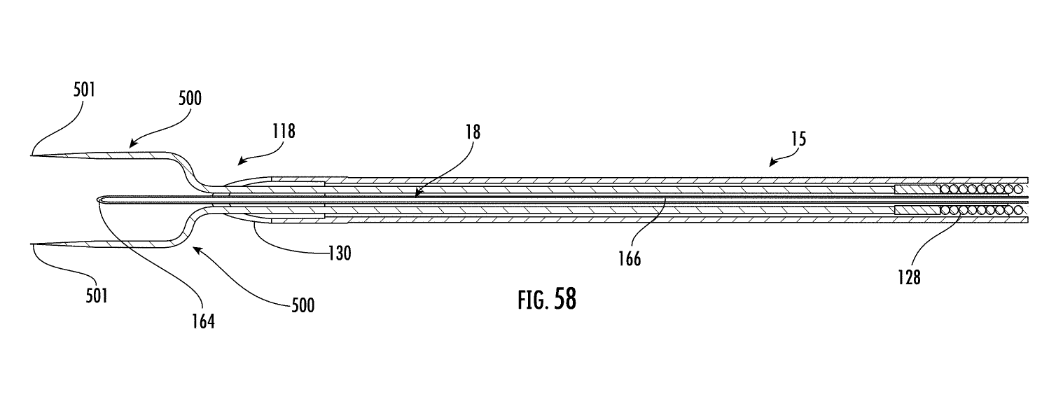

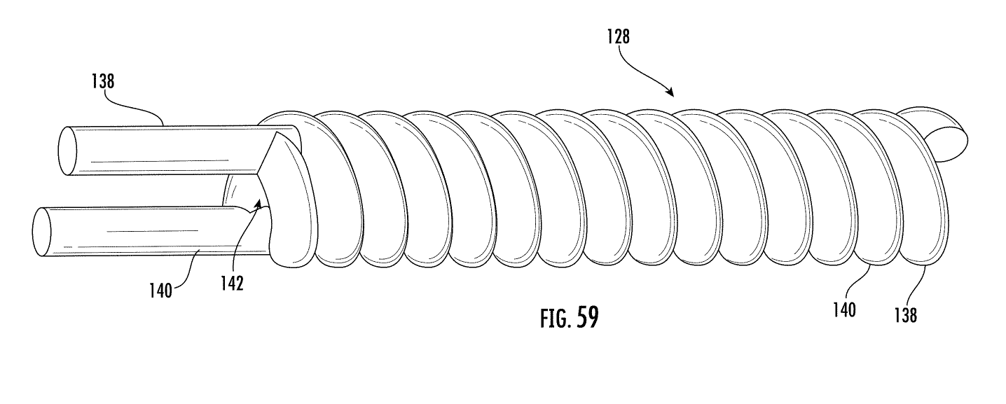

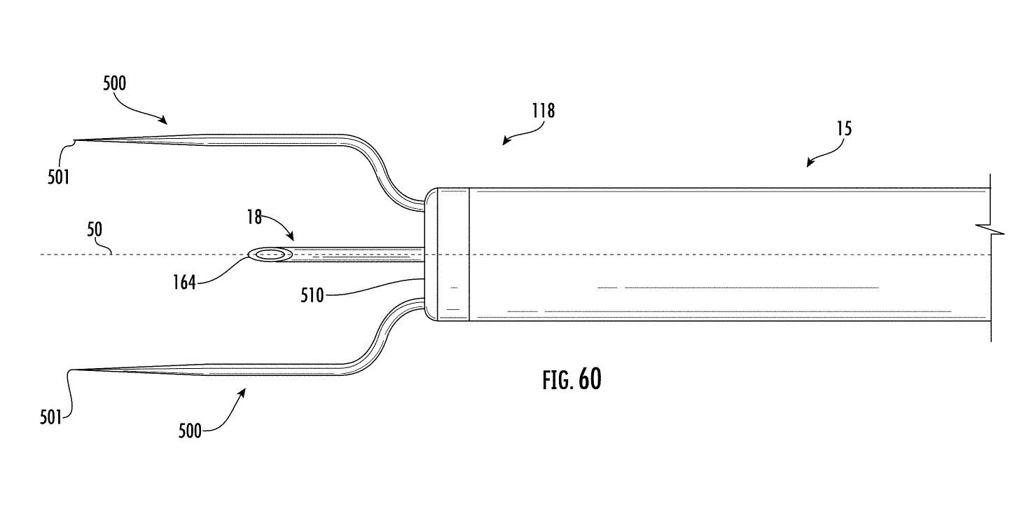

In some embodiments, the applicator 14 of the system 10 may be used to administer one or more treatment agents (e.g., a drug and/or plasmid). For example, the applicator may include an insertion tube 15 serving as a delivery path for the treatment agent(s). In some examples, and as described in greater detail elsewhere in the application, a designated drug delivery channel 18 may be included within the insertion tube 15 for administration of treatment agents (e.g., as shown in FIGS. 47-67). The drug delivery channel 18 may extend through the applicator 14 for co-localization of the electrodes and treatment agent(s). The drug delivery channel 18 may terminate at the electrodes 100 adjacent the electroporation site to administer the one or more treatment agents adjacent to or as close as possible to the cells being electroporated. In some examples, the drug delivery channel may terminate slightly proximal to the electrode tips. In still other examples, the delivery channel may also have a shape suitable for insertion into the tissue to be electroporated, such as a needle, such that the delivery channel extends at or distal to the electrode tips.

In some embodiments, the electroporation system 10 may further include a drug delivery device 16 for administering one or more treatment agents (e.g., a drug and/or plasmid) to the electroporation site. FIG. 1 illustrates some examples of how drug delivery device 16 may be positioned in the system, and in a larger context includes dashed arrows to indicate fluid flow paths and solid arrows to indicate electrical connections. With reference to FIG. 1, the drug delivery device 16 may define a syringe having a distal tube or needle for administering the treatment agent. In some embodiments, the drug delivery device 16 may include at least one reservoir, configured to receive the one or more treatment agents, and at least one pump configured to deliver the treatment agents to the electroporation site. In some embodiments, the drug delivery device 16 administers the one or more treatment agents directly to the target site while the applicator 14 is used to perform electroporation at the target site. In some embodiments, the drug delivery device 16 administers the one or more treatment agents to the applicator 14, which in turn, directly administers the treatment agent to the target site. In this manner, the applicator 14 is used for treatment agent administration and for performance of electroporation. In some examples, the treatment agent is delivered through a drug delivery channel 18 within the applicator 14.

In some embodiments, and as discussed elsewhere herein, the one or more treatment agents may be administered via a separate drug delivery applicator 19 (e.g., a long distal needle, a conduit passing through an endoscopic instrument, or the like) instead of being administered through the applicator 14 itself, as shown in FIG. 1. Still further, the drug delivery applicator 19 may deliver at least one of the treatment agent(s) systemically rather than directly to the electroporation site. The separate drug delivery applicator 19 (or other administration device) may be used sequentially with the electroporation applicator 14 to administer the one or more treatment agents to the electroporation site. In some examples, the drug delivery applicator 19 alone is used to administer the one or more treatment agents. In other examples, the drug delivery device 16 is used in conjunction with the drug delivery applicator 19 to administer the one or more treatment agents, as shown in FIG. 1. In these examples, the applicator 14 separately performs electroporation.

### Example System Architecture

In some embodiments, the generator 12 and applicator 14 are controlled by one or more controllers 24, which includes at least a processor 30 and memory 36. In some embodiments, the controller 24 may be disposed in the generator 12 and may control the applicator 14 therewith. In embodiments in which the drug delivery device 16 requires electronic control, one or more controllers may operate the drug delivery device, and in embodiments in which the drug delivery device 16 has no electronic control, the drug delivery device may be manually operated (e.g., by depressing a syringe). In some embodiments, electronic control may be in the form of robotics, described elsewhere herein. In some embodiments, each of the generator 12, applicator 14, and drug delivery device 16 may have its own controller. In some embodiments, one or more of the controllers may be controlled by another controller (e.g., in a master-slave relationship). In some embodiments, each controller 24 may be embodied as a single device or as a distributed processing system, some or all of which may be remote from the respective device that it controls. Examples of an electroporation system and corresponding electronic control methods, signals, and apparatus; treatment agents; and therapies are described in U.S. Pat. Nos. 7,412,284 and 9,020,605 and International Application No. WO2016/161201, each of which is incorporated by reference herein in its entirety.

With continued reference to FIG. 1, in some embodiments, the generator 12 may be a low-voltage generator for administering the electroporation therapy and/or performing electrochemical impedance spectroscopy (EIS) as described herein. In some embodiments, the generator 12 may include pulse circuitry 33 configured to generate waveforms for excitation of the electrodes during electroporation. In some embodiments, the generator 12 is configured solely to perform electroporation therapy. In some embodiments, the generator 12 may include sensing circuitry 31 configured to receive signals from the electrodes 100 (e.g., EIS signals described herein) and facilitate analysis of the properties of the target tissue. As described herein, in some embodiments, the generator 12 may control the pulses output from the pulse circuitry 33 in response to the sensed parameters of the target tissue and the treatment agent determined by the sensing circuitry 31. In embodiments of the system with sensing circuitry 31, the circuitry may be toggled to activate or deactivate control of the parameters of the electroporation therapy based on the analysis of the EIS signals received by the system. In this manner, if the circuitry is toggled off, the therapy will maintain a preset voltage and pulse duration (or a predetermined voltage and pulse duration pattern) irrespective of any variation in impedance reported to the system by the sensors.

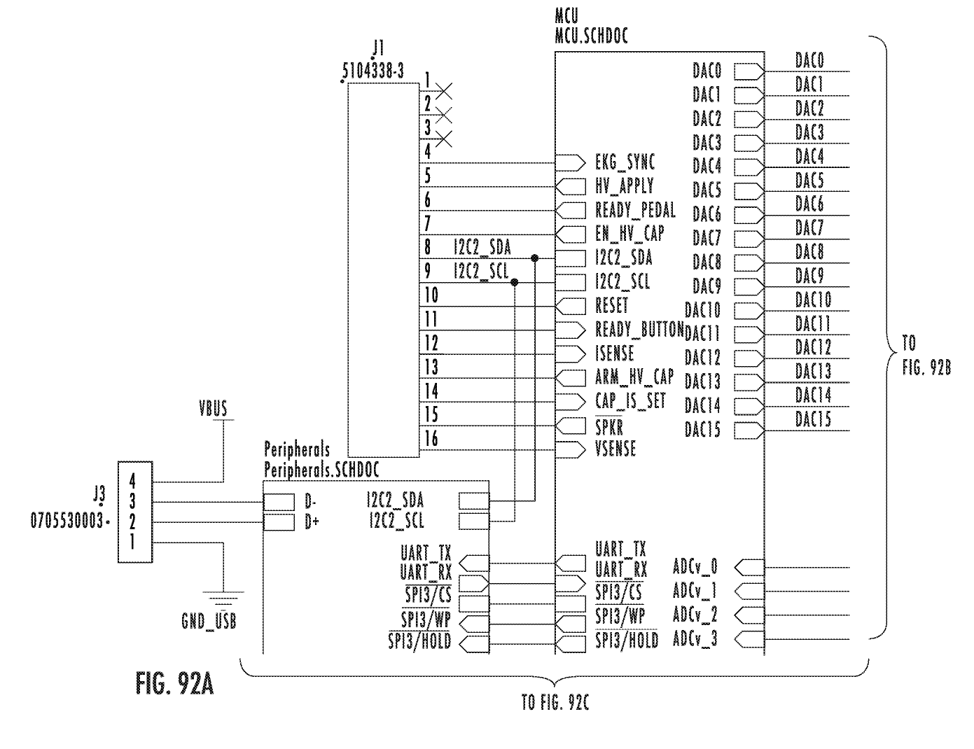

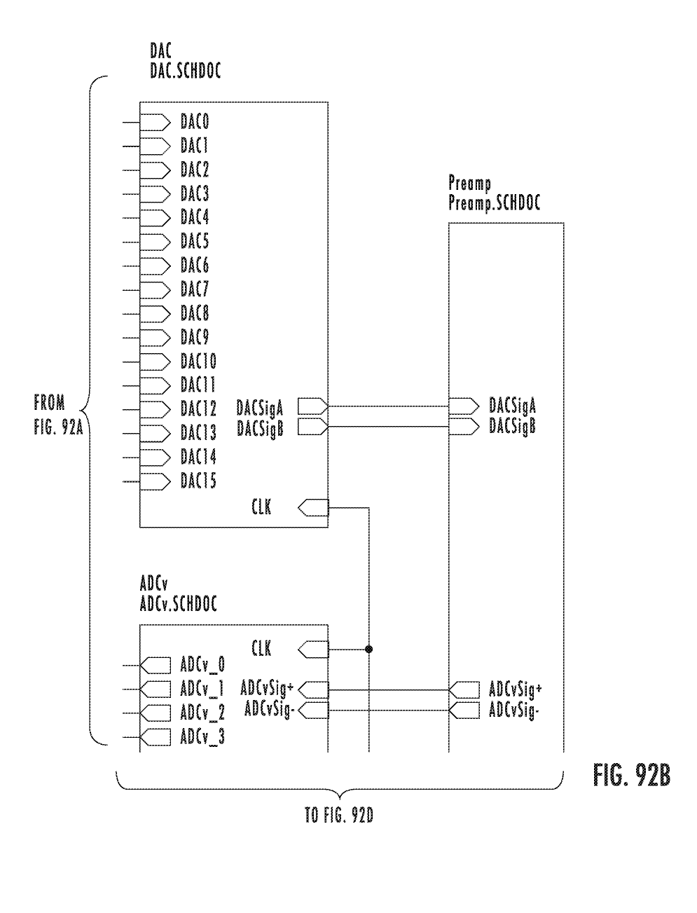

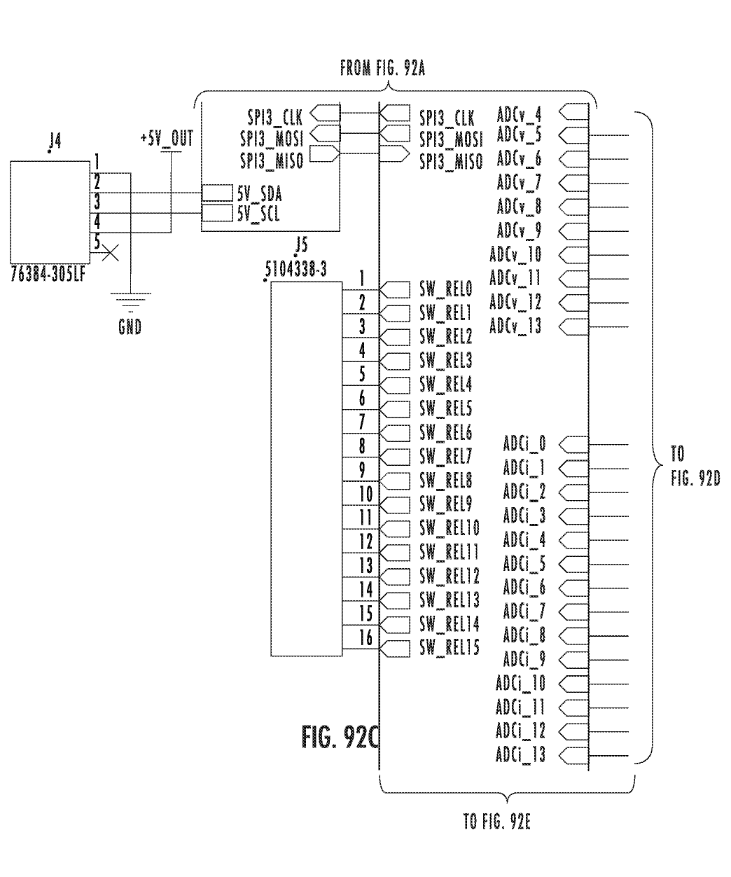

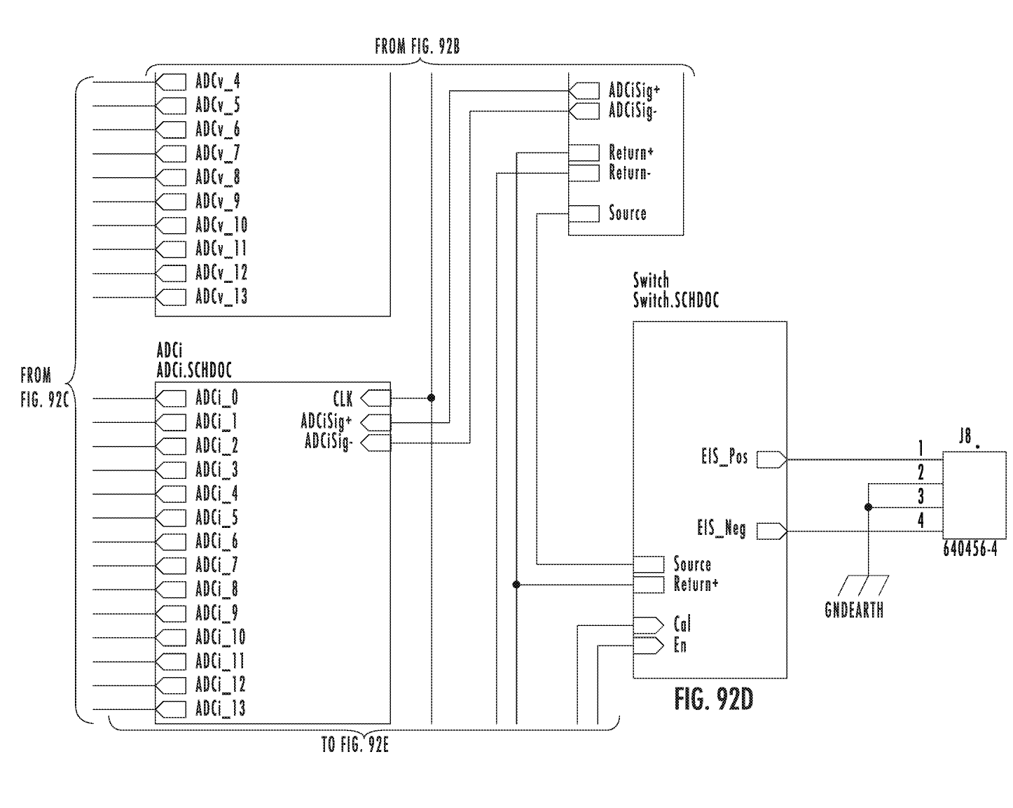

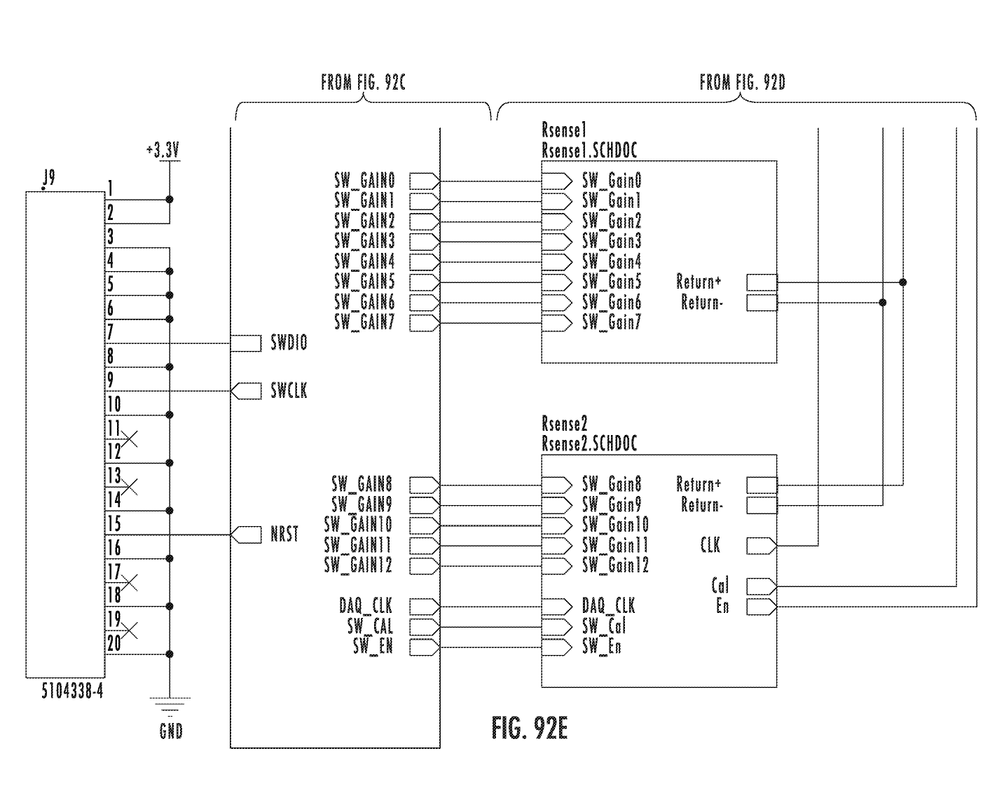

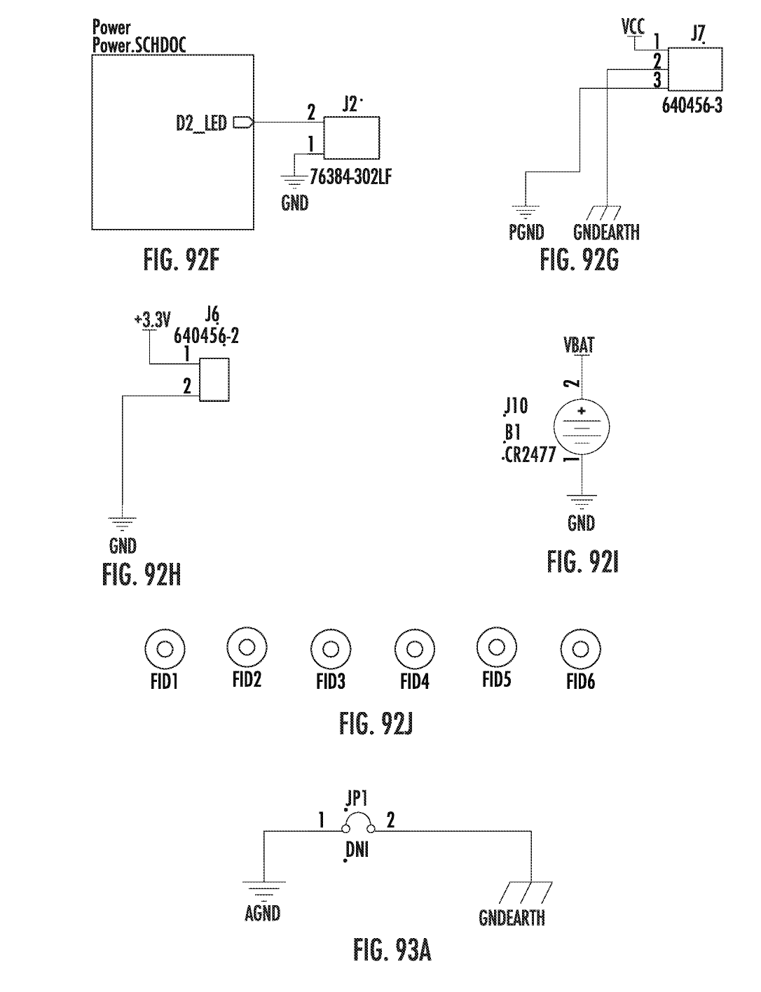

Turning to the structure of the generator of the system, in some embodiments, the low voltage generator includes a digital board and a power generation board. Details of the low voltage generator including the respective boards are illustrated in FIGS. 92A-109C. The digital board provides the central computing system by which signal processing, peripheral outputs, and safety features for the generator are implemented, while the power generation board contains all of the electrical components for pulse delivery during an electroporation treatment.

The digital board includes a microcontroller (MC), a digital-analog convertor (DAC), two analog-digital convertors (ADCs), resistor bank circuits, preamplifier circuits, and peripheral circuits. Each of these components contribute to the output of the device and signal processing for EIS. The MC also computes the software-based safety features to prevent delivery of unsafe therapy.

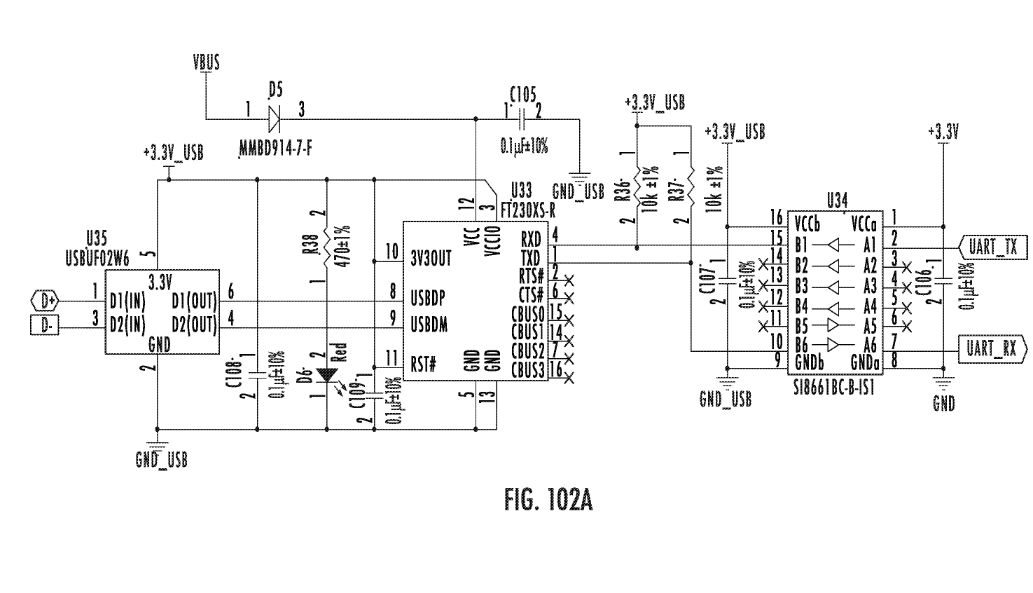

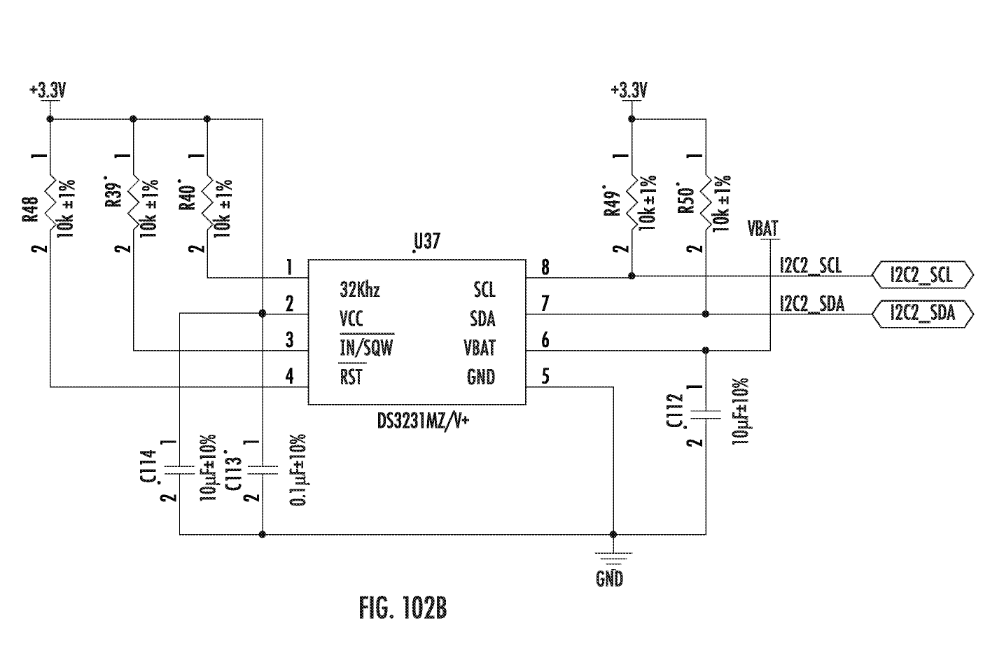

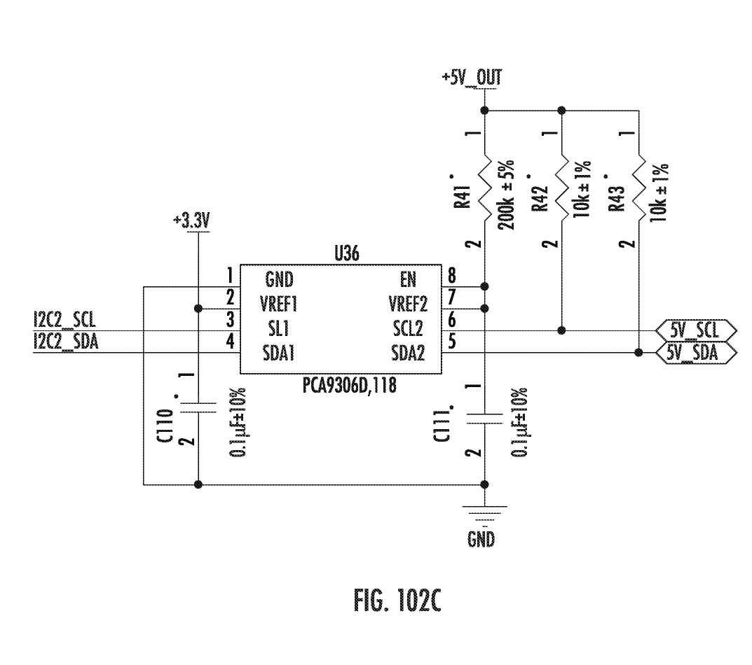

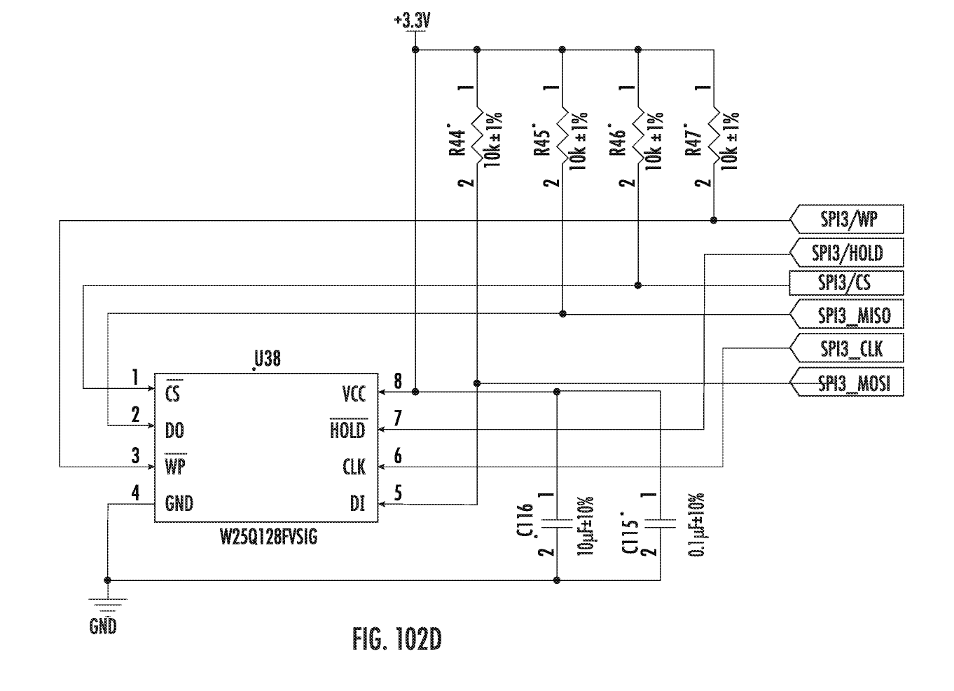

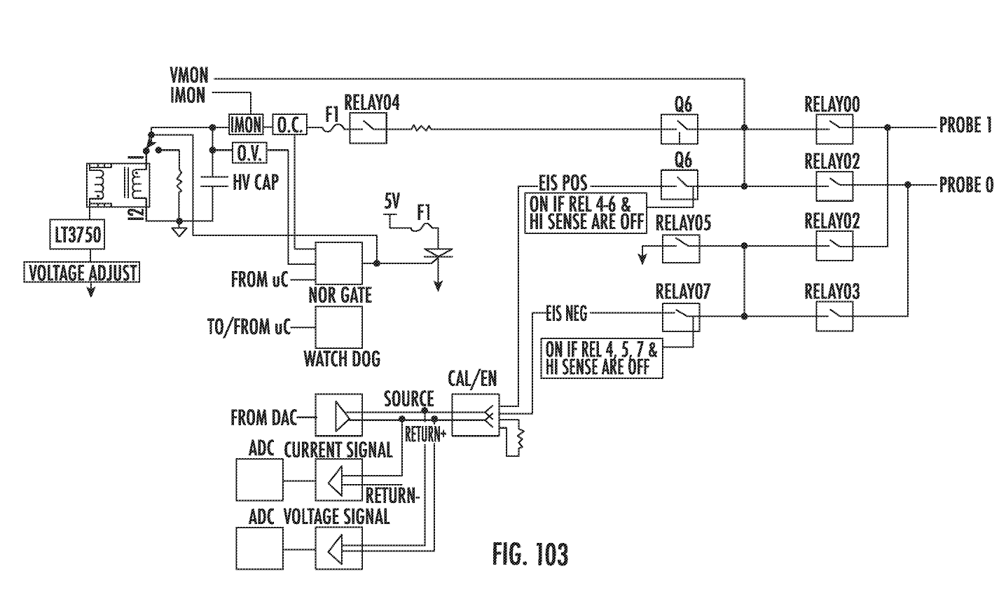

A schematic that outlines the entire digital board is shown in FIG. 92A-92J. All of the high-level circuits are shown in a grey shade. Each of the high-level circuits is tied to the MC for digital signal processing and operation of peripheral components used during operation of the generator. The peripheral components include those shown in FIGS. 102A-102D.

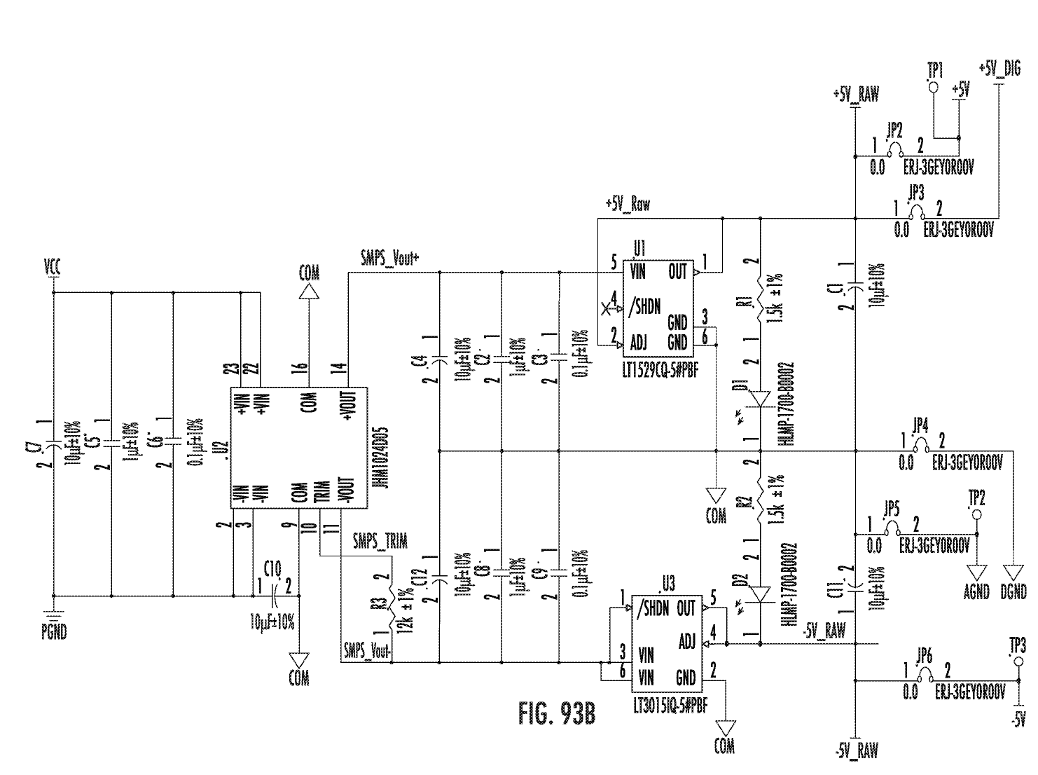

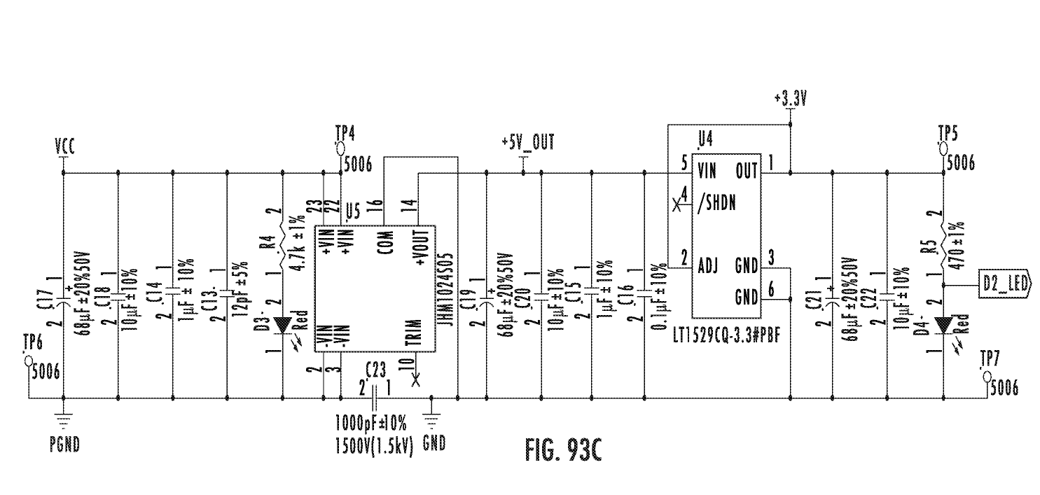

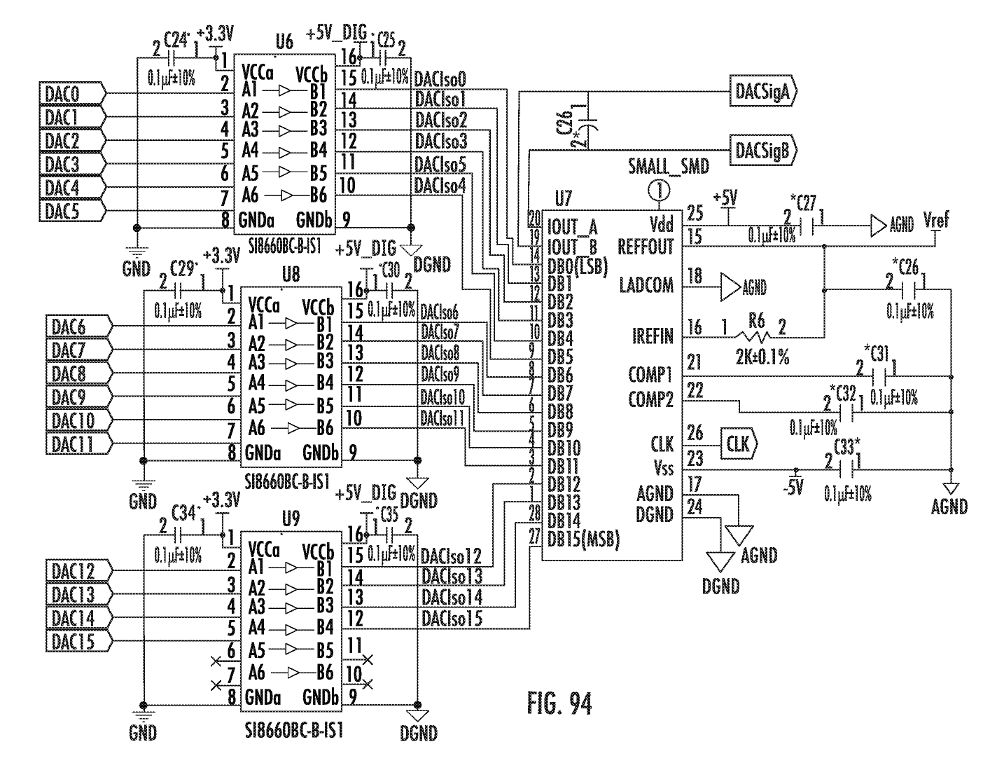

The power circuit shown in FIGS. 93A-93C provides voltage to the digital board components including the MC and various peripherals. The circuit distributes 3.3V to most of the digital board, but also steps up to 5V for corresponding component requirements. Various test points and LED lights allow for board troubleshooting.

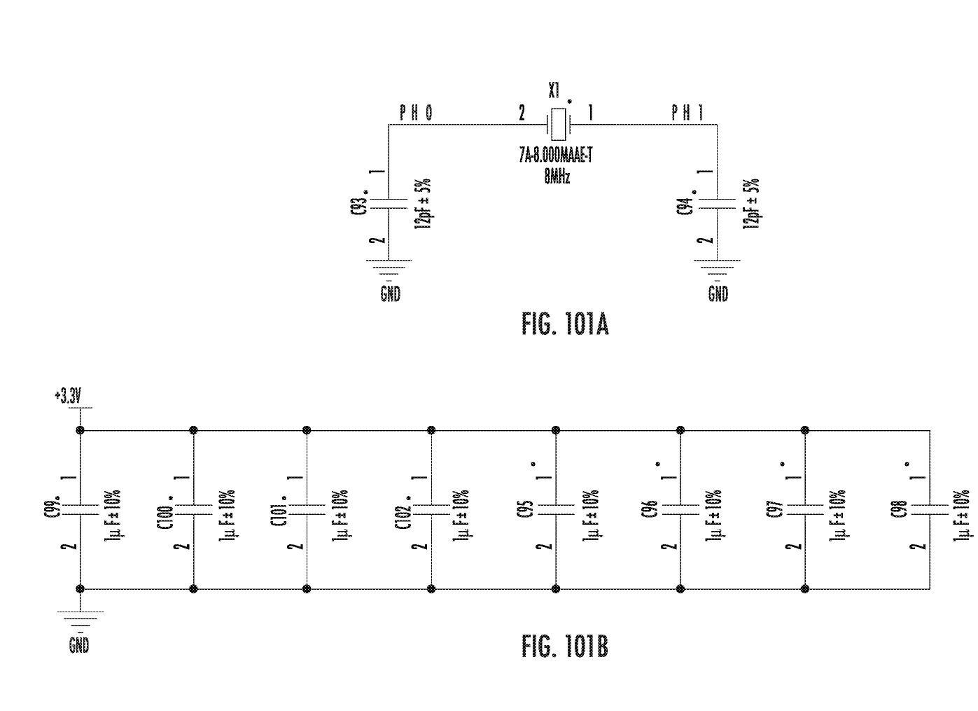

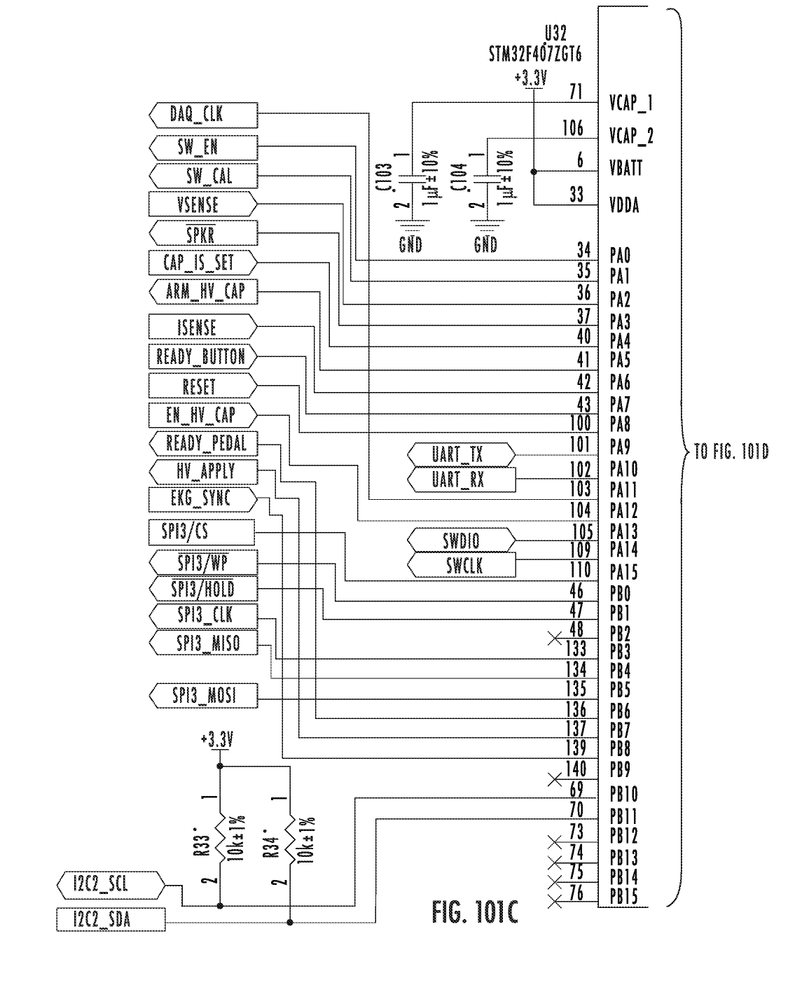

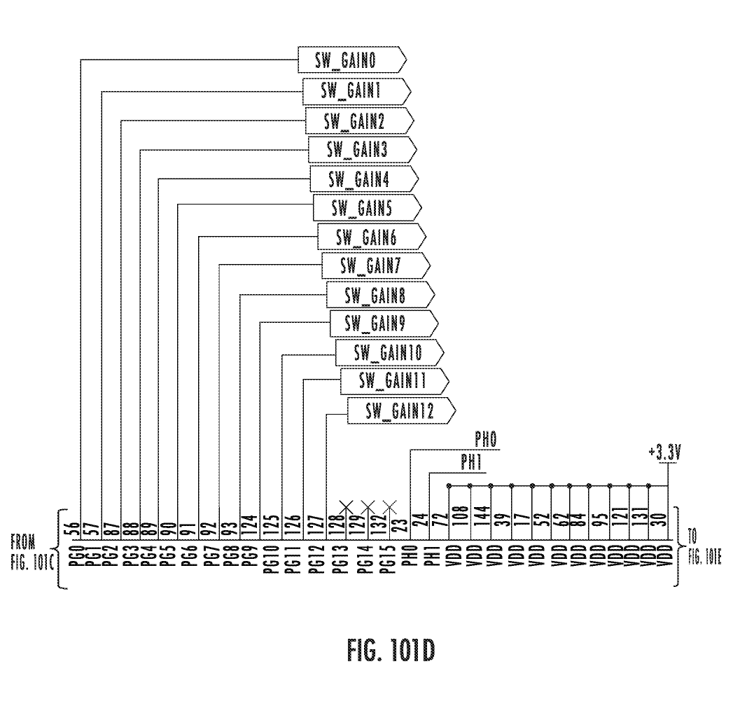

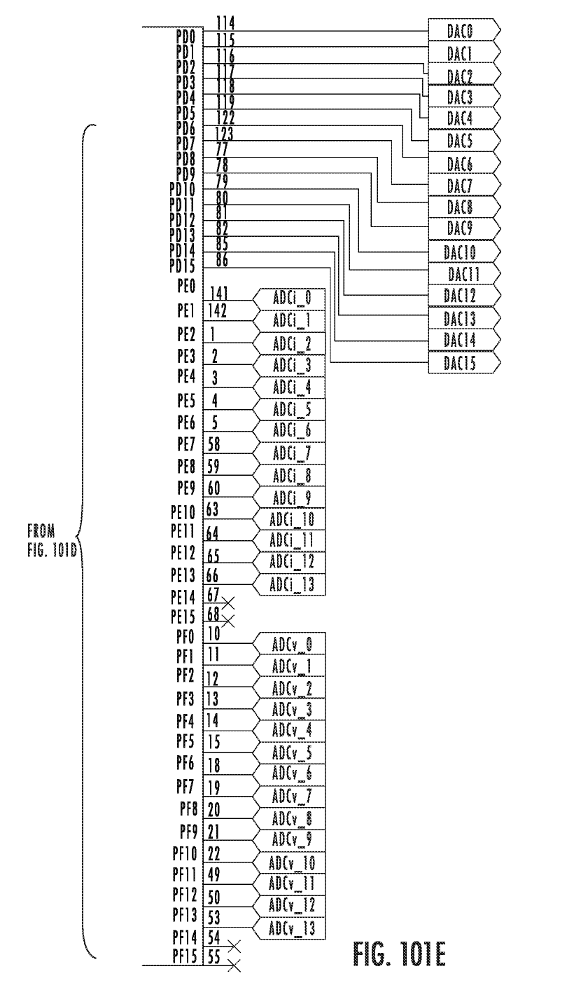

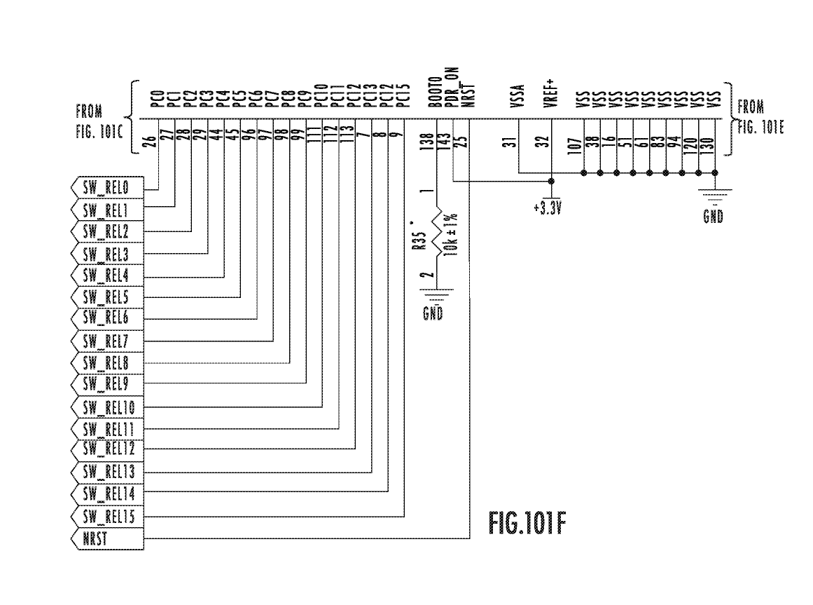

The MC, shown in FIGS. 101A-101F, is the central processor providing control over both the digital board and power generation board. Turning to other elements of the digital board, the DAC controls EIS signal generation. The ADCs include ADCi and ADCv. ADCi measures voltage across the resistor bank and ADCv measures voltage across the electrode leads, which are along the right side of the MC. The SW Relay controls shown in FIGS. 101A-101F provide precise control over each relay switch for output pulse delivery. The I2C bus provides regulation of the I/O ports, EEPROM read/write, Rheostat and Non-volatile Memory. Also shown in FIGS. 101A-101F is the resistor bank logic which isolates the specific frequencies and voltages used in the EIS signal when cycling through the different resistances. The resistor bank circuits are also used in calibration of the EIS signal.

The MC is used to implement software-based safety features through EIS signal processing. The voltage and current information measured across the electrode leads is used to identify load/tissue conditions and prevents delivery of therapy upon detection of unsafe parameters.

The DAC circuit, shown in FIG. 94, allows for EIS AC signal generation with specific frequencies and voltage, which are defined by the MC digital input. A high-frequency differential instrumentation amplifier is used with a high-order cutoff frequency set at 2.5 MHz to drive a differential output and remove any switching noise.

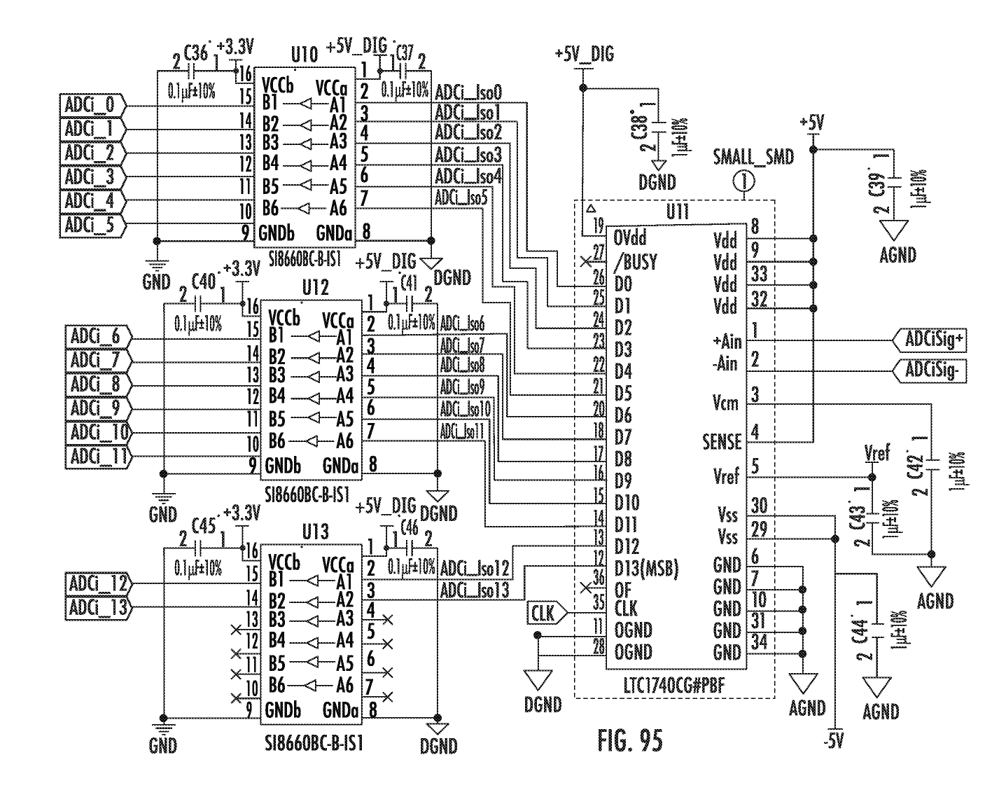

The ADCi circuit shown in FIG. 95 is an external component that processes the analog signals received through the electrode leads for current and directly measures the voltage across the resistor bank to process information to calculate load/tissue properties. The current is computed by measuring the potential drop across the current sense resistors and compensated according to the resistor/gain value. A high-frequency differential instrumentation amplifier is used with a high-order cutoff frequency set at 2.5 MHz. An additional 2nd order low-pass anti-alias filtering is used between the output of the instrumentation filter and the input of the 14-bit ADC. An additional 2nd-order low-pass anti-aliasing filter with a cut-off frequency of 15.9 kHz is used between the output of the differential instrumentation amplifier and the input of the 14-bit ADC.

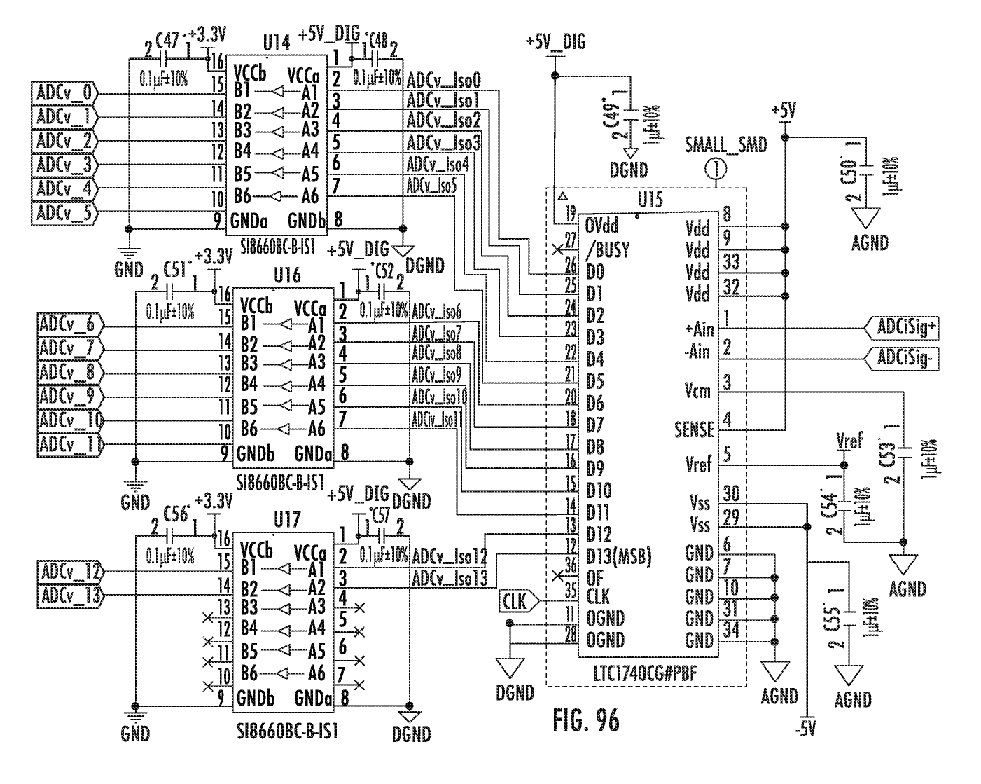

The ADCv circuit shown in FIG. 96 is an external component that processes the analog signals received through the electrode leads for voltage and directly measures the voltage across the electrode load to process the information to calculate load/tissue properties. The voltage is computed by measuring the potential drop across the positive output of the DAC instrumentation amplifier and the high-end of the current-sense resistor. A high-frequency differential instrumentation amplifier is used with a high-order cutoff frequency set at 2.5 MHz. An additional 2nd order low-pass anti-alias filtering is used between the output of the instrumentation filter and the input of the 14-bit ADC. An additional 2nd-order low-pass anti-aliasing filter with a cut-off frequency of 15.9 kHz is used between the output of the differential instrumentation amplifier and the input of the 14-bit ADC.

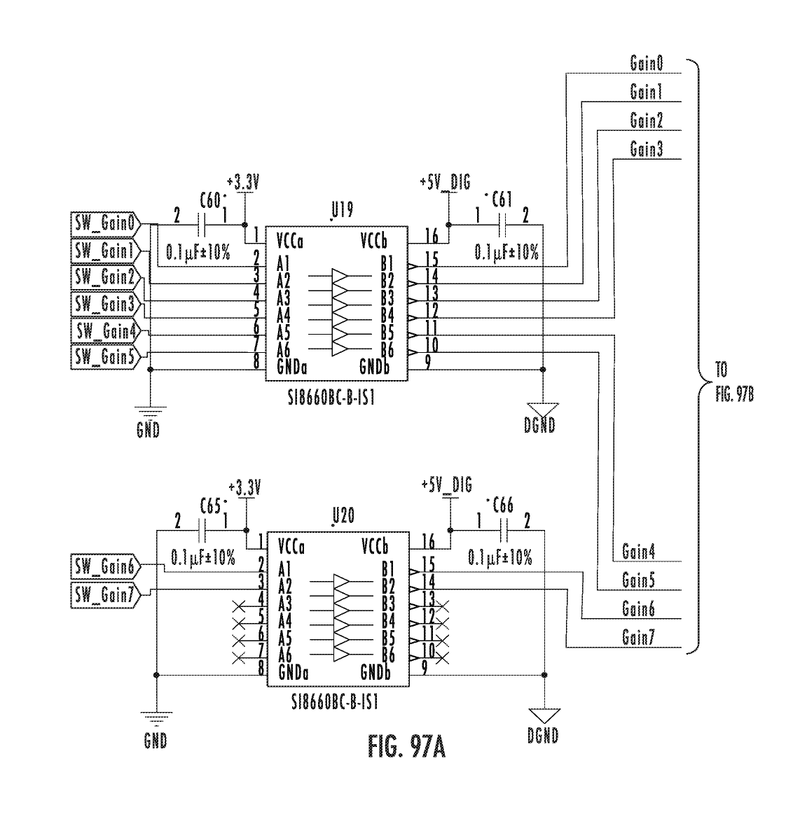

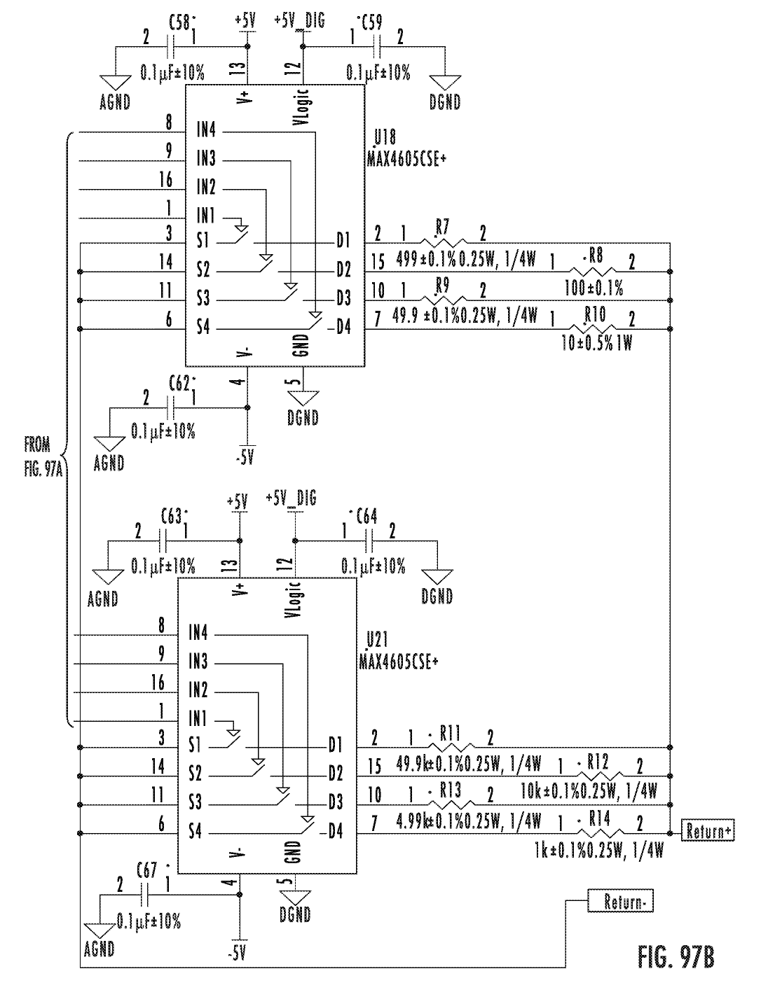

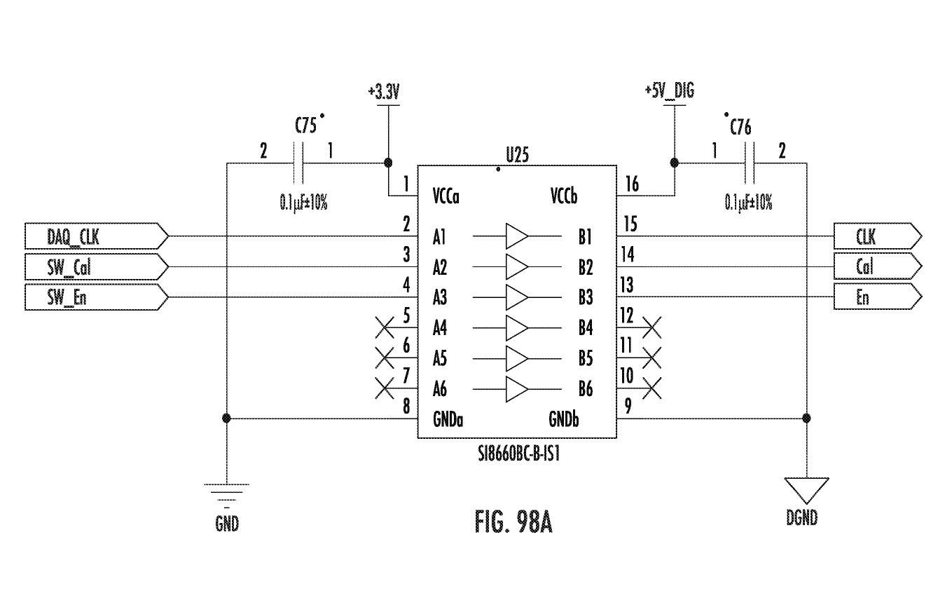

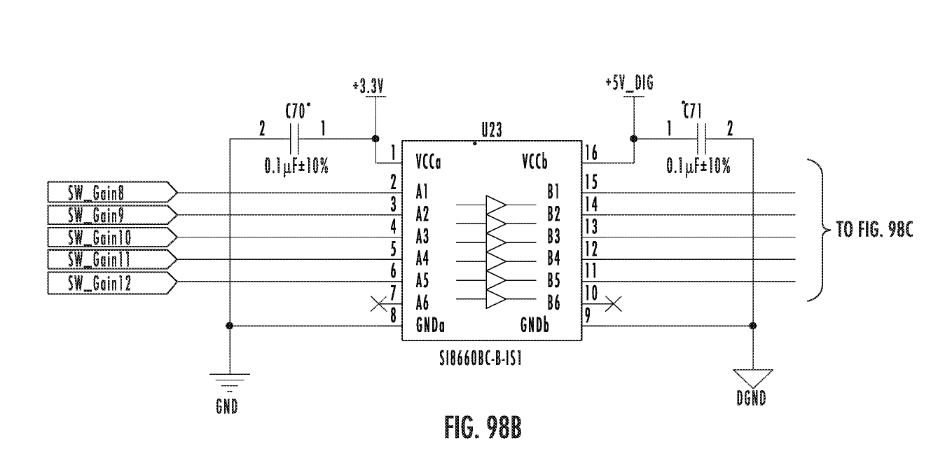

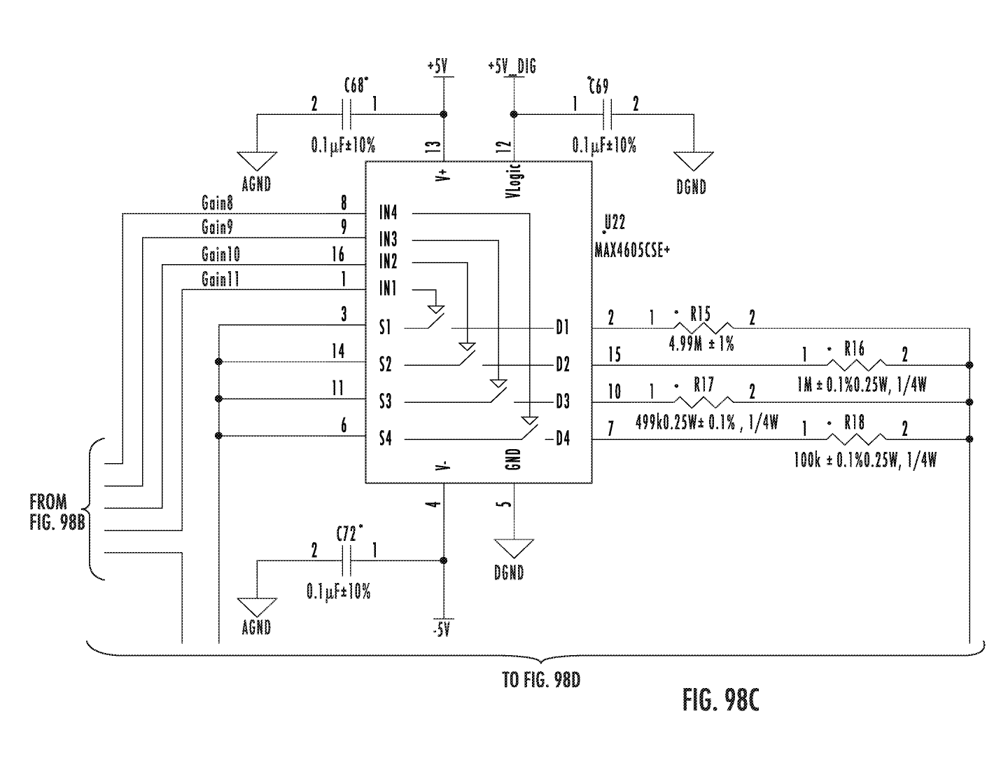

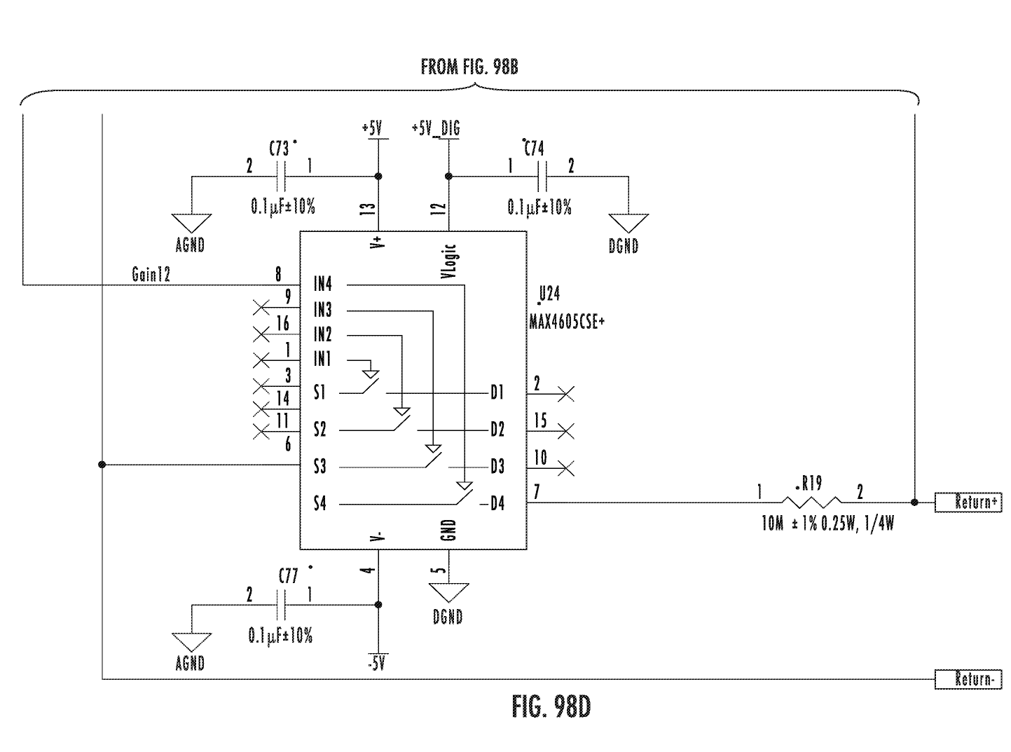

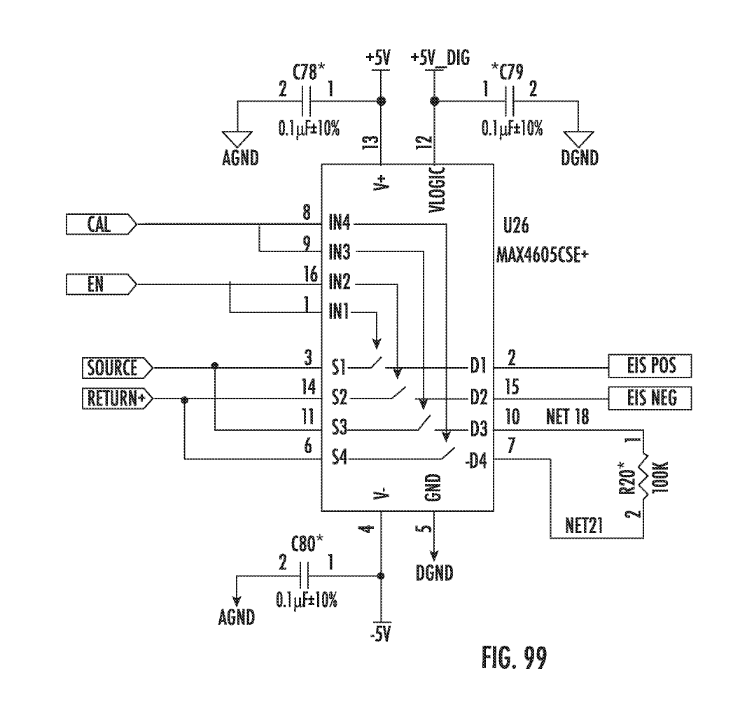

Two resistor bank circuits, shown in FIGS. 97A-98D, are used when cycling through EIS signal processing. There is a set of 13 different current sense resistors ranging from 10 Ohms to 10M Ohms with a tolerance of 0.1% that are enabled by the MCU through optically isolated I/O ports PG0-PG12. The resistors are connected on the return path of the instrumentation amplifier associated with the DAC. The resistors are selected to be 10.0Ω, 47.0Ω, 100Ω, 470Ω, 1.00 kΩ, 4.70 kΩ, 10.0 kΩ, 47.0 kΩ, 100 kΩ, 470 kΩ, 1.00 MΩ, 4.7 MΩ, 10.0 MΩ. These resistors are set using SW_GAIN0 through SW_GAIN12, respectively. A combination of these resistors in parallel are used to generate the following table:

The internal calibration resistor shown in FIG. 99, with a hard-set value of 100 k Ohms, is used to calibrate the EIS signal to give a reference signal used in determining the magnitude of output and input values.

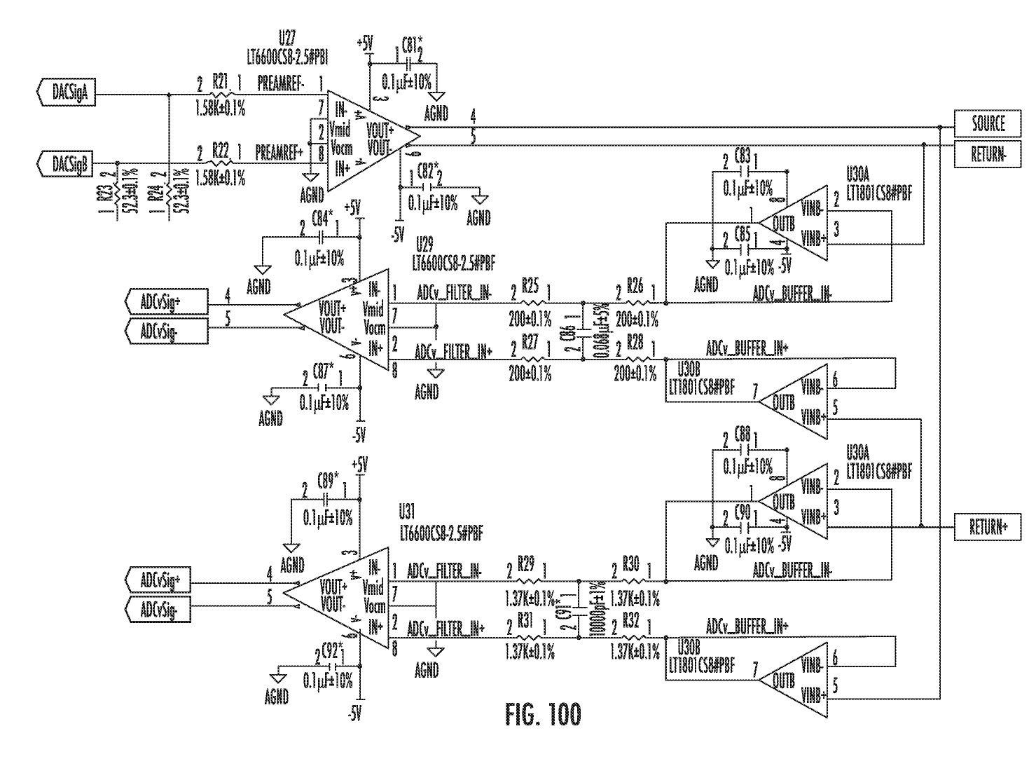

The preamplifier circuit shown in FIG. 100 outlines the path of the EIS signal generation from the amplifier circuit from the DAC through the load and back through the return lines. The return signals are processed through ADCs. Data obtained through such processing (e.g., signal values, such as voltage and current response) are used in circuit model computations for load/tissue analysis to determine safety features which can prevent short circuit delivery. The load/tissue analysis provides significant advantages including tissue property identification and electrical output optimization.

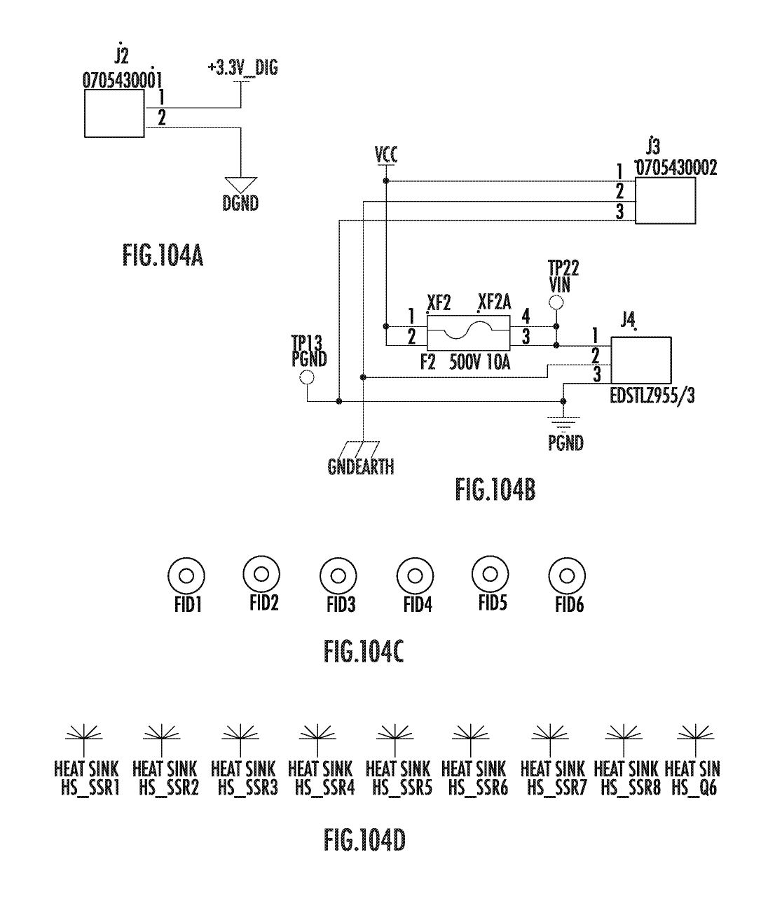

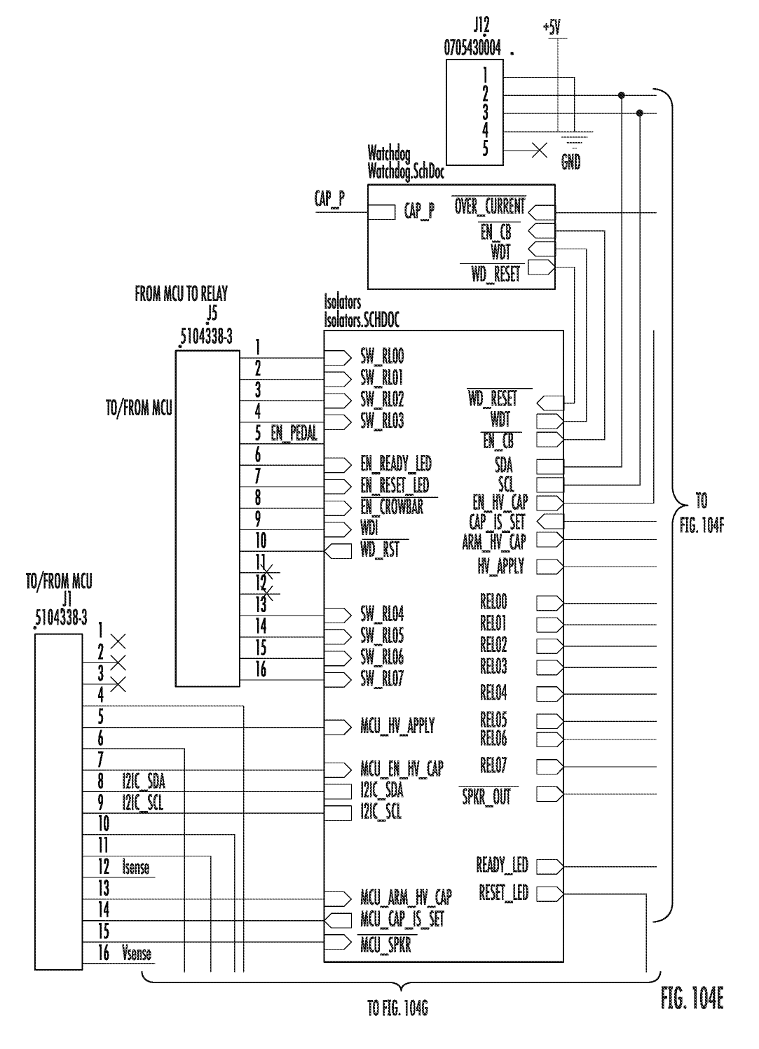

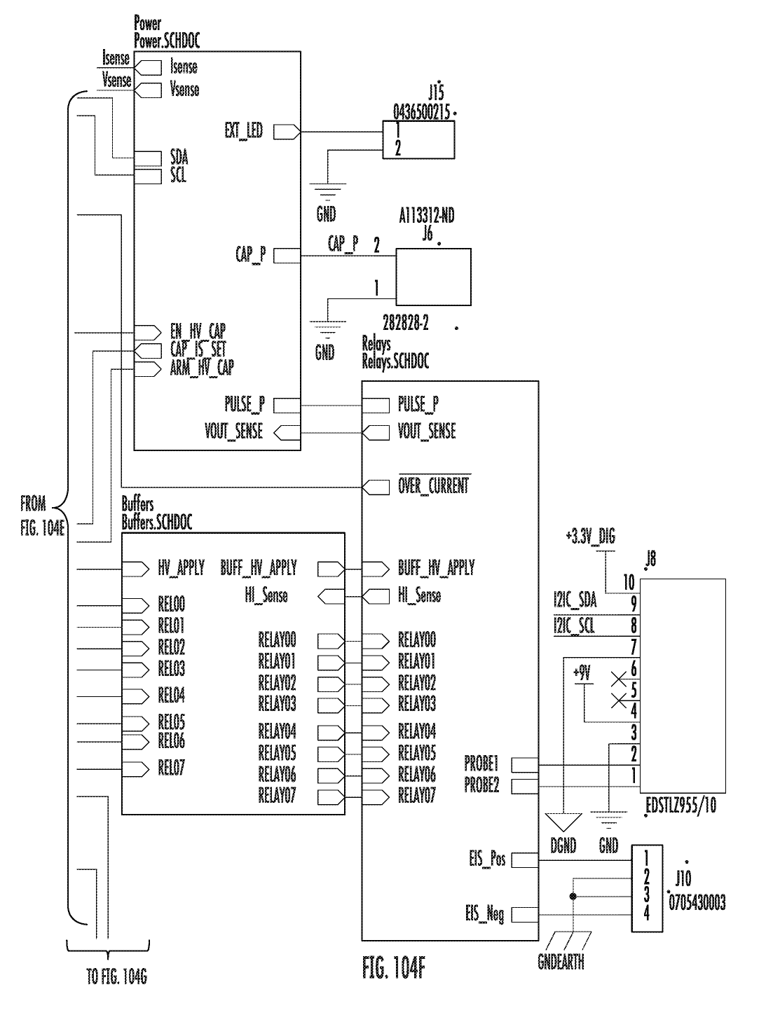

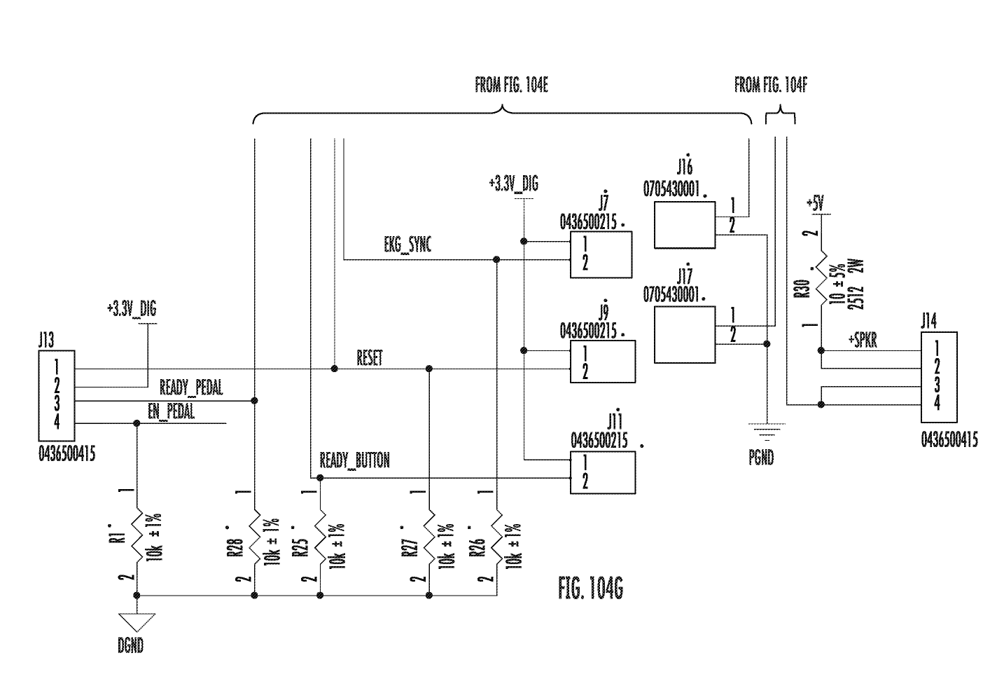

Turning to the power generation board, FIGS. 103-104G show details of the board in a block diagram and a schematic, respectively. The power generation board can be organizationally divided between several different sub-boards, each of which represent a unique function. For instance, the power generation board may include a main charging circuit, isolation wall, relay control circuitry, therapy output circuitry, and crowbar and watchdog circuitry. The main charging circuit may supply the therapy voltage via a flyback converter circuit and a 10 millifarad capacitor. The isolation wall may include multiple solid state digital isolators which buffer any digital signals to the analog side of the PCBA. The relay control circuitry may control the delivery of low voltage pulsing and includes several monitoring feedback loops. The watchdog and crowbar circuitry may include several functions such as watchdog timer and the mechanism to trigger and disable to high voltage line.

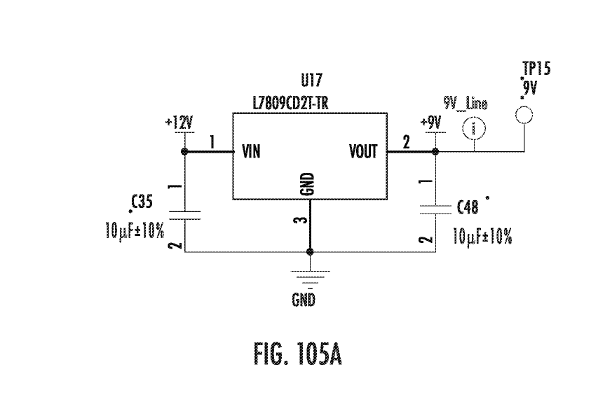

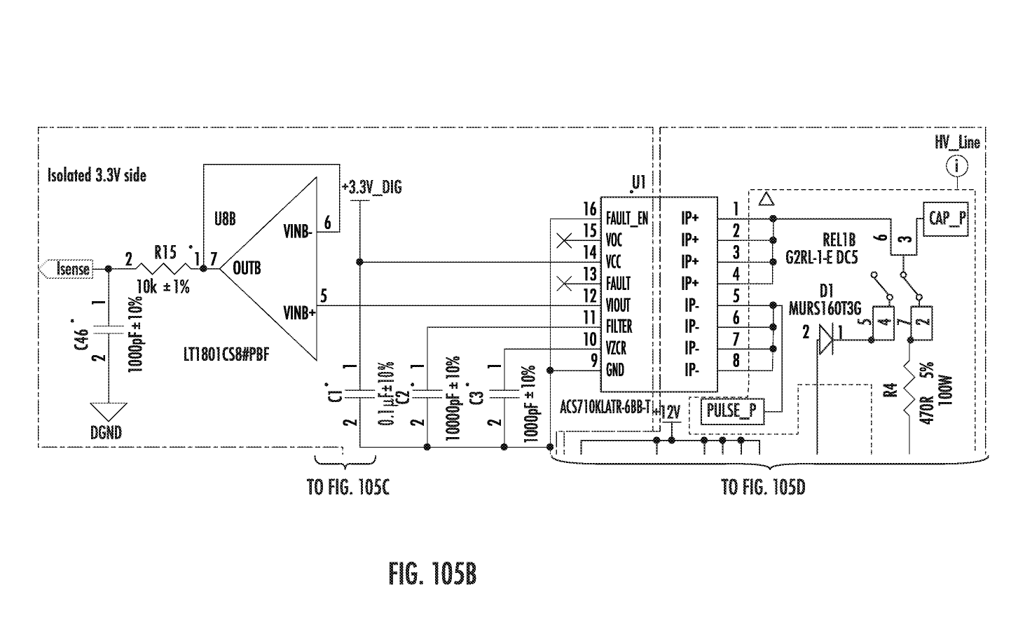

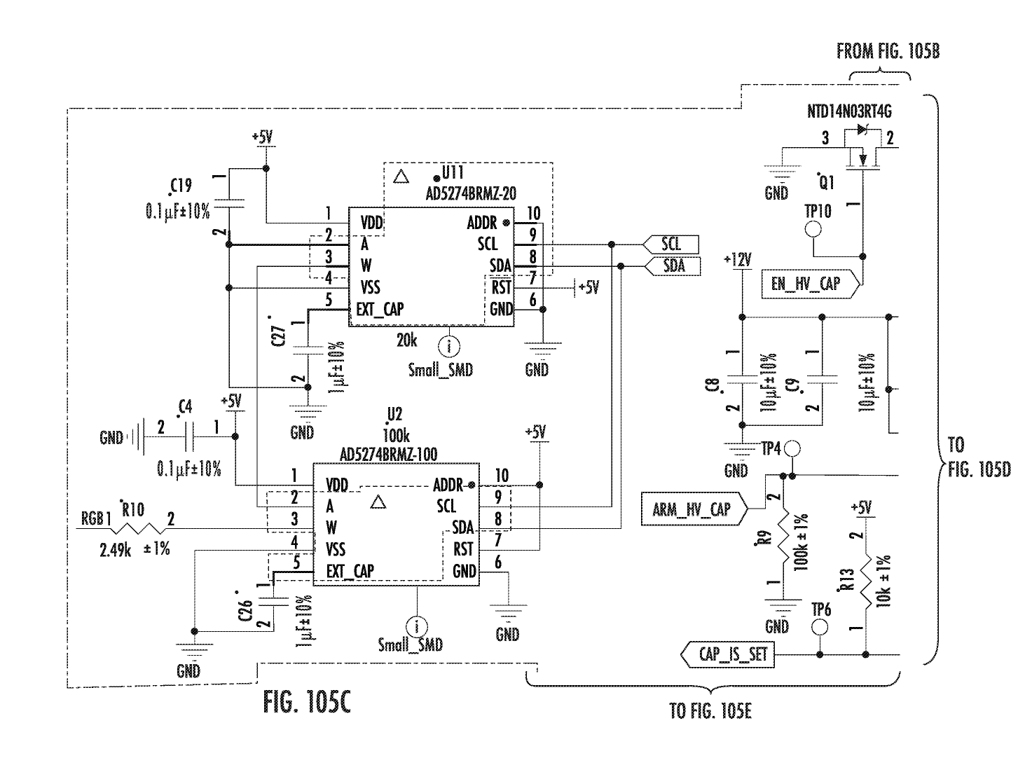

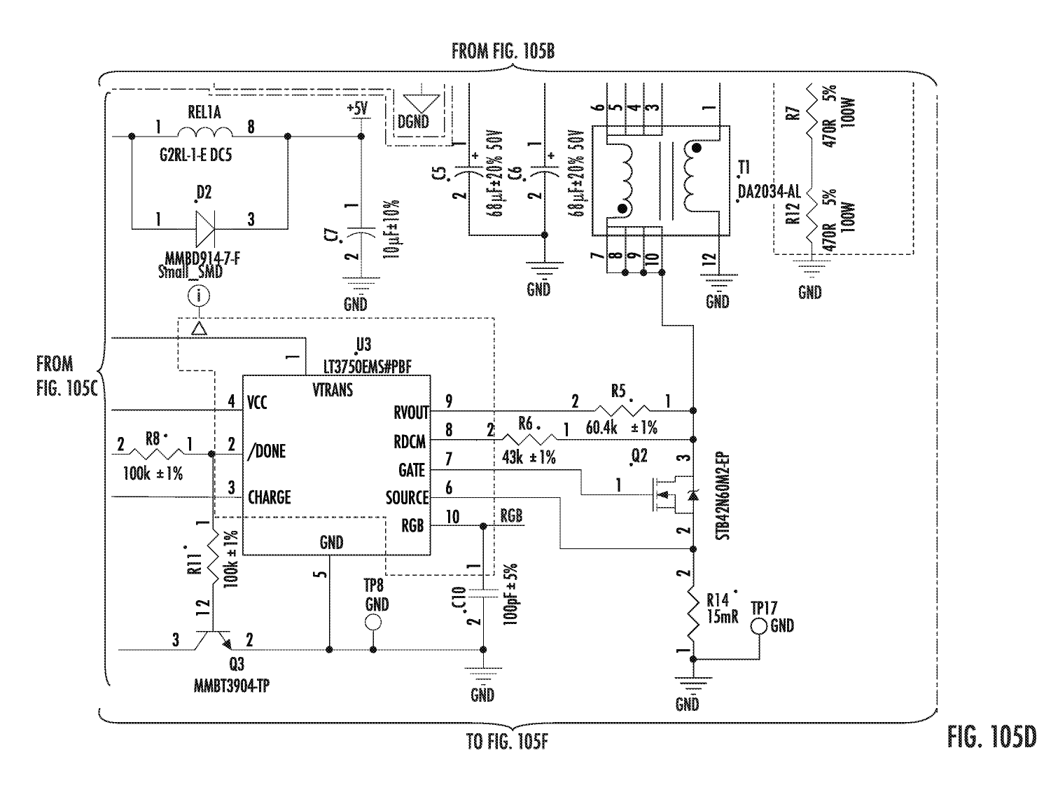

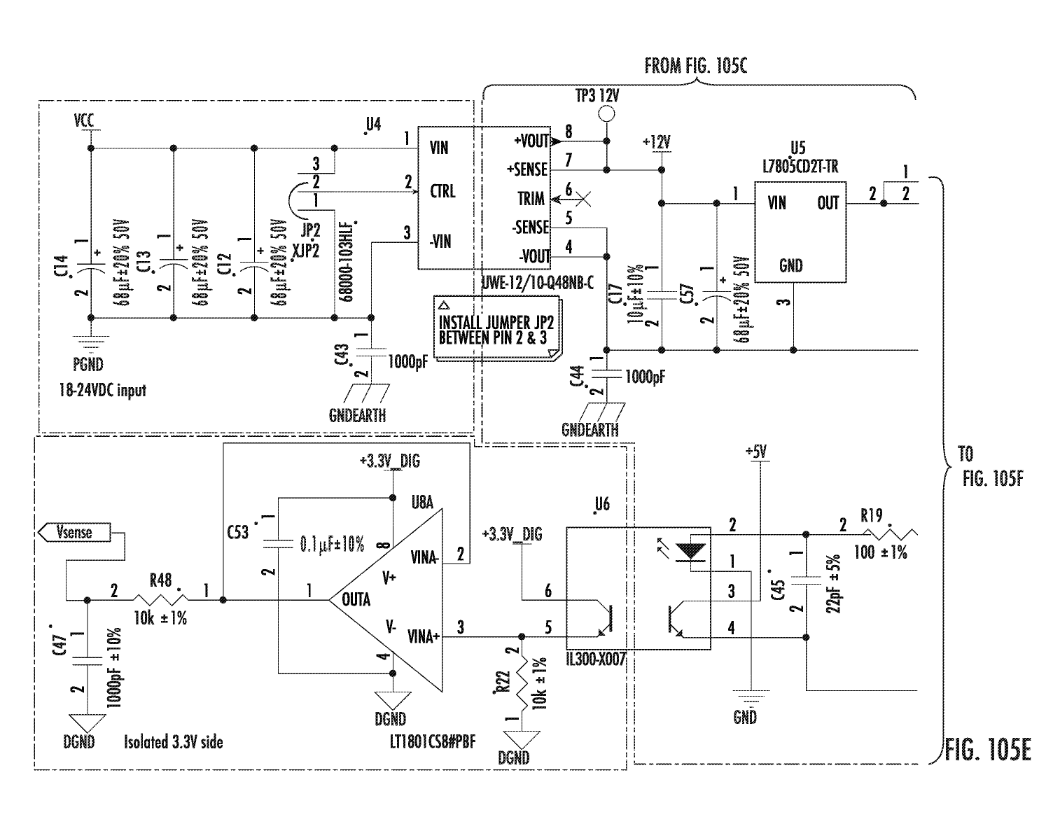

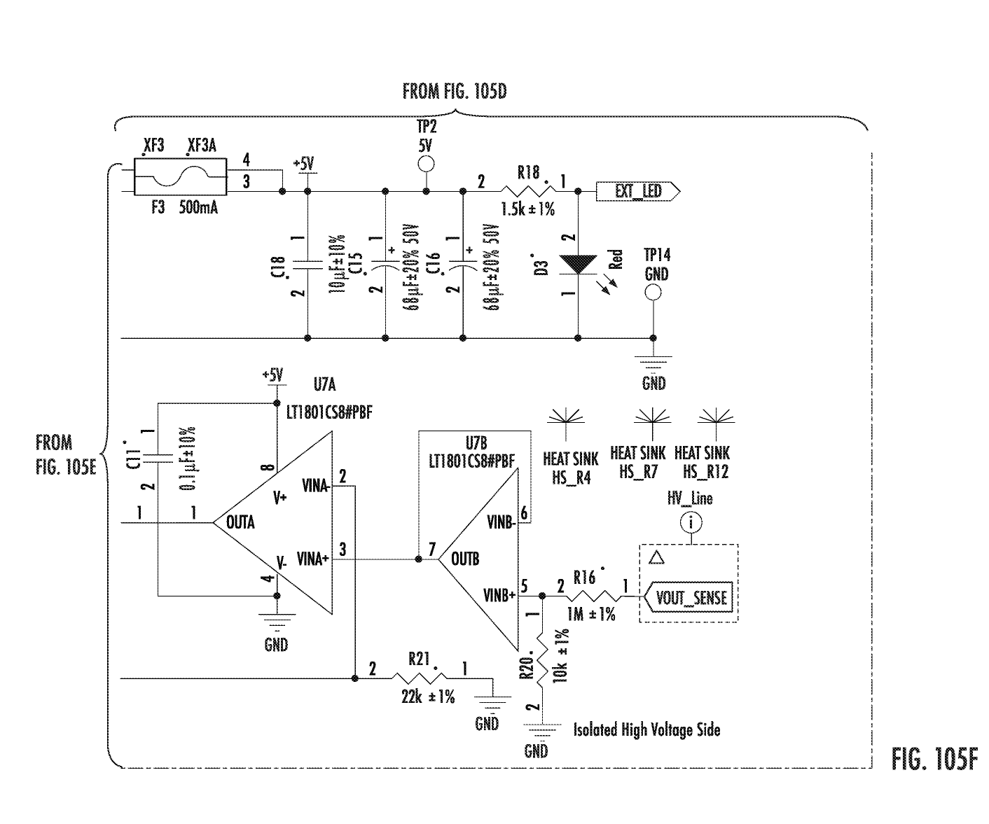

A main charging circuit of the power generation board is shown in FIGS. 105A-105F. The core of the circuit lies within the LT3750 Capacitor Charger Controller which, in conjunction with the DA2034 Flyback Transformer, STB42N60M2 Power MOSFET, and MURS 160T3G Power Rectifier Diode, form the essential flyback converter capacitor charging circuit (also referred to herein as the “flyback circuit”). The LT3750 Capacitor Charger Controller may be supplied by Linear Technologies, Inc., for example.

The operation of a flyback converter capacitor charging circuit involves two phases of operation: energy storage and flyback. In the energy storage phase, the NMOS is in active-mode and primary current is ramping. Energy is being stored in the transformer. The secondary voltage is negative so D1 is reverse-biased, which isolates the capacitor. In the depicted embodiment, D1 is a rectifier diode. The D1 may operate in the context of the flyback converter circuit and may prevent energy from being transferred to the capacitor when the MOSFET is OFF. If current is allowed to flow in the secondary loop of the flyback converter circuit then no energy is being stored in the transformer. In flyback phase, the NMOS is in cut-off-mode and the primary current is falling off. In the flyback phase, the stored energy now charges the capacitor. In this circuit there are additional feedback loops which regulate the Gate voltage (current limiting functionality) and a DCM (discontinuous mode) functionality which modulates the primary current amplitude to meet the demands of the load (amplitude modulation). In order to achieve DCM the LT3750 controller studies the NMOS Drain-Source voltage to determine when the Drain-Source voltage is equivalent to the input voltage before switching from the flyback phase to the energy storage phase (turning the NMOS back on), thus minimizing the energy loss across the NMOS by ensuring there is no primary current.

Also note that AD5274BRMZ (U11 and U2) includes digital rheostats designed to set the Output Voltage Sense Pin (RBG) on the LT3750 controller. The flyback converter charging circuit includes rheostats that allow a range of voltages (0-300 volts) to develop on the output capacitor. The monitoring signal VOUT_SENSE feeds into a buffer/comparators (U7A/U7B). The analog signal is filtered across to the digital board to feed into the STM32 main microcontroller.

In the present disclosure, the power generation board has three 470 Ohm, 100 W resistors with heatsinks. The effect is that the current discharges quickly, in about 14 seconds (e.g., 1410 Ohms*10 mF=14.1 seconds), eliminating the possible risk factor at higher speeds. The power generation board includes a Hall Effect sensor U27 for secondary current sense to potentially use for the crowbar overcurrent circuit. Additionally, voltage monitor, U23, is included to potentially use for crowbar overvoltage. A watchdog circuit U22 (and supporting components) is included to monitor the microcontroller, 5V, +12V, and 9V rails.

The circuit is advantageous in that the inclusion of the DA2034 transformer has been found to have improved responsiveness to hand-soldering and ultrasonic cleaning. Further, the primary current in transformer (T1) is 5.2 Amps with an R14 sense resistor having a current limit of 78 V/R sense. The current sensing circuit brings an additional layer of safety by limiting the current on the high voltage line. The current sensing circuit monitors the high voltage capacitor line to the output of the device. The current sensing circuit generates a voltage (VIOUT) which is proportional to the sensed current, which is then filtered and sent to the STM32 main microcontroller.

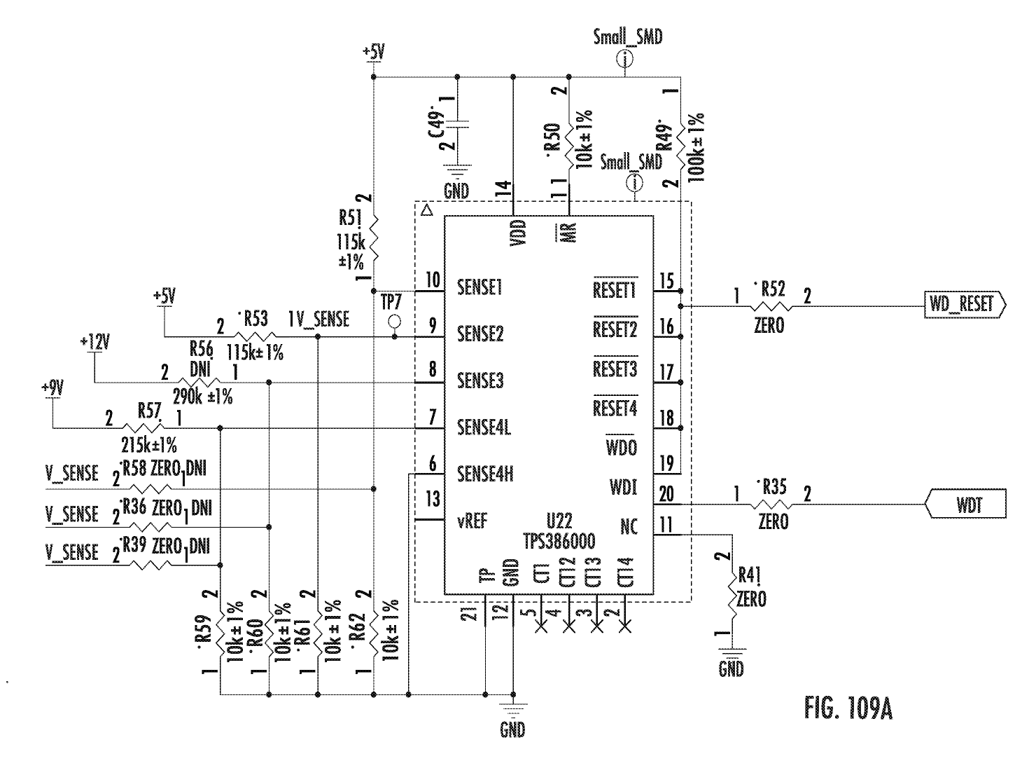

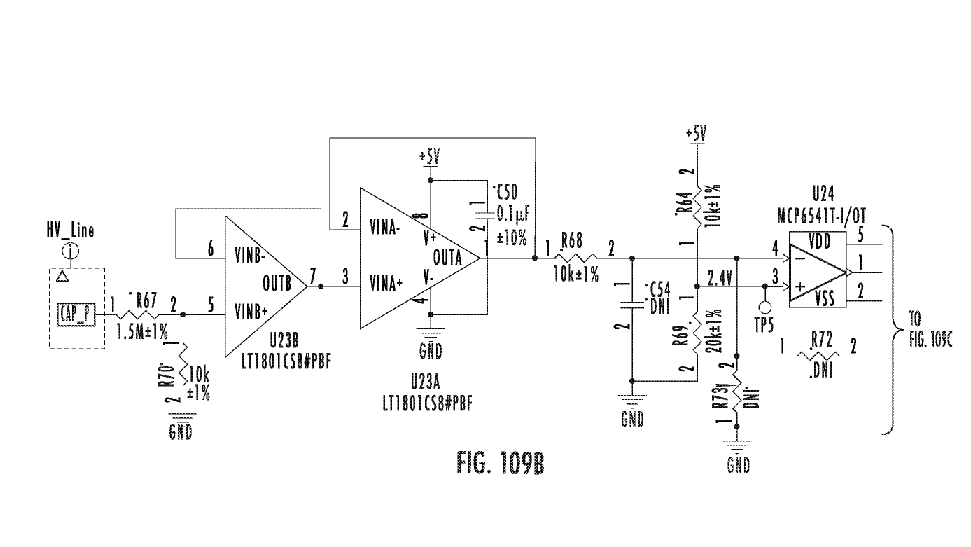

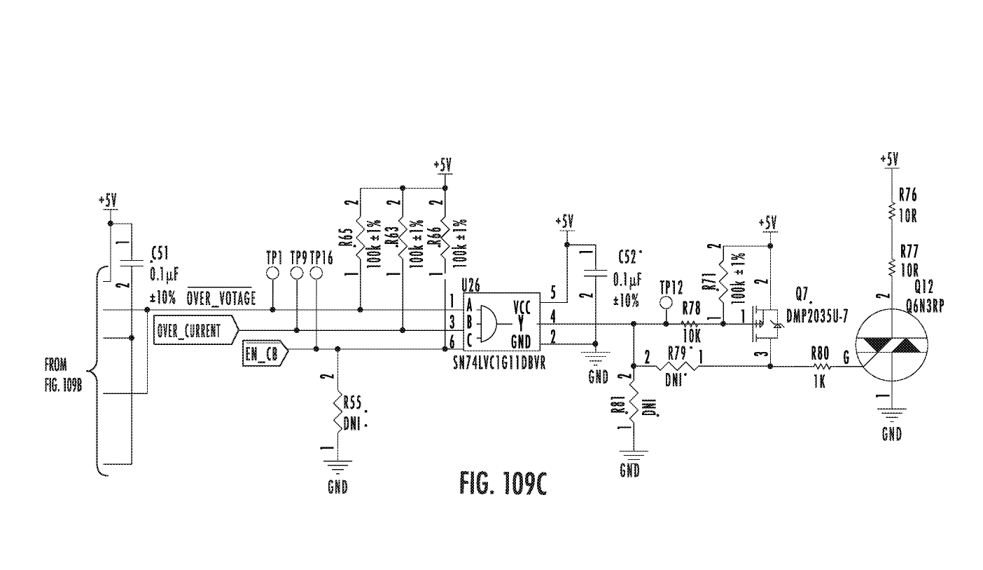

Another advantage results from the crowbar protection circuit, thyristor Q12 (Q6N3RP), shown in FIGS. 109A-109C. The thyristor is connected across the +5V power rail and ground. When activated by the appropriate current/voltage-sensing analog/digital signals, the thyristor latches into a conducting state. The +5V power rail is now conducting across R76 and R77 which represent 20 Ohms. This increased current blows out the fuse F3 in FIGS. 105A-105F (XF3) which has a 500 mA current limit. The result of this isolates the entire+5V power rail from its supply (L7805CD2T voltage regulator (U5)), which effectively shuts off the high voltage circuitry and most importantly, resets the relay REL1B (G2RL-1-E DC5) to its normal state of conducting the high voltage line directly to ground via high-wattage resistors R4, R7, and R12. The effect is that the high voltage capacitor is discharged quickly to ground, eliminating a possible risk factor.

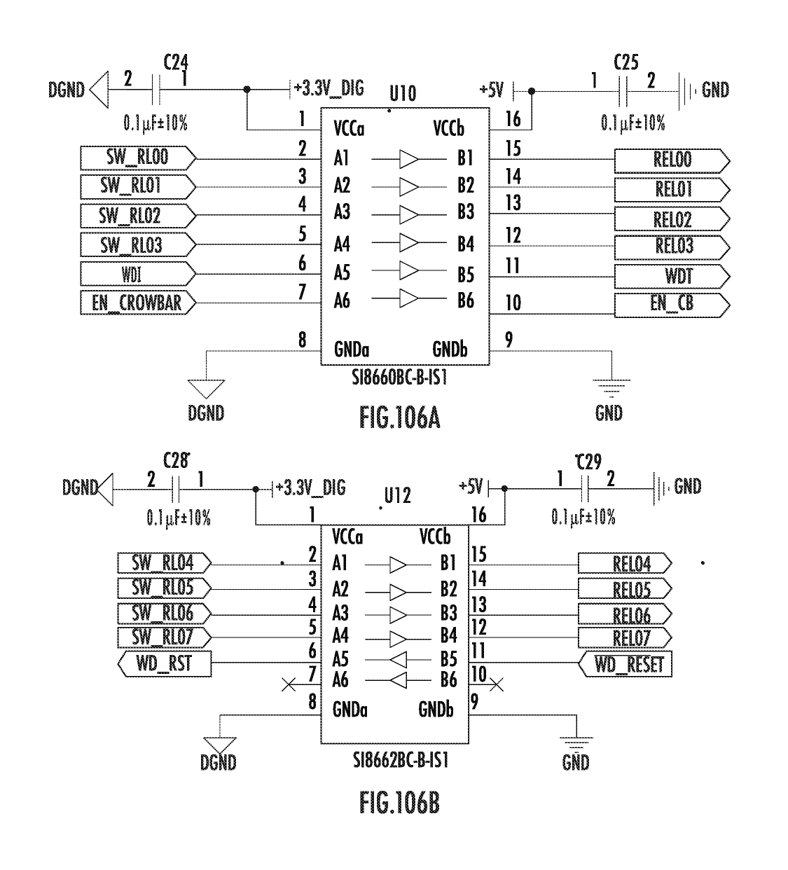

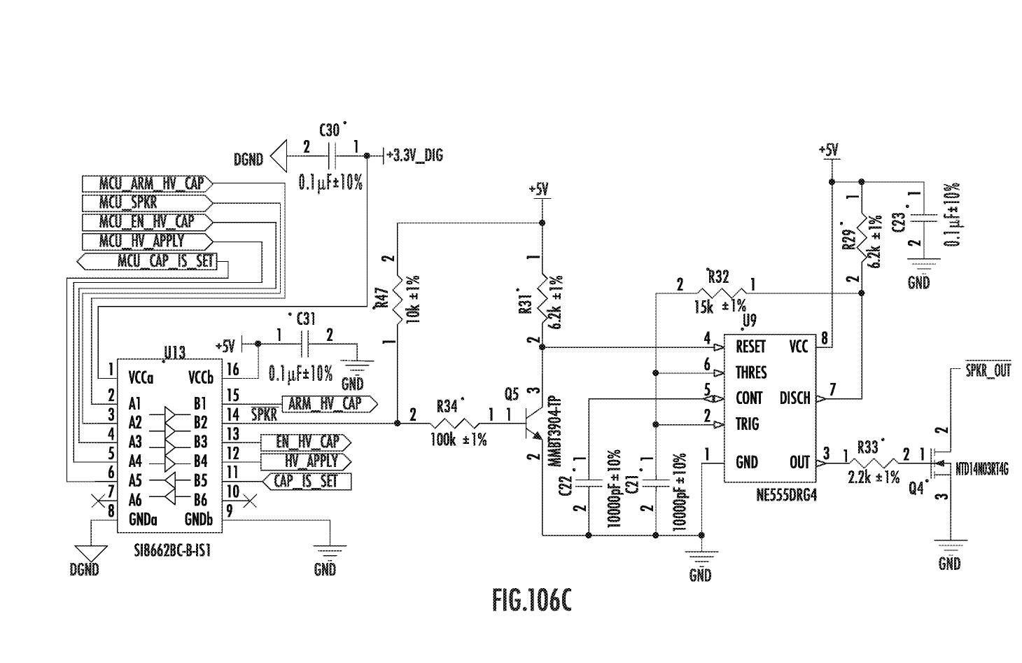

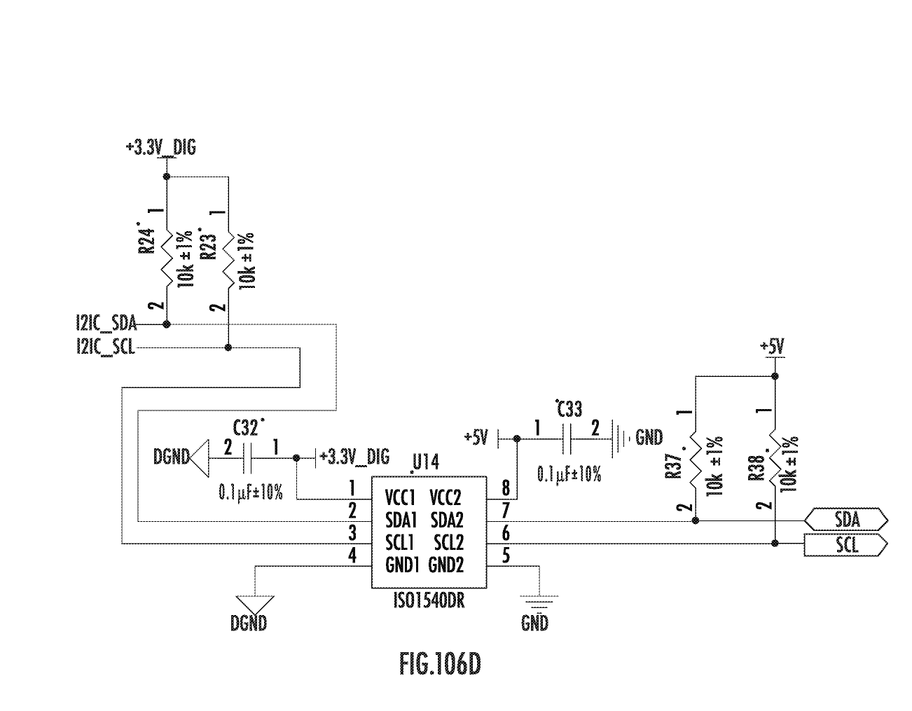

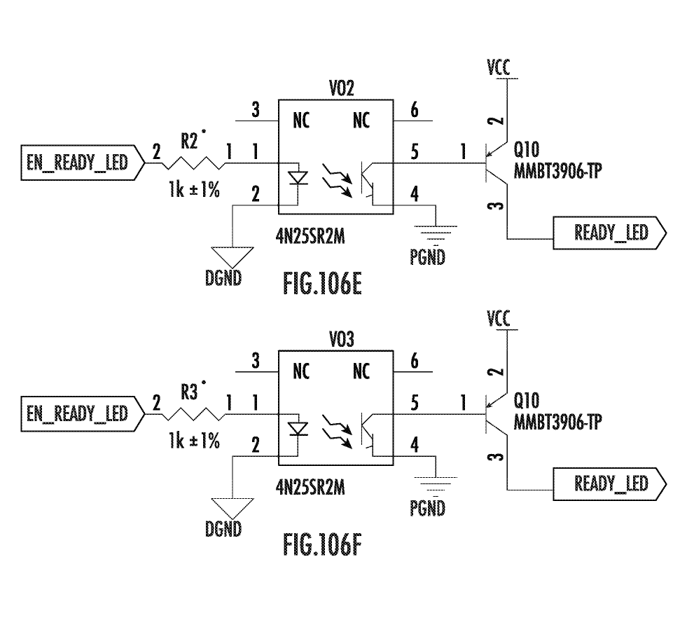

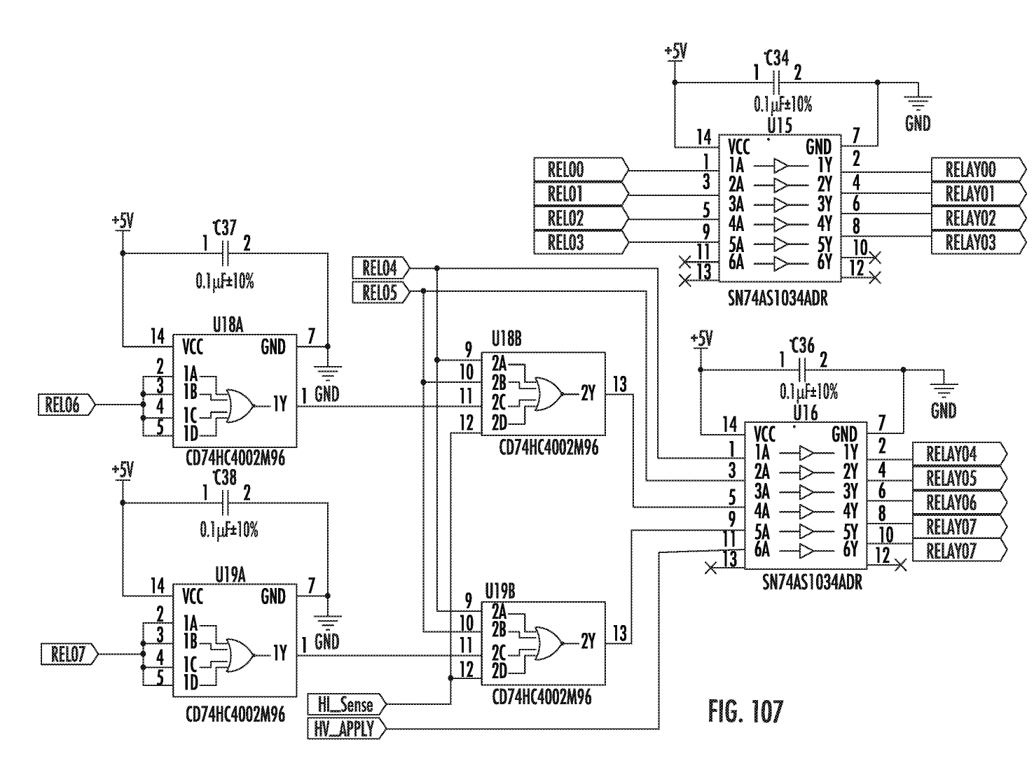

FIGS. 106A-106F detail the isolation wall which buffers and drives the 3.3V signals coming from the digital board to 5V which is used to power the logic circuitry (See FIG. 107) on the power board.

FIG. 107 details the relay control circuitry which buffers and drives the digital signals from the isolation circuitry to relay control signals. NOR gates U18A, U18B, U19A, U19B synchronize the relays to open and close in a predetermined firing pattern to enable pulsing.

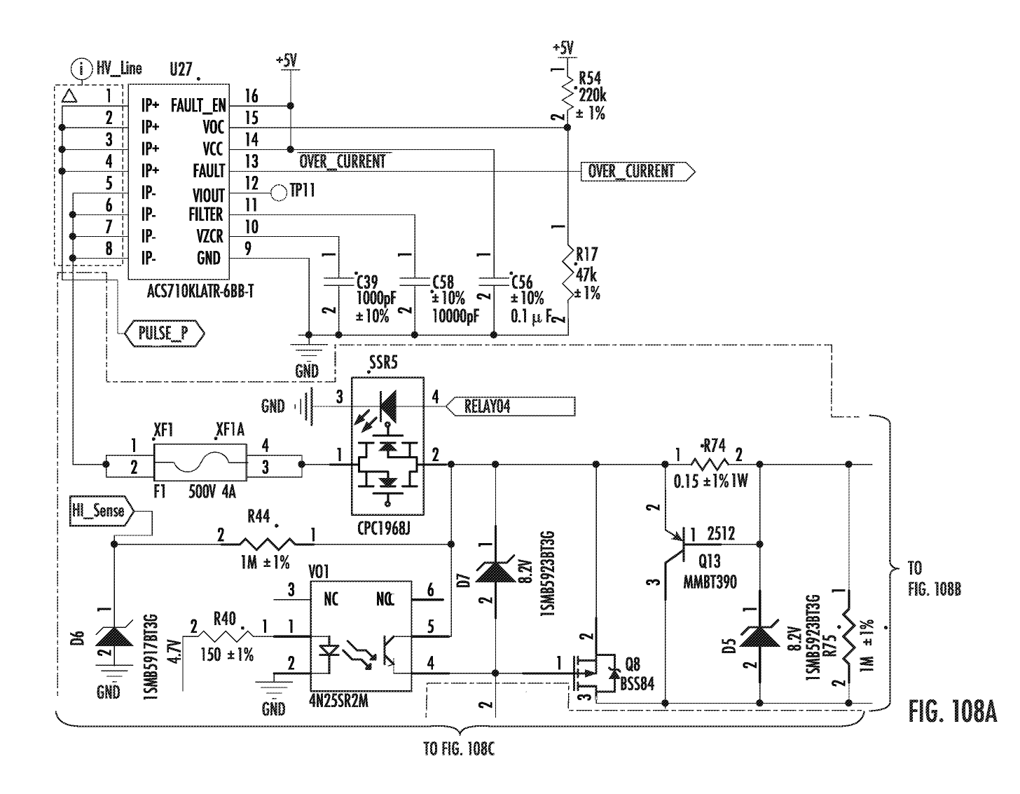

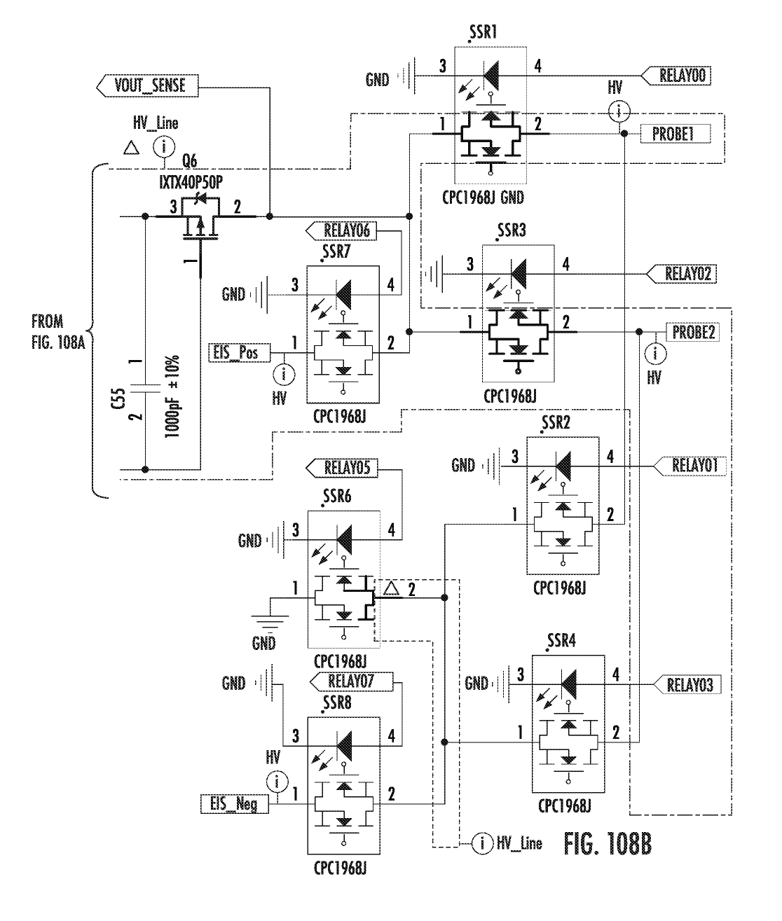

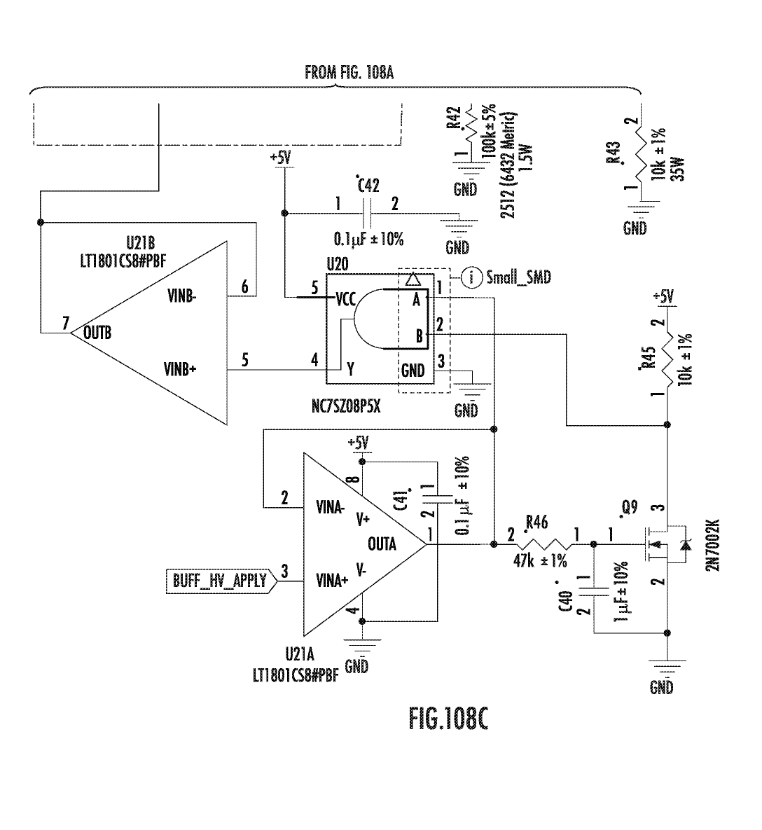

FIGS. 108A-108C detail the application of the high voltage line through the relays to the treatment output of the device of the present disclosure. Starting from the left of the figure, the PULSE_P signal goes through the ACS710 Hall Effect Current Sensor (U27), which monitors the capacitor current of the high voltage line (in addition to U1 on FIGS. 105A-105F), and is capable of sending a signal (OVER_CURRENT) to the crowbar circuit. The high voltage line goes through relay SSR5 and fuse F1, two safety measures before the high voltage line is connected to therapy output. Furthermore, R74 is a current sense resistor which is used in the feedback loop of Power MOSFET Q6. The purpose of Q6 is to limit the current output (as defined by R74) by operating in the linear region. This active circuitry limits the therapy current to a set value by dropping voltage across Q6. The gate of Q6 is enabled by Q8, which is driven by BUFF_HV_APPLY. This signal enables the application of the high voltage therapy pulsing. Q9 is an additional safety feature which automatically disables the pulse enable signal if the pulse enable signal has been on longer than the discharge time of the C40 capacitor. Finally, looking at the relays which dictate the firing patterns, it is of note that the EIS signals and high voltage signals coincide at the same two electrodes. The synchronization of the relays ensure that the high voltage signals and EIS signals are directed properly through the circuitry.

FIGS. 109A-109C detail the watchdog circuit and the crowbar circuit of the power generation board. The crowbar circuit enables multiple signals to trip the Q12 thyristor which spurs a chain of events which effectively “crowbar” the high voltage line. The watchdog circuit, through TPS386000 Voltage Supervisor (U22), monitors the power rails and can detect software hang-ups and send reset signals to the main microcontroller. The main microcontroller also checks the status of U22.

Combined, the digital board integrates both data acquisition components with the microcontroller unit to increase signal integrity by forgoing the cable assemblies between the two boards.

In some embodiments, the generator 12 may include a power supply 29 configured to receive power from the electrical mains and supply electrical energy to the system 10. In some embodiments, the generator 12 connects to the applicator via a wired connection, such as cable 136 shown in FIG. 51 and described elsewhere in the present disclosure. In some embodiments, a connection between the generator 12 and the applicator is a wireless connection. In some examples, the wireless connection may utilize low-energy communication with the respective elements being configured to send and receive signals. The low-energy communication technology may be Bluetooth®. In some embodiments, the generator may be a high voltage generator.

The processor 30 may be embodied in a number of different ways. For example, the processor 30 may be embodied as various processing means such as one or more of a microprocessor or other processing element, a coprocessor, a controller, or various other computing or processing devices including integrated circuits such as, for example, an ASIC (application specific integrated circuit), an FPGA (field programmable gate array), or the like. Although illustrated as a single processor, it will be appreciated that the processor 30 may comprise a plurality of processors in each device of the system or a single or plurality of centralized processors for multiple devices. The processor may be in operative communication with and may be configured to perform one or more functionalities for the devices of the electroporation system 10 as described herein. The processor may be embodied on a single computing device or distributed across a plurality of computing devices collectively configured to function as a controller 24. For example, a user device such as a smart phone, tablet, personal computer and/or the like may be configured to communicate with a detection device linked with the processor via means such as by Bluetooth™ communication or over a local area network. Additionally or alternatively, a remote server device may perform some of the operations described herein, such as processing data collected by any of the sensors, and providing or communicating resultant data to other devices accordingly.

In some example embodiments, the processor 30, may be configured to execute instructions stored in the memory 36 or otherwise accessible to the processor. As such, whether configured by hardware or by a combination of hardware and software, the processor 30 may represent entities (e.g., physically embodied in circuitry—in the form of processing circuitry) capable of performing operations according to embodiments of the present invention while configured accordingly. Thus, for example, when the processor 30 is embodied as an ASIC, FPGA, or the like, the processor 30 may be specifically configured hardware for conducting the operations described herein. Alternatively, as another example, when the processor 30 is embodied as an executor of software instructions, the instructions may specifically configure the processor 30 to perform one or more operations described herein.

In some embodiments, the applicator 14 may further include a memory 38 that stores information relating to the applicator. The controller 24 may interrogate the memory 38 of the applicator and identify the applicator and any necessary steps or instructions to execute electroporation based on the data stored in the memory 38. In this manner, the controller 24 may identify the applicator 14 before beginning electroporation. In some embodiments, the memory 38 may be disposed in the cable assembly (e.g., cable 76 and connector 78 shown in FIG. 19).

In some example embodiments, the memory 36, 38 of the generator and applicator, respectively, may include one or more non-transitory memory devices such as, for example, volatile and/or non-volatile memory that may be either fixed or removable. In this regard, each memory 36, 38 may comprise non-transitory computer-readable storage media. It will be appreciated that while each memory 36, 38 is illustrated as a single memory in each device, each memory 36, 38 may comprise a plurality of memories in one or more devices or a single memory or centralized memory or plurality of memories for multiple devices. The centralized memory may be embodied on a single computing device or may be distributed across a plurality of computing devices. Each memory 36, 38 (or centralized memory(ies)) may be configured to store information, data, applications, computer program code, instructions and/or the like for enabling the electroporation system 10 to carry out various functions in accordance with one or more example embodiments.

Each memory 36, 38 (or any centralized memory or the like) may be configured to buffer input data for processing by the processor 30. Additionally or alternatively, such memory may be configured to store instructions for execution by the processor 30. In some embodiments, such memory may include one or more databases that may store a variety of files, contents, or data sets. For instance, among the contents of each memory 36, 38, applications may be stored for execution by the processor 30 to carry out the functionality associated with each respective application. As a further example, each memory 36, 38 may store data detected by a sensor(s) of the detection device, and/or application code for processing such data according to example embodiments. In some cases, each memory 36, 38 may be in communication with one or more of the processor 30, the electrodes 100, the generator 12, the drug delivery device 16, and/or other apparatus and sensors. In some embodiments, each memory 36, 38 may store step by step commands for specific surgical procedures that may be executed by the processor. For example, this may include details to navigate the applicator to a target site for a bronchoscopy. In a further example, such details may be used as commands for a robot to move the applicator to a target site and/or perform a procedure (in such an instance, a centralized memory or memories may be preferred, and such memory may even be included in the robot itself). This type of storage is also contemplated for other procedures as described elsewhere in the disclosure. In some embodiments, one or more of the memory 36, 38 may comprise an electrically erasable programmable read-only memory (EEPROM). In some embodiments, the applicator 14 memory 38 may include an EEPROM chip.

With reference to FIG. 3, an example generator 12 and simplified applicator 14 are shown. The generator may generate electrical signals to electroporate the target tissues. The generator 12 may regulate the properties of the electrical signals (e.g., voltage, amplitude, frequency, duration, and the like) to cause reversible electroporation of the tissues without damaging the target tissues. In some embodiments, the generator 12 may include a foot pedal 58 for allowing a user to actuate and operate the generator and electroporation. The foot pedal 58 may be connected to the generator via a wired connection or via a low energy wireless connection, such as Bluetooth®. Where a wireless connection is used, each of the foot pedal 58 and the generator may include sensors to send and receive signals communicating changes in the status of the foot pedal 58. Operation of the generator may be aided or fully controlled by a robotic system. For example, a robotic arm may be configured to control the generator to achieve desired electrical parameters for electroporation. Examples of an electroporation system and corresponding electronic control methods, signals, and apparatus are described in U.S. Pat. Nos. 7,412,284 and 9,020,605 and International Application No. WO2016/161201, each of which is incorporated by reference herein in its entirety.

### Example Electroporation Applicator

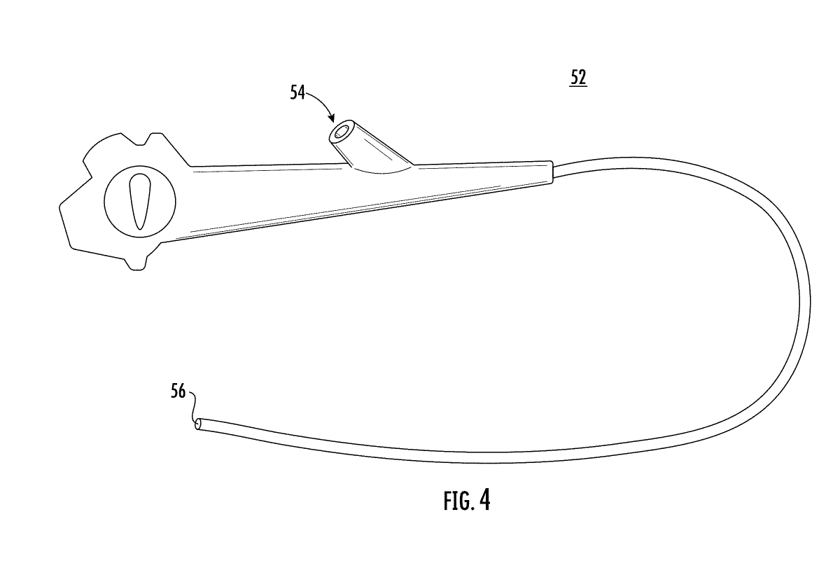

In some embodiments, the electroporation system 10 may be operable for use with access instrumentation, such as an endoscope or the like. Endoscopy involves inserting an endoscope into a cavity of the patient and administering at least some of the treatment locally using the endoscope (e.g., endoscope 52 shown in FIG. 4). Endoscopes may be rigid (e.g., a trocar) or flexible, and may include imaging, illumination, or operative features to assist the surgeon with the endoscopy. One example of an endoscope that may be incorporated into the electroporation system 10 is described in U.S. Pat. No. 6,181,964, hereby incorporated by reference herein in its entirety. With reference to FIG. 4, in some embodiments, endoscopes 52 also include a working channel 54 that extends from an upper or proximal end of the endoscope (e.g., a control section that is actuated by the user) to a distal end 56 of the endoscope through which one or more instruments, such as applicator 14, may be inserted to conduct the endoscopic procedure. In some instances, a flexible endoscope may have a narrower working channel than a rigid endoscope. As is known in the art, a flexible endoscope is typically used for procedures where the access pathway is via a conduit, such as in an esophageal approach to reach the lungs, while a rigid endoscope is typically used for procedures where the access pathway is a “line of sight” into the patient and to the particular tissue, such as is used in many abdominal procedures.

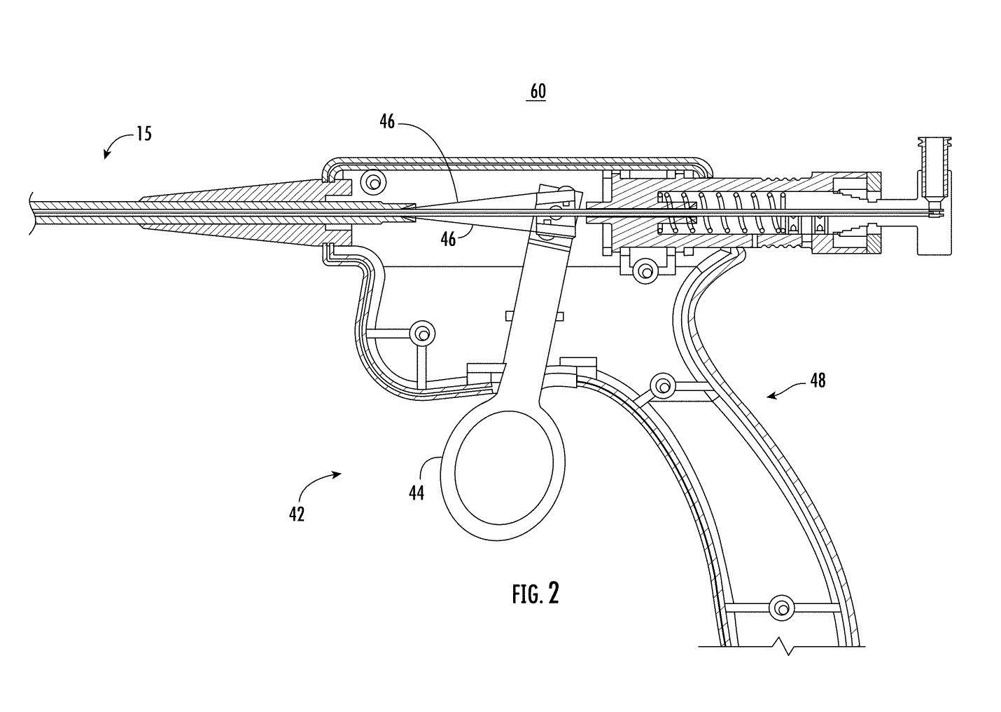

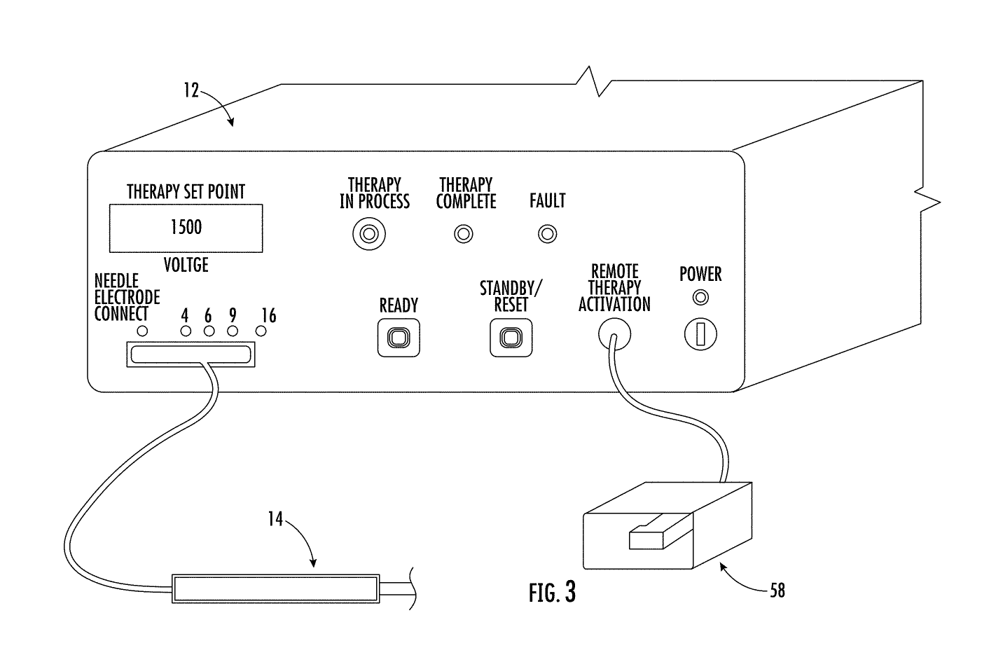

Endoscopic electroporation may involve inserting at least a portion of an applicator (e.g., the insertion tube 15 of the applicator 60 shown in FIG. 2; the insertion tube 15 of the applicator 70 shown in FIG. 19; or the insertion tube 15 of the applicator 110 shown in FIG. 47), with the electrodes (e.g., electrodes 100) at a distal end of the applicator, through the working channel of the endoscope to apply an electric field to the tissue adjacent to the distal end of the endoscope. In some examples, the slidable connection holding the applicator and the endoscope together may be controllable such that once the endoscope is advanced to a location in the body approaching the target site for the electroporation therapy, the applicator may be controllably advanced relative to the endoscope so that a distal end of the applicator reaches the target site while the endoscope remains at a distance relative to the target site. As discussed elsewhere herein, embodiments of the applicator may be mechanically steerable such that the tip may be steered to the target site via controls at or proximate the handle of the applicator. The control mechanism may be established based on direct visualization (e.g., a camera associated with the endoscope), surgical navigation, manual guidance based on the expected friction between the applicator surface and the interior surface of the endoscope, or other parameters as may be applicable for the particular structures included in the system. This controllable advancement of the applicator relative to the endoscope is of particular advantage where access to the target site involves passage through an internal vessel that is small in diameter. In such circumstances, the smaller diameter of the applicator relative to the endoscope allows the applicator to be advanced independently at lesser risk to the patient. This circumstance may arise, for example, where a tumor to be treated is in the cerebrum and intra-cranial blood vessels must be traversed to reach the tumor.

The electroporation system 10 can be used in any endoscopic access approach desired to fulfill its use and purpose. For example, in some embodiments, the electroporation system 10 may be used with an Olympus® EBUS Bronchoscope for performing bronchoscopy. In some embodiments, a flexible laparoscopic instrument may be used with the insertion tube of the applicator disposed therein. Further, in some embodiments, the applicator may be inserted directly into a keyhole opening in the patient (e.g., with the laparoscopic device shown in FIG. 86). In this arrangement, the keyhole opening in the body of the patient operates as the working channel during the electroporation procedure. Thus, in some examples, the system may include an applicator with an insertion end that is configured to be advanced to the target site unenclosed by an insertion device. In some examples, the properties and structure of the insertion tube may be modified to accommodate use of the applicator as a standalone access element in the procedure. In the aforementioned examples, the system is complete without an endoscope, though it may be used with any type of endoscopic instrument desired. Further, in some examples of the aforementioned systems, applicator 14, 60, 70, 110, 1000 may be the applicator of the system.

In some examples, the electroporation system 10 may include an integral, “all-in-one” system having any combination of one or more of an endoscope, drug delivery channel or applicator, electroporation applicator, steering system, vision system, and/or imaging system (e.g., ultrasound). Embodiments of each of the foregoing components may include those discussed elsewhere herein. In such embodiments, the applicator (e.g., including electrodes and/or a drug delivery channel) may be any of the applicators 14, 60, 70, 110, 1000 disclosed herein. In some embodiments, the applicator may be a retractable portion of the all-in-one system.

Turning now to the structure of the applicator itself, with reference to FIG. 2, an example applicator 60 is shown having an insertion tube 15, an actuator 42, and a control portion 48. The insertion tube 15 may have a diameter less than an internal diameter of the working channel of an endoscope (e.g., working channel 54 of endoscope 52 shown in FIG. 4) so that the insertion tube may be inserted into the working channel and may extend from the control portion 48 outside the endoscope at the external end (e.g., the end outside the patient) to the endoscopic site within the patient at the distal end of the endoscope. The insertion tube 15 may be longer than the working channel of the endoscope. The insertion tube 15 may also include one or more channels extending therethrough to allow the various components described herein to extend into the patient for treatment. For example, the actuator 42 may be movably engaged with at least a portion of the control portion 48 and may extend through the insertion tube 15 to interact with the electrodes to allow a user to apply a force from the trigger 44 to deploy the electrodes at the distal end of the insertion tube 15 as described herein. In the embodiment depicted in FIG. 2, the actuator 42 includes a trigger 44 pivotally attached to the control portion 48 and a pushing element 46 connecting the trigger 44 to the electrodes such that pushing element 46 moves axially along the insertion tube 15, to move the electrodes, when the trigger is actuated.

In some examples, the electrodes are biased so that when no force is applied to the trigger 44, the electrodes are in a retracted position. In some examples, the trigger 44 must be held to maintain deployment of the electrodes such that anytime the trigger is released, the electrodes return to their retracted state. In some examples, the actuator may be modified to include a lock to hold the trigger in the actuated position or to include a slow release so that after the force applied to the trigger has ceased, the retraction of the electrodes is delayed and/or controlled. It is contemplated that these principles may be applied to other actuators as well, both those requiring physical movement and others that operate solely by control of an electrical connection. In some examples, each of the control portion 48, insertion tube 15 and actuator 42 are separate elements. In other examples, two or more of the control portion, insertion tube and actuator are integral with one another.

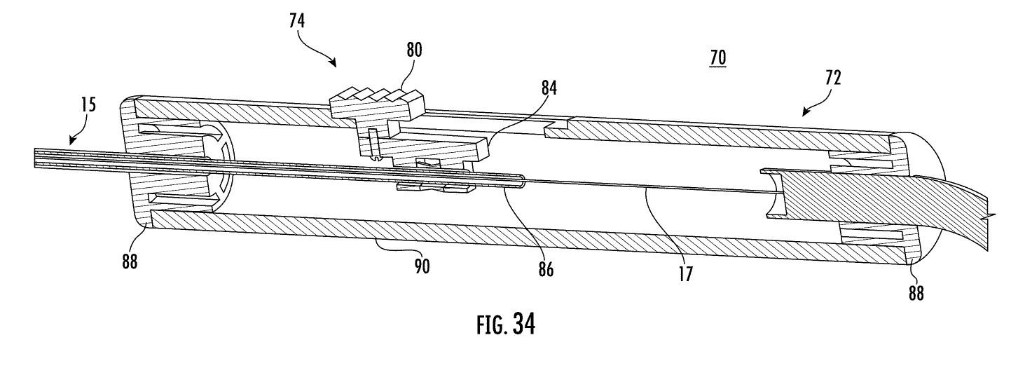

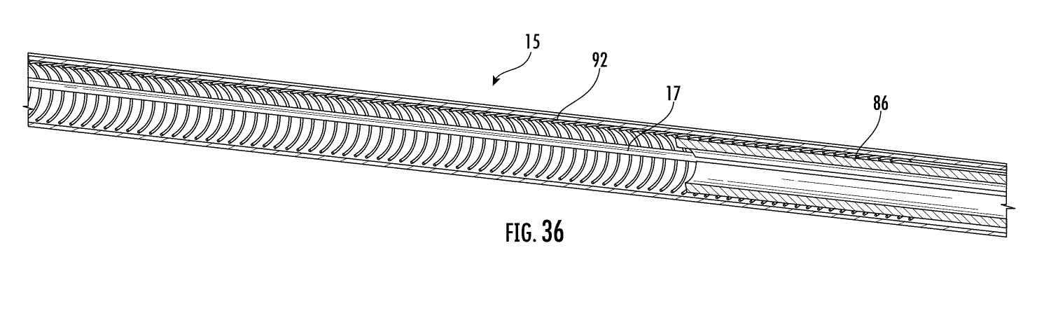

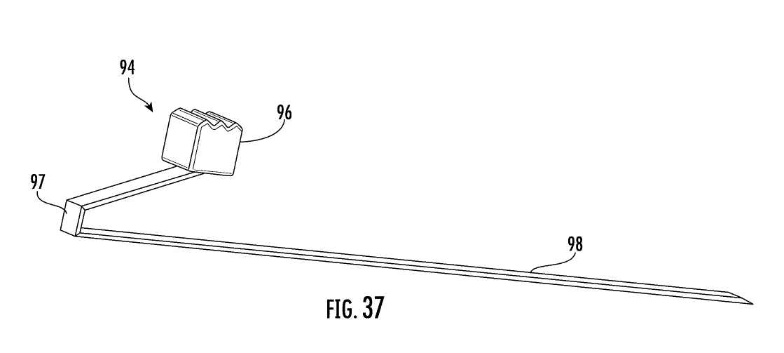

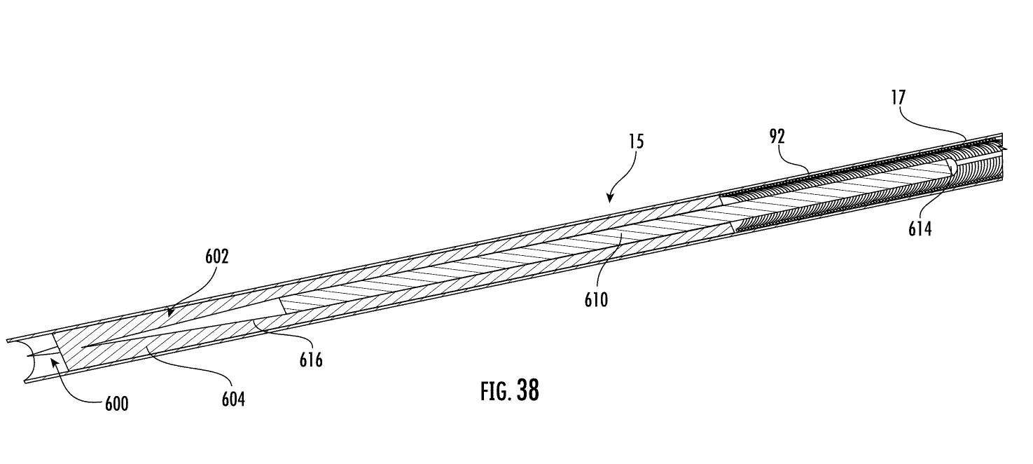

With reference to FIG. 19, another example applicator 70 is shown having an insertion tube 15, an actuator 74, and a control portion 72. The insertion tube 15 may have a diameter less than an internal diameter of the working channel of an endoscope (e.g., working channel 54 of endoscope 52 shown in FIG. 4) so that the insertion tube may be inserted into the working channel of the endoscope and may extend from the control portion 72 outside the endoscope at the external end (e.g., the end outside the patient) to the endoscopic site within the patient at the distal end of the endoscope. The insertion tube 15 may be longer than the working channel of the endoscope. Further, at least a portion of the insertion tube 15 may be flexible to, for example, allow for passage through a flexible endoscope already positioned through the tortuous pathway from the nose or mouth to the lungs, or may be rigid such that it is more suitable for passage through a rigid cannula, or further, for passage into the body of a patient without the need for an access instrument of any kind, or of course, for use with a rigid endoscope. These configurations of insertion tube 15 and access instrument are examples only as, of course, a configuration with a flexible insertion tube 15 could be used with a rigid cannula, such as a rigid endoscope. The insertion tube 15 may also include one or more channels extending therethrough to allow the various components described herein to extend into the patient for treatment. For example, the actuator 74 may be movably engaged with at least a portion of the control portion 72 and may extend through the insertion tube 15 to allow a user to apply a manual force from the control portion 72 (for example, via a switch 80) to deploy the electrodes at the distal end of the insertion tube 15 as described herein. The control portion 72 may include a body 90 and at least one end cap 88, which may support the insertion tube 15 and/or the cables 76 therein. In the embodiment depicted in FIGS. 19, 27, and 34, the actuator 74 includes a thumb switch 80 that is slidingly attached to the control portion 72 and engaged with a hollow mandrel 86 via a connector 84. With reference to FIG. 36, the mandrel 86 may be attached to a pushing element 92 (e.g., by crimping), such that when the actuator 74 is slid forward on the control portion 72 by a user sliding switch 80, the switch 80 pushes the hollow mandrel 86 axially forward, which drives the pushing element 92 axially forward to extend the electrodes 100 from the insertion tube 15 (e.g., either directly or by driving an electrode carrier 206, 602, 802 or other intermediate component, such as a balloon 302). Such a manual actuation mechanism for electrode deployment may be any structure desired other than the thumb switch 80 illustrated, for example, switch 80 could be a thumb wheel, a push button, a trigger mechanism, or the like.

With reference to FIG. 47, yet another example applicator 110 is shown having an insertion tube 15, an actuator 112, and a control portion 114. In some examples, the insertion tube 15 may have a diameter less than an internal diameter of the working channel of a cannulated access instrument, such as an endoscope (e.g., working channel 54 of endoscope 52 shown in FIG. 4), so that the insertion tube may be inserted into the working channel and may extend from the control portion 114 to a position outside the endoscope at the external end (e.g., the end outside the patient) to the endoscopic site within the patient at the distal end of the endoscope. The insertion tube 15 may be longer than the working channel of the endoscope. The insertion tube 15 may also include one or more channels extending therethrough to allow the various components described herein to extend into the patient for treatment. For example, the actuator 112 may be movably engaged with at least a portion of the control portion 114 and a portion of the actuator may extend into the insertion tube 15 to allow a user to apply a force from a switch 116 to deploy the electrodes at the distal end 118 of the insertion tube 15 as described herein. The control portion 114 may include a body 120 and at least one end cap 122, which may support the insertion tube 15 therein. In the embodiment depicted in FIGS. 47, 51, 52, and 57, the actuator 112 includes a thumb switch 116 that is slidingly attached to the control portion 114 and engaged with a hollow mandrel 124 of the actuator via a connector 126 (e.g., a lure lock). The mandrel 124 may be attached to a pushing element 128 (e.g., by crimping as shown in the embodiment of FIG. 36), such that when the actuator 112 is slid forward on the control portion 114 by a user sliding switch 116, the switch 116 pushes the hollow mandrel 124 axially forward, which drives the pushing element 128 axially forward to extend the electrodes 100 from the insertion tube 15 (e.g., either directly or by driving an electrode carrier 206, 602, 802 or other intermediate component, such as a balloon 302). In this manner, the actuator 112, including the switch 116, mandrel 124, and pushing element 128, may extend at least partially into the insertion tube 15 to drive the electrodes (e.g. electrodes 100).

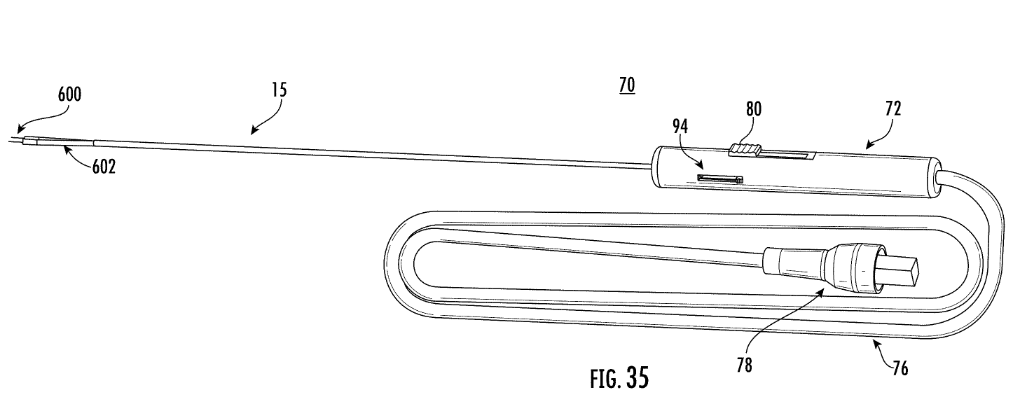

The applicator 14, 110 may further include the actuator 42, 74 structure, second actuator 94 (FIG. 35), described in greater detail elsewhere in the present disclosure, secondary button 82, and/or any of the other features from the applicators 14, 60, 70, 1000 described herein as if each individual feature had been described with respect to each embodiment, and such features may operate in accordance with their intended purpose in such combined embodiments. Similarly, in some embodiments, the features of any one applicator 14, 60, 70, 110, 1000 may be included in one of the other applicators.

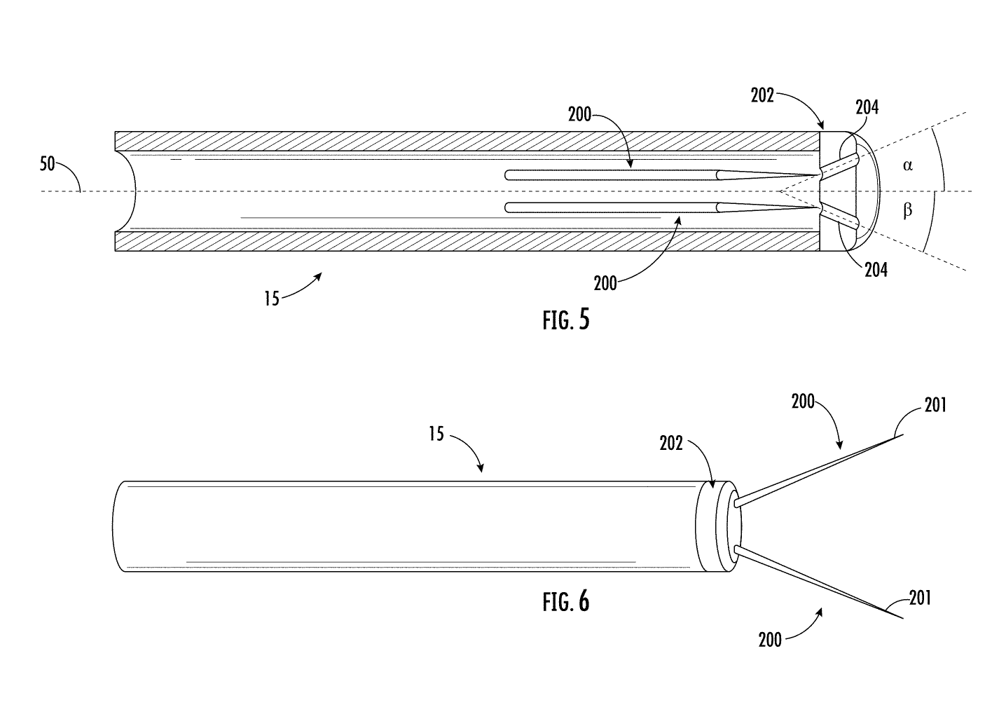

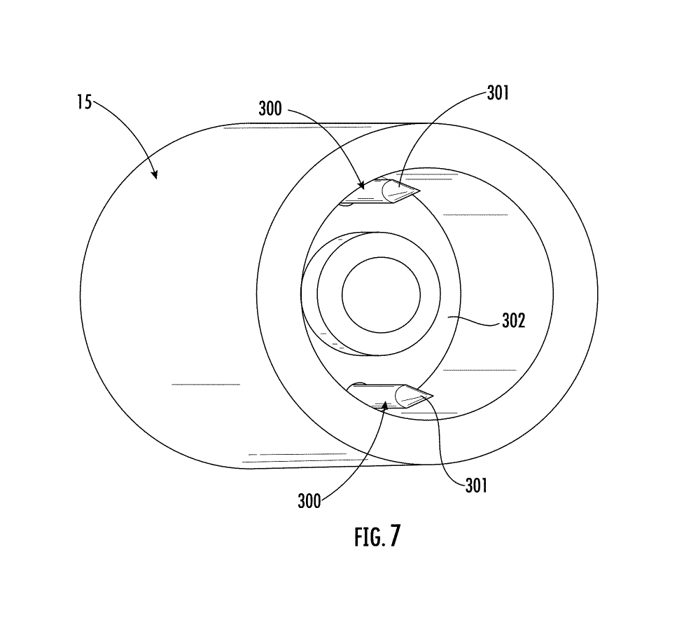

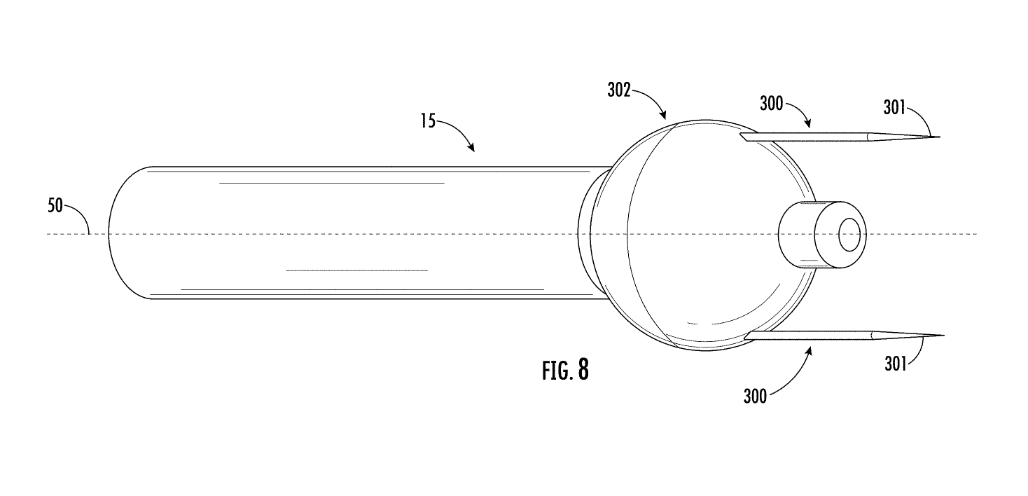

With reference to FIGS. 49, 50, and 56, the applicator 110 may define a piercing tip 130 at the distal end 118 of the insertion tube 15. The piercing tip 130 may define a generally needle-shaped projection having a pointed end 132 and a hollow core through which the electrodes (e.g., electrodes 100) and/or drug delivery channel 18 may pass. The piercing tip 130 may be configured to puncture body tissue to reach a target site before deploying the electrodes (e.g., electrodes 100) and/or treatment agent. For example, the piercing tip 130 may be used to pierce a patient's stomach liner to reach nearby organs such as the pancreas or liver. In some embodiments, the distal end 118 may comprise a flat, non-piercing tip according to other embodiments discussed herein, such as is illustrated in FIGS. 5 and 6.

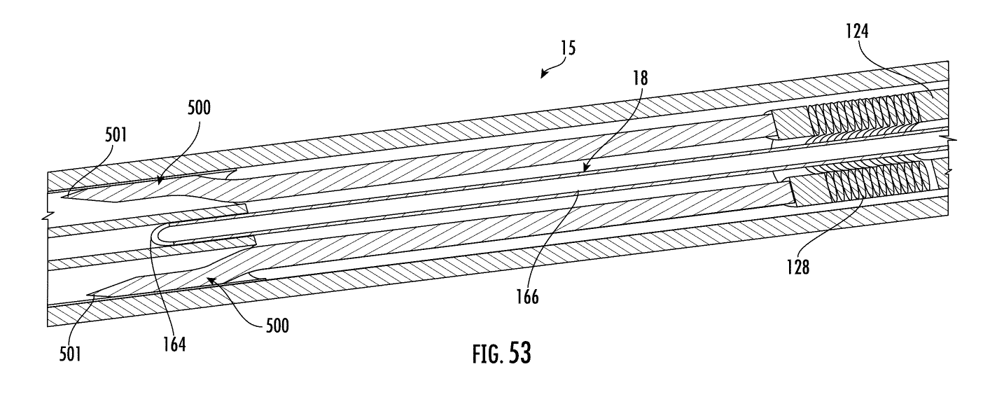

With reference to FIGS. 53, 58, 59, in some embodiments, the pushing element 128 may comprise a portion of the wiring for the electrodes. The generator (e.g., generator 12 shown in FIG. 1) may supply electrical impulses via a cable that enters the body 120 of the control portion 114 via a cable opening 134, as shown in FIGS. 51 and 57. With reference to FIG. 51, the cable 136 may pass through the cable opening 134 and connect to the mandrel or one or more wires therein (e.g., wires 17 shown in FIG. 36). The wires may transmit electrical impulses to the pushing element 128 from the cable 136, and to the electrodes 500 from the pushing element 128, as shown in FIG. 53.

With reference to the embodiment illustrated in FIG. 59, the pushing element 128 may comprise two coiled and electrically isolated wires 138, 140 that carry the impulses directed to two respective electrodes (e.g., the electrodes 100 discussed herein). The coiled wires 138, 140 may be insulated, for example, with an insulating casing (e.g., made of polyethylene, PVC, rubber-like polymers, etc.) and may have conductive cores passing therethrough. The coiled wires 138, 140 may be insulated so that the respective opposing signals of the electrodes (e.g., positive and negative electrical contacts) do not short. The pushing element 128 and mandrel 124 may define a central cavity 142 through which a drug delivery channel (e.g., drug delivery channel 18), or additional treatment-related device may pass. The ends of the coiled wires 138, 140 closest to the control portion 114 of the applicator may electrically connect to corresponding electrical wires (e.g., wires of the cable 136). These corresponding electrical wires of the cable 136 may run from the coiled wires 138, 140, along the mandrel 124 (e.g., floating outside of the mandrel), and out the applicator via cable opening 134.

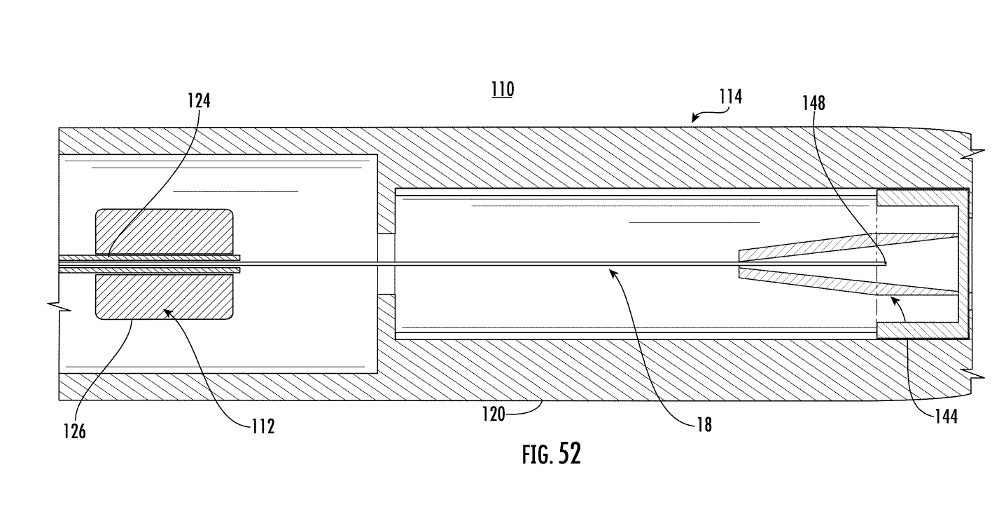

Turning to FIGS. 1, 51-53 and 56-58, the applicator 110 may include a drug delivery channel 18 configured to direct fluid from a drug delivery device 16 (shown in FIG. 1) to a target site (e.g., a tumor or lesion) in the patient. The drug delivery device 16 (shown in FIG. 1) may couple to a shroud 144 of the applicator 110 (e.g., via a threaded connection 146), which shroud 144 may engage a second distal end 148 of the drug delivery channel 18. In an alternative configuration of the system, the treatment agent may be supplied directly into the drug delivery channel 18 via the second distal end 148. The drug delivery channel 18 may extend from the second distal end 148 at the shroud 144 to a first distal end 164 through which the one or more treatment agents may be delivered. The drug delivery channel 18 may be coupled to the actuator 112 at the connector 126, mandrel 124, and/or pushing element 128, and the drug delivery channel 18 may travel axially with the actuator 112 relative to the insertion tube 15. In some embodiment the drug delivery channel 18 may be bonded to the pushing element 128. In some embodiments, for example as shown in FIGS. 51-53 and 56-58, the shroud 144 may be attached to and travel with the drug delivery channel 18. In some embodiments, the drug delivery channel 18 may be disposed in the central cavity 142 of the mandrel and pushing element 128. The drug delivery channel 18 may include a delivery channel 166 extending from the first distal end 164 to the second distal end 148 through which the one or more treatment agents may be delivered from the shroud 144 to the treatment site, as shown in FIGS. 52-53. The first distal end 164 of the drug delivery channel 18 may be pointed to pierce the tissue at the target site, or alternatively may have a blunt end for atraumatic delivery to the tissue at the target site. The drug delivery tube 18 may be flexible such that the tube can extend from the control portion 114 down into the target tissue in any direction desired.

In some embodiments, the drug delivery channel may have a non-circular cross-sectional shape. For instance, the shape may be polygonal, rectangular, oblong, elliptical, and so on. In some embodiments, the delivery channel 18 may be positioned on a periphery of the path through the insertion tube 15. In some examples, the delivery channel 18 may be positioned outside of a path of the electrodes. In some examples, the delivery channel 18 abuts an inner wall of the insertion tube 18. In some examples, the delivery channel 18 is formed with the inner wall of the insertion tube 18 and includes a further tube passing therethrough to advance out of the insertion tube for drug delivery during performance of the method. In some embodiments, the drug delivery channel 18 may be a hypotube.

In some embodiments, the drug delivery channel 18 may be made of a non-conductive material. In some embodiments, the drug delivery channel 18 may be made of a ceramic material. In some embodiments, the drug delivery channel 18 may be made of stainless steel. In a conductive embodiment (e.g., stainless steel), the distal end of the drug delivery channel 18, adjacent to the electrodes, may be coated in a non-conductive material (e.g., non-conductive ceramic). In some embodiments, the drug delivery channel 18 may be made of plastic. In some examples, the drug delivery channel may define a diameter of about 0.025 inches. The drug delivery channel 18 is advantageous in that it provides a protected structure within the applicator to deliver a treatment agent. Thus, the electrodes for electroporation and the treatment agent may all be safely carried within one structure, simplifying the surgical procedure.

With reference to FIGS. 53, 56, and 58, the electrodes 500 (e.g., any of the electrodes 100 discussed herein) and the drug delivery channel 18 may both be actuated simultaneously by the actuator 112. In some embodiments, the electrodes 500 (e.g., any of the electrodes 100, 200, 300, 400, 600, 700, 800 discussed herein) and the drug delivery channel 18 may move as a single unit. In some examples, the electrodes and the drug delivery channel move as a single unit where the electrodes are fixed relative to the drug delivery channel 18. In other examples, drug delivery channel may be movable independent of the electrodes and the applicator may include separate actuation mechanisms accessible to or otherwise controllable by a user for each of the drug delivery channel and the electrodes (and similarly, the electrodes can be actuated collectively and simultaneously or actuated individually by separate actuation actions). In this manner, the applicator may be configured so that deployment of the drug delivery channel may occur independently from deployment of the electrodes, such that the user can decide to actuate both simultaneously or sequentially. The first distal end 164 of the drug delivery channel 18 may be offset from the tips 501 of the electrodes, such that, given a flat planar target site, the electrodes pierce the target site before the drug delivery channel 18. In other examples, the first distal end 164 of the drug delivery channel 18 may be close to the tips 501 of the electrodes 500. In some embodiments, the first distal end 164 of the drug delivery channel 18 is positioned immediately inside an outward face of end cap 510 and remains stationary when the electrodes 500 are deployed.

In an alternative embodiment, the drug delivery channel may be integral with one of more of the electrodes, such that the electrode(s) is/are cannulated to provide a flow path for the treatment agent(s). In such an alternative configuration, the electrode(s) would be positioned in the target tissue first, and then the treatment agent(s) would be delivered to the tissue via the cannulated pathway through the electrode(s).

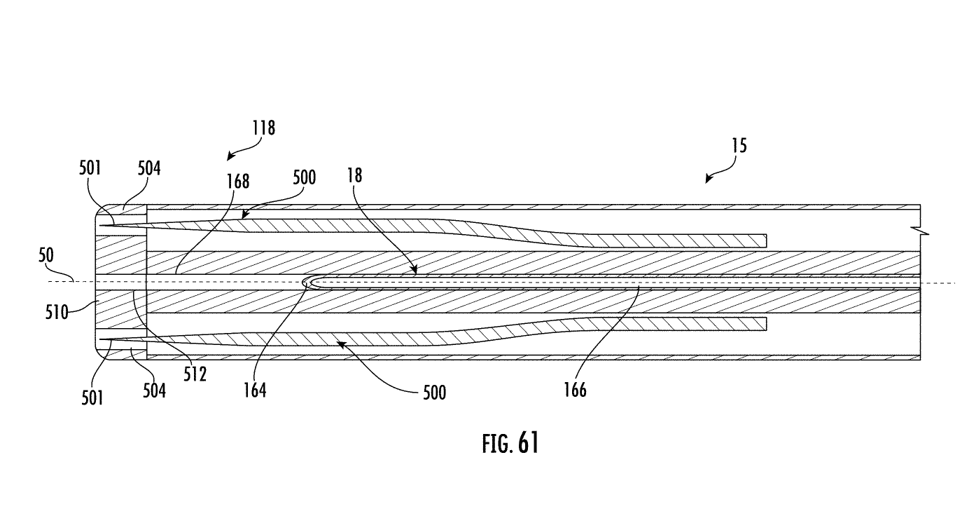

With reference to FIG. 61, the distal end 118 of the insertion tube 15 may include an alignment channel 168 and/or an end cap 510 comprising an alignment opening 512, in each instance for aligning and positioning the drug delivery channel 18 during operation. As shown in FIG. 53, the alignment channel 168 may engage the drug delivery channel 18 throughout its full range of travel to prevent misalignment. Similarly, the alignment channel 168 may have a length representing only a fraction of the insertion tube 15 or it may extend over a significant majority of the length. In some embodiments the alignment channel 168 and/or the end cap 510 may seal the end of the insertion tube 15 to prevent treatment agent or bodily fluid from entering the applicator 110.

Turning to FIG. 86, another example applicator 1000 is shown having a steerable insertion tube 1015. The applicator 1000 includes a steering mechanism to provide additional control of the applicator, particularly where applicator has a flexible body. For example, applicator 1000 may include one or more cables extending from the control portion 1014 to the distal end 1018 of the insertion tube 1015 to allow a user to steer the distal end 1018, the electrodes 500 and the delivery channel 18 to the target site within the patient. The insertion tube 1015 may include a flexible portion 1005 and a rigid portion 1010 to allow only the desired portions of the applicator to bend during steering (e.g., the cables may be offset from the axial center of the insertion tube such that applying a force to one or more cables bends the flexible portion 1005 in the direction of the cable(s)). The cables may be attached to the applicator at or near the control portion 1014 and between the rigid portion 1010 and the first distal end 1018 to bend the flexible portion 1005 upon application of a force to the cables from the control portion.

The applicator 1000 may include electrodes 500, a delivery channel 18, a control portion 1014, and an actuator 1012, which may include the features, structure, and operation of any of the electrodes, control portions, actuators, and delivery channels described herein, such as those of applicators 14, 60, 70, 110, and which may cooperate with the other components of an electroporation system disclosed herein including a generator and drug delivery device. The insertion tube 1015 and steerable components may be substituted for the insertion tubes 15 of any other embodiment discussed herein as if each individual feature had been described with respect to each embodiment, and such features may operate in accordance with their intended purpose in such combined embodiments. The insertion tube 1015 may comprise any of the dimensions or configurations of the insertion tubes 15 described herein with the addition of steerable components.

In some embodiments, the applicator 1000 may be a steerable laparoscopic applicator. As described herein, a steerable laparoscopic applicator can be used an alternative to an endoscopic applicator. For example, in some embodiments, the applicator 1000 may gain access to the interior anatomy via a trocar. The rigid portion 1010 of the insertion tube 1015 may allow for easy maneuverability, while the flexible portion 1005 enables steering via the cables. The applicator 1000 may have a knob that can be rotated which triggers movement of the tip of the applicator up and down to 120 degrees or less relative to the rigid portion 1010 in each direction. In some embodiments, the steerable tip may move 90 degrees or more in two or more directions (e.g., up and down).

In some embodiments, as discussed herein, the endoscope may be a trocar, flexible cannula, or other insertion instrument for insertion into a patient. In some embodiments, the applicator 14 may be a steerable device (e.g., the laparoscopic applicator 1000 shown in FIG. 86) that may be inserted into a patient without a separate insertion device. In some embodiments, the applicator may be radiopaque at its distal end.

The working channels of endoscopes used for various endoscopies (e.g., working channel 54 of endoscope 52 shown in FIG. 4) may have a limited diameter through which one or more portions of the electroporation system 10 may be inserted to reach the endoscopic site (e.g., adjacent distal end 56 of the endoscope 52 shown in FIG. 4). In embodiments that include an endoscope as part of the system, the portions of the electroporation system 10 that extend into the endoscope must fit within the working channel of the endoscope. For example, in some instances, such as with bronchoscopy, the working channel of the endoscope may be 2.2 mm or smaller in diameter, and the portions of the electroporation system 10 that enter the endoscope (e.g., the insertion tube 15) may be 2.0 mm or smaller in diameter. In some embodiments, the working channel of the endoscope may be 4 mm or smaller in diameter. In some embodiments, the insertion tube 15 is flexible to follow any curves or bends in the working channel of the endoscope.

In some embodiments, the applicator 14 may include at least two electrodes 100 at the distal end of the insertion tube 15 (e.g., the end opposite the control portion 48, 72, 114) with one or more wires or other conductive material extending from the generator 12 (shown in FIG. 1) to the electrodes 100 via the insertion tube 15. In some embodiments (e.g., as described below with respect to FIGS. 47-67), the applicator 14 may also include other components, such as a drug delivery channel 18, that extend through the insertion tube 15 from a drug delivery device 16 (shown in FIG. 1) to the distal end of the insertion tube 15. In such embodiments, the wiring for the electrodes 100 and the drug delivery channel 18 may run parallel to each other down the insertion tube 15 from the control portion (e.g., control portion 48 shown in FIG. 2; control portion 72 shown in FIG. 19; or control portion 114 shown in FIG. 47) of the applicator 14 to the distal end. In some embodiments, applicator 60, 70, 110, 1000 may include the aforementioned features.

In some embodiments, the applicator 14 may include at least two electrodes 100 that extend through the insertion tube 15 to the distal end, and a separate drug delivery applicator 19 may deliver a plasmid, drug, and/or other treatment agent to the electroporation site. The drug delivery applicator 19 may administer the one or more treatment agents sequentially with the electroporation or concurrently through different channels or vectors. In some embodiments, applicator 60, 70, 110, 1000 may include the aforementioned features.

For example, in a system with an endoscope, once the endoscope is in position within the patient, the drug delivery applicator 19 may first be inserted into the endoscope until a distal end of the drug delivery applicator 19 reaches the target electroporation site (e.g., a tumor or other visceral lesion) at or adjacent to the distal end of the endoscope, after which the treatment agent(s) may be administered. The drug delivery applicator 19 may then be removed and replaced in the endoscope by the applicator 14 for electroporation, and the target electroporation site may be electroporated to facilitate permeation of the treatment agent(s) into the cells.

In some embodiments, one or more treatment agents may be administered through other means instead of or in addition to administering treatment agent(s) via the endoscope or drug delivery applicator 19. For example, one or more treatment agents may be administered via intramuscular (IM), intrathecal (IT), or intravenous (IV) injections before, during, or after electroporation.

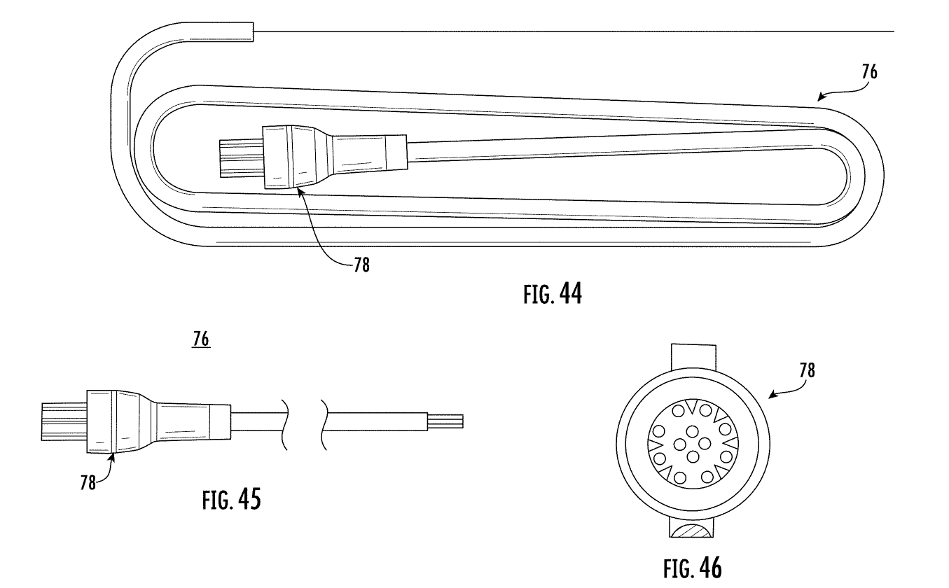

With reference to FIGS. 44-46, a cable 76 and corresponding connector 78 are shown for connecting an applicator 14, 60, 70, 110, 1000 to a generator 12.

In some embodiments, an applicator may include an actuator that remains physically stationary when actuated. For example, the actuator may be a button on a touchscreen display that is operable to control deployment of the electrodes within the insertion tube. The touchscreen may include a sensor (e.g., a pressure, capacitive touch, and/or gesture sensors) to detect contact with the screen and thereby control whether a circuit linked to a control element in the applicator causes the control element to move axially in response to opening and closing of the circuit. The element may be physically associated with the electrodes so that axial movement of the control element occurs with axial movement of the electrodes. In some examples, the circuit may be configured to cause the electrodes to move directly in response to opening and closing of the circuit. In some embodiments, actuation of the applicator may occur on a remote device linked to the applicator via a wireless connection. In this arrangement, a signal from the actuator is received in the applicator to control movement of the electrodes. In some examples, a drug delivery channel axially fixed relative to the electrodes may be simultaneously controlled through this electronic actuation means. In other examples, a second electrical control (e.g., touchscreen) may be included to control deployment of the drug delivery channel separately from the electrodes.

### Electrode Deployment

During electroporation, the distance between electrodes (e.g., electrodes 100) may affect the size of the treatment area and the required amplitude, frequency, and/or wavelength of the electrical signals needed for electroporation. The working channel size in the endoscope or in the insertion tube of the applicator may limit the spacing between electrodes because the electrodes must fit within the working channel, and thus the size of the electroporation treatment area may be restricted during endoscopic therapies in ways not required in non-endoscopic methods and apparatus or non-minimally-invasive procedures.

In some embodiments of the present disclosure, the applicator 14 and electrodes 100 may be structured such that the electrodes are able to be deployed to a spacing wider than the working channel in an instance in which the electrodes are able to clear the distal end of the endoscope. In some embodiments, the electrodes 100 may expand wider than an opening (e.g., a keyhole opening) at a point of access in the patient. In some embodiments, the electrodes 100 may expand wider than a distal end of the insertion tube 15. In some embodiments, the electrodes 100 may expand wider than one or more channels (e.g., channels 204, 404, etc.) in the insertion tube 15. In some embodiments, the electrodes may expand to a spacing about equal to the distal end of the insertion tube 15 or about equal to a width of the one or more channels. In some embodiments, the electrodes may expand to a spacing less than the distal end of the insertion tube 15. In some embodiments, an actuator 42, 74, 112 may extend through or onto the insertion tube 15 of the applicator 14 and may be configured to apply an axial force (e.g., a force having a component along the longitudinal axis of the insertion tube 15) to the electrodes 100. This axial force may cause the electrodes to extend axially and/or radially outwardly from the distal end of the insertion tube 15 of the applicator 14 to electroporate the target tissue at the electroporation site. In some examples, the manner of expansion of the electrodes may be a function of the space available in view of the cross-sectional size of the insertion tube and the electrode position within the tube in the retracted position. In one specific example, an applicator with electrodes very close together in the retracted position may include a radially expanding deployment of such electrodes so that the electrode tips reach a spacing necessary for the safe and effective operation of the applicator upon deployment (e.g., minimize the possibility of electrical arcing between the electrodes).

In some embodiments, insertion tube 15 may define a diameter of about 2 mm. In a retracted position, stored within the insertion tube 15, the tips of the electrodes 100 may be spaced about 1.8 mm apart. In the deployed position, the tips of the electrodes 100 may be spaced about 3 mm apart. In some embodiments, in the deployed position, the tips of the electrodes 100 may be spaced greater than the external diameter of the distal end of the insertion tube. In some embodiments, in the deployed position, the tips of the electrodes 100 may be spaced greater than the external diameter of a distal end of the insertion device (e.g., endoscope). In some embodiments, in the deployed position, the tips of the electrodes 100 may be spaced greater than 2 mm. In some embodiments, in the deployed position, the tips of the electrodes 100 may be spaced greater than 3 mm. In some embodiments, in the deployed position, the tips of the electrodes 100 may be spaced from 2 mm to 3 mm. In some embodiments, in the deployed position, the tips of the electrodes 100 may be spaced about 4 mm. In some embodiments, in the deployed position, the tips of the electrodes 100 may be spaced greater than 4 mm. In some embodiments, in the deployed position, the tips of the electrodes 100 may be spaced less than 4 mm. In some embodiments, in the deployed position, the tips of the electrodes 100 may be spaced greater than 5 mm. In some embodiments, in the deployed position, the tips of the electrodes 100 may be spaced from 3 mm to 5 mm. In some embodiments, in the deployed position, the tips of the electrodes 100 may be spaced from 2 mm to 5 mm. In some embodiments, in the deployed position, the tips of the electrodes 100 may be spaced about 5 mm. In some embodiments, in the deployed position, the tips of the electrodes 100 may be spaced less than 5 mm. In one particular example, the electrode spacing may preferably be about 5 mm or less for an applicator described in conjunction with low voltage generator electroporation. In any of the above configurations, either low or high voltage electroporation may be performed.

In some embodiments, the electrodes 100 may be made of stainless steel and coated with gold. In some embodiments, the electrodes 100 may be substantially flexible, having a similar structure to acupuncture needles. The electrodes 100 may be 0.25 mm in diameter in some embodiments. The electrodes 100 may extend about 6 mm in length in some embodiments. In some embodiments, the diameter and length of the electrodes may vary from the specific dimensions described herein. In some embodiments, the actuator 42, 74, 112 and remaining non-metallic components of the applicator 14, 60, 70, 110, 1000, such as the body 90, 120 and end caps 88, 122, may be made of a plastic material (e.g., high-density polyethylene, braided polyurethane (FEP, PEEK, etc.), etc.).

With reference to FIGS. 2, 19, 47, and 86, as detailed above, the applicator 14, 60, 70, 110, 1000 may include the insertion tube 15, 1015, the control portion 48, 72, 114, 1014, and the actuator 42, 74, 112, 1012. The actuator 42, 74, 112, 1012 may include a trigger 44, switch 80, 116 or other actuating element and a pushing element 46, 92, 128 that may be rigid in some embodiments, and sufficiently flexible to bend with a flexible endoscope in some embodiments. For example, in some laryngeal applications, the insertion tube 15 and actuator 42, 74, 112 may be rigid. With reference to FIG. 2, the trigger 44 may be pivotally attached to the control portion 48 and the pushing element 46 such that pulling the trigger forces the pushing element 46 along the insertion tube 15 of the applicator 60 towards the endoscopic site at the distal end of the insertion tube 15, and extending the trigger 44 (e.g., moving the trigger back to the position shown in FIG. 2) will retract the pushing element 46 back towards the control portion 48. With reference to FIG. 19, the switch 80 may be slidingly attached to the control portion 72 and the pushing element 92 via hollow mandrel 86 such that sliding the switch forces the pushing element 92 along the insertion tube 15 of the applicator 70 towards the endoscopic site at the distal end of the insertion tube 15, and retracting the switch 80 (e.g., moving the switch back towards the user) will retract the pushing element 92 back towards the control portion 72. With reference to FIG. 47, the switch 116 may be slidingly attached to the control portion 126 and hollow mandrel 124 such that sliding the switch forces the pushing element 128 along the insertion tube 15 of the applicator 110 towards the endoscopic site at the distal end of the insertion tube 15, and retracting the switch 116 (e.g., moving the switch back towards the user) will retract the pushing element 128 back towards the control portion 114.

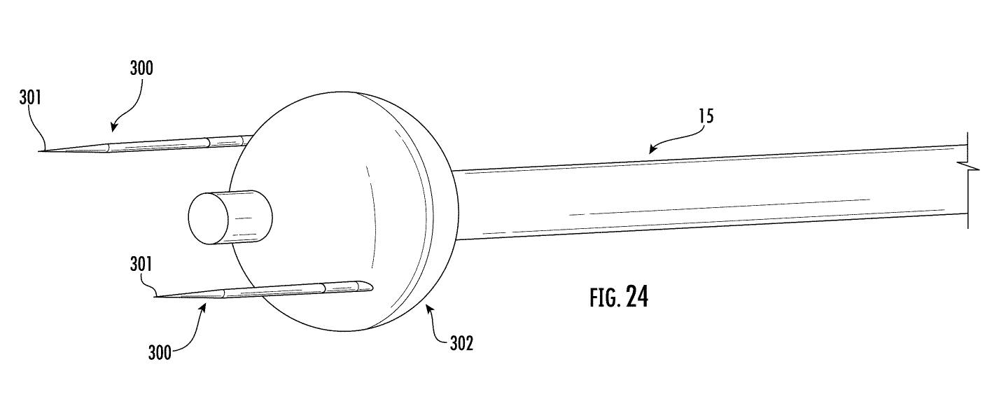

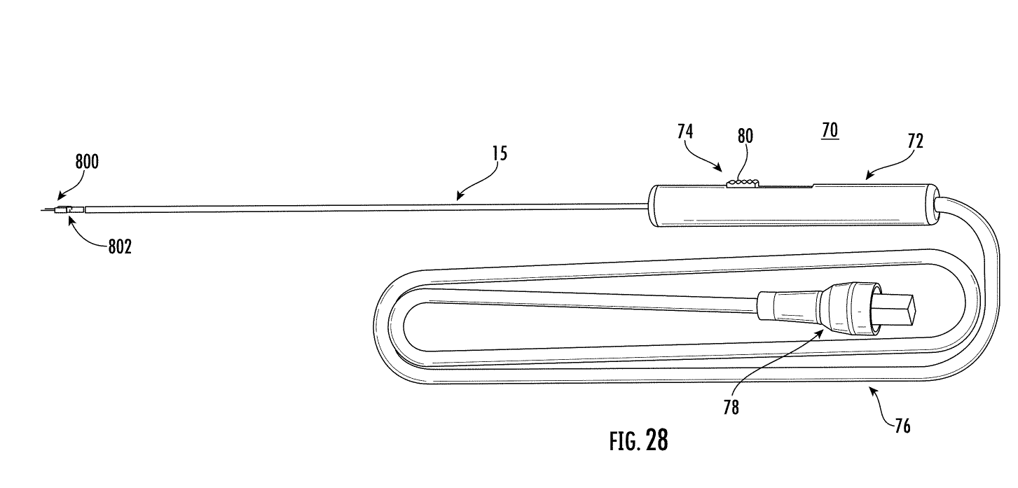

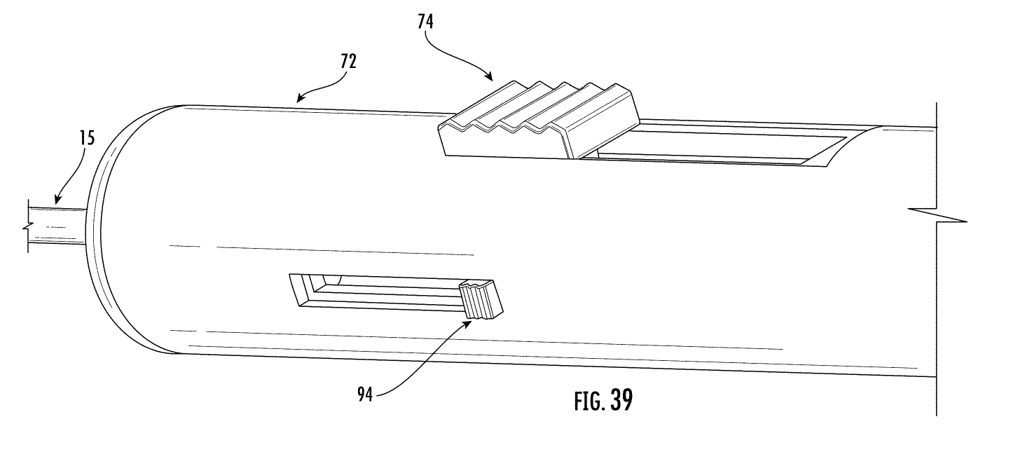

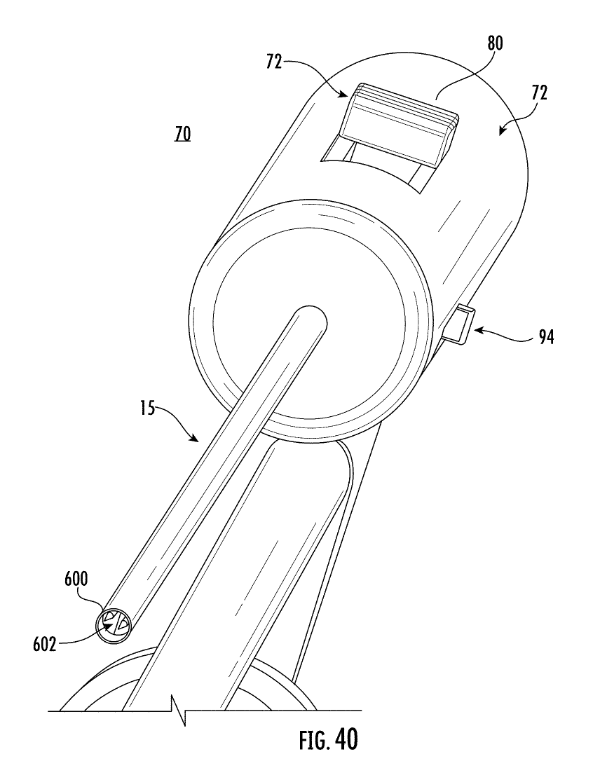

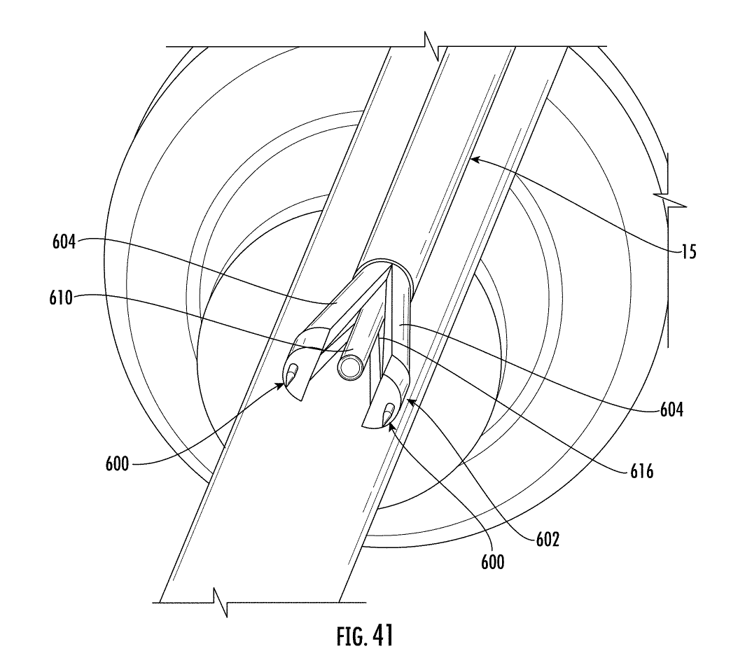

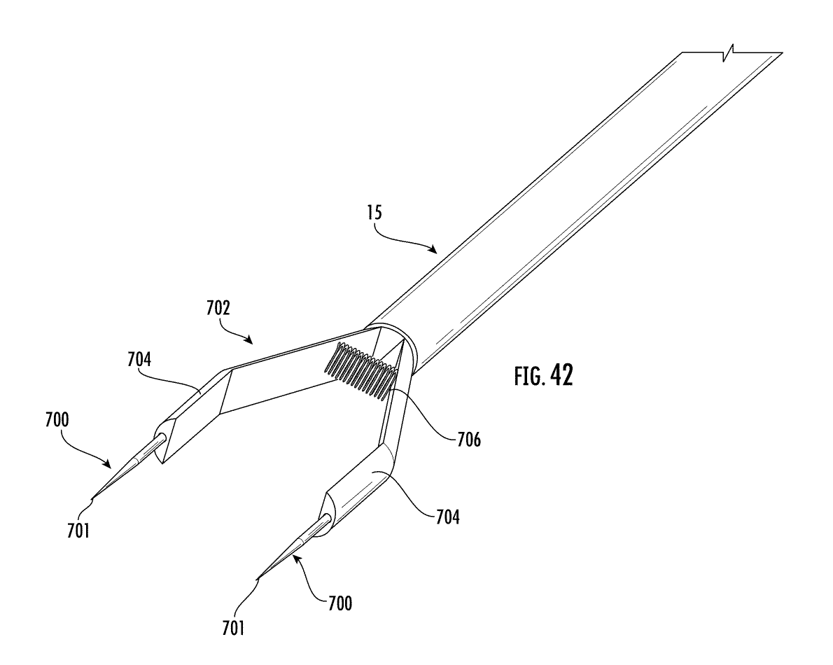

Turning to FIGS. 5-41 and 60-66, several embodiments of the distal end assemblies of the insertion tube 15 of the applicator 14 are shown. In each embodiment, the electrodes may be driven axially and radially outwardly to create a greater spacing between the ends of the electrodes. In some embodiments, when moved to a deployed position, the ends of the electrodes are spaced farther apart than the external diameter of the insertion tube 15 of the actuator 14. In some embodiments, when moved to a deployed position, the ends of the electrodes are spaced farther apart than the internal diameter of the working channel (e.g., working channel 54 of the endoscope 52 shown in FIG. 4). In each embodiment, the electrodes may be directly or indirectly actuated by the actuator via the pushing element in both the outward (e.g., deploying) and inward (e.g., retracting) directions.

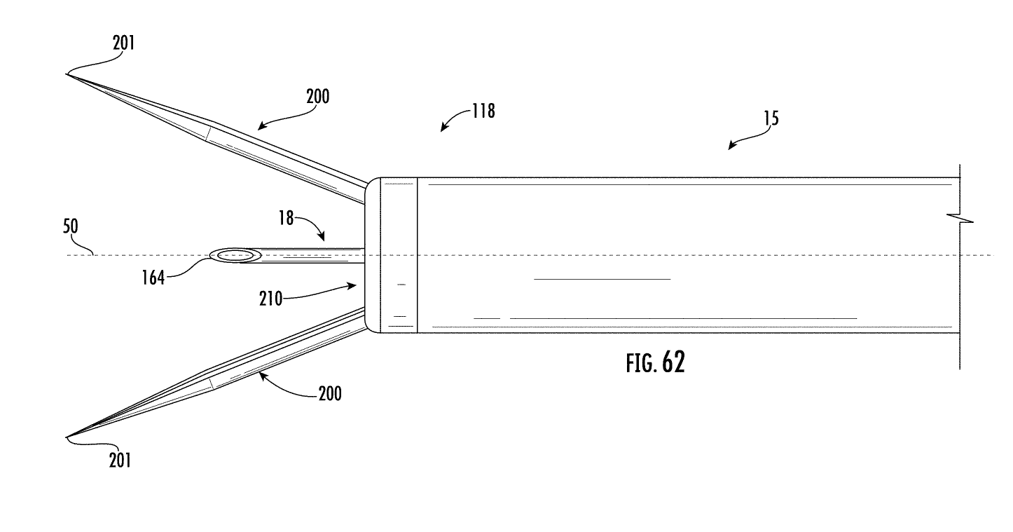

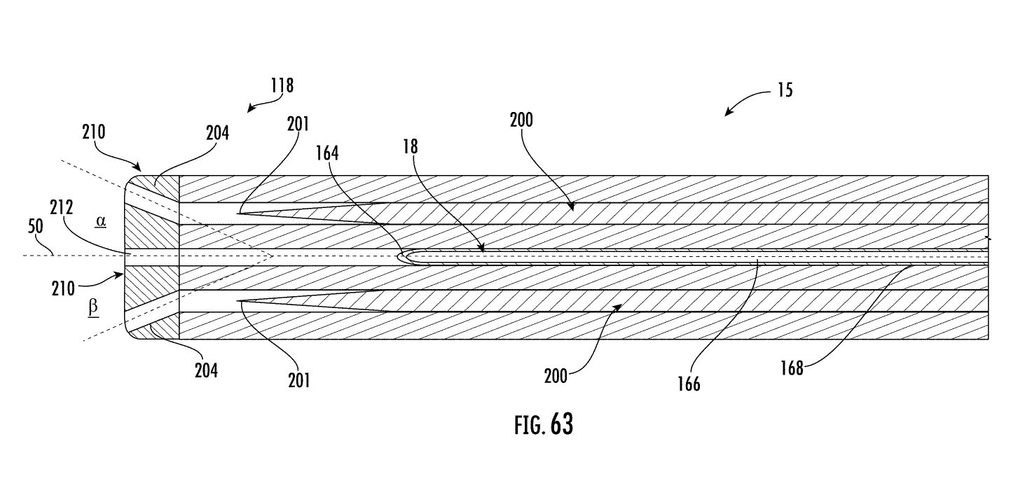

With reference to FIGS. 5, 6, 20-21, 62, 63, a pair of electrodes 200 are shown having a retracted position (FIGS. 5, 21, 63) and a deployed position (FIGS. 6, 20, 62) in accordance with some embodiments described herein. The electrodes 200 may each include a tip 201 at a distal end thereof opposite the insertion tube 15. The tip 201 of the electrodes 200 may define a pointed end configured to pierce the target tissue for electroporation. In the depicted embodiment, the applicator 14, 110 includes an end cap 202, 210 at the distal end of the insertion tube 15 having at least two angled channels 204 defined therein. The two angled channels 204 in the depicted embodiment are configured to angle the electrodes outwardly in the deployed position (FIGS. 6, 20, 62) so that the spacing between the ends of the electrodes increases. The embodiment of FIGS. 62 and 63 depicts another embodiment of the insertion tube 15 and end cap 210 through which the electrodes 200 may extend via the angled channels 204, and which also depict an alignment opening 212 and alignment channel 168 to support a drug delivery channel 18 therein. The embodiment of FIGS. 62-63 depicts the embodiment of FIGS. 5, 6, 20-21 having an insertion tube 15 with a drug delivery channel 18 extending therethrough. The drug delivery channel 18 and electrodes 200 may be operated and structured in accordance with any of the embodiments herein.

With reference to FIGS. 5 and 63, the angled channels 204 are oriented at respective angles α, β relative to the longitudinal axis 50. In some embodiments, the angles α, β may be equal, such that the electrodes 200 are oriented at substantially mirrored angles relative to the axis 50 in the deployed position. In some embodiments, the angles α, β may be slightly different, but extend in different directions relative to axis 50. In some embodiments, the angles α, β are each acute, such that when the pushing element 46 applies an axial force, directly or indirectly, on the electrodes 200 towards the end cap 202, 210, the angle of the channels 204 pivots the electrodes to angle the electrodes in the direction of the channels 204 as the electrodes extend outwardly from the end cap 202 into the deployed position shown in FIGS. 6 and 62. Similarly, when the pushing element 46, 128 retracts back towards the control portion 48, 114 as described above, the electrodes 200 may be pulled back into the end cap 202, 210 of the applicator 14, 110 and into the internal cavity of the insertion tube 15, allowing the electrodes to reorient within the insertion tube 15. Thus, in the retracted position (FIGS. 5, 21, 63), the electrodes 200 are substantially parallel to each other, and in the deployed position, at least a portion of the electrodes 200 are at an angle (e.g., α+β) to each other as defined by the angled channels 204 as a result of the actuator pushing the electrodes into the angled channels.

In any of the embodiments discussed herein, the electrodes (e.g., a needle) may be made of a sufficiently flexible material to allow the electrodes to bend when moving between the retracted and the deployed positions. In some embodiments, the electrodes 100 may be made of stainless steel and coated with gold. For example, in some embodiments, the electrodes may be substantially the same as acupuncture needles. With reference to FIG. 21, a carrier 206 may fixedly hold the electrodes 200 such that the electrodes protrude a predetermined distance from the carrier (e.g., 5 mm). In such embodiments, the electrodes 200 may bend when passing through the end cap 202, 210 along the angled channels 204 such that the distal end of the electrodes is oriented in the direction of the angled channels while the bases (opposite the distal end) of the electrodes remain parallel. In any of the embodiments herein including an electrode carrier, the carrier may include passages for disposal of electrodes therein and a further passage for the disposal of a drug delivery channel.

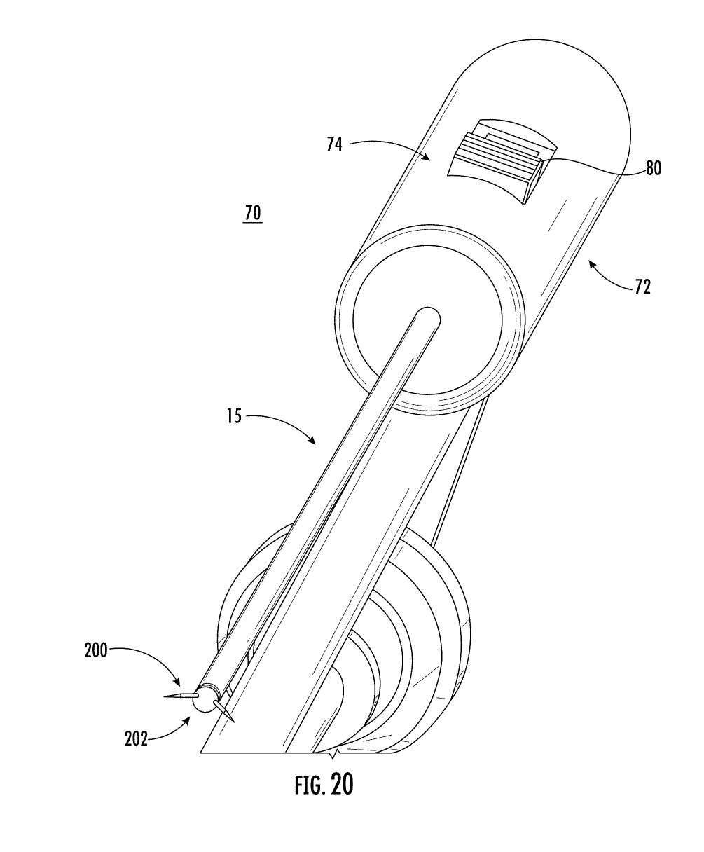

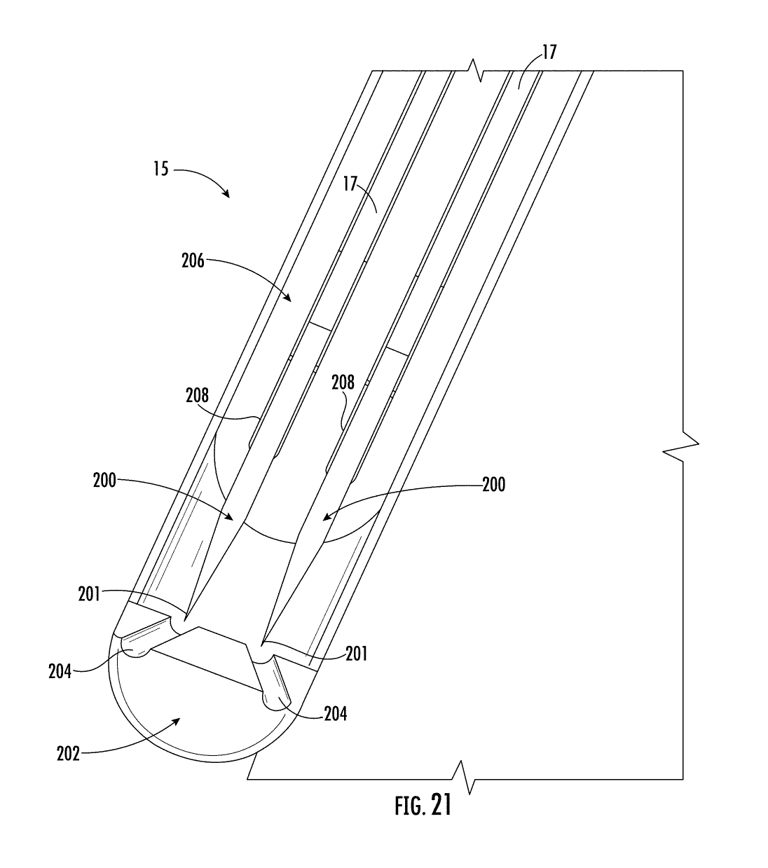

In the embodiment shown in FIGS. 20-21, the carrier 206 is actuated between the retracted and deployed positions by the pushing element 92 (shown in FIGS. 33, 36), which pushing element may abut a proximate, rear surface of the carrier opposite the distal end. The electric wires 17 which supply the electric signals to the electrodes 200 may pass through a channel 208 in the carrier 206 to connect the generator to the electrodes. In some embodiments, the pushing element 92 may be fixed to the carrier 206.

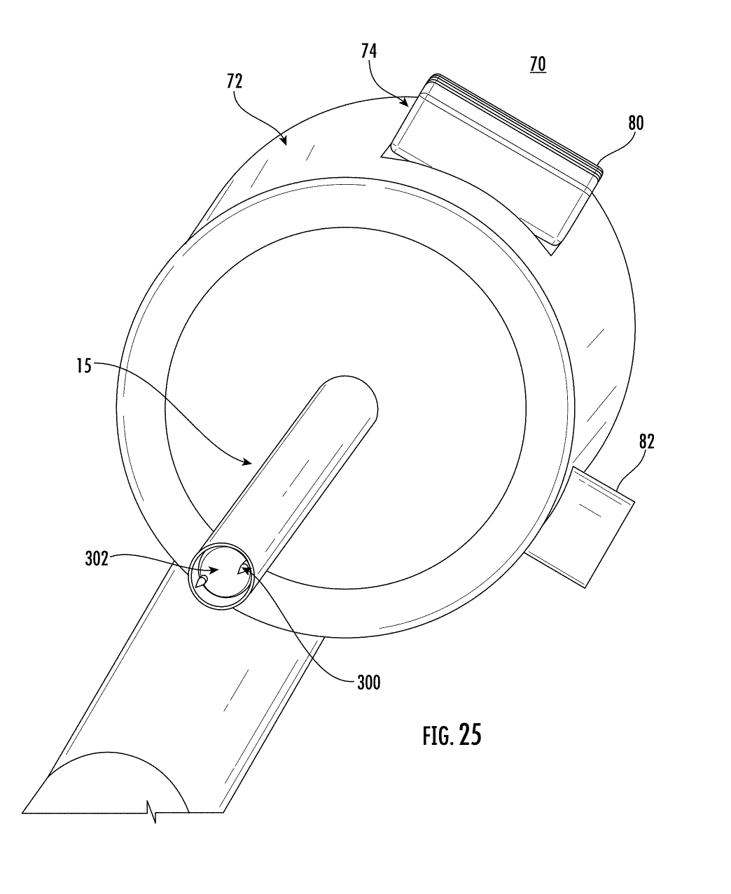

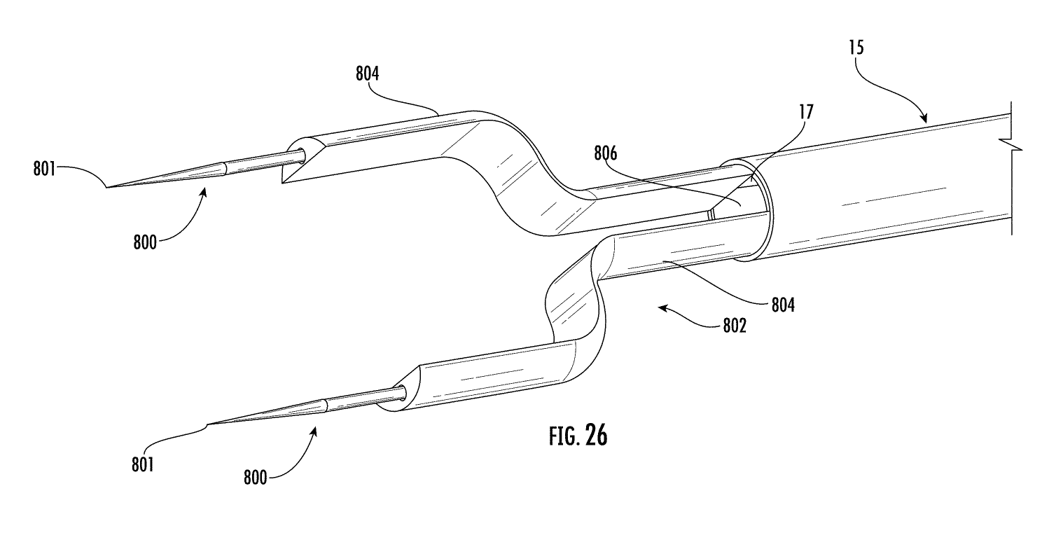

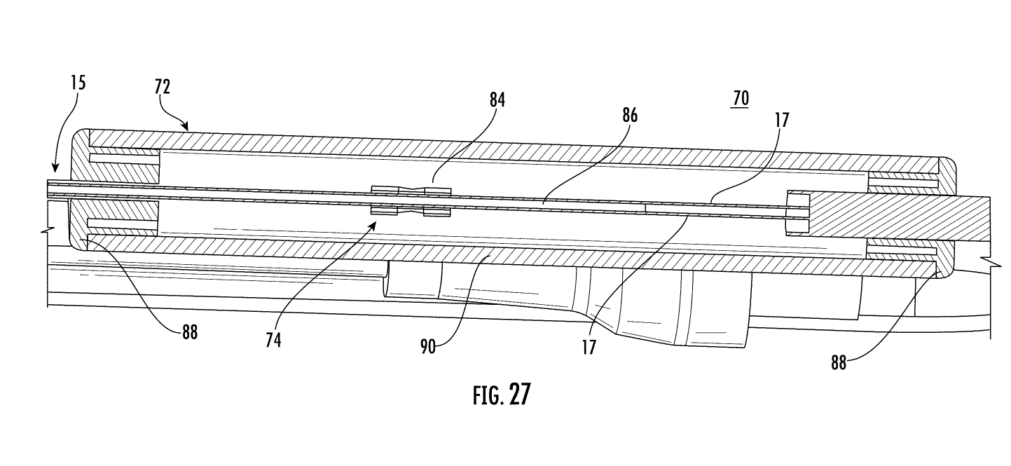

With reference to FIGS. 7, 8, and 22-25, a pair of electrodes 300 are shown having a retracted position (FIGS. 7, 25) and a deployed position (FIGS. 8, 22, 24) in accordance with some embodiments described herein. The electrodes 300 may each include a tip 301 at a distal end thereof opposite the insertion tube 15. The tip 301 of the electrodes 300 may define a pointed end configured to pierce the target tissue for electroporation. In the depicted embodiment, the applicator 14, 60, 70, 110, 1000 includes an expandable bladder 302 in which ends of the electrodes 300 are embedded. In some embodiments, the bladder may be made of a flexible, elastic material such as rubber. In use, the bladder 302 may be retracted and compressed within the insertion tube 15 the retracted position (FIG. 7, 25). In the retracted position, the electrodes 300 are positioned close together at a distance less than the internal diameter of the insertion tube 15 because the bladder 302 is compressed radially inwardly by the insertion tube 15.

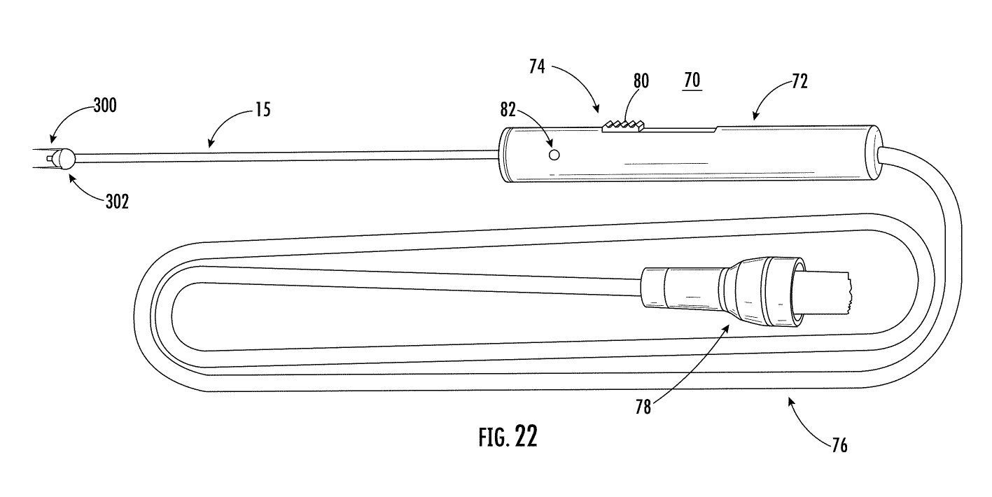

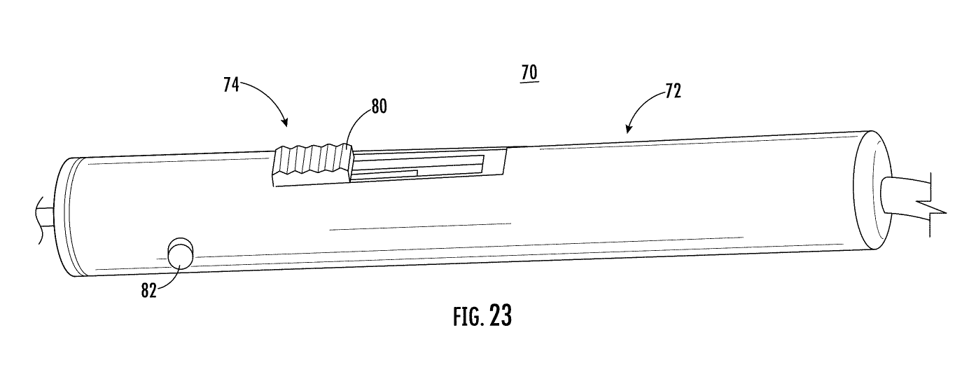

In operation, the pushing element 46, 92 applies an axial force, directly or indirectly, on the bladder 302 and causes the bladder to exit the distal end of the insertion tube 15. Upon clearing the distal end of the insertion tube 15, the bladder 302 may expand into a deployed shape (e.g., a substantially spherical shape). In some embodiments, the bladder 302 may expand by pneumatic pressure supplied from an air supply upstream of the bladder 302 (e.g., via a conduit running through the applicator). For example, with reference to FIGS. 22-23, the control portion 72 may include a secondary button 82 to activate a pneumatic supply to inflate the bladder 302. In some embodiments, the bladder 302 may expand mechanically due to the elastic restorative force of the bladder returning to its natural, expanded shape with or without pneumatic assistance. Similarly, when the pushing element 46 retracts back towards the control portion 48 as described above, the electrodes 300 may be pulled back into the insertion tube 15 of the applicator 14, causing the bladder 302 to recompress and deform and causing the electrodes 300 to move closer together.

The electrodes 300 may be parallel in both the retracted (FIG. 7) and expanded (FIG. 8) positions. In some embodiments, the electrodes 300 may be angled in either or both of the retracted and expanded positions. For example, the electrodes may be mounted at any position on the bladder 302 and at any desired orientation (e.g., angled outwardly, similar to the embodiment of FIGS. 5-6).

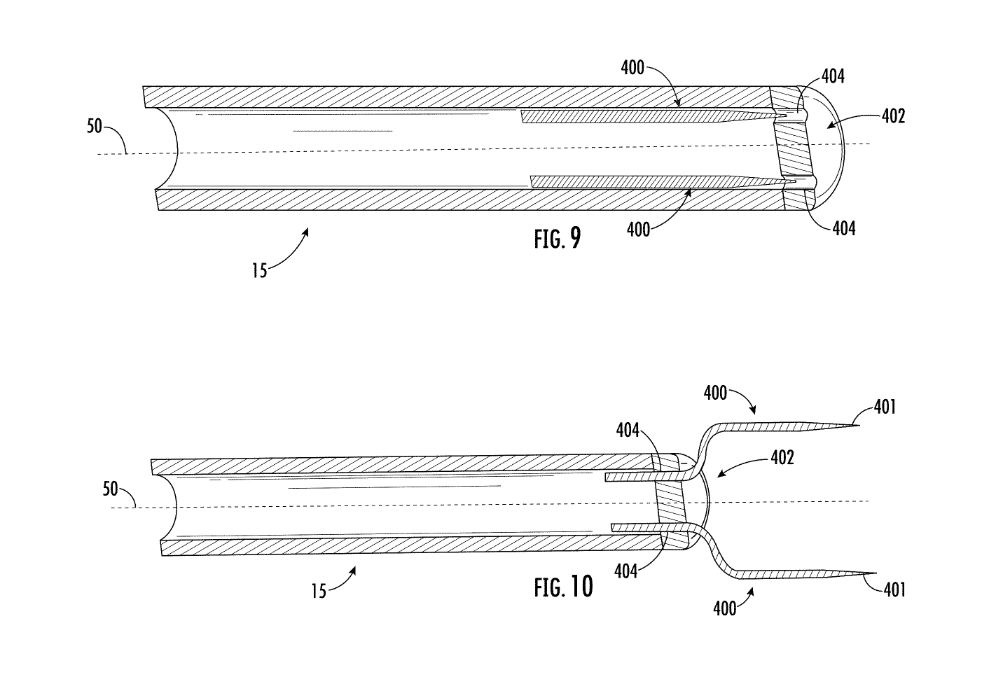

Turning to FIGS. 9, 10, 64, and 65, another embodiment of the electrodes 400 is shown in which the electrodes 400 are made of Nickel Titanium (Nitinol). Nitinol is a shape memory alloy capable of “remembering” a programmed shape and returning to the programmed shape under certain temperature conditions. Nitinol may be programmed to a specific shape by holding the nitinol in a predetermined position (e.g., the “S” shape shown in FIG. 10) and heating the nitinol to about 500° C. (932° F.) to set the shape of the nitinol. After shape setting, the nitinol may be cooled to room temperature and mechanically deformed into a second shape (e.g., the straight shape shown in FIG. 9). During use, when the nitinol is heated above a transformation temperature, the nitinol returns to its programmed shape. The electrodes 400 may each include a tip 401 at a distal end thereof opposite the insertion tube 15. The tip 401 of the electrodes 400 may define a pointed end configured to pierce the target tissue for electroporation.

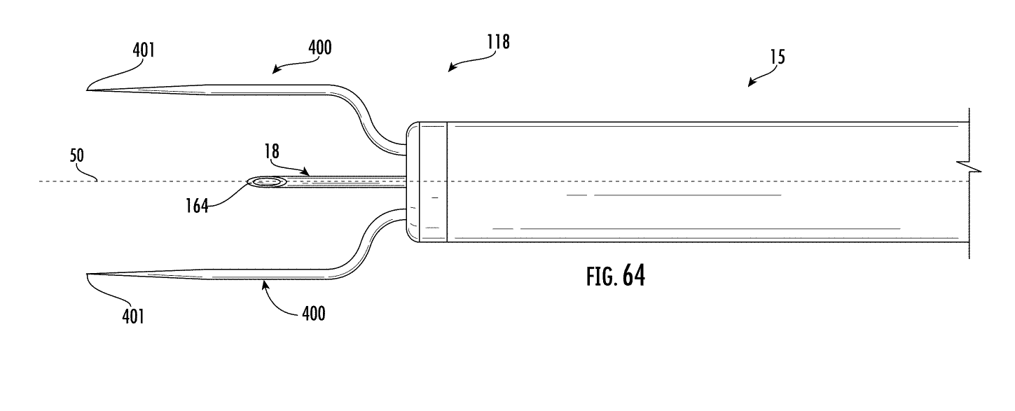

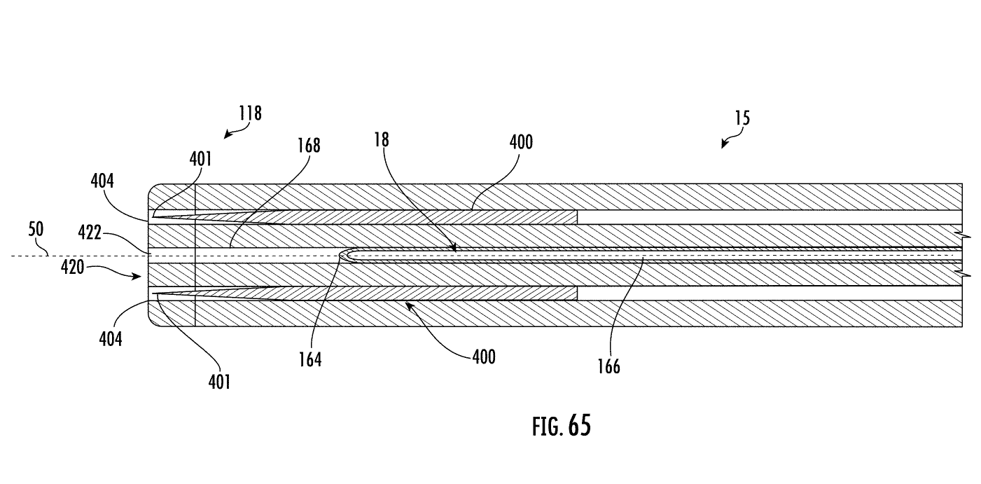

By adjusting the proportions of nickel and titanium in the Nitinol, the transformation temperature (e.g., the temperature at which 50% of the nitinol changes from the shape shown in FIGS. 9, 65 to the position shown in FIGS. 10, 64) of the nitinol may be tuned relative to human body temperature, such that the Nitinol changes shape upon coming into contact with the temperature of the patient's body tissue. In use, nitinol may have a “start” temperature and a “finish” temperature at which the transformation begins and ends, respectively. In some embodiments, the finish temperature may be less than or equal to body temperature. For example, in some embodiments, the nitinol may include 54.5% nickel and 45.5% titanium, which may have a transformation temperature of 60° Celsius. In some embodiments, the transition temperature of the Nitinol may be human body temperature. Alternatively, rather than relying on the body temperature of the patient to warm the Nitinol, the electrodes 400 may instead change shape upon a voltage passing through it, whether it be the actual voltage being used for electroporation, or some amount of pre-voltage, such as a smaller voltage with a sole intended use of assisting the electrodes to change shape. Once the shape has been changed, the standard voltage may be passed through the electrodes.

The pushing element 46, 128 may deploy the electrodes 400 by applying an axial force, directly or indirectly, on the electrodes 400 towards the distal end and end cap 402, 420 when the electrodes 400 are in their deformed, substantially straight shape (e.g., the shape shown in FIGS. 9, 65). The pushing element 46, 128 may cause the electrodes 400 to translate axially through the channels 404 end cap 402, 420 until a portion of the electrodes extends from the distal end of the applicator 14. In some embodiments, the channels 404 may be substantially parallel to the axis 50 of the applicator 14. Upon changing temperature above the transformation temperature, the electrodes 400 may change shape to their programmed shape in which the electrodes are curved outwardly to widen the spacing between the ends of the electrodes as shown in FIGS. 10, 64. The embodiment of FIGS. 64 and 65 depicts another embodiment of the insertion tube 15 and end cap 420 through which the electrodes 400 may extend via the channels 404, and which also depict an alignment opening 422 and alignment channel 168 to support a drug delivery channel 18 therein. The embodiment of FIGS. 64 and 65 depicts the embodiment of FIGS. 9-10 having an insertion tube 15 with a drug delivery channel 18 extending therethrough. The drug delivery channel 18 and electrodes 400 may be operated and structured in accordance with any of the embodiments herein.

In some embodiments, the tips 401 of the electrodes 400 may be substantially parallel to each other in both the retracted (FIGS. 9, 65) and deployed (FIGS. 10, 64) positions, while the middle sections of the electrodes curve into an “S” shape when transitioning from the retracted position to the deployed position. Similarly, when the pushing element 46, 128 retracts back towards the control portion 48, 114 as described above, the electrodes 400 may be pulled back into the end cap 402 of the applicator 14 and into the cavity of the insertion tube 15, causing the nitinol to mechanically deform back into a substantially straight position when the nitinol is forced against the channels 404.

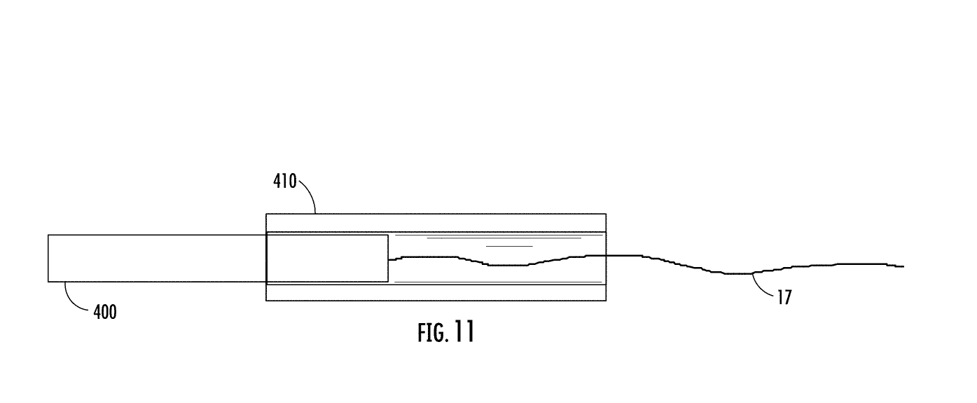

With reference to FIG. 11, in some embodiments, the electrodes 400 may engage an outer nitinol sleeve 410 and a wire 17 (e.g., separate wires 17 or wires connected to a conducting pushing element 128) running through the sleeve. For example, the electrodes 400 may be rigid needles affixed to the nitinol sleeve 410 at one end (e.g., the distal end when exiting the end cap 402) and the wire 17 may connect the electrodes to the generator (e.g., generator 12 described herein). In such embodiments, the nitinol is not required to carry the electrical signals for electroporation and instead forms a shape-changing sleeve around the conductive elements. In some embodiments, the electrodes 400 may be made of nitinol coated in a conductive material to carry an electrical signal thereon. For example, the electrodes 400 may have a nitinol structure with a nickel base coating and a gold conductive coating over the nickel coating.

FIGS. 26-30 another embodiment in accordance with the disclosure of FIG. 11. In particular, the embodiment of FIGS. 26-30 include electrodes 800 and a nitinol carrier 802 (also referred to as a sleeve) having two at least partially cylindrical halves 804 that change shape in substantially the same manner as described with respect to the embodiments of FIGS. 9-11 to position the electrodes 800 in a wider position when deployed by returning to a pre-programmed “S” shape at or above body temperature, after the actuator 74 deploys the electrodes. In some embodiments, the electrodes 800 may be attached to a straight portion of the carrier halves 804 with the wire 17 being disposed in the shape-changing portions of the carrier 802. The electrodes 800 may include tips 801 configured to extend into the target tissue.

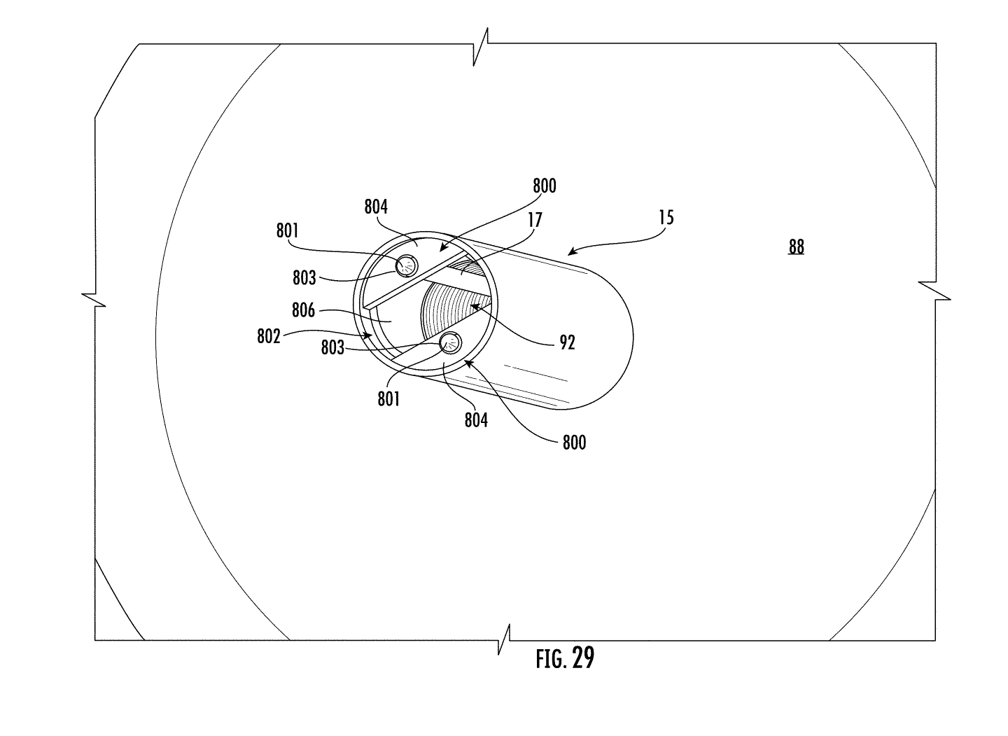

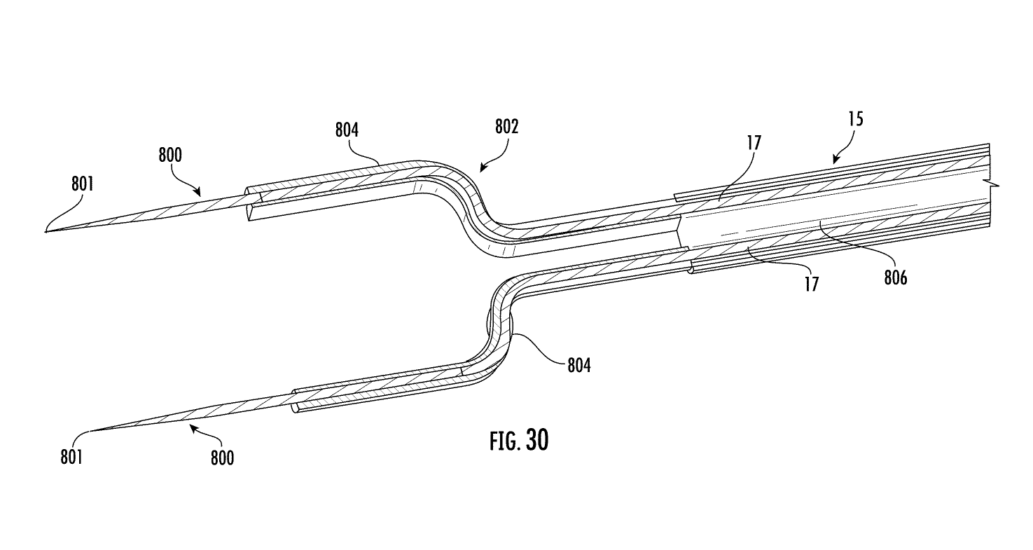

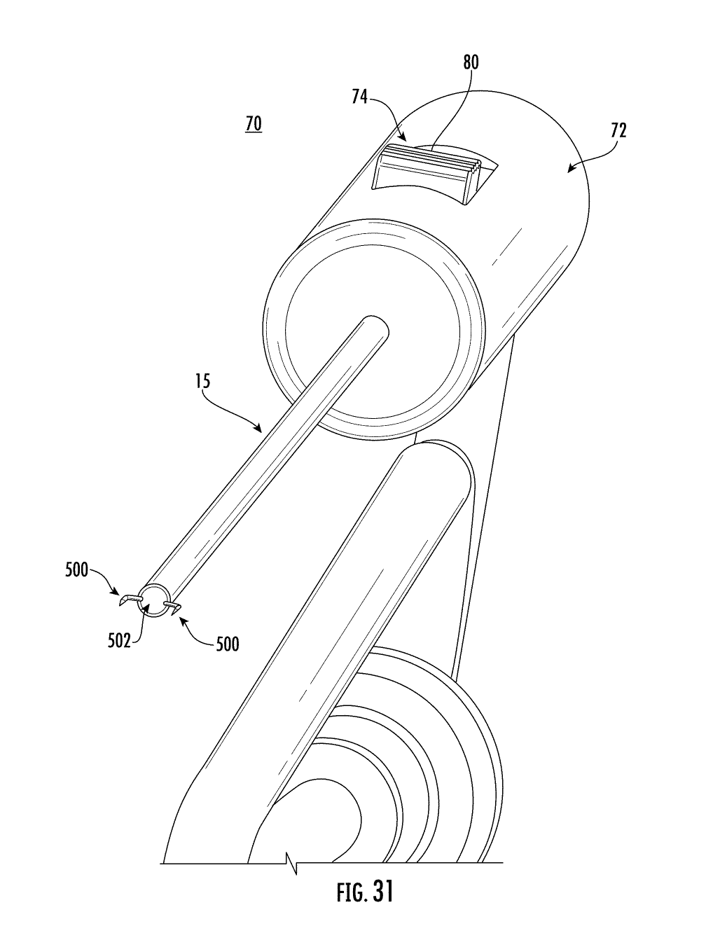

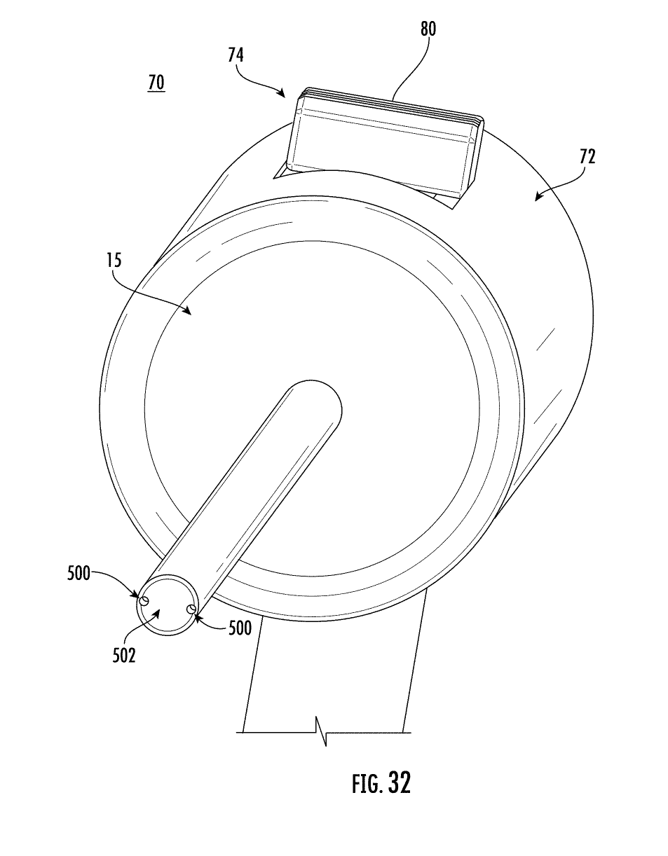

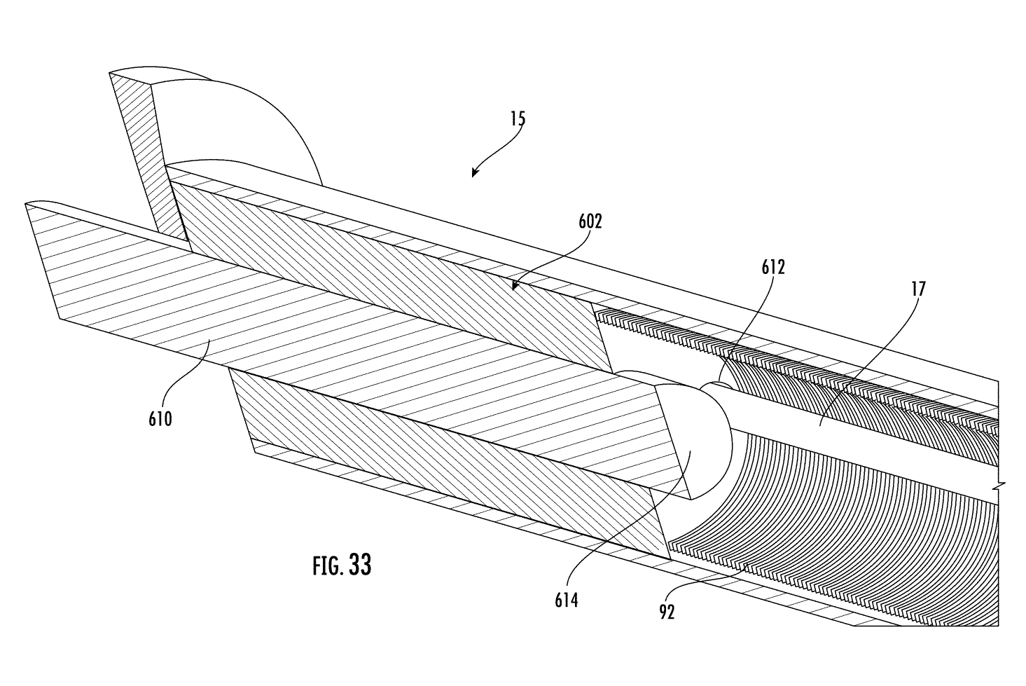

The carrier 802 may include a cylindrical portion 806 connecting the two halves 804. With reference to FIGS. 29-30, the pushing element 92 may engage the cylindrical portion 806 of the carrier 802 to actuate the electrodes 800, which electrodes may be fixedly attached to the carrier. In some embodiments, the cylindrical portion 806 may be fixed to the pushing element 92. In the depicted embodiment, the wires 17 for supplying the electrical signals from the generator may pass through the nitinol carrier 802 and may be connected to the electrodes 800 (as shown in FIG. 30). In some embodiments, the wires 17 may not be attached to the carrier 802 such that the wires may slide relative to the carrier when the carrier halves 804 change shape. In some embodiments, the nitinol carrier 802 may be 20-25 mm in length when straightened out. In some embodiments, the pushing element 92 may be fixed to the nitinol carrier 802 at a base end of the nitinol carrier.

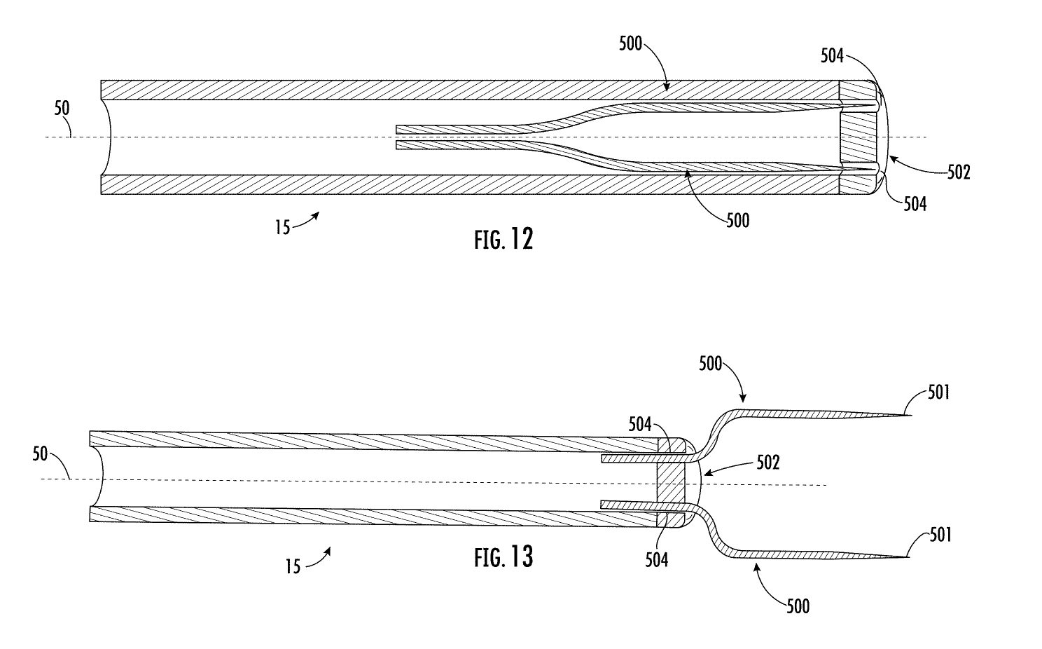

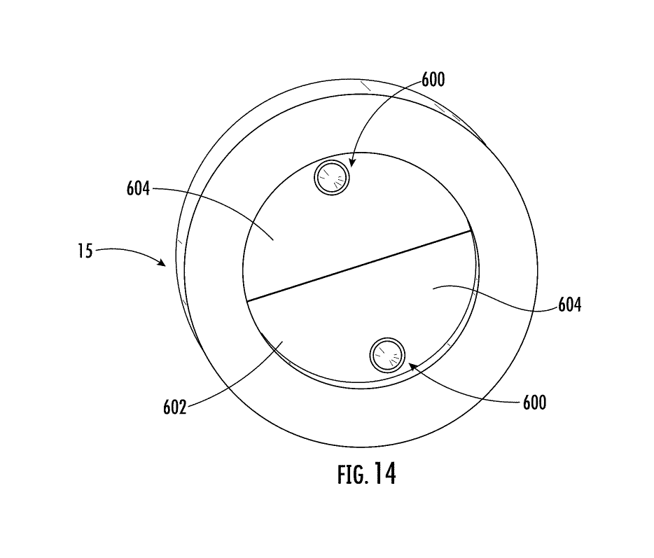

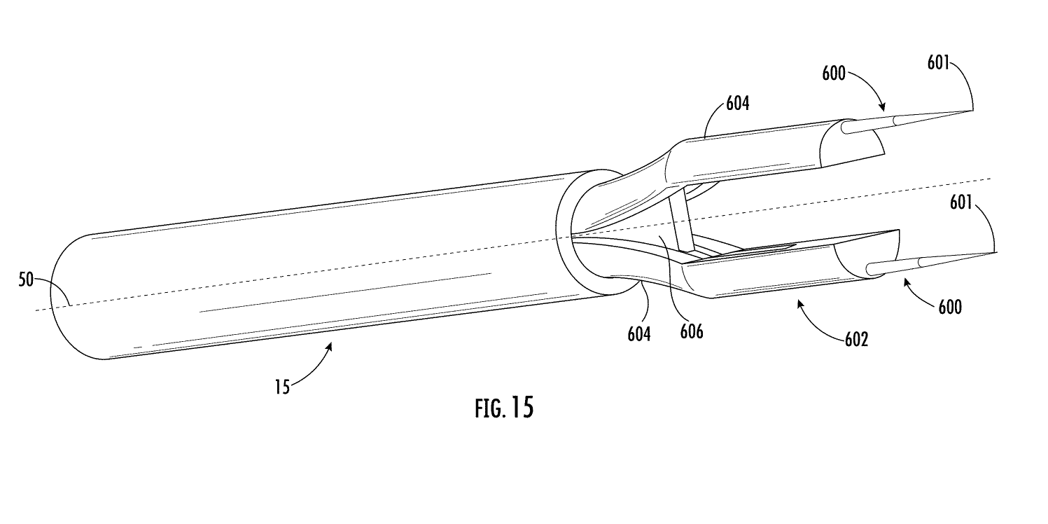

Turning to FIGS. 12, 13, 31, 32, 60, and 61, an embodiment of the electrodes 500 is shown having substantially the same deployed shape (shown in FIGS. 13, 31, 60) as the Nitinol electrodes 400 shown in FIG. 10. The electrodes 500 may each include a tip 501 at a distal end thereof opposite the insertion tube 15. The tip 501 of the electrodes 500 may define a pointed end configured to pierce the target tissue for electroporation. In the embodiment of FIGS. 12, 13, 31, 32, 60, and 61, the electrodes 500 are made of traditional conductive materials, which may be somewhat flexible, but elastically return to their original shape when stressing forces are removed. For example, as discussed above, the electrodes 500 may be made of a flexible needle having the properties of an acupuncture needle. In the depicted embodiment, the electrodes 500 are compressed radially inward in the retracted position (FIG. 12, 32, 61) and are then able to expand outwardly in the deployed position (FIG. 13, 31, 62).

The insertion tube 15 of the applicator 14, 60, 70, 110, 1000 may include an end cap 502, 510 defining channels 504 therein through which the electrodes 500 may extend. In the depicted embodiment, the electrodes 500 have a curved, “S” shape at all times, and forcing the electrodes through the end cap 502, 510 may require some deformation of the electrodes. The pushing element 46, 92, 128 may deploy the electrodes 500 by applying an axial force, directly or indirectly, towards the distal end and end cap 502, 510 of the insertion tube 15. The pushing element 46, 92, 128 may force the electrodes 500 through the end cap 502, 510, and allow the electrodes 500 to expand to their final width in the deployed position. In some embodiments, the ends of the electrodes 500 may be substantially parallel at least in the deployed position. The pushing element 46, 92, 128 may then retract the electrodes 500 by pulling the electrodes back into the insertion tube 15. In some embodiments, a carrier (e.g., carrier 206 shown in FIG. 21) may engage the electrodes 500 and the pushing element 46, 92, 128 to transmit the axial force from the actuator 42, 74, 112 to the electrodes. The embodiment of FIGS. 60 and 61 depicts another embodiment of the insertion tube 15 and end cap 510 through which the electrodes 500 may extend via the channels 504, and which also depict an alignment opening 512 and alignment channel 168 to support a drug delivery channel 18 therein. The embodiment of FIGS. 60 and 61 depicts the embodiment of FIGS. 12, 13, 31, and 32 having an insertion tube 15 with a drug delivery channel 18 extending therethrough. The drug delivery channel 18 and electrodes 500 may be operated and structured in accordance with any of the embodiments herein.

In any of the embodiments of the electrodes 100 described herein, the portion of the electrodes 100 closest to the tip may be defined parallel to each other in both the deployed and retracted positions. In some embodiments, the portion of the electrodes 100 farthest from the tip may also be parallel in both the deployed and retracted positions, and at least a part of this farthest portion may remain within the insertion tube 15 in both the deployed and retracted positions. Between the farthest portion from the tip and the closest portion to the tip, the electrodes may include 100 a straight or curved portion of electrode. For example, the “S” shaped curve may be defined between the respective end portions of the electrode. In some embodiments, the middle portion of the electrode may be straight in the retracted position and curved in the deployed position.

With reference to FIGS. 14, 15, 33-41, and 66, an embodiment of the electrodes 600 is shown disposed in an expandable center carrier 602 from which the electrodes extend. The electrodes 600 may each include a tip 601 at a distal end thereof opposite the insertion tube 15. The tip 601 of the electrodes 600 may define a pointed end configured to pierce the target tissue for electroporation. In some embodiments, in the retracted position (FIG. 14), the electrodes 600 may be withdrawn into the carrier 602 and the carrier may be withdrawn into the distal end of the insertion tube 15. In some embodiments (FIGS. 33-41), the electrodes 600 may be fixed to the carrier 602 and the carrier may be withdrawn in to the distal end of the insertion tube 15 in the retracted position (FIGS. 38, 40). With reference to FIG. 33, in some embodiments, wires 17 may pass through the carrier 602 to the electrodes 600 via channels 612.

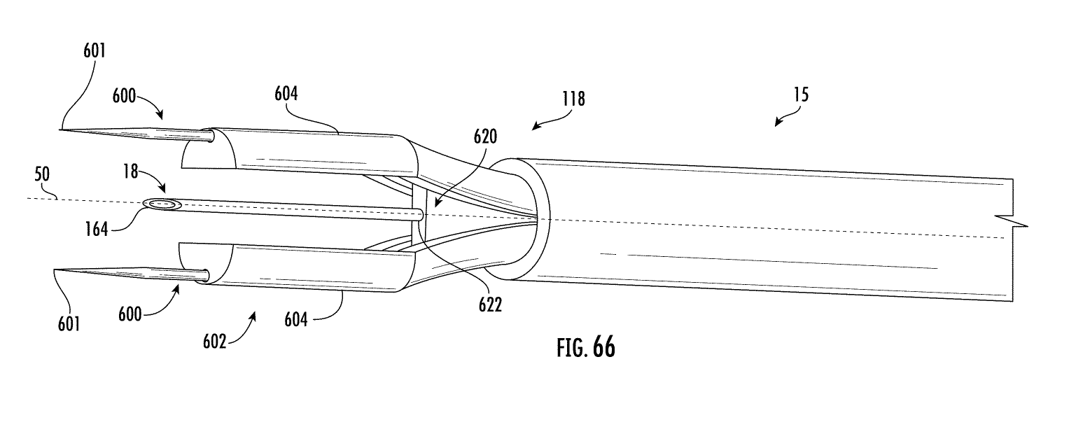

In some embodiments, the pushing element 46, 92, 128 may apply an axial force, directly or indirectly, to an inner member 606, 610, 620, which may separate the halves 604 of the carrier 602 to spread the electrodes 600 outwardly. In some embodiments, the inner member may be a wedge 606 (shown in FIG. 15) within the carrier 602. In some embodiments, the inner member may be a cylinder 610 (shown in FIGS. 33, 38). In some embodiments, the inner member 606, 610 may translate axially 50 relative to the carrier 602, while also pushing the carrier at least partially out of the distal end of the insertion tube 15. The embodiment of FIG. 66 depicts another embodiment of the insertion tube 15 and inner member 620 which may deploy the carrier 602 and electrodes 600. The embodiment of FIG. 66 depicts the embodiment of FIGS. 14, 15, and 33-41 having an insertion tube 15 and inner member 620 with a drug delivery channel 18 extending therethrough. The drug delivery channel 18, inner member 620, and electrodes 600 may be operated and structured in accordance with any of the embodiments herein.

In some embodiments, the inner member 606, 610 may be separately actuated by a second actuator 94 (shown in FIGS. 35, 37, 39, and 40). In operation, with reference to FIGS. 35, 37, 39, and 40, after the actuator 74 deploys the carrier 802 forwards from the distal end of the insertion tube 15, the second actuator 94 may be pressed inwardly into the body 90 of the control portion 72 to align a distal end 98 of the second actuator with an opening in the hollow mandrel 86 (e.g., along axis 50 shown in FIG. 14), with the second actuator having a bent portion 97 to allow the distal end to reach deeper into the hollow mandrel. The actuation of the hollow mandrel 86 by the actuator 74 may allow the second actuator 94 to fit behind the hollow mandrel in line with its opening. The inner member 606, 610 (FIGS. 15, 41) may be configured to translate relative to the hollow mandrel 86 from a position within the hollow mandrel, such that a user may actuate the second actuator 94 by sliding the second switch 96 axially forward (e.g., towards the distal end of the insertion tube 15) such that the distal end 98 of the second actuator engages a base surface 614 (shown in FIGS. 33, 38) of the inner member 606, 610. The second actuator 94 may thereby cause the halves 604 of the carrier 602 to separate (as shown in FIGS. 15 and 41) by actuating the inner member 606, 610 through the hollow mandrel 86 after the carrier 602 has been actuated by the actuator 74 (e.g., after the carrier 602 has been advanced axially from within the insertion tube 15 by actuation of the first actuator).

The relative axial movement between the inner member 606, 610 and the carrier 602 may apply a radial force on a ramped surface within two halves 604 of the carrier, to cause the halves 604 to expand radially outwardly. For example, with reference to FIG. 38, the carrier 602 may include a tapered surface 616 in its interior that, when operated on by the inner member 606, 610, causes the halves 604 of the carrier to expand outwardly. Although FIGS. 15, 35, and 41 depict a portion of the carrier 602 and electrodes 600 being articulated substantially parallel to each other in the deployed position, in some embodiments, the carrier 602 and electrodes 600 may curve radially outwardly (e.g., similar to the angles of FIG. 5) in response to the actuation of the wedge 606 with only the halves 604 of the carrier 602 being a substantially contiguous piece of material.

In some embodiments, the carrier 602 may only define two halves 604 near the distal end, and a remaining portion of the carrier may be a single, solid piece, such that the two halves are still affixed to each other (e.g., cylindrical portion 606).

In some embodiments, with reference to FIG. 41, the inner member 606, 610 may define a needle fluidly connected to the drug delivery device (e.g., drug delivery device 16 shown in FIG. 1), such that the inner member administers the treatment agent to the target area after the halves 604 of the carrier 602 separate. In such embodiments, the treatment agent may be delivered via a drug delivery channel (e.g., drug delivery channel 18 shown in FIG. 1) extending through the insertion tube 15 as described herein.

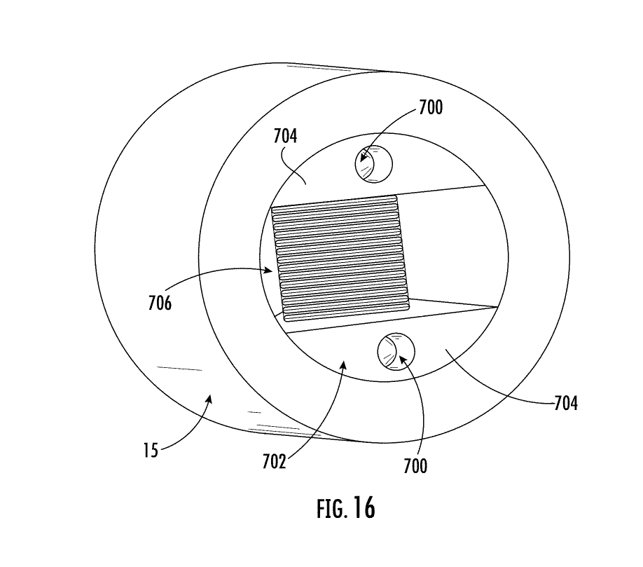

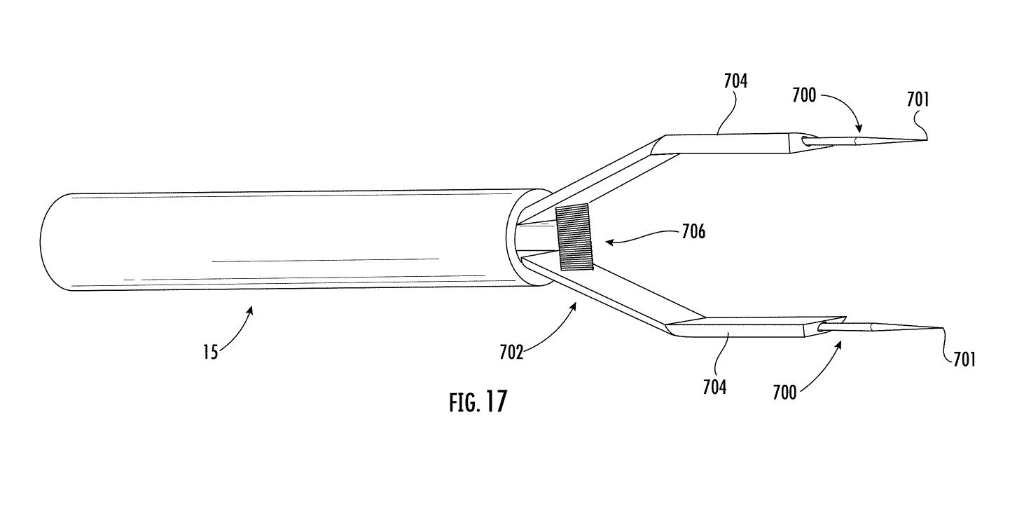

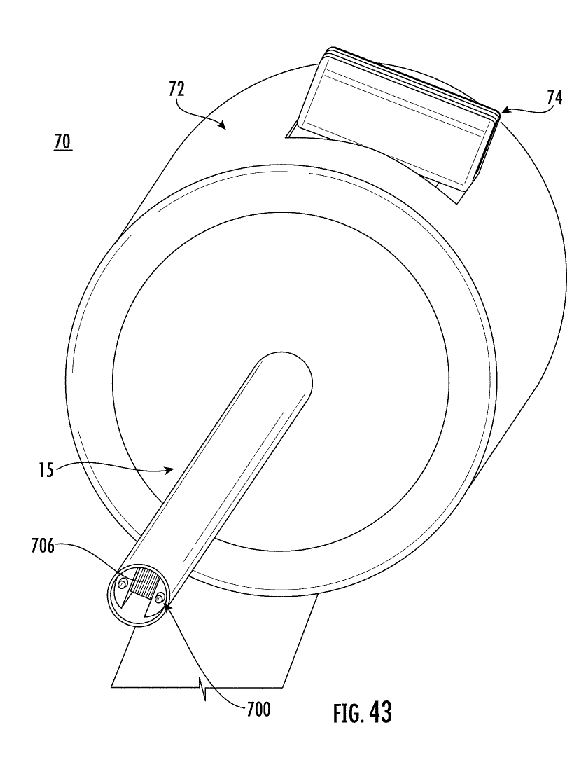

Turning to FIGS. 16, 17, 42, and 43, another embodiment of the electrodes 700 is shown. In the depicted embodiment, the electrodes 700, carrier 702, and applicator 14, 60, 70 may operate in substantially the same manner as the embodiment of FIGS. 14, 15, and 33-41, except that the inner member (e.g., wedge 606 or cylinder 610) and second actuator 94 are replaced with a spring 706 that expands the carrier halves 704 radially outwardly, while the pushing element 46, 92 directly or indirectly drives the electrodes 700 and carrier 702 axially out of the applicator 14, 60, 70 and into a deployed position (FIGS. 17, 42). The electrodes 700 may each include a tip 701 at a distal end thereof opposite the insertion tube 15. The tip 701 of the electrodes 700 may define a pointed end configured to pierce the target tissue for electroporation. In some examples, the spring 706 may be biased so that upon deployment from the insertion tube 15, the spring expands to its biased position and thereby spreads apart the electrodes and electrode tips 701.

Additionally, or alternatively, the pusher member may similarly be spring-biased such that, upon actuation by the user, the electrodes are forced into a deployed position by the spring-loaded actuator. Then, if present, the spring 706 may simultaneously expand the electrodes away from one another (or another mechanism as discussed above may complete this action).

While in most of the described embodiments herein, the electrodes are in the shape of needles with pointed tips, capable of piercing tissue to be treated, in other embodiments, the electrodes may take on the shape of something other than a needle which may or may not include a tip capable of piercing tissue. For instance, one or more the electrodes may have a blunt tip, or further, may have a flat shape, rounded shape, or the like, that simply presses against the tissue to be treated rather than piercing the tissue to be treated. In such instances, as the electrodes are atraumatic, the electrodes need not necessarily be actuatable, but instead can be positioned in a fixed location relative one another. Of course, in instances where the applicator is sized for passage through an access instrument, such as an endoscope, actuation of at least one of the electrodes may be necessary to allow for adequate spacing between the electrodes on the tissue to be treated. As such, at least one of the electrodes may be fixed while at least one of the other electrodes may be actuatable or, as discussed above, each of the electrodes may be independently or collectively actuatable.

In this manner, as discussed previously, in certain embodiments, one or more of the electrodes may have the needle shape or some other projected shape suitable of pressing or piercing tissue to be treated, while the other electrode (e.g., the return or negative electrode) may be positioned on, or actually be, the distal tip of the applicator or endoscope which is positioned adjacent the tissue to be treated, and thus could be suitable for acting as an electrode. Furthermore, in this exemplary embodiment, the one or more positive electrodes need not be actuatable, but instead, can merely be positioned in a fixed location so as to project distally to a position sufficiently apart from the distal tip of the applicator (or to be positioned a suitable distance from the distal end of the endoscope or other access instrument) to allow for supply of an electrical pulse, as described herein.

In some embodiments, the actuation mechanism to control deployment of the electrodes may be passive (e.g., shape memory material for electrodes 400, spring 706 for electrodes 700). In some embodiments, the actuation mechanism to control deployment of the electrodes may be active (e.g., advancement of inner member 606, 610 through second actuator to cause electrodes 600 to move apart).

In some embodiments, an applicator may include a plurality of electrodes that are at an operative spacing for electroporation both before and after deployment from the applicator. In this manner, a spacing between the electrodes remains the same before and after deployment. The effect of deployment in this configuration is simply to axially advance the electrodes relative to the insertion tube of the applicator.

In some embodiments, applicators as described in the various embodiments of the application may include three electrodes, four electrodes, or more. Illustrative examples of these arrangements are provided elsewhere in the present disclosure. For each applicator, it is contemplated that the higher number of electrodes may be incorporated following the structural configuration of the existing design. Thus, for example, insertion tube 15 shown in FIG. 21 includes channels 204 at the tip that are angled outward from a centerline of the tube 15. In a variation of this embodiment with three electrodes, three channels 204 may be included, each equally spaced and extending away from the tube centerline toward an outer perimeter of the tube.

In some examples, an applicator may include four electrodes. The applicator may be rectangular in shape with electrodes spaced about 5 mm apart. In some examples, an applicator may include six electrodes positioned peripherally about a circumference with a diameter of about 5 mm. The two preceding arrangements were used in electroporation procedures under both high and low voltage conditions as part of a study. Details of the treatment performed and the results illustrative of the advantages of low voltage electroporation are found in Burkart et al., Improving therapeutic efficacy of IL-12 intratumoral gene electrotransfer through novel plasmid design and modified parameters, Gene Therapy, 25, 93-103 (9 Mar. 2018), incorporated by reference herein in its entirety.

In any of the above-noted embodiments, the one or more electrodes may be deployed simultaneously and collectively with all electrodes or any portion of the total number of electrodes. Alternatively, each individual electrode may be actuated and deployed independently of the others.

In yet another embodiment, the electrodes may operate as a harpoon, whereby each electrode is inserted into the tissue such that each electrode separates from the applicator 14, tethered only by the wire or like structure which provides an electrical connection to the electrode. As such, each electrode can be positioned into the tissue at any location desired. For example, each electrode is deployed one at a time from the applicator at various locations in and around the target tissue. Each electrode remains tethered to the applicator and/or another electrode. Upon completion of the procedure, each electrode is drawn back to the applicator, whether by a spooling reel, a pulling of the wire, a magnetic attraction between the applicator and the electrode, or the like.

As discussed above, the electrodes are typically connected to a power source via a wire, though also present in most embodiments is a pusher member and an insertion tube. In some embodiments, the pusher member or the insertion tube could operate as the electrical connection to at least one of the electrodes, thereby eliminating the need for at least one of the wires. As one example, in an instance with two electrodes, the positive connection to one of the electrodes could be via the pusher member, while the negative or return connection to the other electrode could be the insertion tube body. Of course, adequate insulation of these structures would be required to avoid arcing of the electrodes and/or injury to the user.

In still another embodiment, the electrical connection between the electrical source and the at least one electrode could be wireless, for example, via the use of inductive power transfer via an electromagnetic field. Such a power connection could be completed transdermally, such that a wire would not be required to pass between the target tissue and the power source. Continuing with such an electrical connection, in certain embodiments, the harpoon-like electrode mentioned previously could be positioned in the target tissue, which would not be connected via wires to an electrical source. In this way, the drug delivery could occur by any desired procedure, and the electroporation could occur without being in a surgical setting. For example, once the electrodes are implanted into the target tissue, and whether or not treatment agents have been supplied to the patient and/or the target tissue, the patient could be removed from the operating room and the treatment could be supplied one or more times outside of the surgical setting using a drug delivery device such as a needle or the like, and a transdermal power delivery to the electrodes. The electrodes may then be removed at a later date or may be biodegradable, or if they are of a shape that is atraumatic (e.g., a disc-shaped electrode sutured to tissue) or is otherwise secured in the patient without fear of coming loose, the implant may remain inside the patient indefinitely.

### Example Electrical Parameters

The nature of the electric field to be generated by the generator 12 is determined by the nature of the tissue, the size of the selected tissue and its location. It is desirable that the field be as homogenous as possible and of the correct amplitude. Excessive field strength results in lysing of cells, whereas a low field strength results in reduced efficacy. The electrodes may be mounted and manipulated in many ways including but not limited to those described herein. Using the system 10 described herein, the parameters of the electroporation (e.g., voltage, pulse duration, etc.) are all programmable and optimizable (e.g., via the one or more controllers described herein). In some embodiments, the parameters of the pulses are predetermined and employed in a consistent manner throughout the electroporation procedure. In some embodiments, the parameters of the pulses may be determined using a feedback mechanism while electricity is supplied to the applicator to continually adjust the parameters of the pulses during electroporation (e.g., EIS).

In some instances, electroporation uses high voltages and short pulse durations for treatment of tumors. The electrical field conditions of 1200-1300 V/cm and 100 μs have been used in vitro and in vivo with anticancer drugs like bleomycin, cisplatin, peplomycin, mitomycin c and carboplatin. These results refer to in vitro and in vivo work. Although such electrical conditions may be tolerated by patients in clinical situations, such treatments will typically produce muscle twitch and occasional discomfort to patients, and may produce worse results with certain treatment agents (e.g., larger molecules). Some of these problems could be considerably reduced by using low voltage high pulse durations for electrochemotherapy. Low voltage electroporation as contemplated by the present disclosure involves utilization of application of a voltage of about 600 V or lower, an electrical field of about 700 V/cm or lower, and a pulse length of between about 0.5 ms and about 1 s. In some examples, an electrical field of 400 V/cm or less may be utilized in a low-voltage generator configuration. In some embodiments, the generator 12 may apply a voltage of 300 V or less to the electrodes 100. In some embodiments, the generator 12 may apply a voltage of 60-300 V to the electrodes 100. In some embodiments, the generator 12 may apply a voltage of 150-200 V. In some embodiments, high voltages of greater than 1000V may cause irreversible electroporation (IRE). Thus, electroporation systems incorporating a low voltage generator are advantageous in that a risk of IRE is low compared with treatments employing a higher voltage.

The waveform of the electrical signal provided by the generator 12 can be an exponentially decaying pulse, a square pulse, a unipolar oscillating pulse train, a bipolar oscillating pulse train, or a combination of any of these forms. In some embodiments, the electrical parameters for the generator, encompassing a range for both low and high voltage generators, may encompass a nominal electric field strength from about 10 V/cm to about 20 kV/cm (the nominal electric field strength is determined by computing the voltage between electrode needles divided by the distance between the needles). In some embodiments encompassing a range for both low and high voltage generators, the pulse length can be about 10 is to about 100 ms. In some embodiments encompassing a range for low voltage generators, the pulse length can be about 1 ms to about 1 s. There can be any desired number of pulses, typically one to 100 pulses per second. The wait between pulses sets can be any desired time, such as one second. The waveform, electric field strength and pulse duration may also depend upon the type of cells and the type of molecules that are to enter the cells via electroporation. The various parameters including electric field strengths required for the electroporation of any known cell is generally available from the many research papers reporting on the subject. An overview of the relationship between pulse strength and duration is described in Weaver et al., A brief overview of electroporation pulse strength-duration space: A region where additional intracellular effects are expected, Bioelectrochemistry, 2012 October; 87: 236-243. doi:10.1016/j.bioelechem.2012.02.007, which is incorporated by reference herein in its entirety. In some embodiments, any number of pulses may be used in a treatment. In some embodiments, 6 pulses are used. In some embodiments, 8 pulses are used. In some embodiments, 10 pulses are used.

In the depicted embodiments, the nominal electric field can be designated either “high” or “low”. The following paragraphs describe electrical parameters for system including a high voltage generator followed by a system including a low voltage generator.

Turning to high voltage systems specifically, i.e., those having a high electric field, in some embodiments, it is preferable that the nominal electric field is from about 700 V/cm to 1500 V/cm. In some embodiments, it is further preferable that the nominal electric field is from about 1000 V/cm to 1500 V/cm. In some embodiments, the high electric field may be about 1500 V/cm. With regard to pulse duration for high voltage systems, in some embodiments, a pulse duration of less than 1 ms may be used. In some embodiments, a pulse duration between 100 μs and 1 ms may be used.

Turning to low voltage systems specifically, in some embodiments, the generator may be a low-voltage generator. The electroporation therapy may be administered using the low-voltage generator producing an electric field of 700 V/cm or less, 600 V/cm or less, 500 V/cm or less, 400V/cm or less, 300V/cm or less, 200V/cm or less, or 100V/cm or less. The electroporation therapy may be administered using the low-voltage generator producing an electric field from 700 V/cm to 10 V/cm. The electroporation therapy may be administered using the low-voltage generator producing an electric field from 600 V/cm to 10 V/cm. The electroporation therapy may be administered using the low-voltage generator producing an electric field from 500 V/cm to 10 V/cm. The electroporation therapy may be administered using the low-voltage generator producing an electric field from 400 V/cm to 10 V/cm. The electroporation therapy may be administered using the low-voltage generator producing an electric field from 300 V/cm to 10 V/cm. The electroporation therapy may be administered using the low-voltage generator producing an electric field from 700 V/cm to 60 V/cm. The electroporation therapy may be administered using the low-voltage generator producing an electric field from 600 V/cm to 60 V/cm. The electroporation therapy may be administered using the low-voltage generator producing an electric field from 500 V/cm to 60 V/cm. The electroporation therapy may be administered using the low-voltage generator producing an electric field from 400 V/cm to 60 V/cm. The electroporation therapy may be administered using the low-voltage generator producing an electric field from 300 V/cm to 60 V/cm. The electroporation therapy may be administered using the low-voltage generator producing an electric field from 700 V/cm to 100 V/cm. The electroporation therapy may be administered using the low-voltage generator producing an electric field from 600 V/cm to 100 V/cm. The electroporation therapy may be administered using the low-voltage generator producing an electric field from 500 V/cm to 100 V/cm. The electroporation therapy may be administered using the low-voltage generator producing an electric field from 400 V/cm to 100 V/cm. The electroporation therapy may be administered using the low-voltage generator producing an electric field from 300 V/cm to 100 V/cm. The electroporation therapy may be administered using the low-voltage generator producing an electric field from 300 V/cm to 200 V/cm. The electroporation therapy may be administered using the low-voltage generator producing an electric field from 400 V/cm to 300 V/cm. In some embodiments, the pulse duration of the low-voltage generator may be from 1 millisecond (ms) to 1 second (s).

Preferably, when low fields are used, the nominal electric field is from about 10 V/cm to 400 V/cm. In some embodiments, the nominal electric field may be from about 25 V/cm to 75 V/cm. In some embodiments, the low nominal electric field may be about 400 V/cm. In a particular embodiment, it is preferred that when the electric field is low, the pulse length is long relative to a high field pulse. For example, when the nominal electric field is in the “low” range discussed herein, it is preferred that the pulse length is about 10 msec.

With continuing reference to a system with a low voltage generator, in some embodiments, the low-voltage generator may produce a voltage ranging from 600V to 5V. In some embodiments, the low-voltage generator may produce a voltage ranging from 500V to 5V. In some embodiments, the low-voltage generator may produce a voltage ranging from 400V to 5V. In some embodiments, the low-voltage generator may produce a voltage ranging from 300V to 5V. In some embodiments, the low-voltage generator may produce a voltage ranging from 200V to 5V. In some embodiments, the low-voltage generator may produce a voltage ranging from 100V to 5V. In some embodiments, the low-voltage generator may produce a voltage ranging from 600V to 10V. In some embodiments, the low-voltage generator may produce a voltage ranging from 500V to 10V. In some embodiments, the low-voltage generator may produce a voltage ranging from 400V to 10V. In some embodiments, the low-voltage generator may produce a voltage ranging from 300V to 10V. In some embodiments, the low-voltage generator may produce a voltage ranging from 200V to 10V. In some embodiments, the low-voltage generator may produce a voltage ranging from 100V to 10V. In some embodiments, the low-voltage generator may produce a voltage ranging from 600V to 50V. In some embodiments, the low-voltage generator may produce a voltage ranging from 500V to 50V. In some embodiments, the low-voltage generator may produce a voltage ranging from 400V to 50V. In some embodiments, the low-voltage generator may produce a voltage ranging from 300V to 50V. In some embodiments, the low-voltage generator may produce a voltage ranging from 200V to 50V. In some embodiments, the low-voltage generator may produce a voltage ranging from 100V to 50V. In some embodiments, the low-voltage generator may produce a voltage ranging from 600V to 100V. In some embodiments, the low-voltage generator may produce a voltage ranging from 500V to 100V. In some embodiments, the low-voltage generator may produce a voltage ranging from 400V to 100V. In some embodiments, the low-voltage generator may produce a voltage ranging from 300V to 1001V. In some embodiments, the low-voltage generator may produce a voltage ranging from 200V to 100V. In some embodiments, the low-voltage generator may produce a voltage ranging from 600V to 200V. In some embodiments, the low-voltage generator may produce a voltage ranging from 500V to 200V. In some embodiments, the low-voltage generator may produce a voltage ranging from 400V to 200V. In some embodiments, the low-voltage generator may produce a voltage ranging from 300V to 200V. In some embodiments, the low-voltage generator may produce a voltage ranging from 600V to 300V. In some embodiments, the low-voltage generator may produce a voltage ranging from 500V to 300V. In some embodiments, the low-voltage generator may produce a voltage ranging from 400V to 300V. In some embodiments, the low-voltage generator may produce a voltage ranging from 600V to 400V. In some embodiments, the low-voltage generator may produce a voltage ranging from 500V to 400V. In some embodiments, the low-voltage generator may produce a voltage ranging from 600V to 500V.

Advantages of the low voltage generator may include an improved expression of therapeutic agents transfected over that of a high voltage generator. In some embodiments, the presence of a tissue sensing system as described elsewhere herein may further improve performance over that of another generator. Tissue sensing accomplished through the low voltage generator output may allow for characterization of the treatment site. In particular, the potential to gather feedback from therapy in order to determine unsafe treatment and potentially optimize therapy conditions may be highly comprehensive. Thus, following a series of pulses in a treatment, the expression of therapeutic agents may be significantly higher and more durable under the example embodiments described herein. Additionally, as noted elsewhere in the disclosure, production of a voltage below 600 V that produces an electric field below 700 V/cm with a low voltage generator mitigates the risk of irreversible electroporation which may cause damage to tissue in and around the target location for treatment. Moreover, electroporation with these parameters allows for an overall longer treatment duration, thereby increasing the likelihood of successful delivery of the treatment agent.

Preferably, the therapeutic method of the invention utilizes the systems described herein which may include an applicator, a plurality of electrodes configured to extend from the applicator, and a generator for applying an electric signal to the electrodes. In some embodiments, the system may also include an insertion device as described elsewhere in the application, such as an endoscope. In some embodiments, the electric pulses from the generator may be proportionate to the distance between said electrodes for generating an electric field of a predetermined strength, such that field strength for a particular surgery is higher for systems that include an applicator with electrode tips at a greater distance from one another. In some embodiments, a system that includes a low voltage generator may include an applicator with electrodes that have tips spaced apart about 4 mm. In some embodiments, the above electrical parameters, whether for systems with high voltage or low voltage generators, may be employed without using feedback from sensing circuitry to control and otherwise update the applied voltage during an electroporation procedure.

In some embodiments, the electrical pulses may be controlled via feedback from the sensing circuitry 31, which may measure the parameters of the electrodes 100 and target tissue continually during electroporation. In some embodiments, a sensing pulse may be transmitted between electroporation pulses, such that the generator quickly alternates between applying therapeutic electroporation and sensing the parameters of the electrodes and tissues. In some embodiments, an adaptive control method may be used to set the electroporation parameters in real time. One way in which the generator (e.g., via sensing circuitry 31, pulse circuitry 33, and controller 24) may measure the electroporation parameters and control the pulses of the generator is via Electrochemical Impedance Spectroscopy (EIS). In some embodiments, EIS may be used with a low-voltage generator.

An adaptive control method for controlling electroporation pulse parameters during electroporation of cells or tissues using the electroporation system 10 includes providing a system (e.g., generator 12 and its corresponding circuitry) for adaptive control to optimize electroporation pulse parameters including electroporation pulse parameters, applying voltage and current excitation signals to the cells (e.g., via pulse circuitry 33), obtaining data from the current and voltage measurements (e.g., via sensing circuitry 31), and processing the data to separate the desirable data from the undesirable data (e.g., via controller 24 and processor 30), extracting relevant features from the desirable data (e.g., via controller 24 and processor 30), applying at least a portion of the relevant features to a trained diagnostic model, also referred to herein as “trained model” (e.g., via controller 24 and processor 30), estimating electroporation pulse parameters based on an outcome of the applied relevant features (e.g., via controller 24 and processor 30), where the initialized electroporation pulse parameters are based on the trained model and the relevant features, to optimize the electroporation pulse parameters, and applying, by the generator, a first electroporation pulse based on the first pulsing parameters.

To maximize the efficacy of electroporation, a quantifiable metric of membrane integrity that is measurable in real-time is desirable. As described herein, EIS is a method for the characterization of physiologic and chemical systems and can be performed with any of the standard electroporation, also referred to throughout the disclosure as “EP”, electrodes described herein. This technique measures the electrical response of a system over a range of frequencies to reveal energy storage and dissipation properties. In biologic systems the extracellular and intracellular matrix resist current flow and therefore can be electrically represented as resistors. The lipids of intact cell membranes and organelles store energy and are represented as capacitors. Electrical impedance is the sum of these resistive and capacitive elements over a range of frequencies. To quantify each of these parameters, tissue impedance data can be fit to an equivalent circuit model. Real-time monitoring of electrical properties of tissues will enable feedback control over electroporation parameters and lead to optimum transfection in heterogeneous tumors. Using EIS feedback, will allow (1) delivery parameters to be adjusted in real-time, (2) delivery of only the pulses necessary to generate a therapeutic response, and (3) reduce the overall EP-mediated tissue damage as a result.

In addition, in some embodiments, these EIS measurements can be used to determine ideal electroporation conditions described herein. In some embodiments, the method of the present invention may include contacting the tissue in the target site with a pair of electrodes 100. A low voltage power supply electrically connected to the electrodes 100 may be used to apply a low voltage excitation signal to the electrodes. Methods for sensing the impedance and/or capacitance may include but are not limited to waveforms such as phase locked loops, square wave pulses, high frequency pulses, and chirp pulses. A voltage sensor and a current sensor are used to sense a voltage drop and current flowing through the circuit, and these parameters may then be processed by the controller 24, as illustrated in FIG. 1, to determine an average impedance for all cells in the measured area. This detected impedance may then (e.g., via the trained model discussed above) determine any necessary changes to the electroporation parameters.

In some embodiments, the generator 12 (e.g., via sensing circuitry 31) is configured to measure dielectric and conductive properties of cells and tissues, and includes a voltage sensor to measure voltages across the tissue resulting from each of an excitation signal for sensing purposes and/or an electroporation pulse applied to the tissue, and a current sensor to measure current across the tissue resulting from each of the excitation signal for sensing purposes and/or the at least one applied electroporation pulse.

The pulsing circuitry 33 may include an initializing module configured to initialize electroporation pulsing parameters for performing electroporation in the cells or tissue, where initialized electroporation pulsing parameters are based at least in part on at least one trained model, such as the trained model described elsewhere in the present disclosure. In some embodiments, the controller 24 may direct the output of the pulsing circuitry 33. The generator 12 is configured to apply at least one of the excitation signals and/or the electroporation pulse to the tissue. The voltage sensor and current sensor of the sensing circuitry 31 may measure voltage and current across the cells of the tissue in response to the application of the excitation signals. The controller 24 may be configured to receive a signal relating to the measured sensor data from the sensing circuitry 31, corresponding to at least one of the excitation signal and the electroporation pulse, to fit the data to at least one trained model and to process the data into diagnostics and updated control parameters.

In the low voltage operation, the generator may output any of the parameters described herein, including, for example, a minimum of 10 V and maximum of 300 V with pulse durations ranging from 100 to 10 ms. EIS may be data captured before and between pulses and obtained by the generator 12 over a range of 100 Hz to 10 kHz with 10 data points acquired per decade. Acquisition of EIS data over this spectra is accomplished in 250 ms, which is rapid enough to: (1) execute routines to determine a time constant for the next pulse; (2) store EIS data for post analysis; and (3) not interrupt clinically used electroporation conditions. The generator may be capable of a minimum output load impedance of 20 ohms and a maximum load impedance of an open circuit. To allow hands-free operation of the generator a foot pedal (e.g., foot pedal 58) may be added to trigger, pause, or abort the electroporation process.

The controller 24 may include a pre-processing module to receive the signal relating to the data from the current and voltage measurements, and process the data to separate desirable data from undesirable data, a feature extraction module to extract relevant features from the desirable data, a diagnostic module to apply at least a portion of the relevant features of the desirable data to at least one trained diagnostic model, and a pulse parameter estimation module to estimate at least one of initialized pulsing parameters and subsequent pulsing parameters based on an outcome of at least one of the measured data, the diagnostic module and the feature extraction module. The memory 36 stores the desirable and undesirable data, sensor data and the trained models for feature extraction by the controller.

### Methods of Operation

Various methods associated with the electroporation system 10 will now be described. In any of the embodiments described herein, such methods can be used for treatment of one or more cancers, and more specifically, can be used to treat a tumor or other visceral lesion, particularly those found within a patient and which are not superficial or in the dermal layers. Such tumors or other lesions may be either primary or metastatic malignancies.

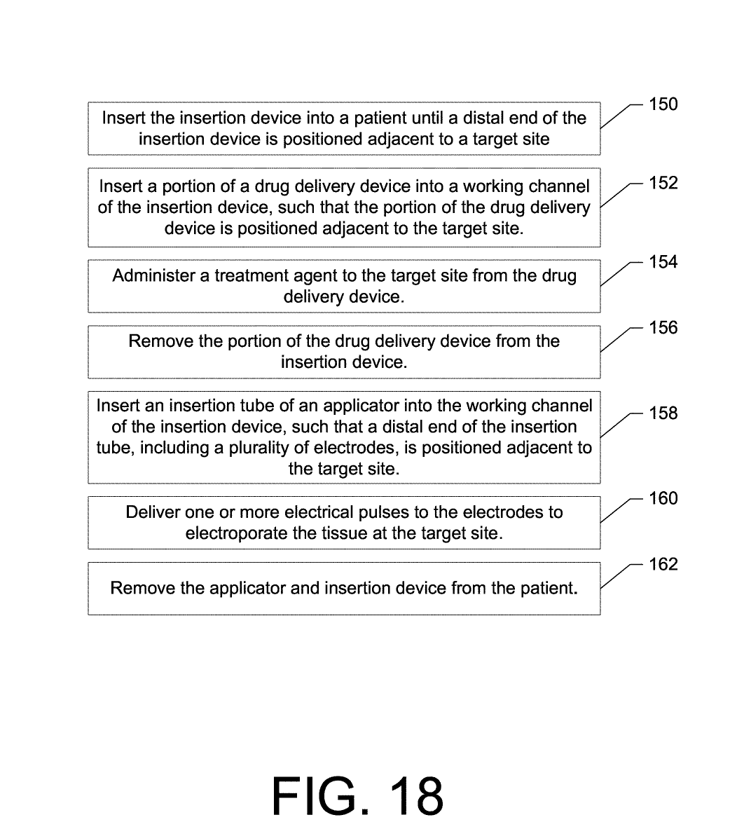

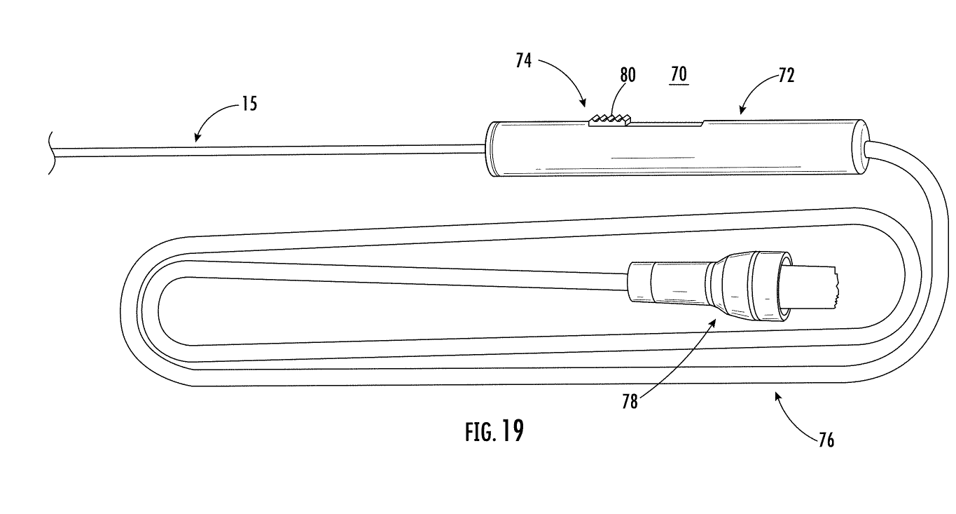

With reference to FIG. 18, an example method of using the electroporation system 10 described herein is shown. In some embodiments, the method of FIG. 18 is used for treatment of one or more cancers. In some embodiments, the method of FIG. 18 is used to treat a tumor or other visceral lesion. At depicted step 150, the method may include inserting an insertion device into a patient until a distal end of the insertion device is positioned adjacent to a target site. The insertion device may be advanced through an internal passage in a variety of ways as described, for instance, in the specific examples below. In some embodiments, the insertion device may be an endoscope, including flexible endoscopes or rigid endoscopes, such as a trocar. In some embodiments, the applicator may be inserted itself with no insertion device. At depicted step 152, the method may include inserting a portion of a drug delivery device into a working channel of the insertion device, such that the portion of the drug delivery device is positioned adjacent to the target site. At depicted step 154, the method may include administering a treatment agent to the target site from the drug delivery device. At depicted step 156, the method may include removing the portion of the drug delivery device from the insertion device. At depicted step 158, the method may include inserting an insertion tube of an applicator into the working channel of the insertion device, such that a distal end of the insertion tube, including a plurality of electrodes, is positioned adjacent to the target site. At depicted step 160, the method may include delivering one or more electrical pulses to the electrodes to electroporate the tissue at the target site. At step 162, the method may include removing the applicator and insertion device from the patient. In some embodiments, the applicator may include a piercing tip 130 such that the method may further include piercing one or more tissues of the patient prior to delivering the electrical impulse and/or treatment agents. In some embodiments, as described above, the drug and/or plasmid may be administered through any of a number of means, including IM, IT, and IV delivery. In embodiments in which the drug delivery device operates through the applicator, steps 152-156 may be combined with steps 158-162. In the above described method, a low voltage or high voltage generator may be used, including the particular configurations described herein. The method may be performed with or without EIS. In one example of the method performed with a low voltage generator and without EIS, the voltage applied may be the same for each pulse of the treatment, irrespective of the characteristics of the tissue encountered (e.g., the variable impedance of the tissue that may be encountered through performance of the method) and a result should be obtained that is not affected by the characteristics of the tissue. Further, as noted elsewhere in the disclosure, treatment using this approach has been shown to be successful and to possess advantages relative to treatment that employs a high voltage generator.

Advantages of performing the method using a low voltage generator and the applicator as described herein include that less heat stress is applied to the cells at the target site during electroporation, thereby increasing the likelihood that the cells will survive throughout and after the treatment. Additionally, with a lower voltage, electrical pulses may be delivered over a longer period of time compared to a high voltage electroporation procedure. With a longer duration treatment, the cells are kept open for a longer period and a greater amount of the treatment agent may be absorbed by the cells, increasing the likelihood of successful treatment.

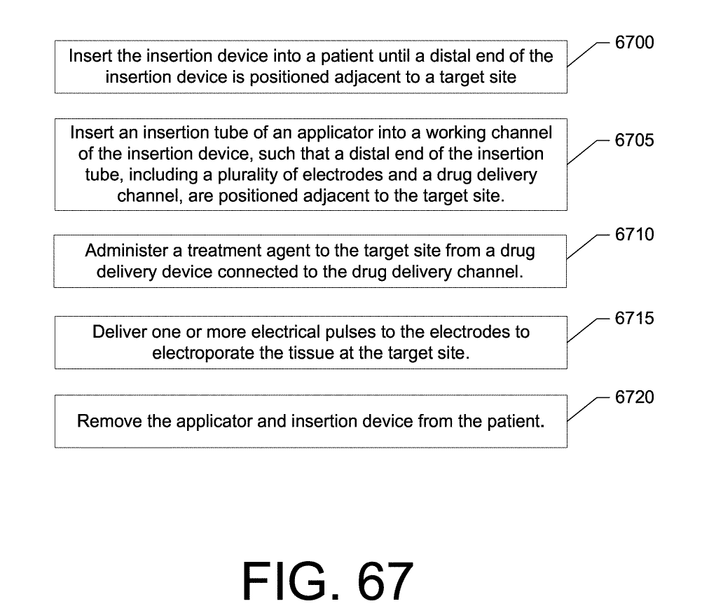

With reference to FIG. 67, another example method of using the electroporation system 10 described herein is shown. In some embodiments, the method of FIG. 67 is used for treatment of one or more cancers. In some embodiments, the method of FIG. 67 is used to treat a tumor or other visceral lesion. At depicted step 6700, the method may include inserting the insertion device into a patient until a distal end of the insertion device is positioned adjacent to a target site. In some embodiments, the insertion device may be an endoscope, including flexible endoscopes or rigid endoscopes, such as a trocar. Alternatively, the applicator may be inserted itself with no insertion device. At depicted step 6705, the method may include inserting an insertion tube of an applicator into the working channel of the insertion device, such that a distal end of the insertion tube, including a plurality of electrodes and a drug delivery channel, are positioned adjacent to the target site. At depicted step 6710, the method may include administering a treatment agent to the target site from a drug delivery device connected to the drug delivery channel. At depicted step 6715, the method may include delivering one or more electrical pulses to the electrodes to electroporate the tissue at the target site. At step 6720, the method may include removing the applicator and insertion device from the patient. In some embodiments, the applicator may include a piercing tip 130 such that the method may further include piercing one or more tissues of the patient prior to delivering the electrical impulse and/or treatment agents. In some embodiments, as described above, the drug and/or plasmid may be administered through any of a number of means, including IM, IT, and IV delivery. In the above described method, a low voltage or high voltage generator may be used, including the particular configurations described herein. The method may be performed with or without EIS.

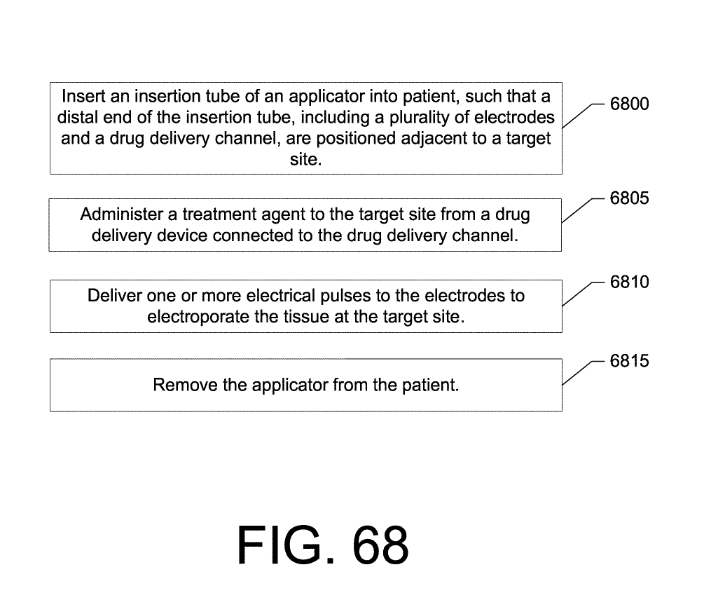

With reference to FIG. 68, another example method of using the electroporation system 10 described herein is shown. In some embodiments, the method of FIG. 68 is used for treatment of one or more cancers. In some embodiments, the method of FIG. 68 is used to treat a tumor or other visceral lesion. At depicted step 6800, the method includes inserting an insertion tube of an applicator into the patient, such that a distal end of the insertion tube, including a plurality of electrodes and a drug delivery channel, are positioned adjacent to a target site. At depicted step 6805, the method includes administering a treatment agent to the target site from a drug delivery device connected to the drug delivery channel. At depicted step 6810, the method includes delivering one or more electrical pulses to the electrodes to electroporate the tissue at the target site. At step 6815, the method includes removing the applicator from the patient. Steps 6805 and 6810 may occur simultaneously, or step 6805 may occur prior to step 6810. In some embodiments, the applicator may include a piercing tip 130 such that the method may further include piercing one or more tissues of the patient prior to delivering the electrical impulse and/or treatment agents. In some embodiments, as described above, the drug and/or plasmid may be administered through any of a number of means, including IM, IT, and IV delivery.

The methods, systems, and apparatus described herein may be used with a number of endoscopic procedures, including but not limited to procedures in the respiratory tract (e.g., rhinoscopy or bronchoscopy), the abdominal cavity, general soft tissue and/or bone, the gastrointestinal tract (e.g., enteroscopy, rectoscopy, colonoscopy, anoscopy, sigmoidoscopy, or esophagogastroduodenoscopy), the urinary system and in the cerebrum. Examples of the application of the method in these procedures is provided in greater detail below. It should be appreciated that in these and other procedures described throughout the disclosure, references to diseased tissue includes, but is not limited to, tumors, cancerous cells, and other lesions in general. Cancers treated may include soft tissue sarcomas. Tumors contemplated for treatment through the methods of the present disclosure include, for example, primary tumors, metastatic tumors, or both.

In some embodiments, the present disclosure relates to a method of treating diseased tissue (e.g., primary and/or metastatic tumors) in the respiratory tract. In some embodiments of the method, the lung may be accessed using bronchoscopy. In some embodiments, prior to performance of surgery, pre-operative planning may be performed to confirm the specific location of the diseased tissue and to perform applicator advancement path or endoscopic path planning. Pre-operative surgical planning may involve capturing images using cone beam computed tomography (CBCT) and using such images to generate a 3D model of the patient's lungs. Other techniques may also be used to capture images, including computed tomography, magnetic resonance, positron emission tomography, fluoroscopy and x-rays. The image data taken from any number of the above modalities may be extrapolated to create the 3D model of the patient anatomy. An analysis of the 3D model is then performed to identify the location of the diseased tissue. Once identified, a surgical plan may be developed for access to the diseased tissue. Based on an identified target site, details of an approach to the site may be established. In some embodiments, pre-operative planning may involve other known approaches to identifying diseased tissue. For example, where the diseased tissue is closer to an orifice, a surgical plan may be established without the creation of a 3D model. In other examples, it may be sufficient to use one or more of the modalities for capturing images of the patient without analysis and extrapolation to identify a location of diseased tissue and to establish a path of access.

Turning to the performance of the bronchoscopy, in some embodiments, the patient is adjusted to a sitting or supine position. Then, the applicator is inserted into an endoscope or bronchoscope in preparation for advancement into the patient. In particular, the insertion tube of the applicator is inserted into the endoscope. The endoscope may be flexible or rigid. Using the established pre-operative surgical plan, the endoscope is inserted through the nose or mouth into and through the upper airway, trachea, and into the bronchial system, and then into, in some examples, the lungs. Visualization tools included with the endoscope are used to aid in reaching the diseased tissue at the target site. The endoscope is advanced until its distal tip is proximal to or contacts the target site. In some embodiments, the advancement of the endoscope may be monitored with a connected navigation system. Where pre-operative planning includes the generation of a 3D model, additional images may be taken during the advancement steps at the discretion of the surgeon to make any adjustments based on actual conditions if evidence suggests that conditions have changed since the original images were taken to create the 3D model. In some embodiments, the visualization tools described herein may be used with embodiments of a separate drug delivery applicator (e.g., the separate drug delivery applicator 19 discussed herein) to facilitate identification of the injection site and alignment of the applicators (e.g., applicator 14 and separate applicator 19) for collocating delivery of the drug and electroporation.

With the distal end of the applicator located at the target site, electroporation and/or drug delivery may commence in a manner as described in any of the embodiments set forth herein. In some embodiments, electroporation and delivery of the treatment agent(s) may be simultaneous or otherwise occur at about the same time. In some embodiments, electroporation may commence prior to delivery of the treatment agent(s). In some embodiments, delivery of the treatment agent(s) is followed by electroporation.

In some examples, the bronchoscopy procedure described may be similarly employed in a rhinoscopy procedure or other procedure in the respiratory tract.

In some examples, the method of treating diseased tissue in the respiratory tract may be performed with the aid of robotics. For instance, the applicator may be used with a robotic system to perform the bronchoscopy. In particular, the applicator may be advanced through the body of the patient and/or the electrodes of the applicator may be deployed through control of the robotic device of the robotic system. To perform these functions, for example, an arm of the robotic device may be manipulated to rotate and position the applicator during the procedure. Similarly, the arm of the robotic device may be manipulated to control electricity flow into the applicator. In some examples, other steps of the method may also be aided by the use of the robotic system.

In some embodiments, the present disclosure relates to a method of treating diseased tissue in the abdominal cavity. In some embodiments, the method may commence with pre-operative surgical planning as described in detail above. With a location of the diseased tissue and a path to access the diseased tissue identified, access to the target site and treatment may commence. In preparation for entry, the applicator may be inserted into an endoscope, though the endoscope may be positioned at least partially into the patient prior to inserting the applicator therethrough.

In some embodiments, the applicator used includes a sharp tip, such as tip 130 on applicator 110, for example. Initially, the endoscope is positioned through a mouth of the patient, through the esophagus and into the stomach. From within the stomach, the applicator is advanced to a stomach wall to create a gastric opening using tip 130, thereby advancing the endoscope with applicator therein into the peritoneal cavity. Alternatively, a standard trocar or other instrument may be used to pierce the stomach wall. Visualization aids accompanying the endoscope, in conjunction with optional navigation system and imaging information may then be used to direct the endoscope and applicator to the target site on a wall of the peritoneal cavity under guided imagery.

With the distal end of the endoscope located at the target site, electroporation and/or drug delivery may commence in a manner as described in any of the embodiments set forth herein. In some embodiments, electroporation and delivery of the treatment agent(s) may be simultaneous or otherwise occur at about the same time. In some embodiments, electroporation may commence prior to delivery of the treatment agent(s). In some embodiments, delivery of the treatment agent(s) is followed by electroporation.

In another embodiment, a method for treating diseased tissue in the abdomen may be performed using a laparoscope, whereby one or more keyhole cuts may be formed in the patient through which a laparoscope and the applicator are passed and navigated to the target tissue. As discussed above, drug delivery can be performed using the applicator, or alternatively, a separate instrument can be used to deliver the treatment agent(s) to the target tissue. At least one additional cannula may be used to provide a passageway for the applicator and/or drug delivery device to the target tissue. Typically, rigid cannula(e) are used, and thus, an applicator with a rigid insertion tube may also be used.

In some examples, the method of treating diseased tissue in the abdomen may be performed with the aid of robotics. For instance, the applicator may be used with a robotic system to perform the procedure. In particular, the applicator may be advanced through the body of the patient and/or the electrodes of the applicator may be deployed through control of the robotic device of the robotic system. To perform these functions, for example, an arm of the robotic device may be manipulated to rotate and position the applicator during the procedure. Similarly, the arm of the robotic device may be manipulated to control electricity flow into the applicator. In some examples, other steps of the method may also be aided by the use of the robotic system.

In some embodiments, the present disclosure relates to a method of treating diseased tissue in the gastrointestinal tract, such as in the pancreas. In some embodiments of this method, an ultrasound endoscope is used with the applicator inserted therein. The ultrasound endoscope uses high frequency sound waves to produce detailed images of anatomy, including lining and walls of the stomach and pancreas. As described above, in some embodiments, pre-operative surgical planning may be performed to identify a specific location of the diseased tissue and to evaluate the intended insertion path for the applicator and/or endoscope. Once ready for surgery, the applicator is inserted into the ultrasound endoscope, though the endoscope may be positioned at least partially into the patient prior to inserting the applicator therethrough. Note than an ultrasound endoscope may also be utilized in the other methods described herein in which an endoscope or other endoscopic-type instruments, such as bronchoscopes and laparoscopes, are used.

To access the diseased tissue target site, the ultrasound endoscope is inserted through the mouth and into the stomach. Using the images generated through the ultrasound as well as the information harnessed through pre-surgical planning, if used, the endoscope is manipulated within the stomach so that its distal tip faces a stomach wall abutting the portion of the pancreas having the diseased tissue. Then, the applicator is advanced from the endoscope so that a pointed tip on the applicator may penetrate the stomach wall and thereby reach a location abutting the target site on the pancreas. Alternatively, a standard trocar or other instrument may be used to pierce the stomach wall. In circumstances where the target site on the pancreas does not abut the stomach, the endoscope may be guided further once in the peritoneal cavity to direct the applicator to the target site. Additionally, visualization aids may accompany the endoscope, along with an optional navigation system and imaging information from pre-operative planning, to aid in the direction of the applicator to the target site.

In some examples, and as described elsewhere in the disclosure, an endoscope can be positioned through the mouth into the stomach/small intestine, where the applicator, with a flexible body, can be guided into pancreatic lesions, for sequential plasmid injection and electroporation. The flexible body (e.g., insertion tube 15) may have a length of approximately 100 cm to allow for navigation toward the target lesions via an endoscope or laparoscope, depending on the specific application and/or tumor indication.

With the distal end of the endoscope located at the target site, electroporation and/or drug delivery may commence in a manner as described in any of the embodiments set forth herein. In some embodiments, electroporation and delivery of the treatment agent(s) may be simultaneous or otherwise occur at about the same time. In some embodiments, electroporation may commence prior to delivery of the treatment agent. In some embodiments, delivery of the treatment agent is followed by electroporation. Upon completion of the electroporation, the applicator, and as applicable guiding device such as an endoscope, are removed and, when applicable, the stomach incision is closed as appropriate.

It is further contemplated that the procedure described above for the pancreas may also be similarly performed for a colonoscopy.

In some examples, the method of treating diseased tissue in the gastrointestinal tract may be performed with the aid of robotics. For instance, the applicator may be used with a robotic system to perform a procedure to reach the pancreas with an ultrasound endoscope or the like. In particular, the applicator may be advanced through the body of the patient and/or the electrodes of the applicator may be deployed through control of the robotic device of the robotic system. To perform these functions, for example, an arm of the robotic device may be manipulated to rotate and position the applicator during the procedure. Similarly, the arm of the robotic device may be manipulated to control electricity flow into the applicator. In some examples, other steps of the method may also be aided by the use of the robotic system.

In some embodiments, the present disclosure relates to a method of treating diseased tissue in the urinary system, such as in the urethra or the bladder. In some embodiments an endoscope is used with an applicator inserted therein. In some embodiments, the endoscope is rigid, while in others, it is flexible. In some embodiments, a urethral catheter is used with an applicator. In some embodiments, an applicator is used by itself without any guiding device. As described above, in some embodiments, pre-operative surgical planning may be performed to identify a specific location of the diseased tissue and to evaluate the intended insertion path for the applicator and/or endoscope. Once ready for surgery, the applicator is inserted into the endoscope or urethral catheter, or if the applicator is being used on its own, it is ready for use on its own. As with the other exemplary methods discussed above, the applicator need not be positioned in the endoscope or urethral catheter prior to insertion of either access instrument into the patient (assuming an access instrument of some type is being used at all).

In some embodiments, the endoscope (or urethral catheter) is advanced directly into the urethra from outside the patient and the tip of the endoscope is directed to the diseased tissue. In some embodiments, the endoscope is advanced into the urethra from outside the patient and from the urethra into the bladder. From within the bladder, the endoscope tip is directed to a diseased tissue on the bladder. Whether in the urethra or bladder, the applicator is advanced from within the endoscope so that the applicator is in position for the electroporation procedure. Additionally, visualization aids may accompany the endoscope, along with an optional navigation system and imaging information from pre-operative planning, to aid in the advancement of the applicator to the diseased tissue.

With the distal end of the endoscope located at the target site, electroporation and/or drug delivery may commence in a manner as described in any of the embodiments set forth herein. In some embodiments, electroporation and delivery of the treatment agent(s) may be simultaneous or otherwise occur at about the same time. In some embodiments, electroporation may commence prior to delivery of the treatment agent(s). In some embodiments, delivery of the treatment agent(s) is followed by electroporation.

In some examples, the method of treating diseased tissue in the urinary system may be performed with the aid of robotics. For instance, the applicator may be used with a robotic system to perform the procedure. In particular, the applicator may be advanced through the body of the patient and/or the electrodes of the applicator may be deployed through control of the robotic device of the robotic system. To perform these functions, for example, an arm of the robotic device may be manipulated to rotate and position the applicator during the procedure. Similarly, the arm of the robotic device may be manipulated to control electricity flow into the applicator. In some examples, other steps of the method may also be aided by the use of the robotic system.

In some embodiments, the present disclosure relates to a method of treating diseased tissue in the brain through a neurosurgical procedure. In some examples, the procedure may be used to treat various types of tumors in the brain or in the neurological system more generally. In some embodiments an endoscope is used with an applicator inserted therethrough. In some embodiments, a catheter is used with an applicator. In some embodiments, an applicator is used by itself without any access device. As described above, in some embodiments, pre-operative surgical planning may be performed to identify a specific location of the diseased tissue and to evaluate the intended insertion path for the applicator and/or endoscope.

In some embodiments, an endovascular approach to the diseased tissue in the brain is used. This approach may be used to treat a glioblastoma, glioblastoma multiforme, or the like, for instance. In one example, the applicator, disposed in a catheter or an endoscope, is introduced percutaneously into the body of the patient through the femoral artery, then steered superiorly through the aorta, vena cava, carotid or vertebral artery. Other access points are also suitable for an approach into the cerebrum. Alternatively, the catheter or endoscope is positioned in the patient's vasculature first, prior to positioning the applicator therein. To determine where to steer the applicator from the carotid or vertebral artery, the location of the diseased tissue is compared with the location of the applicator. The applicator is then advanced through the appropriate blood vessels of the brain. In some unique circumstances, it may be possible to further steer the applicator through intra-cranial blood vessels if necessary. However, prior to doing so, the surgeon will assess whether such access is feasible by comparing an outer diameter of the endoscope or catheter compared with the intra-cranial blood vessels to be traversed. In some examples, the applicator may be configured to be advancable relative to the endoscope or catheter, thereby reducing the minimum diameter necessary for access of the device for electroporation. Additionally, visualization aids may accompany the endoscope, along with an optional navigation system and imaging information from pre-operative planning, to aid in the advancement of the applicator to the diseased tissue. Once advancement of the applicator to the diseased tissue at the target site is complete, electroporation may be performed.

In some embodiments, areas around the brain may be accessed through the nose through a transsphenoidal procedure. This may be desirable when the diseased tissue is on or near the pituitary gland or when the diseased tissue is a tumor that grows from the dura (membrane surrounding the brain). Thus, the procedure may be used to treat, for example, pituitary adenoma, craniopharyngioma, rathke's cleft cyst, meningioma and chordoma. In some examples, the applicator is disposed in an endoscope or a catheter and then advanced through the nose and the sphenoid sinus to reach the diseased tissue for the performance of electroporation. In some embodiments, a small incision may be made in one or more of the nasal septum, sphenoid sinus and the sella to reach the diseased tissue. A similar approach involving the creation of small holes in the nasal area may also be used to access the diseased tissue through the mouth. In some examples of the above embodiments, a microscope may also be used to complement the applicator in a procedure.

In each of the described methods of accessing tissue in and around the cerebrum, once the distal end of the applicator is positioned at the target site, electroporation and/or drug delivery may commence in a manner as described in any of the embodiments set forth herein. In some embodiments, electroporation and delivery of the treatment agent(s) may be simultaneous or otherwise occur at about the same time. In some embodiments, electroporation may commence prior to delivery of the treatment agent(s). In some embodiments, delivery of the treatment agent(s) is followed by electroporation.

In some examples, the method of treating diseased tissue in the cerebrum may be performed with the aid of robotics. For instance, the applicator may be used with a robotic system to perform the procedure. In particular, the applicator may be advanced through the body of the patient and/or the electrodes of the applicator may be deployed through control of the robotic device of the robotic system. To perform these functions, for example, an arm of the robotic device may be manipulated to rotate and position the applicator during the procedure. Similarly, the arm of the robotic device may be manipulated to control electricity flow into the applicator. In some examples, other steps of the method may also be aided by the use of the robotic system.

The above described methods demonstrate that the electroporation technology and systems described herein may be employed in a wide variety of surgical applications. The specific examples outlined are intended to demonstrate how the system may be employed in specific applications, and in no way are intended to be limiting in any way. To be clear, further to use of the system to access diseased tissue with the applicator alone, with an endoscope, or with a catheter, it is further contemplated that a trocar may be used to access a target site to perform electroporation. A trocar may be advantageous to provide direct access into bone malignancies, for example, such as primary or secondary sarcomas.

In some embodiments, the methods described herein may be used in combination with tissue imaging procedures in addition to those described elsewhere in the application. For example, procedures including fluorescence imaging, white light imaging, or a combination thereof may be used. In some examples, fluorescence imaging may employ the use of an agent or a dye. Well known examples of such agents include indocyanine green. Such fluorescence imaging agent and visualization capabilities may be used to direct the electroporation applicator to the target tissue. In some instances, the blood flow through a tumor may cause an incidence of dye in the tumor, illuminating the tumor under visualization. Such a process may increase the effectiveness of electroporation as the operator can see and thus treat areas of the tumor which may have not been seen under normal white light visualization.

In some embodiments, the methods, systems, and apparatus described herein may be used with other surgical procedures, including laparoscopies. The methods, systems, and apparatus described herein described herein may also be used with a number of treatments including but not limited to gene therapies (e.g., plasmid therapies) or drug treatments for any of a number of cancers and other diseases.

Referring back to FIG. 1, in some embodiments, the electrodes 100 may be used to detect an impedance of the body tissue between the electrodes at the electroporation site. In particular, the electrical responses of a tissue may be measured over a range of interrogation frequencies transmitted through the electrodes via electrochemical impedance spectroscopy. The collected data may then be fit to equivalent circuit models to determine the electrical properties of the tissue. In some embodiments, the electrical pulses of any of the methods and apparatus disclosed herein may be supplied by a low-voltage generator.

The controller 24 that controls the electroporation process may interface with the generator 12 to provide a feedback loop that fine tunes the generator output to a desired level based on the impedance detected at the electrodes. This process may be implemented for any of the electrode and electroporation systems, methods, and apparatus discussed herein.

Accordingly, blocks of the flowcharts support combinations of means for performing the specified functions and combinations of operations for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

Methods of Treatment

The electroporation devices described herein may be used in therapeutic treatments and in the delivery of treatment agents. In some embodiments, therapeutic treatments include electrotherapy, also referred to herein as electroporation therapy (EPT), using the described apparatuses for the delivery of one or more treatment agents (e.g., molecules) to a cell, group of cells, or tissue and for performing electroporation on the cell, group of cells, or tissue. In some embodiments, the molecule or treatment agent is a drug (i.e., active pharmaceutical ingredient). Combining any of the treatment agent(s) discussed herein or otherwise generally known in the art with EPT, as discussed herein, may provide an effective treatment even in patients who did not respond to the treatment agent(s) on their own. In some embodiments, the drug is a small molecule. In some embodiments, the drug is a macromolecule. A drug can be, but is not limited to, a chemotherapeutic agent. A macromolecule can be, but is not limited to, a chemotherapeutic agent, nucleic acid (such as, but not limited to, polynucleotide, oligonucleotide, DNA, cDNA, RNA, peptide nucleic acid, antisense oligonucleotides, siRNA, miRNA, ribozyme, plasmid, and expression vector), and polypeptide (such as, but not limited to, peptide, antibody, and protein). In some embodiments, therapeutic treatments include delivery of a therapeutic electric pulse to a cell, group of cells, or tissue using any of the described electroporation devices. The cell, group of cells, or tissue may be, but is not limited to, a tumor cell or tumor tissue.

Drugs or treatment agents contemplated for use with the methods include chemotherapeutic agents having an antitumor or cytotoxic effect. A drug can be an exogenous agent or an endogenous agent. In some embodiments, the drug is a small molecule exogenous agent. Small molecule exogenous agent agents include, but are not limited to, bleomycin, neocarcinostatin, suramin, doxorubicin, carboplatin, taxol, mitomycin C and cisplatin. Other chemotherapeutic agents will be known to those of skill in the art (see, for example, The Merck Index). In some embodiments, the drug is a membrane-acting agents. “Membrane acting” agents act primarily by damaging the cell membrane. Non-limiting examples of membrane-acting agents include, N-alkylmelamide and para-chloro mercury benzoate. In some embodiments, the drug is a cytokine, chemokine, lymphokine, or hormone. In some embodiments, the drug is a nucleic acid. In some embodiments, the nucleic acid encodes one or more cytokines, chemokines, lymphokines, therapeutic polypeptide, adjuvant, or a combination thereof.

The molecule or treatment agent can be administered to a subject before, during, or after administration of the electric pulse. The molecule can be administered at or near the cell, group of cells or tissue in a patient. In some embodiments, the molecule can be co-localized with the electric pulse using an applicator having electrodes and a drug delivery channel extending therethrough (e.g., applicator 110; electrodes 100, 200, 400, 500, 600; and drug delivery channel 18 shown in FIGS. 47-66). The chemical composition of the treatment agent will dictate the most appropriate time to administer the agent in relation to the administration of the electric pulse. For example, while not wanting to be bound by a particular theory, it is believed that a drug having a low isoelectric point (e.g., neocarcinostatin, IEP=3.78), would likely be more effective if administered post-electroporation in order to avoid electrostatic interaction of the highly charged drug within the field. Further, such drugs as bleomycin, which have a very negative log P, (P being the partition coefficient between octanol and water), are very large in size (MW=1400), and are hydrophilic, thereby associating closely with the lipid membrane, diffuse very slowly into a tumor cell and are typically administered prior to or substantially simultaneous with the electric pulse. In addition, certain treatment agents may require modification in order to allow more efficient entry into the cell. For example, an agent such as taxol can be modified to increase solubility in water which would allow more efficient entry into the cell. In some embodiments, electroporation facilitates entry of the molecule into a cell by creating pores in the cell membrane.

In some embodiments, the molecule or treatment agent is delivered to modulate expression of a gene. The term “modulate” envisions the decrease (suppression) or increase (stimulation) of expression of a gene. Where a cell proliferative disorder is associated with the expression of a gene, nucleic acid sequences that interfere with the gene's expression at the translational level can be used. In some embodiments, one or more antisense nucleic acids, ribozymes, siRNAs, miRNA, triplex agents, or the like are delivered via electroporation to block transcription or translation of a specific mRNA. In some embodiments, a nucleic acid is delivered to express an RNA or polypeptide. The nucleic acid can be recombinant, single stranded or double stranded, DNA or RNA or a combination of DNA and RNA, circular or linear, and/or supercoiled or relaxed. The nucleic acid can also be associated with one or more of proteins, lipids, virus, viral vector, chimeric virus, or viral particle. The nucleic acid can also be naked. A virus can be, but is not limited, adenovirus, herpes virus, vaccinia, DNA virus, RNA virus, retrovirus, murine retrovirus, avian retrovirus, Moloney murine leukemia virus (MoMuLV), Harvey murine sarcoma virus (HaMuSV), murine mammary tumor virus (MuMTV), Rous Sarcoma Virus (RSV), gibbon ape leukemia virus (GaLV) can be utilized. Similarly a viral vector, chimeric virus, and/or viral particle can be derived from any of the above described viruses.

### Therapeutic Polypeptides

Therapeutic polypeptides (one type of treatment agent listed above) include, but are not limited to, immunomodulatory agents, biological response modifiers, co-stimulatory molecule, metabolic enzymes and proteins, antibodies, checkpoint inhibitors, and adjuvants.

The term “immunomodulatory agents” is meant to encompass substances which are involved in modifying an immune response. Examples of immune response modifiers include, but are not limited to, cytokines, chemokines, lymphokines, and antigen binding polypeptides. Lymphokines can be, but not limited to, tumor necrosis factor, interleukins (IL, such as, but not limited to IL-1, IL-2, IL-3, IL-12, IL-15), lymphotoxin, macrophage activating factor, migration inhibition factor, colony stimulating factor, and alpha-interferon, beta-interferon, gamma-interferon, and their subtypes. In some embodiments, the immune response modifier comprises a nucleic acid encoding one or more cytokines, chemokines, lymphokines or subunits of cytokines, chemokines, and lymphokines. In some embodiments, the immunomodulatory agent is an immune stimulator. Non-limiting examples of immune stimulators include, IL-33, flagellin, IL-10 receptor, sting receptor, IRF3. The term “cytokine” is used as a generic name for a diverse group of soluble proteins and peptides which act as humoral regulators at nano- to picomolar concentrations and which, either under normal or pathological conditions, modulate the functional activities of individual cells and tissues. As used herein an “immunostimulatory cytokine” includes cytokines that mediate or enhance the immune response to a foreign antigen, including viral, bacterial, or tumor antigens. Immunostimulatory cytokines include, but are not limited to, TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, and TGFβ. In some embodiments, the immunostimulatory cytokine is a nucleic acid encoding one or more of TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, and TGFβ.

Another treatment agent, a “co-stimulator,” refers to any of a group of immune cell surface receptor/ligands which engage between T cells and antigen presenting cells and generate a stimulatory signal in T cells which combines with the stimulatory signal (i.e., “co-stimulation”) in T cells that results from T cell receptor (“TCR”) recognition of antigen on antigen presenting cells. Co-stimulatory activation can be measured for T cells by the production of cytokines. As used herein the term “co-stimulatory molecules” includes a soluble co-stimulator or agonists of co-stimulators. Co-stimulatory molecules include, but are not limited to, agonists of GITR, CD137, CD134, CD40L, CD27, and the like. Co-stimulator agonists include, but are not limited to, agonistic antibodies, co-stimulator ligands, including multimeric soluble and transmembrane co-stimulator ligands, co-stimulator ligand peptides, co-stimulator ligand mimetics, and other molecules that engage and induce biological activity of a co-stimulator. In some embodiments, a soluble co-stimulatory molecules derived from an antigen presenting cell may be, but is not limited to, GITR-L, CD137-L, CD134-L (a.k.a. OX40-L), CD40, CD28. Agonists of co-stimulatory molecules may be soluble molecules such as soluble GITR-L, which comprises at least the extracellular domain (ECD) of GITR-L. The soluble form of a co-stimulatory molecule derived from an antigen presenting cell retains the ability of the native co-stimulatory molecule to bind to its cognate receptor/ligand on T cells and stimulate T cell activation. Other co-stimulatory molecules will similarly lack transmembrane and intracellular domains, but are capable of binding to their binding partners and eliciting a biological effect. In some embodiments, for intratumoral delivery by electroporation, the co-stimulator molecule is encoded in an expression vector that is expressed in a tumor cell. In some embodiments, the co-stimulatory molecule is a nucleic acid encoding one or more of GITR, GITR-L, CD137, CD137-L, CD134, CD134-L, CD40, CD40L, CD27, and D28, and the like or a functional fragment thereof. A co-stimulatory molecule includes a molecule that has biological function as co-stimulatory molecule and shares at least 80% amino acid sequence identity, at least 90% sequence identity, at least 95% sequence identity, or at least 98% sequence identity GITR, GITR-L, CD137, CD137-L, CD134, CD134-L, CD40, CD40L, CD27, or D28 or a functional fragment thereof. In some embodiments, a co-stimulatory agonist can be in the form of antibodies or antibody fragments, both of which can be encoded in a plasmid and delivered to the tumor by electroporation.

Other treatment agents, such as metabolic enzymes and proteins, include, but are not limited to, antiangiogenesis compounds. Antiangiogenesis compounds include, but are not limited to, Factor VIII and Factor IX. In some embodiments, the metabolic enzyme or protein comprises a nucleic acid encoding one or more metabolic enzyme or protein comprises or functional fragments thereof.

The term “antibody” as used herein is another treatment agent including immunoglobulins, which are the product of B cells and variants thereof as well as the T cell receptor (TcR), which is the product of T cells, and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, IgG3, and IgG4 subclass. Antibodies exist as full-length intact antibodies or as a number of well-characterized fragments thereof. Antibody fragments can be produced by the modification of whole antibodies or synthesized de novo or antibodies and fragments obtained by using recombinant DNA methodologies. Antibody fragments include, but are not limited to, F(ab′)2, and Fab′, scFv, and ByTE fragments. In some embodiments, antibody comprises a nucleic acid encoding one or more antibodies or antibody fragments.

An “adjuvant,” yet another treatment agent, is a substance that enhances an immune response to an antigen. In some embodiments, adjuvants include, but are not limited to, Freund's adjuvant (complete and incomplete), mineral salts such as aluminum hydroxide or aluminum phosphate, various cytokines, surface active substances such as lysolecithin, pluronic polyols, polyanions, peptides, oil emulsions, and potentially useful human adjuvants such as BCG (bacille Calmette-Guerin) and Corynebacterium parvum. In some embodiments, an adjuvant is or comprised keyhole limpet hemocyanin, tetanus toxoid, diphtheria toxoid, ovalbumin, cholera toxin or functional fragments thereof. In some embodiments, an adjuvant is or comprises Granulocyte-macrophage colony-stimulating factor (GM-CSF), Flt3 ligand. LAMP1, calreticulin, human heat shock protein 96, CSF Receptor 1 or a functional fragment thereof. In some embodiments, an adjuvant comprises a nucleic acid encoding one or more adjuvants or adjuvant fragments (i.e., genetic adjuvants). In some embodiments, a genetic adjuvant is fused to an antigen. An antigen can be, but is not limited to, a tumor antigen, shared tumor antigen or viral antigen. Non-limiting examples of antigens include, NY-ESO-1 or a fragment thereof, MAGE-A1, MAGE-A2, MAGE-A3, MAGE-A10, SSX-2, MART-1, Tyrosinase, Gp100, Survivin, hTERT, PRS pan-DR, B7-H6, HPV-7, HPV16 E6/E7, HPV11 E6, HPV6b/11 E7, HCV-NS3, Influenza HA, Influenza NA, and polyomavirus. In some embodiments, a genetic adjuvant is fused to a cytokine, or co-stimulatory molecule.

Another treatment agent, an immune checkpoint molecule, refers to any of a group of immune cell surface receptor/ligands which induce T cell dysfunction or apoptosis. These immune inhibitory targets attenuate excessive immune reactions and ensure self-tolerance. As used herein “checkpoint inhibitor” comprises a molecules that prevent immune suppression by blocking the effects of an immune checkpoint molecule. Checkpoint inhibitors include, but are not limited to, antibodies and antibody fragments, nanobodies, diabodies, soluble binding partners of checkpoint molecules, small molecule therapeutics, peptide antagonists, etc. In some embodiments, a checkpoint inhibitor can be, but is not limited to, CTLA-4 antagonist, PD-1 antagonist, PD-L1 antagonist, LAG-3 antagonist, TIM3 antagonist, KIR antagonist, BTLA antagonist, A2aR antagonist, HVEM antagonist. In some embodiments the checkpoint inhibitor is selected from the group comprising: nivolumab (ONO-4538/BMS-936558, MDX1 106, OPDIVO), pembrolizumab (MK-3475, KEYTRUDA), pidilizumab (CT-011), and MPDL3280A (ROCHE). In some embodiments, a checkpoint inhibitor polypeptide can be encoded by a nucleic acid that is delivery to a tumor.

### Expression Vectors

Any of the described polypeptides may be encoded on nucleic acid, to form yet another treatment agent. The nucleic acid can be, but is not limited to, an expression vector or plasmid. The term “plasmid” or “vector” includes any known delivery vector including a bacterial delivery vector, a viral vector delivery vector, an episomal plasmid, an integrative plasmid, or a phage vector. The term “vector” refers to a construct which is capable of expressing one or more polypeptides in a cell.

An encoded polypeptide may be linked, in an expression vector to a sequence encoding a second polypeptide. In some embodiments, an expression vector encodes a fusion protein. The term “fusion protein” refers to a protein comprising two or more polypeptides linked together by peptide bonds or other chemical bonds. In some embodiments, a fusion protein is be recombinantly expressed as a single-chain polypeptide containing the two polypeptides. The two or more polypeptides can be linked directly or via a linker comprising one or more amino acids.

In some embodiments, the nucleic acid (i.e., expression vector) encodes two polypeptides expressed from a single promoter, with an intervening exon skipping motif that allows both polypeptides to be expressed from a single polycistronic message. In some embodiments, the expression vector comprises:
P-A-T-C, P-C-T-A, or P-A-T-B
   wherein P is a promoter, A, B, and C are nucleic acid sequences encoding therapeutic polypeptides, and T is a translation modification element. A translation modification element can be, but is not limited to, an internal ribosome entry site (IRES) and a ribosomal skipping modulators, such as, but not limited to P2A, T2A, E2A or F2A. In some embodiments, A and B comprise nucleic acid sequences encoding immunomodulatory molecules. In some embodiments, A and B encode cytokines or cytokine subunits, such as, but not limited to, IL-12 p35 and IL-12 p40.

In some embodiments, the nucleic acid (i.e., expression vector) encodes three polypeptides expressed from a single promoter, with intervening ribosome skipping motifs to allow all three proteins to be expressed from a single polycistronic message. In some embodiments, the expression vector comprises:
P-A-T-B-T-C or P-C-T-A-T-B
   wherein P is a promoter, A, B, and C are nucleic acid sequences encoding therapeutic polypeptides, and T is a translation modification element. A translation modification element includes, but is not limited to, an internal ribosome entry site (IRES) and a ribosomal skipping modulators, such as, but not limited to P2A, T2A, E2A or F2A. In some embodiments, A and B comprise nucleic acid sequences encoding immunomodulatory molecules and/or co-stimulatory molecules, or subunits thereof. In some embodiments, A and B encode chains of a heterdimeric cytokine. In some embodiments, C comprises a nucleic acid sequence encoding a costimulatory molecule, genetic adjuvant, antigen, a genetic adjuvant-antigen fusion polypeptide, chemokine, or antigen binding polypeptide. Chemokines include, but are not limited to CXCL9. An antigen binding polypeptide can be, but is not limited to, a scFv. A scFv can be, but is not limited to, an anti-CD3 scFv and an anti-CTLA-4 scFv.

The promoter can be, but is not limited to, human CMV promoter, simian CMV promoter, SV-40 promoter, mPGK promoter, and β-Actin promoter.

In some embodiments, A encodes an IL-12 p35, IL-23p19, EBI3, or IL-15, and B encodes an IL-12 p40, IL-27p28, or IL-15Rt.

In some embodiments, the genetic adjuvant comprises Flt3 ligand; LAMP-1; Calreticulin; Human heat shock protein 96; GM-CSF; and CSF Receptor 1.

In some embodiments, the antigen comprises: NYESO-1, OVA, RNEU, MAGE-A1, MAGE-A2, Mage-A10, SSX-2, Melan-A, MART-1, Tyr, Gp100, LAGE-1, Survivin, PRS pan-DR, CEA peptide CAP-1, OVA, HCV-NS3, and an HPV vaccine peptide.

The IL-12 p35 and IL-12 p40 polypeptide may be mouse or human IL-12 p35 and IL-12 p40.

In some embodiments P is a CMV promoter, A encodes an IL-12 p35 polypeptide, T is an IRES and B encodes an IL-12 p40 polypeptide.

In some embodiments P is a CMV promoter, A encodes an IL-12 p35 polypeptide, T is P2A element, and B encodes an IL-12 p40 polypeptide.

In some embodiments P is a CMV promoter, A encodes a human IL-12 p35 (h IL-12 p35) polypeptide, T is an IRES and B encodes a human IL-12 p40 (hIL-12 p40) polypeptide.

In some embodiments P is a CMV promoter, A encodes a human IL-12 p35 polypeptide, T is P2A element, and B encodes a human IL-12 p40 polypeptide.

In some embodiments, A encodes an IL-12 p35, B encodes an IL-12 p40 polypeptide and C encodes a co-stimulatory polypeptide.

In some embodiments, A encodes an IL-12 p35, B encodes an IL-12 p40 polypeptide and C encodes a NY-ESO1-Flt3L or Flt3L-NY-ESO1 fusion polypeptide.

In some embodiments, A encodes a hIL-12 p35 polypeptide, T is a P2A element, B encodes a hIL-12 p40 polypeptide and C encodes a FLT3L-NYESO1 fusion polypeptide.

In some embodiments, A encodes a hIL-12 p35 polypeptide, T is an IRES element, B encodes a hIL-12 p40 polypeptide and C encodes a FLT3L-NYESO1 fusion polypeptide.

In some embodiments, P is a CMV promoter, A encodes a hIL-12 p35 polypeptide, T is a P2A element, B encodes a hIL-12 p40 polypeptide and C encodes a FLT3L-NYESO1 fusion polypeptide.

In some embodiments, P is a CMV promoter, A encodes a hIL-12 p35 polypeptide, T is an IRES element, B encodes a hIL-12 p40 polypeptide and C encodes a FLT3L-NYESO1 fusion polypeptide.

In some embodiments, A encodes an IL-12 p35, B encodes an IL-12 p40 polypeptide and C encodes a polypeptide comprising an anti-CD3 scFv. In some embodiments, A encodes a hIL-12 p35 polypeptide, T is a P2A element, B encodes a hIL-12 p40 polypeptide and C encodes a polypeptide comprising an anti-CD3 scFv. In some embodiments, A encodes a hIL-12 p35 polypeptide, T is an IRES element, B encodes a hIL-12 p40 polypeptide and C encodes a polypeptide comprising an anti-CD3 scFv. In some embodiments, P is a CMV promoter, A encodes a hIL-12 p35 polypeptide, T is a P2A element, B encodes a hIL-12 p40 polypeptide and C encodes a polypeptide comprising an anti-CD3 scFv. In some embodiments, P is a CMV promoter, A encodes a hIL-12 p35 polypeptide, T is an IRES element, B encodes a hIL-12 p40 polypeptide and C encodes a polypeptide comprising an anti-CD3 scFv.

In some embodiments, A encodes an IL-12 p35, B encodes an IL-12 p40 polypeptide and C encodes a CXCL9. In some embodiments, A encodes a hIL-12 p35 polypeptide, T is a P2A element, B encodes a hIL-12 p40 polypeptide and C encodes a CXCL9. In some embodiments, A encodes a hIL-12 p35 polypeptide, T is an IRES element, B encodes a hIL-12 p40 polypeptide and C encodes a CXCL9. In some embodiments, P is a CMV promoter, A encodes a hIL-12 p35 polypeptide, T is a P2A element, B encodes a hIL-12 p40 polypeptide and C encodes a CXCL9. In some embodiments, P is a CMV promoter, A encodes a hIL-12 p35 polypeptide, T is an IRES element, B encodes a hIL-12 p40 polypeptide and C encodes a CXCL9.

In some embodiments, A encodes an IL-12 p35, B encodes an IL-12 p40 polypeptide and C encodes a CTLA-4 scFv. In some embodiments, A encodes a hIL-12 p35 polypeptide, T is a P2A element, B encodes a hIL-12 p40 polypeptide and C encodes a CTLA-4 scFv. In some embodiments, A encodes a hIL-12 p35 polypeptide, T is an IRES element, B encodes a hIL-12 p40 polypeptide and C encodes a CTLA-4 scFv. In some embodiments, P is a CMV promoter, A encodes a hIL-12 p35 polypeptide, T is a P2A element, B encodes a hIL-12 p40 polypeptide and C encodes a CTLA-4 scFv. In some embodiments, P is a CMV promoter, A encodes a hIL-12 p35 polypeptide, T is an IRES element, B encodes a hIL-12 p40 polypeptide and C encodes a CTLA-4 scFv.

Described are methods for the treatment of malignancies, wherein the administration of a plasmid or expression vector encoding one or more therapeutic polypeptides, in combination with electroporation has a therapeutic effect on lesions (e.g., primary or secondary tumors). Also described are methods for the treatment of malignancies, wherein the administration of a plasmid or expression vector encoding one or more therapeutic polypeptides, in combination with electroporation has a therapeutic effect on primary tumors as well as distant tumors and metastases. In some embodiments, the plasmid or expression vector encodes one or more of immunomodulatory agents, biological response modifiers, co-stimulatory molecule, metabolic enzymes and proteins, antibodies, checkpoint inhibitors, and/or adjuvants.

In some embodiments, the plasmid or expression vector encodes at least one immunostimulatory cytokine, chosen from IL-12, IL-15, and a combination of IL-12 and IL-15.

In some embodiments, the plasmid or expression vector encodes a co-stimulatory molecule. The co-stimulatory molecule can be, but is not limited to, GITR, CD137, CD134, CD40L, and CD27 agonists. Co-stimulatory agonists may be in the form of antibodies or antibody fragments, both of which can be encoded in a plasmid or expression vector and delivered to the tumor by electroporation.

In some embodiments, the plasmid or expression vector encodes CXCL9, anti-CD3 scFv, or anti-CTLA-4 scFv.

Described are methods of treating a cancer comprising administering to a subject, by electroporation using the described electroporation systems and applicators, a therapeutically effective amount one or more of the described expression vectors. The one or more expression vectors are injected into a tumor, tumor microenvironment, tumor margin tissue, peritumoral region, lymph node, intradermal region, and/or muscle, and electroporation therapy is applied to the tumor, tumor microenvironment, tumor margin tissue, peritumoral region, lymph node, intradermal region, and/or muscle. The electroporation therapy may be applied by the described electroporation systems and/or applicators. The described expression vectors, when delivered using the described electroporation systems and applicators, result in local expression of the encoded proteins, leading to T cell recruitment and anti-tumor activity. In some embodiments, the methods also result in abscopal effects, i.e., regression of one or more untreated tumors. In some embodiments, regression includes debulking of a solid tumor.

In some embodiments, therapy is achieved by intratumoral delivery of plasmids or expression vectors encoding therapeutic polypeptides using electroporation.

### Combination Therapy

In some embodiments, a therapeutic method includes a combination therapy. A combination therapy comprises a combination of therapeutic molecules or treatments. Therapeutic treatments include, but are not limited to, electric pulse (i.e., electroporation), radiation, antibody therapy, and chemotherapy. In some embodiments, administration of a combination therapy is achieved by electroporation alone. In some embodiments, administration of a combination therapy is achieved by a combination of electroporation and systemic delivery. In some embodiments, a plasmid expressing one or more immunomodulatory peptides is administered by intratumoral electroporation and a checkpoint inhibitor is administered systemically. In some embodiments, the immunomodulatory peptide is IL-12, CD3 half-BiTE, CXCL9, or CTLA-4 scFv. In some embodiments, the one or more immunomodulatory peptides included IL-12 and CD3 half-BiTE, CXCL9, or CTLA-4 scFv. In some embodiments, administration of a combination therapy is achieved by a combination of electroporation and radiation. Therapeutic electroporation can be combined with, or administered with, one or more additional therapeutic treatments. The one or more additional therapeutics can be delivered by systemic delivery, intratumoral delivery, and/or radiation. The one or more additional therapeutics can be administered prior to, concurrent with, or subsequent to the electroporation therapy. In some embodiments, the therapeutics (i.e., a treatment agent) can be administered co-locally with the electric pulse or other treatment using an applicator having both electrodes and a drug delivery channel extending therethrough (e.g., applicator 110; electrodes 100, 200, 400, 500, 600; and drug delivery channel 18 shown in FIGS. 47-66). In such embodiments, the generator may deliver an electrical pulse to the electrodes to electroporate target tissue to allow the treatment agent administered via the drug delivery channel to permeate and treat the target tissue.

In some embodiments, intratumoral electroporation of an expression vector encoding a co-stimulatory agonist can be administered with other therapeutic entities, all of which can be treatment agents. In some embodiments, the co-stimulatory molecule is combined with one or more of: CTLA4, cytokines (i.e. IL-12 or IL-2), tumor vaccine, small molecule drug, small molecule inhibitor, targeted radiation, anti-PD1 antagonist, and anti-PDL1 antagonist Ab. A small molecule drug can be, but is not limited to, bleomycin, gemzar, cytozan, 5-fluoro-uracil, adriamycin, and other chemotherapeutic drug agent. A small molecule inhibitor can be, but is not limited to: Sunitinib, Imatinib, Vemurafenib, Bevacizumab, Cetuximb, rapamycin, Bortezomib, PI3K-AKT inhibitors, and IAP inhibitors. In some embodiments, the co-stimulatory molecule can is combined with one or more of: TLR agonists (e.g., Flagellin, CpG); IL-10 antagonists (e.g., anti-IL-10 or anti-IL-10R antibodies); TGFβ antagonists (e.g., anti-TGFβ antibodies); PGE2 inhibitors; Cbl-b (E3 ligase) inhibitors; CD3 agonists; telomerase antagonists, and the like. In particular, various combinations of IL-12, IL-15/IL-15Ra, and/or GITR-L are contemplated. IL-12 and IL-15 have been shown to have synergistic anti-tumor effects. In some embodiments, two or more therapeutic polypeptides are delivered by intratumoral electroporation therapy. The therapeutic polypeptides can be expressed from a single expression vector or plasmid or multiple expression vectors or plasmids.

In some embodiments, combination therapy comprises administration of treatment agents including a checkpoint inhibitor and an immunostimulatory cytokine. In some embodiments, the checkpoint inhibitor is encoded on an expression vector and delivered to a tumor by electroporation therapy. In some embodiments, the immunostimulatory cytokine is encoded on an expression vector and delivered to a tumor by electroporation therapy. In some embodiments, the checkpoint inhibitor and the immunostimulatory cytokine are encoded on an expression vector, wherein expression is driven by a single promoter, and delivered to the cancerous tumor by electroporation therapy. In some embodiments, the checkpoint inhibitor is a systemically administered polypeptide and the immunostimulatory cytokine is administered by intratumoral electroporation of an expression vector encoding the immunostimulatory cytokine. In some embodiments, the expression vector encoding the immunostimulatory cytokine further encodes a CD3 half-BiTE, CXCL9 or CTLA-4 scFv.

Checkpoint inhibitor therapy may occur before, during, or after intratumoral delivery by electroporation of an immunostimulatory cytokine. A checkpoint inhibitor may be in the form of antibodies or antibody fragments, both of which can be encoded in a plasmid and delivered to the tumor by electroporation, or delivered as proteins/peptides systemically. In some embodiments, the checkpoint inhibitor is encoded on an expression vector and delivered to the tumor by electroporation therapy. In some embodiments, the checkpoint inhibitor is administered after electroporation of the immunostimulatory cytokine, whereby administration of certain treatment agents are staggered and administered at different times relative to the electroporation step.

### Treatment

The term “treatment” includes, but is not limited to, inhibition or reduction of proliferation of cancer cells, destruction of cancer cells, prevention of proliferation of cancer cells or prevention of initiation of malignant cells or arrest or reversal of the progression of transformed premalignant cells to malignant disease, or amelioration of the disease.

In some embodiments, methods are provided for reducing the size of a tumor or inhibiting the growth of cancer cells in a subject, or reducing or inhibiting the development of metastatic cancer in a subject suffering from cancer.

In some embodiments, one or more of the methods comprises, treating a subject having a cancerous tumor comprising: injecting the cancerous tumor with an effective dose of a therapeutic molecule or treatment agent; and administering electroporation therapy to the tumor. In some embodiments, one or more of the methods comprises, treating a subject having a cancerous tumor comprising: injecting the cancerous tumor with an effective dose of an expression plasmid encoding a therapeutic polypeptide; and administering electroporation therapy to the tumor.

In some embodiments, the described devices can be used for the therapeutic application of an electric pulse to a cell, groups of cells, or tissue of a subject for damaging or killing cells therein. In some embodiment the cell is a cancer cell. In some embodiments, the cancer cell is malignant.

In some embodiments, the described devices can be used for the therapeutic application of an electric pulse to a cell, groups of cells, or tissue of a subject thereby facilitating entry of a therapeutic molecule into the cell, groups of cells, or tissue. In some embodiments, the described devices can administer the therapeutic molecule to the cell, groups of cells, or tissue. In some embodiments, the described devices may be used both for the therapeutic application of an electrical pulse and for administration of the therapeutic molecules, such that the electrical pulse and the therapeutic molecules are co-localized at the same cell, groups of cells, or tissue without having to reposition the applicator or change the treatment apparatus. In some embodiments the cell is a cancer cell. In some embodiments, the cancer cell is malignant.

In some embodiments, the therapeutic molecule or expression vector is administered substantially contemporaneously with the electroporation treatment. The term “substantially contemporaneously” means that the molecule and the electroporation treatment are administered reasonably close together with respect to time, i.e., before the effect of the electrical pulses on the cells diminishes. The administration of the molecule or therapeutic agent depends upon such factors as, for example, the nature of the tumor, the condition of the patient, the size and chemical characteristics of the molecule and half-life of the molecule.

In some embodiments of the treatment agent, the molecule is combined with one or more pharmaceutically acceptable excipients. Pharmaceutically acceptable excipients (excipients) are substances other than an active pharmaceutical ingredient (API, therapeutic product) that are intentionally included with the API (molecule). Excipients do not exert or are not intended to exert a therapeutic effect at the intended dosage. Excipients may act to a) aid in processing of the API during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance. Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The described electroporation devices and methods can be used to treat a cell, group of cells, or tissue. In some embodiments, the described electroporation devices and methods can be used to treat one or more lesions. In some embodiments, the described electroporation devices and methods can be used to treat tumor cells. The tumor cells can be, but are not limited to cancer cells. The term “cancer” includes a myriad of diseases generally characterized by inappropriate cellular proliferation, abnormal or excessive cellular proliferation. The cancer can be, but is not limited to, solid cancer, sarcoma, carcinoma, and lymphoma. The cancer can also be, but is not limited to, pancreas, skin, brain, liver, gall bladder, stomach, lymph node, breast, lung, head and neck, larynx, pharynx, lip, throat, heart, kidney, muscle, colon, prostate, thymus, testis, uterine, ovary, cutaneous and subcutaneous cancers. Skin cancer can be, but is not limited to, melanoma and basal cell carcinoma. Melanoma can be, but is not limited to, cutaneous and subcutaneous melanoma. Breast cancer can be, but is not limited to, ER positive breast cancer, ER negative breast cancer, and triple negative breast cancer. In some embodiments the tumor cells may include glioblastoma. The cancer can be, but is not limited to, a cutaneous lesion or subcutaneous lesion. In some embodiments, the described devices and methods can be used to treat are used to treat cell proliferative disorders. The term “cell proliferative disorder” denotes malignant as well as non-malignant cell populations which often appear to differ from the surrounding tissue both morphologically and genotypically. In some embodiments, the described devices and methods can be used to treat a human. In some embodiments, the described devices and methods can be used to treat non-human animals or mammals. A non-human mammal can be, but is not limited to, mouse, rat, rabbit, dog, cat, pig, cow, sheep and horse. The administration of the molecule or therapeutic agent and electroporation can occur at any interval, depending upon such factors, for example, as the nature of the tumor, the condition of the patient, the size and chemical characteristics of the molecule and half-life of the molecule.

The described electroporation devices and methods are contemplated for use in patients afflicted with cancer or other non-cancerous (benign) growths. These growths may manifest themselves as any of a lesion, polyp, neoplasm (e.g. papillary urothelial neoplasm), papilloma, malignancy, tumor (e.g. Klatskin tumor, hilar tumor, noninvasive papillary urothelial tumor, germ cell tumor, Ewing's tumor, Askin's tumor, primitive neuroectodermal tumor, Leydig cell tumor, Wilms' tumor, Sertoli cell tumor), sarcoma, carcinoma (e.g. squamous cell carcinoma, cloacogenic carcinoma, adenocarcinoma, adenosquamous carcinoma, cholangiocarcinoma, hepatocellular carcinoma, invasive papillary urothelial carcinoma, flat urothelial carcinoma), lump, or any other type of cancerous or non-cancerous growth. Tumors treated with the devices and methods of the present embodiment may be any of noninvasive, invasive, superficial, papillary, flat, metastatic, localized, unicentric, multicentric, low grade, and high grade.

The described electroporation devices and methods are contemplated for use in numerous types of malignant tumors (i.e. cancer) and benign tumors. For example, the devices and methods described herein are contemplated for use in adrenal cortical cancer, anal cancer, bile duct cancer (e.g. periphilar cancer, distal bile duct cancer, intrahepatic bile duct cancer) bladder cancer, benign and cancerous bone cancer (e.g. osteoma, osteoid osteoma, osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell) esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer, both melanoma and non-melanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). As described herein, a lesion may be described in relation to the organ or region on or in which it resides. For example, a lesion may be considered “at a lung” if it is attached to, disposed on, or disposed within any portion of the lungs and/or lung tissue or would otherwise be associated with the lung by a person of skill in the art in light of this disclosure.

In some embodiments, an electric pulse of electric energy is applied to tissue near or surrounding the target site (e.g. tumor margin tissue). The electric pulse can be applied to tissue near or surrounding the tumor site either before or after excision of the tumor. The electric pulse and optionally a therapeutic molecule can be applied to tissue near or surrounding the tumor site to kill or damage cancerous cells or to deliver one or more therapeutic molecules. The therapeutic molecule can be administered to a subject or tissue intravenously or by injecting directly onto and around the tumor. The electric pulse and optionally a therapeutic molecule can be delivered to a tumor margin tissue to reduce relapse of growth of tumor cells, tumor branches, and/or microscopic metastases in a mammalian tissue at or adjacent to a localization for a tumor excised from a subject. The therapeutic molecule can be administered to the margin tissue before or simultaneously with administration of an electroporating electrical pulse. The electric pulse and optionally the therapeutic molecule can be administered prior to or after surgical resection or ablation of a tumor. In some embodiments, surgical resection or ablation of the tumor is performed with 24 hours of electroporative electric pulse administration. The tumor margin tissue comprises tissue within 0.5-2.0 cm around the tumor. In some embodiments, the tumor margin tissue comprises an open surgical wound margin.

In some embodiments, methods of treating a subject having a cancerous tumor comprise: a) injecting the cancerous tumor with an effective dose of a therapeutic molecule (e.g., treatment agent), and b) administering an electric pulse to the tumor using a described electroporation device. In some embodiments, therapeutic molecule comprises a nucleic acid. In some embodiments, the therapeutic molecule encodes one or more co-stimulatory molecules, metabolic enzymes, antibodies, checkpoint inhibitors, or adjuvants.

In some embodiments, methods of treating a subject having a cancerous tumor comprise: a) injecting the cancerous tumor with an effective dose of at least one expression vector coding for at least one immunostimulatory cytokine(s) and at least one co-stimulatory molecule; b) administering electroporation therapy to the tumor use a described electroporation device.

In some embodiments, the methods further comprise administering an effective dose of one or more checkpoint inhibitors to the subject. In some embodiments, methods of treating a subject having a cancerous tumor comprise: a) injecting the cancerous tumor with an effective dose of at least one plasmid coding for at least one immunostimulatory cytokine(s); b) administering electroporation therapy to the tumor use a described electroporation device; and c) administering an effective dose of one or more checkpoint inhibitors to the subject.

In some embodiments, the electroporation therapy may be any of the therapies detailed herein. In some embodiments, the electroporation therapy may comprise a low-voltage therapy without the performance of EIS. In some embodiments, the controller of the system may cause the generator to perform EIS between pulses of the low-voltage therapy to determine and optimize the parameters of the generator based on the operating conditions and treatment agents used. For example, the parameters (e.g., voltage, pulse duration, etc.) of the generator may be controlled by the controller to cause optimum permeation of the treatment agent.

In some embodiments, the electroporation therapy comprises the administration of one or more voltage pulses having a duration of approximately 0.1 ms each. The voltage pulse that can be delivered to the tumor may be about 400V/cm for low-voltage generators and 1500V/cm for high-voltage generators. In another embodiment, the checkpoint inhibitor is administered systemically. In some embodiments, either a high or a low voltage may be used with the treatment therapies and apparatus disclosed herein.

### Example A

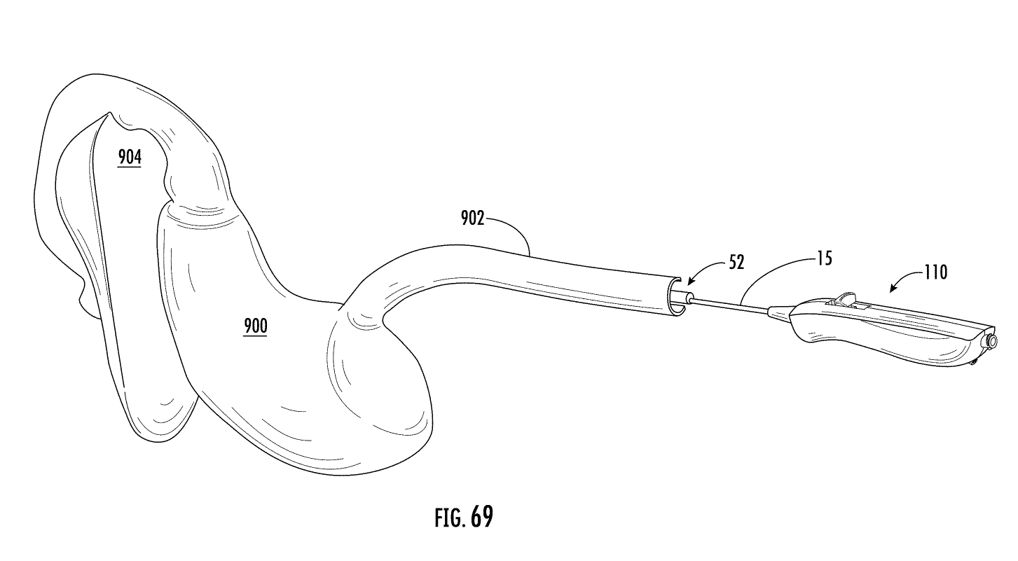

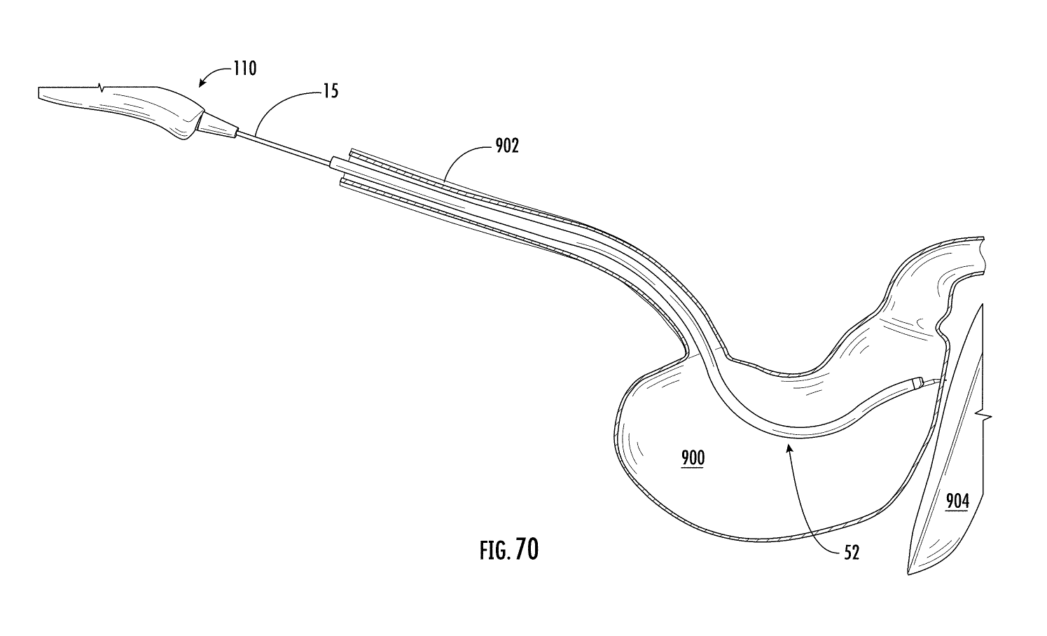

With reference to FIGS. 69-74, an example is shown in which the therapeutic treatments described herein are administered to a lesion on the pancreas, which is accessed via the alimentary canal. With reference to FIGS. 69-70, an applicator 110 is shown having an insertion tube 15 disposed in an endoscope 52. The endoscope 52 and insertion tube 15 are inserted into the stomach 900 via the esophagus 902 to access the stomach wall adjacent to the pancreas 904.

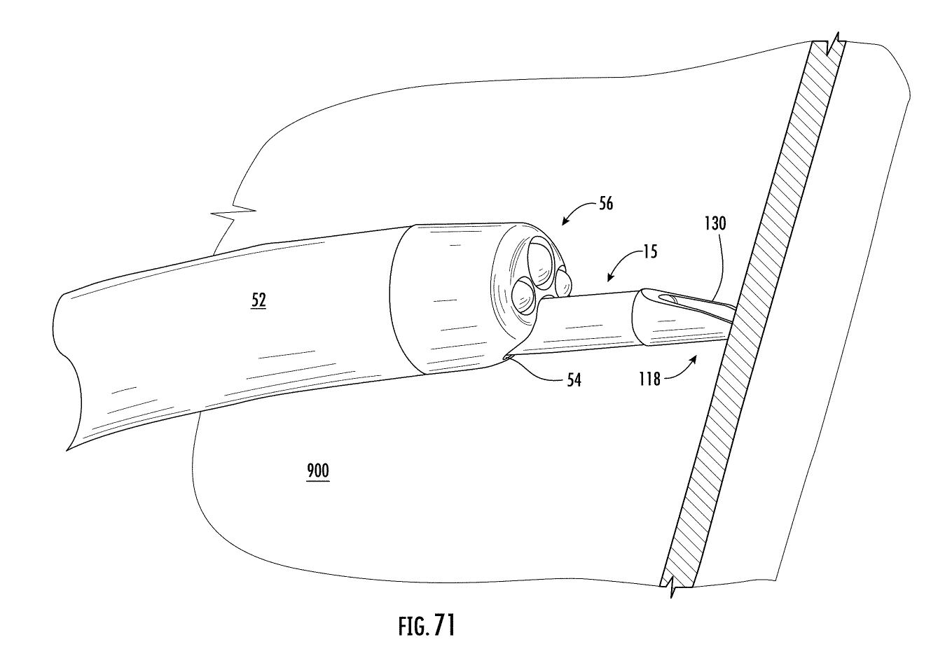

With reference to FIG. 71 a zoomed view of the distal end 56 of the endoscope 52 is shown having the insertion tube 15 of the applicator protruding from the working channel 54 inside the stomach 900. As depicted in FIG. 71, the electrodes and drug delivery channel are in a retracted position within the applicator. The depicted insertion tube 15 includes a piercing tip 130 at its distal end 118 for piercing the stomach wall. Additional features may be included in the remaining portions of the endoscope, such as a lens for imaging, one or more illumination lights, and/or one or more additional working channels. For example, the endoscope 52 shown in FIG. 71 includes a large imaging lens (top center) and two illumination lights (center left and center right) for facilitating the procedures discussed herein.

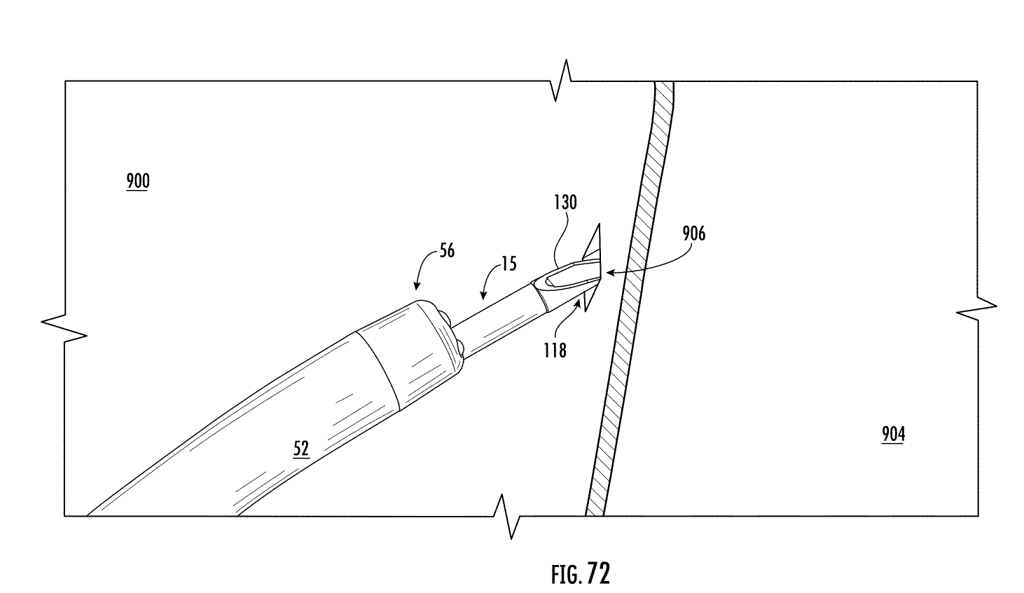

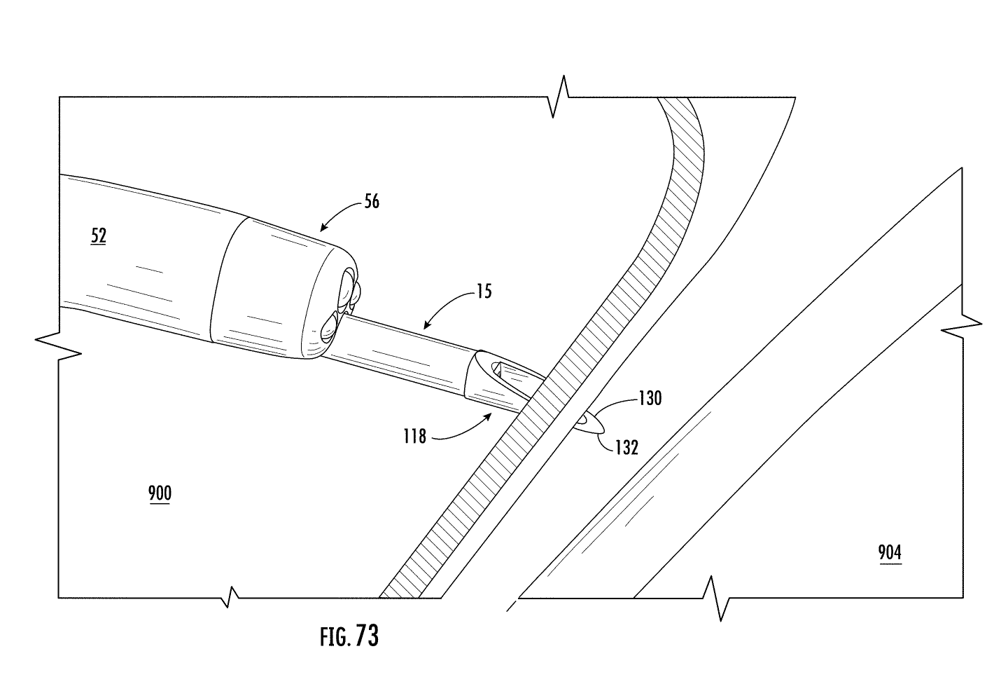

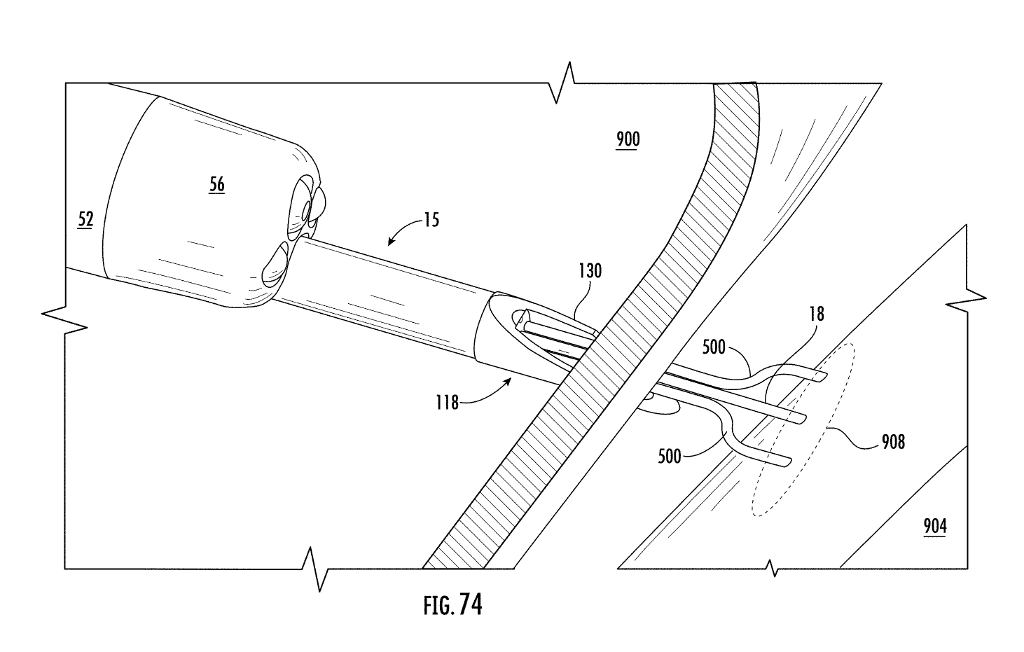

Turning to FIGS. 72-73, a zoomed view of the distal end 56 of the endoscope 52 is shown in which the insertion tube 15 of the applicator is creating a puncture 906 in the wall of the stomach 900 with the piercing tip 130 of the distal end 118. The electrodes and drug delivery channel remain retracted in FIGS. 72-73.

In FIG. 74, the applicator of FIGS. 69-73 is shown extending through the puncture in the stomach 900 with its electrodes 500 and drug delivery channel 18 moved into the deployed position. The depicted electrodes 500 and drug delivery channel 18 are piercing the pancreas 904 at a target site 908 that may be a visceral lesion such as a tumor or other malignancy. From the configuration depicted in FIG. 74, any of the therapies disclosed herein may be administered to the target site 908, including treatment agents, electroporation therapies, and various combination therapies.

### Example B

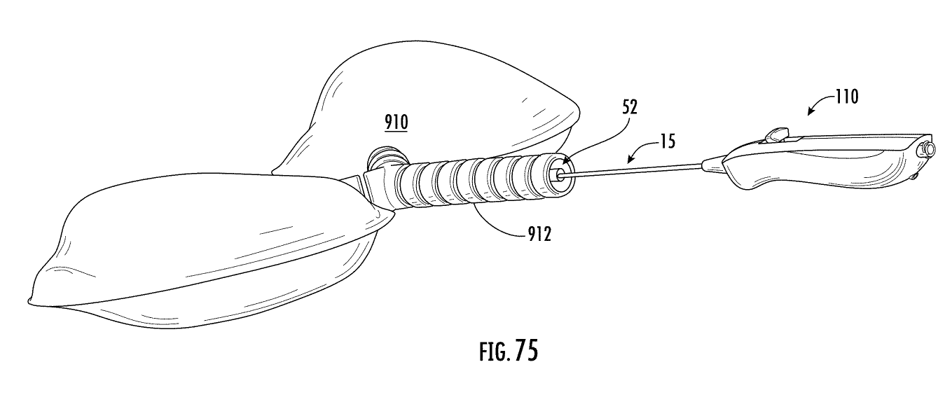

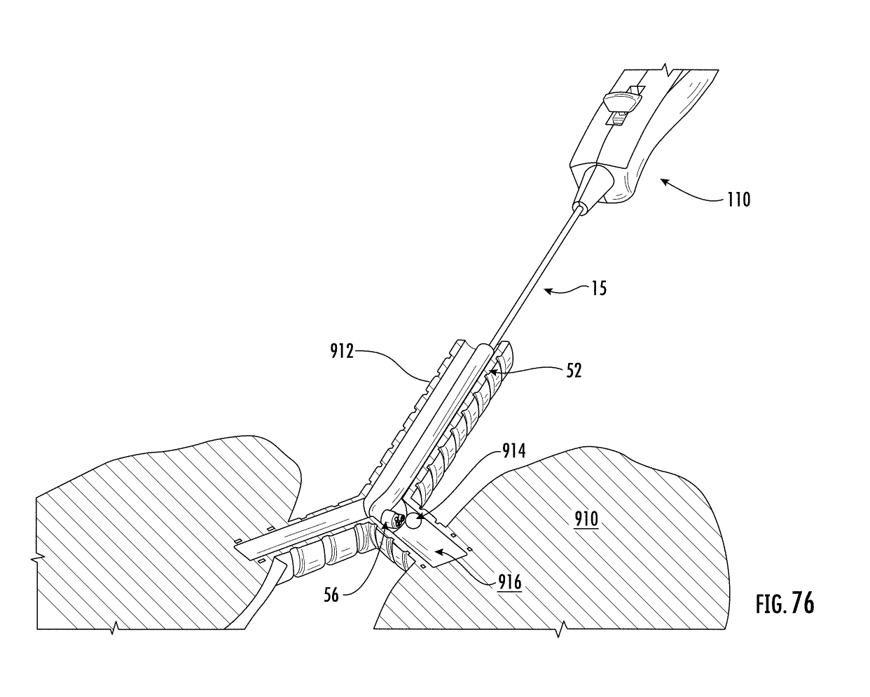

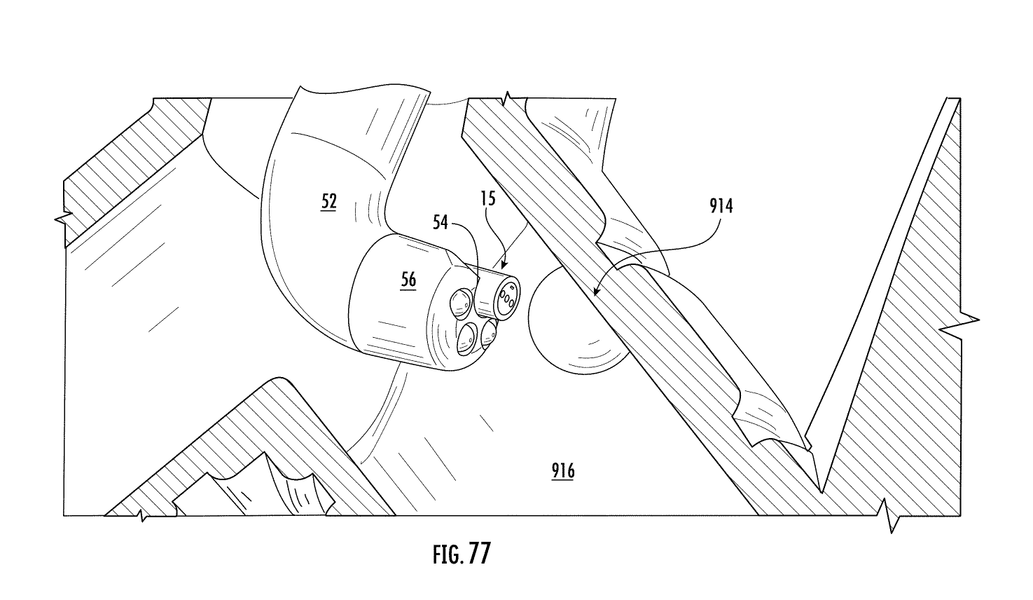

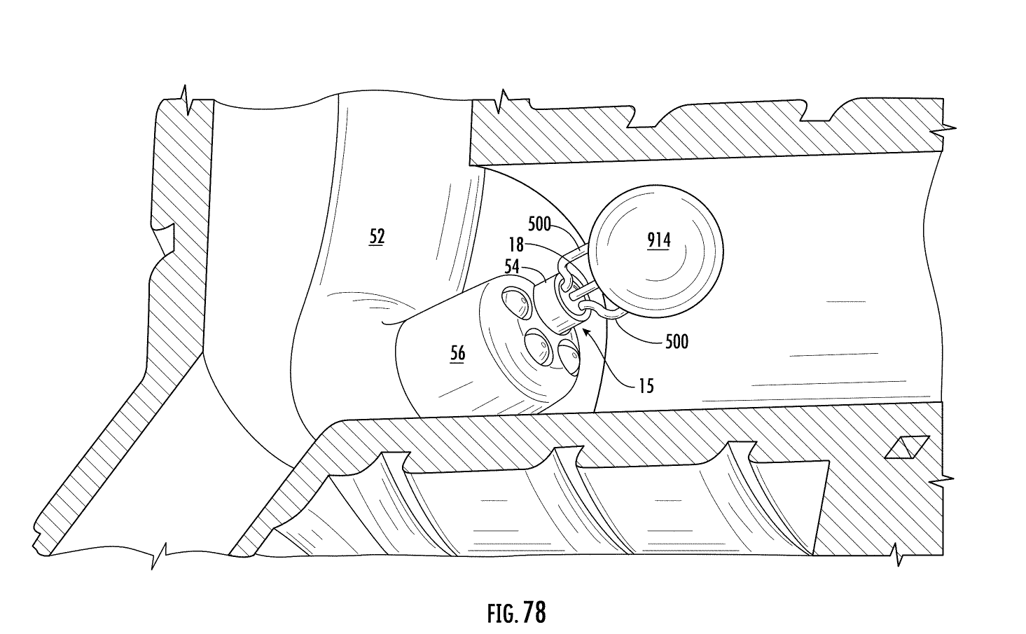

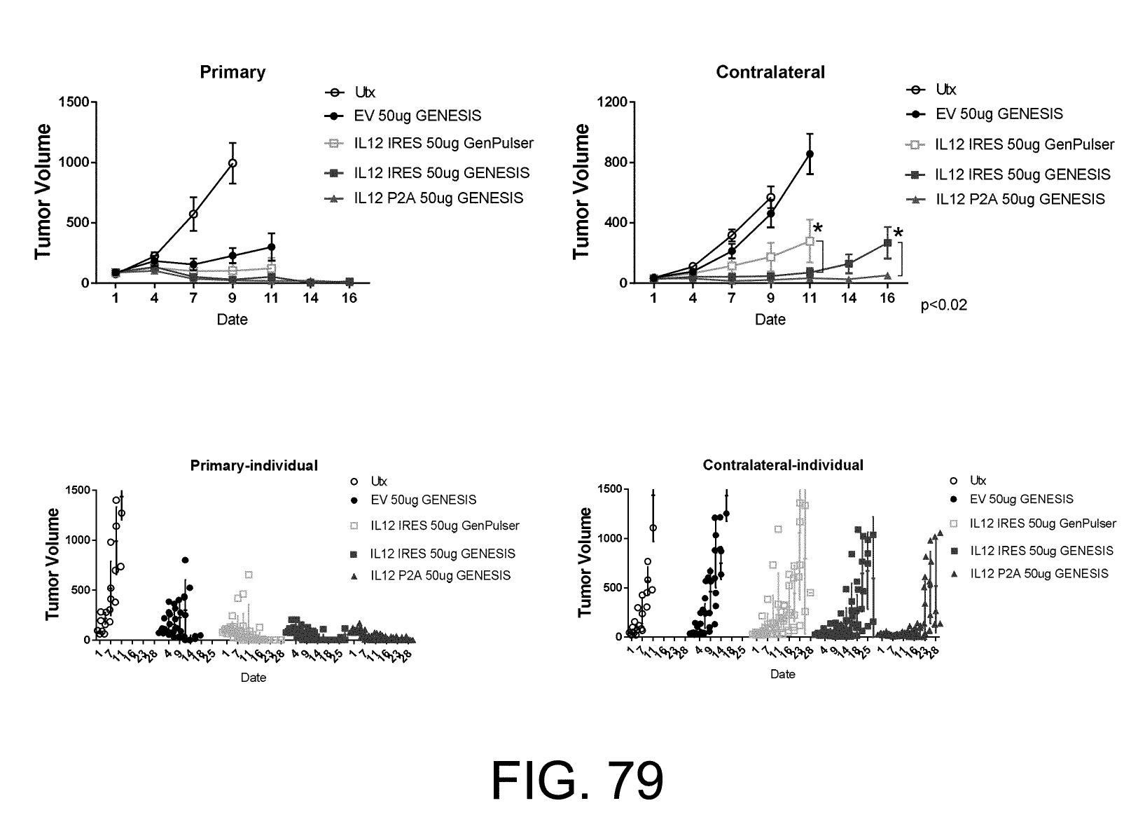

With reference to FIGS. 75-78, another example is shown in which the therapeutic treatments described herein are administered to a lesion in the lungs, which is accessed via the trachea. With reference to FIGS. 75-76, an applicator 110 is shown having an insertion tube 15 disposed in a bronchoscope 52. The bronchoscope 52 and insertion tube 15 are inserted into the lungs 910 via the trachea 912 to access a visceral lesion 914 in a primary bronchus 916.

With reference to FIG. 77 a zoomed view of the distal end 56 of the endoscope 52 is shown having the insertion tube 15 of the applicator protruding from the working channel 54 inside the bronchus 916. As depicted in FIG. 77, the electrodes and drug delivery channel are in a retracted position within the applicator. The depicted insertion tube 15 includes a flat, blunt end with no piercing tip because the lesion 914 is within the bronchus.

Turning to FIG. 78, the insertion tube 15 of the applicator is depicted having the electrodes 500 and drug delivery channel 18 in the deployed position piercing the lesion 914. The depicted electrodes 500 and drug delivery channel 18 are piercing the lesion 914 at the target lesion 914 that may be a visceral lesion such as a tumor or other malignancy. From the configuration depicted in FIG. 78, any of the therapies disclosed herein may be administered to the target lesion 914, including treatment agents, electroporation therapies, and various combination therapies.

### Example C

Several trials were also conducted regarding the efficacy of certain example electroporation systems. With reference to FIG. 79, the results of five trials are shown using various treatment agents and electroporation systems, which are represented in four plots of tumor volume versus time. With reference to the plot legends, the trials included an (1) Untreated Control (Utx); (2) an Empty Vector with low-voltage electroporation (EV 50 ug GENESIS); (3) administering an IL12 IRES plasmid with a high-voltage electroporation (IL12 IRES 50 ug GenPulser); (4) administering an IL12 IRES plasmid with a low-voltage electroporation (IL12 IRES 50 ug GENESIS); and (5) administering an IL12 P2A plasmid with a low-voltage electroporation (IL12 IRES 50 ug GENESIS).

Each trial was run using mice with B16-F10 Tumor cells inoculated in two locations (primary and contralateral) at Day −10 (1×10⁶ on the primary side, 0.25×10⁶ on the contralateral side. At the time of treatment, the primary tumor was 60-120 m³ and the contralateral tumor was 20-50 mm³. The treatment was only applied directly to the primary tumor. Each trial was run using 50 ug of plasmid (if administered) to the primary tumor per treatment. The high-voltage trials applied an electric field of 1500V/cm to the primary tumor in each of six 0.1 ms pulses. The low-voltage trials applied an electric field of 400V/cm to the primary tumor in each of eight 10 ms pulses (i.e., the low-voltage tests were longer and of lesser electric field intensity than the high-voltage tests). Treatments were administered in each study on Day 1, Day 5, and Day 8 of the study.

With continued reference to FIG. 79, it can be seen that each of the electroporation trials (2, 3, 4, 5) produced improved tumor volume changes over the control, with the trial results being ordered 5, 4, 3, 2, 1 from most tumor reduction to least. In this regard, the low-voltage generator showed improved tumor reduction over the high-voltage generator. Thus, in addition to the many advantages described throughout the disclosure, the overall success of tumor treatment is improved when electroporation is performed with a system that includes a low voltage generator.

### Example D

Prior to the above-described studies, Christoph Burkart et al. tested the plasmid and generator combination of trials (3) and (5) from Example C above, and showed substantially the same results with respect to those test parameters, showing that the IL12 P2A plasmid and low-voltage generator produced improved tumor reduction over the IL12 IRES plasmid and high-voltage generator. Absent from the Burkart study, however, was controlling for the plasmid to confirm the benefit of the low-voltage generator in the electroporation system, which additional data was captured in trial (4) of the study above.

Further discussion of a preliminary trial involving the test groups (1), (2), (3), and (5), the testing methods, and the results is included in Burkart et al., Improving therapeutic efficacy of IL-12 intratumoral gene electrotransfer through novel plasmid design and modified parameters, Gene Therapy, 25, 93-103 (9 Mar. 2018), which is incorporated by reference herein in its entirety. In some embodiments, a high voltage generator may be used, and for example, a high voltage generator may be applicable for larger tumor sizes.

### Example E

Female C57Bl/6J or Balb/c mice, 6-8 weeks of age were obtained from Jackson Laboratories and housed in accordance with AALAM guidelines. B16-F10 cells were cultured with McCoy's 5A medium (2 mM L-Glutamine) supplemented with 10% FBS and 50 μg/ml gentamicin. Cells were harvested with 0.25% trypsin and resuspended in Hank's balanced salt solution (HBSS). Anesthetized mice were subcutaneously injected with 1 million cells in a total volume of 0.1 ml into the right flank of each mouse. 0.25 million cells in a total volume of 0.1 ml were injected subcutaneously into the left flank of each mouse. Tumor growth was monitored by digital caliper measurements starting day 8 until average tumor volume reaches ˜100 mm³. Once tumors are staged to the desired volume, mice with very large or small tumors were culled. Remaining mice were divided into groups of 10 mice each, randomized by tumor volume implanted on right flank. Additional tumor cell types were tested including B16OVA in C57Bl/6J mice as well as CT26 and 4T1 in Balb/c mice. Lung metastases were also quantified in Balb/c mice bearing 4T1 tumors.

Mice were anesthetized with isoflurane for treatment. Circular plasmid DNA was diluted to 1 μg/μl in sterile 0.9% saline. 50 μl of plasmid DNA was injected centrally into primary tumors using a 1 ml syringe with a 26 Ga needle. Electroporation was performed immediately after injection. Electroporation of DNA was achieved with 400 V/cm, 10-ms pulses. Tumor volumes were measured twice weekly. Mice were euthanized when the total tumor burden of the primary and contralateral reached 2000 mm³.

Dissociation of Tumors for Flow Cytometric Analysis.

Single cell suspensions were prepared from B16-F10 tumors. Mice were sacrificed with CO₂ and tumors were carefully excised leaving skin and non-tumor tissue behind. The excised tumors were then stored in ice-cold HBSS (Gibco) for further processing. Tumors were minced and incubated with gentle agitation at 37° C. for 20-30 min in 5 ml of HBSS containing 1.25 mg/ml Collagenase IV, 0.125 mg/ml Hyaluronidase and 25 U/ml DNase IV. After enzymatic dissociation, the suspension was passed through a 40 μm nylon cell strainer (Corning) and red blood cells removed with ACK lysis buffer (Quality Biological). Single cells were washed with PBS Flow Buffer (PFB: PBS without Ca⁺⁺ and Mg⁺⁺ containing 2% FCS and 1 mM EDTA) pelleted by centrifugation and resuspended in PFB for immediate flow cytometric analysis.

Tumor Lysis for Protein Extraction.

One, 2 or 7 days after intra-tumoral electroporation (IT-EP) (400 v/cm, 8 10-ms pulses), tumor tissue was isolated from sacrificed mice to determine expression of the transgenes. Tumor were dissected from mice and transferred to a cryotube in liquid nitrogen. The frozen tumor was transferred to a 4 ml tube containing 300 μL of tumor lysis buffer (50 mM TRIS pH 7.5, 150 mM NaCl, 1 mM EDTA, 0.5% Triton X-100, Protease inhibitor cocktail) and placed on ice and homogenized for 30 seconds (LabGen 710 homogenizer). Lysates were transferred to 1.5 ml centrifuge tube and spun at 10,000×g for 10 minutes at 4° C. Supernatants were transferred to a new tube. Spin and transfer procedure was repeated three times. Tumor extracts were analyzed immediately according to manufacturer's instruction (Mouse Cytokine/Chemokine Magnetic Bead Panel MCYTOMAG-70K, Millepore) or frozen at −80° C. Recombinant Flt3L-OVA proteins were detected by standard ELISA protocols (R&D systems) using anti-FLT3L antibody for capture (R&D Systems, Minneapolis Minn. cat. #DY308) and an Ovalbumin antibody for detection (ThermoFisher, cat. #PA1-196).

TABLE 1
Intratumoral expression of hIL-12 cytokine after electroporation of a pOMI
polycistronic plasmid encoding hIL-12 under low voltage conditions.

| | Untreated | | | EP/pOMI-hIL12/hIL15/hINF-γ | | |
|---|---|---|---|---|---|---|
| Recombinant | [Protein] pg/mg | | | [Protein] pg/mg | | |
| protein | Mean +/− SEM n = 2 | | | Mean +/− SEM n = 3 | | |
| detected | Day 1 | Day 2 | Day 7 | Day 1 | Day 2 | Day 7 |
| IL-12 p70 | 0 | 0 | 0 | 3000.5 ± 1872.7 | 2874.7 ± 1459.1 | 19.1 ± 4.2 |

To test for expression and function of the FLT3L-tracking antigen-fusion protein, a fusion of FLT3L (extracellular domain) and peptides from the ovalbumin gene in OMIP2A vectors were constructed and electroporated intratumorally as above.

TABLE 2

| | EP/pUMVC3 control | EP/pOMI-FLT3L-OVA |
|---|---|---|
| Recombinant protein | Mean +/− SEM | Mean +/− SEM |
| construct | pg/ml | pg/ml |
| FLT3L-OVA fusion | 30.6 +/− 1.4 | 441 +/− 102 |

Intratumoral expression of FLT3L-OVA fusion protein (genetic adjuvant with shared tumor antigen) 2 days after electroporation under low voltage conditions as analyzed by ELISA (n = 8).

After intratumoral electroporation of pOMIP2A vectors containing mouse homologs of the immunomodulatory proteins, significant levels of IL-12p70 (Table 1) and FLT3L-OVA recombinant proteins (Table 2) were detectable in tumor homogenates by ELISA.

The protocol described above for creating mice with two tumors on opposite flanks was used as a standard model to test simultaneously for the effect on the treated tumor (primary) and untreated (contralateral). Lung metastases were also quantified in Balb/c mice bearing 4T1 tumors.

TABLE 3

| | Tumor volume (mm³) on Day 16 | |
|---|---|---|
| | Mean+/− SEM, n = 10 | |
| Intratumoral treatment | Primary tumor | Distant tumor |
| Intreated | 1005.2 +/− 107.4 | 626.6 +/− 71.9 |
| pUMVC3/EP 400V/cm 10 ms | 437.3 +/− 130.2 | 943.7 +/− 143.7 |
| pUMVC3-mIL12 400V/cm 10 ms | 131.5 +/− 31.6 | 194.5 +/− 39.6 |

B16-F10 tumor regression for primary and distant tumors after IT-EP at 400 V/cm, 8 10-ms pulses on Day 8, 12, and 15 after tumor cell inoculation.

Data in Table 3 show that when electroporation was performed with low voltage, tumor growth inhibition in both an electroporated tumor lesion as well as a distant untreated lesion was seen.

Different doses of pOMI-IL12P2A plasmid after just one dose on Day 10 after tumor cell inoculation were then tested.

TABLE 4

| | Tumor volume (mm³) on Day 19, | |
|---|---|---|
| Plasmid dose introduced by IT- | Mean+/− SEM, n = 10 | |
| EP | Primary tumor | Distant tumor |
| pUMVC3 control 50 μg | 556.4 +/− 59.0 | 211.3 +/− 46.5 |
| pOMI-mIL I2P2A 1 μg | 546.1 +/− 92.5 | 158.4 +/− 47.1 |
| pOMI-mIL12P2A 10 μg | 398.6 +/− 78.4 | 79.7 +/− 18.7 |
| pOMI-mIL12P2A 50 μg | 373.6 +/− 46.3 | 74.3 +/− 12.1 |

B16-F10 tumor regression for primary and distant tumors after IT-EP with different doses of OMI-mIL I2P2A. Electroporation with the parameters of 400 V/cm, 8 10-ms pulses was performed once, 10 days after implantation.

The extent of regression of both primary, treated and distant, untreated tumors increased with electroporation of increasing dose of pOMI-mIL12P2A plasmid. With pOMI-IL12P2A, 10 μg of plasmid was sufficient for maximal effect and there was significant tumor growth control with a single dose of treatment with the new plasmid design and lower voltage electroporation conditions.

Both the primary (treated) and the contralateral (untreated) tumor in pIL12-P2A+Low Voltage treated mice showed enhanced suppression of tumor growth. The therapeutic effect of intratumoral electroporation pOMI-IL12P2A with EP at low voltage was also reflected in a statistically significant survival advantage (5/6 mice survived until end of study with pOMI-IL 12P2A/lowV).

The ability of IT-EP of pOMI-mIL 12P2A to affect 4T1 primary tumor growth and lung metastases in Balb/c mice was also tested. One million 4T1 cells were injected subcutaneously on the right flank of the mice and 0.25 million 4T1 cells were injected into the left flank. Larger tumors on the right flank were subject to IT-EP with empty vector (pUMVC3, Aldevron) or with pOMI-mIL12P2A. Tumor volumes were measured every two days and on Day 19, mice were sacrificed, and the lungs were excised and weighed.

TABLE 5

| | Primary tumor volume | |
|---|---|---|
| | (mm³) | Lung weight (grams) |
| Treatment | Mean +/− SEM, n = 5 | Mean +/− SEM, n = 5 |
| Untreated | 897 +/− 131 | 0.252 +/− 0.019 |
| EP/pUMVC3 | 593 +/− 27 | 0.228 +/− 0.006 |
| EP/pOMIP2A-mIL12 | 356 +/− 80 | 0.184 +/− 0.004 |

Primary tumor growth and post-mortem weight of lungs of mice electroporated with 400 V/cm, 8 10-ms pulses on day 8, and day 15 post-implantation. Primary tumor volumes were measured on Day 17, and lung weights on Day 18.

Findings indicated that local IT-EP treatment of the tumors also reduced metastasis of these tumor cells to the lung in this model (Table 5).

In addition to B16F10 tumors, electroporation of pOMI-mIL12P2A also resulting in regression of both primary (treated) and contralateral (untreated) B16OVA and CT26 tumors. In the 4T1 tumor model, the primary tumor regressed after EP/pOMI-mIL12P2A, and the mice demonstrated a significant reduction in lung weight, indicating a reduction in lung metastases. The data show that IT-EP of OMI-mIL12P2A can reduce tumor burden in 4 different tumor models in two different strains of mice.

TABLE 6

| | Tumor volume (mm³), Mean +/− SEM, n = 10 | |
|---|---|---|
| Treatment | Primary tumor | Distant tumor |
| EP/pUMVC3 control | 600.7 +/− 113.3 | 383.4 +/− 75.9 |
| EP/pOMI-IL12P2A + | 94.2 +/− 31.7 | 115.7 +/− 42.3 |
| pOMI-FLT3L-OVA | | |

B16-F10 tumor regression for treated and untreated tumors after intratumoral electroporation of pOMIP2A plasmids containing genes encoding mIL-12 and FLT3L-OVA using 400 V/cm, and 8 10-ms pulses on day 7 and 14 after tumor cell inoculation; tumors measurements shown from Day 16.

TABLE 7

| | Tumor volume (mm³), Mean +/− SEM | |
|---|---|---|
| Treatment | Primary tumor | Distant tumor |
| EP/pUMVC3 empty vector n = 9 | 895.94 +/− 94.29 | 459.51 +/− 64.45 |
| EP/pOMI-PIIM n = 7 | 274.70 +/− 36.27 | 140.71 +/− 32.26 |

B16-F10 tumor regression for treated and untreated tumors after IT-EP of pOMI-PIIM (version containing mouse IL-12) using 400 V/cm, and 8 10-ms pulses on day 7 after tumor cell inoculation; tumors measurements shown from Day 15.

Electroporation of a pOMI-PIIM expressing both mouse IL-12 p70 and human FLT3L-NY-ESO-1 fusion protein caused significantly reduced growth of both the primary, treated and the distant, untreated tumors (Table 7) with only a single treatment.

The volume of both primary and contralateral tumors is significantly reduced in mice where immunomodulatory genes were introduced by electroporation as compared with electroporation of empty vector control, indicating not only a local effect within the treated tumor microenvironment, but an increase in systemic immunity as well.

### Example F

Nucleic Acid Vectors Encoding Transgenes are Efficiently Delivered to Tumor Cells In Vivo Using Low Voltage Electroporation.

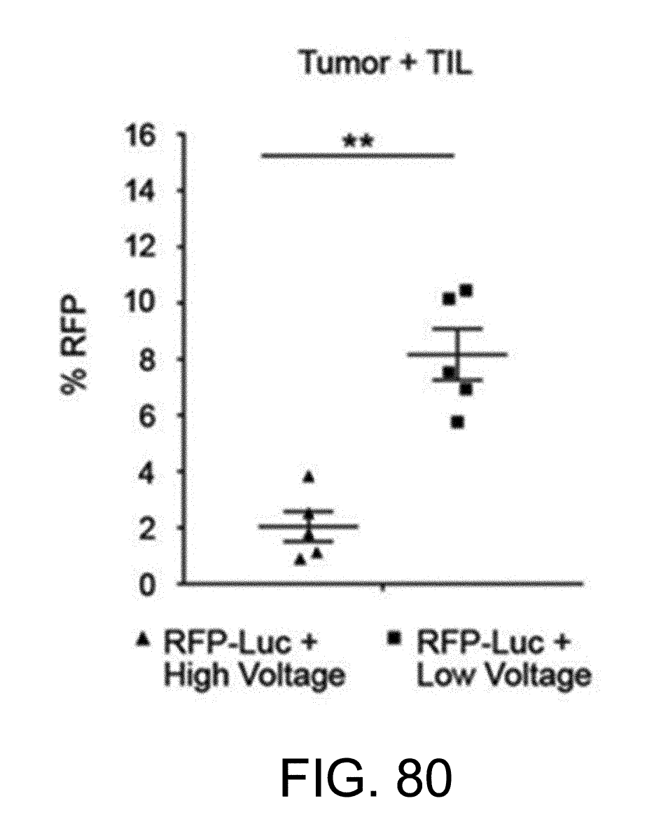

With reference to FIG. 80, an example is shown of transfection using low and high voltage electroporation. Malignant melanoma tumors were allowed to establish in mice. In particular, C57Bl/6 mice were injected subcutaneously (s.c.) with 1×10\hat{ }6 B16-F10 melanoma cells and tumors were allowed to establish.

Upon reaching 75-150 mm\hat{ }3, tumors were injected with plasmid DNA encoding for a red-fluorescent protein variant, known as mCherry (RFP), following by application of an electrical pulse using two different electroporation parameters: High voltage and low voltage. In particular, tumors were injected intratumorally with 50 ug Luciferase-mCherry DNA plasmid followed by electroporation using either high voltage (1500V/cm) or low voltage (400V/cm) conditions. Electroporation was performed using a two-needle (e.g., two electrodes) applicator.

48-hr later, mice were euthanized and the tumors were excised, dissociated using an enzyme cocktail, and made into single cell suspensions for analysis by flow cytometry (FACS). Flow cytometry was performed to count the number of live ‘red’ cells and scored as a percentage of live mCherry⁺ cells. The data shown were normalized to background RFP signals produced by injection of RFP plasmid without electroporation. Since these cells do not normally express red fluorescent protein, all red cells must have been derived from electroporation-mediated cell transfection. Using low voltage electroporation conditions, 8-10% of cells within the tumor were found to be transfected.

### Example G

Low voltage electroporation is effective in delivering various plasmid and expression vectors to tumor cells in vivo.

B16-F10 tumors were formed in mice as described above. Established tumors were injected with the indicated plasmid or expression vector following by application of an intra tumoral electroporation pulse (IT-EP).

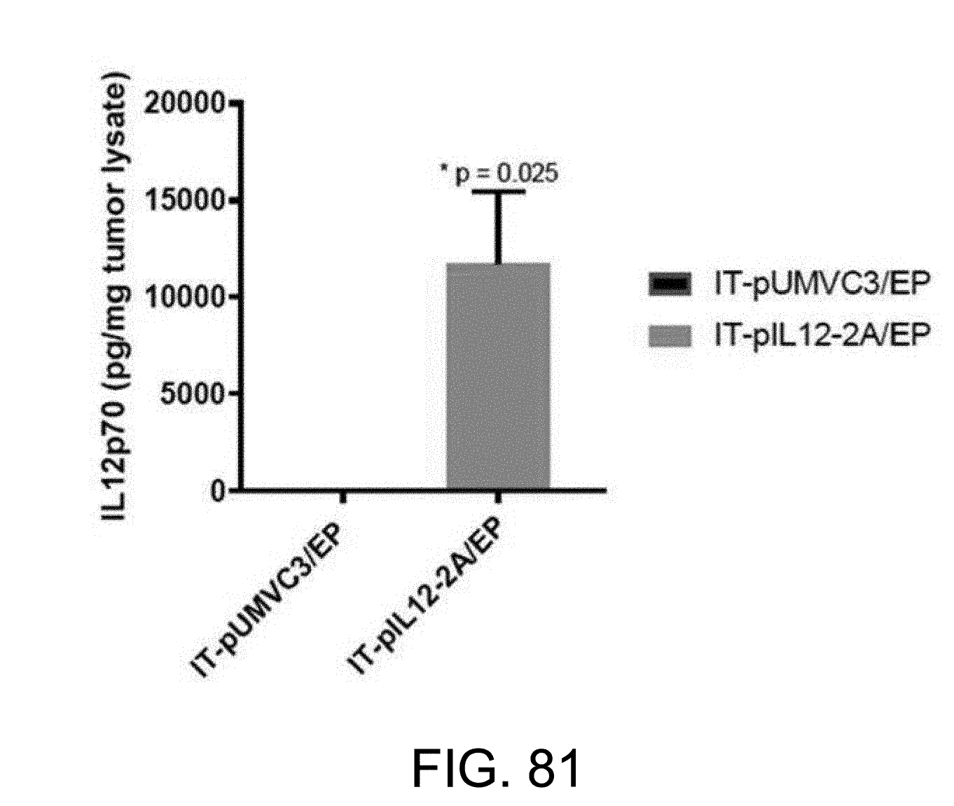

FIG. 81 shows a plot of expression of mIL-12p70 following low voltage (400 V/cm) IT-EP of plasmid into established B16-F10 tumors. The expression of IL-12p70 was detectable 48 hrs post electroporation using a standard R&D Systems IL-12p70 DuoSet ELISA.

Electroporation was performed using a two-needle (e.g., two electrodes) applicator.

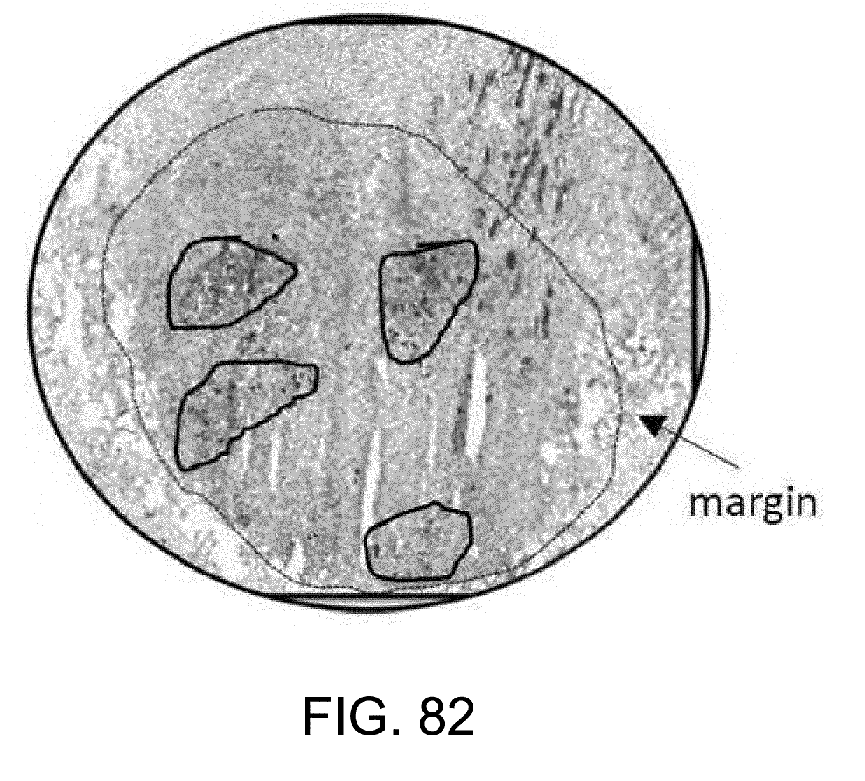

FIG. 82 shows expression of LacZ in established B16-F10 tumors. LacZ staining was performed following low voltage (400V/cm) IT-EP of a Lax Z expressing plasmid into established B16-F10 tumors. Electroporation was performed using a two-needle (e.g., two electrodes) applicator.

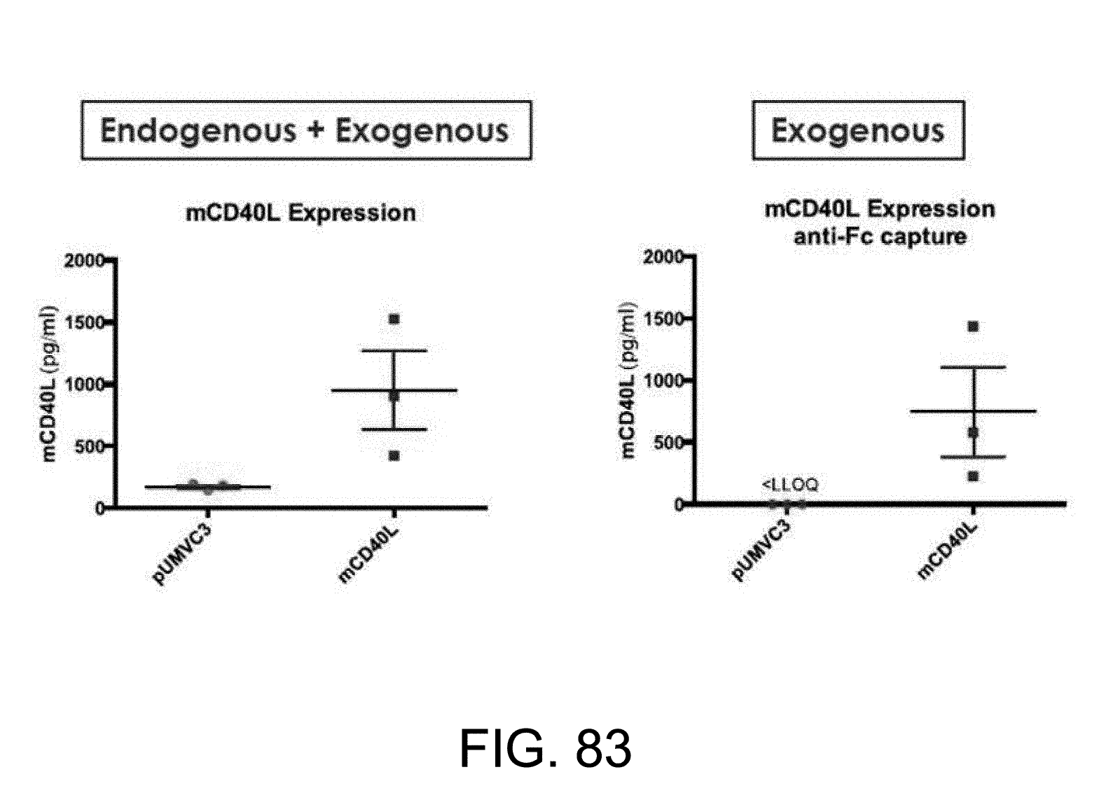

FIG. 83 shows expression of trimeric CD40L in B16-F10 tumors following low voltage (400 V/cm) IT-EP of mCD40L3 plasmid or empty vector (50 μg). The tumors were extracted at 48 hrs and ELISAs were run to determine expression. mCD40L was readily detectable following EP (400V/cm), either by a standard R&D Systems mCD40L ELISA (endogenous+exogenous), or by modifying the ELISA with an anti-hIgG-Fc capture antibody (exogenous only). Electroporation was performed using a two-needle (e.g., two electrodes) applicator.

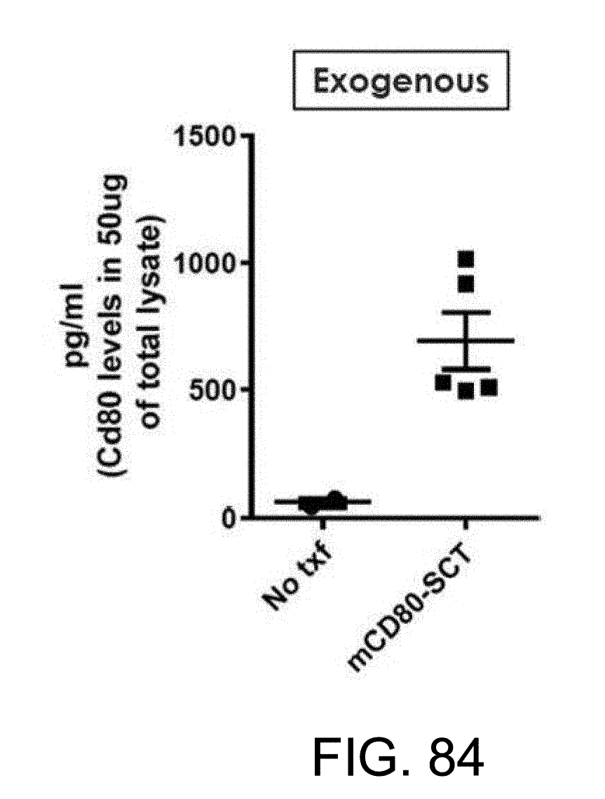

FIG. 84 shows expression of trimeric CD80 in B16-F10 tumors following low voltage (400 V/cm) IT-EP in B16-F10 tumors. In this study, mCD803 or empty vector (50 μg) was electroporated into established B16-F10 tumors. The tumors were extracted at 48 hrs and ELISAs were run to determine expression. mCD80 was readily detectable following EP (400V/cm), using a modified R&D Systems mCD80 with an anti-hIgG-Fc capture antibody. Electroporation was performed using a two-needle (e.g., two electrodes) applicator.

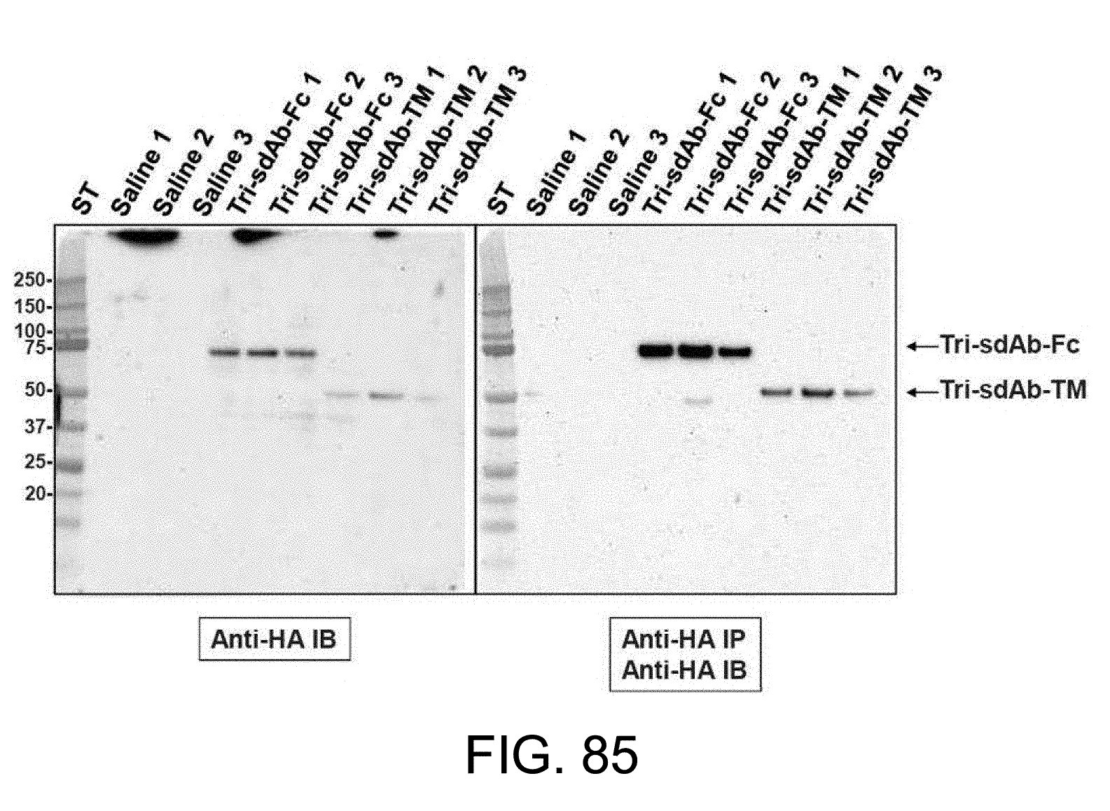

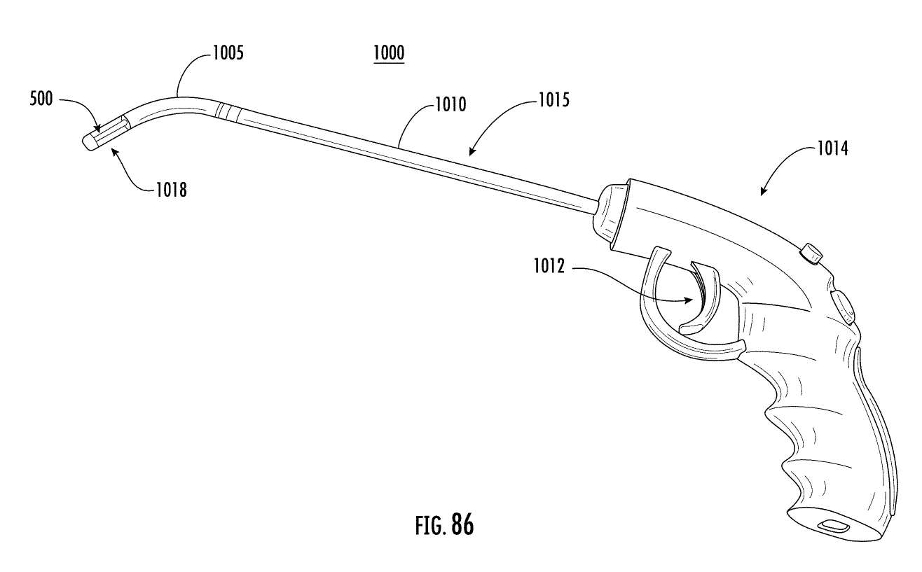

FIG. 85 shows expression of sdAbs in B16-F10 tumor following low voltage (400 V/cm) IT-EP. Multimerized nanobodies were detected in tumor lysates by western blot 48 hrs post-electroporation. Electroporation was performed using a four-needle array.

Thus, in addition to mCherry (RFP) shown in FIG. 80 and Example F, the studies of FIGS. 81-85 show expression in tumors following low voltage electroporation of the following DNA-encodable molecules: (1) mIL12-p70; (2) LacZ; (3) CD40L; (4) CD80; and (5) a nanobody. Tumor cell expression was verified through various techniques including tissue ELISAs, flow cytometry, and western blot.

### Example H

Example H provides one embodiment of an applicator of the present disclosure, and examples of the use and benefits of the applicator.

Liver and pancreatic cancers represent areas of important unmet medical need. In 2018, more than 42,000 patients were diagnosed with liver cancer, the majority of whom had advanced disease not amenable to curative resection. Despite decades of advances and the introduction of multiple localized and targeted therapies in recent years, more than 30,000 patients succumbed to liver cancer. The situation for pancreatic cancer is even more urgent. More than 55,000 patients were diagnosed with pancreatic cancer in 2018, and more than 44,000 patients died from this malignancy. Fewer than 1 in 10 patients diagnosed with pancreatic cancer survive at least 5 years, and this falls to 1 in 20 for patients with unresectable disease. Only approximately 10% of pancreatic cancer cases are diagnosed at a stage when potentially curative resection is possible, and the cancer is generally very aggressive and places a heavy symptom burden on patients as the disease progresses. Embodiments of the systems, associated applicators, generators, and methods disclosed herein may change the treatment paradigm for these patients by delivering potent immunotherapy directly to the tumors and potentially increasing their responses to existing standard of care (e.g., checkpoint inhibitor therapy).

Electroporation is a physical transfection method that may use an electrical pulse to create temporary pores in cell membranes through which substances like nucleic acids can pass into cells. It is a highly efficient strategy for the introduction of foreign nucleic acids into many cell types. During the period when cells are exposed to a brief pulse of energy, the cell membrane becomes highly permeable to exogenous molecules, which pass through pores in the cell membrane (a process known as transfection). The electrical pulse may be at an optimized voltage and may last only a few microseconds to a millisecond. This may disturb the cell membrane, which is an ionized phospholipid bilayer, and results in the formation of temporary pores in this cellular barrier. The electric potential across the cell membrane may simultaneously rise, allowing charged molecules like DNA plasmids to be driven across the membrane. The energy for EP may be applied using an electrode applicator, which can have microneedle electrodes according to any of the embodiments discussed herein, and an electrical pulse generator according to any of the embodiments discussed herein. Needle electrodes enable EP to be performed in vivo, allowing for potential medical application.

EP has important advantages over other methods of cell transfection. The main advantage of EP is its applicability for rapid transfection of all cell types. It is a noninvasive, bioelectronic, nonchemical method that produces limited alterations in the biologic structure and function of the target cells. It is easy to perform and is more rapid than traditional chemical or biologic cell transfection techniques. The process is nontoxic and, because it is a physical method, it can be applied to a broad selection of cell types. Similarly, a wide array of molecules can be transfected, which makes EP highly versatile.

According to some embodiments, EP may be used as a microinjection technique to transfect millions of cells with specific components—immunologically relevant and important components of choice—in order to program the patient's own cells to make these agents on a prolonged basis.

The inventors recognized the distinct advantages of EP and translated it into a powerful tool to deliver potent immunomodulatory agents to treat cancer as described herein. As described above, the clinical use of EP may entail depositing exogenous molecules in the area surrounding cells. During the momentary cell membrane destabilization induced by the externally applied electrical field, the exogenous molecules can pass through membrane pores and, once the electrical field ceases, these molecules may be trapped inside the cell. Plasmid-based DNA, coded to produce immunomodulatory proteins, may be used and then deposited the DNA in the areas surrounding a cell.

Once inside the cell, the DNA plasmids co-opt the cell's function to cause it to make or “express” the immunomodulatory protein. This sequence can be carried out in millions of cells at once, causing sustained intracellular release of the immunomodulatory protein.

EP may efficiently transfect a diversity of exogenous molecules into a wide selection of cell types by a noninvasive, nonchemical method that does not negatively alter the biologic structure or function of the target cells. Cancer immunotherapies may be delivered via the EP of plasmid DNA to use a cancer patient's own tumors to produce a potent yet safe immunotherapy. This causes sustained intracellular release of immunologically relevant proteins, such as the proinflammatory cytokine interleukin (IL)-12. IL-12 is configured to transform immunosuppressed tumors into immunologically active lesions via coordinated innate and adaptive immunity.

Several different types of DNA plasmids encode immunologically relevant genes, such as an investigational human IL-12 (tavokinogene telseplasmid, or TAVO™, OncoSec Medical Incorporated). Using embodiments of the therapeutic system and methods disclosed herein, TAVO is injected into a lesion and expressed through EP pulses. Transfected cells then express and secrete IL-12 protein, which initiates both local and systemic immune responses.

Studies indicate that intratumoral plasmid-based IL-12 delivered via EP can generate local and systemic immune responses that can convert immunologically cold tumors to T-cell-inflamed hot tumors. The applicants have 2 registration-directed clinical trials in advanced melanoma and cervical cancer, and have demonstrated efficacy in other cutaneous tumor indications, including head and neck squamous cell carcinoma, Merkel cell carcinoma, and triple-negative breast cancer (TNBC) via chest-wall lesions.

Derived from significant multitumor clinical trial experience, the investigational TAVO is tumor agnostic and independent of tumor histology, genetic, and/or immunologic status, making it a viable therapy across numerous tumor indications, including, importantly, internal tumors.

In some embodiments, a system has been used to treat cutaneous and subcutaneous tumors. Moreover, embodiments of the system disclosed herein are configured to treat lesions beyond cutaneous and subcutaneous tumors.

The systems disclosed herein include applicators and generators that allow for EP of a wide array of immunologically relevant genes into cells located in visceral lesions, which are tumors located inside the body, including but not limited to gastrointestinal (GI) tumors, pancreatic tumors, and hepatocellular carcinomas (HCC; a “Visceral Lesion Applicator” or “VLA” according to any of the embodiments disclosed herein).

For example, the relevant immune mechanisms associated with clinical progression of HCC include increased tumor-infiltrating regulatory T cells (Tregs) and M2-polarized tumor-associated macrophages (TAMs), which can establish immune suppression both in the tumor microenvironment and peripherally. This immunoinhibitory network, when complexed with additional tumor-intrinsic suppressive mechanisms, has posed a significant challenge to meaningful treatment modalities. However, the emergence of anti-programmed cell death protein 1/ligand 1 (PD-[L]1) therapies, particularly in combination with locoregional therapies that can target these suppressive barriers, may provide meaningful clinical benefit.

The intratumoral IL-12 EP platform disclosed herein not only enhances anti-PD-[L]1 activity (currently treating anti-PD-1-refractory melanoma patients with pembrolizumab [Keytruda®] plus its investigational TAVO in the KEYNOTE-695 study) via recruitment of functional T cells and the induction of adaptive resistance in the tumor microenvironment, but also critically modulates the ratio of CD8+ tumor-infiltrating lymphocytes (TILs) to Tregs as well as M2 macrophages, making this combination especially attractive in this tumor setting.

The applicator may work with embodiments of the generator and applicators disclosed herein to leverage plasmid-optimized EP, enhancing the depth and frequency of transfection and yielding a significant therapeutic benefit in preclinical models. This next step in EP has been further augmented with a next-generation plasmid therapeutic, which drives superior IL-12 expression along with complementary immunomodulatory genes easily coded into this customizable vector backbone.

The systems shown and described herein facilitate, inter alia, plasmid-based immunotherapeutics in small animal models.

Preclinical studies utilizing a miniaturized 2-needle applicator with an electrode width of 1.5 mm yielded IL-12-dependent tumor regression in a difficult-to-treat experimental nodal metastasis model using CT26 colorectal tumors. These preliminary data, coupled with a large body of preclinical studies in multiple tumor models utilizing a 0.5-cm 2-needle applicator, firmly establish the feasibility of moving this miniaturized applicator toward the clinic.

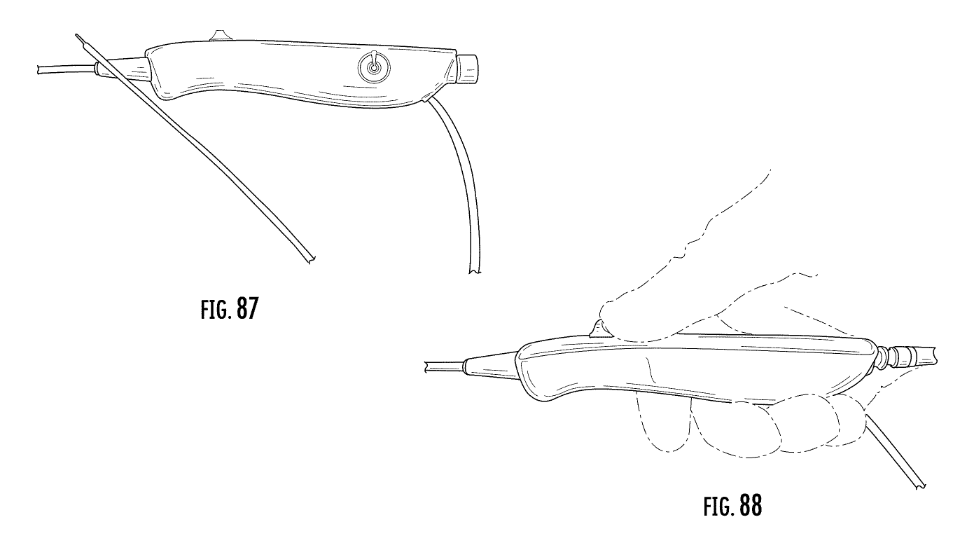

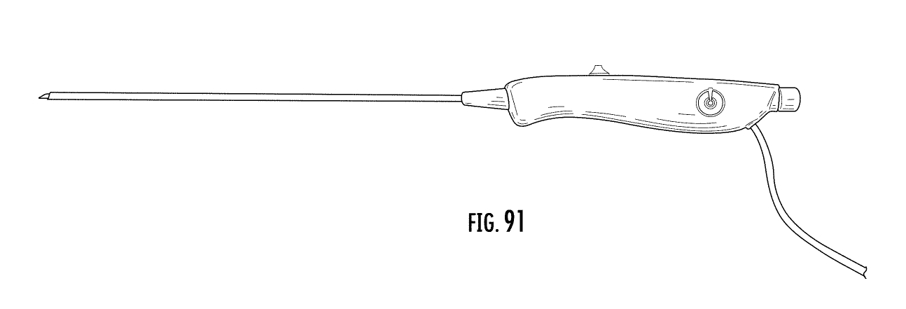

Some embodiments of the applicators disclosed herein have been developed as either a flexible catheter-based applicator (e.g., as shown herein, including FIGS. 87-88) or a more rigid trocar-based applicator (e.g., as shown herein, including FIG. 91) according to any of the embodiments disclosed herein. In some embodiments, the catheter-based applicator may include a flexible body that, with a diameter of 2 mm, is sized for passage through currently available endoscopes, bronchoscopes or laparoscopes.

For example, an endoscope can be positioned through the mouth into the stomach/small intestine, where a flexible applicator can be guided into pancreatic lesions, for sequential plasmid injection and EP. The flexible body (e.g., insertion tube 15) may have a length of approximately 100 cm to allow for navigation toward the target lesions via an endoscope or laparoscope, depending on the application and/or tumor indication.

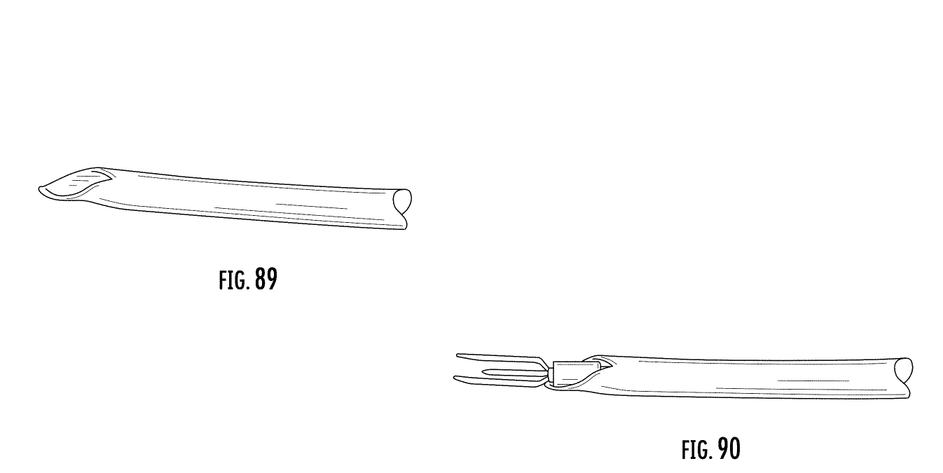

In some embodiments, the applicator may be a handheld instrument with an ergonomic handle at its proximal end as discussed herein. The distal end of the flexible body (e.g., insertion tube) may include a central localized injection needle flanked by dual electrodes. The electrodes and injection needle may be actuated between a retracted position and a deployed position. As illustrated in FIGS. 89-90, the electrodes may be biased away from one another in the deployed position at a spacing of about 3 mm. This spacing may facilitate achieving a wider span of EP while minimizing the chances of electrical arcing between the electrodes. Other advantages are described throughout the disclosure.

Once the distal tip of the applicator is properly positioned at the tumor site, the therapeutic plasmid may be delivered into the lesion via an injection needle housed in the applicator. The co-localized electrodes can then transfer the electrical pulses into the tumor via any of the generators disclosed herein (e.g., a foot pedal-controlled generator). These electrical pulses may allow for transfection of the plasmid into the tumor cells and the subsequent local secretion of the immune-activating cytokine (e.g., as shown in FIGS. 71-74).

In some embodiments a rigid applicator (e.g., as shown in FIG. 91) can also access visceral tumors, but with a slightly different approach. This trocar needle-based visceral lesion applicator may include a rigid body (e.g., insertion tube 15) that may be capable of directly entering soft tissue directly with open or laparoscopic surgery, with ultrasound or computed tomography (CT) guidance to the target lesion. For example, in some embodiments, the insertion tube 15 may have a diameter of 2 mm and a length of 20 cm. Like the catheter-based, flexible applicator, the rigid trocar-based applicator may be operated with an ergonomic handle at its proximal end. Also like the catheter-based applicator, the distal end of the rigid body may include a similar central localized injection needle flanked by dual electrodes having a retracted, compact position and a deployed, expanded position as described herein

In some embodiments, unlike some embodiments of the catheter-based applicator, the trocar-based applicator may access a visceral tumor using a minimally invasive transcutaneous approach, which can be particularly useful for treating liver lesions. When the distal end of the rigid body reaches the tumor site, the electrodes and the injection needle may be actuated to the deployed position and the plasmid may be administered, followed by application of the electrical pulses from the generator via a foot pedal for delivery of the therapeutic EP.

A minimal profile of the applicators may help reduce their “clinical footprint,” and their relative usability, either directly or in combination with common endoscopes and laparoscopes, may make them ideal to address different unmet medical needs in GI-based cancers. These novel applicators may introduce the immunotherapeutic platform described herein to visceral tumor indications, extending the clinical impact of this powerful cytokine-based therapy.

In one example embodiment, a trocar-based applicator may be used to access primary tumors in patients who have unresectable HCC tumors. While HCC patients do present with tumors that appear resectable, often their underlying liver disease (i.e., cirrhosis) excludes these patients as candidates for surgical resection or transplant. For these patients, who represent approximately 70% of the newly diagnosed HCC patients in the Western world, treatment options are limited to transarterial chemoembolization (TACE), radioembolization, and systemic therapy, with many being referred directly to hospice without any intervention. The ability to access these lesions intratumorally with a potent immunotherapy could shift the treatment paradigm for these patients. Embodiments of the trocar-based visceral lesion applicator discussed herein are sufficiently miniaturized to pass through the central lumen of a percutaneous needle commonly used for liver biopsies. This approach allows for the procedure to be done in an interventional suite utilizing CT-guided imagery. While the applicator may be configured for use in a laparoscopic procedure, the percutaneous approach has several advantages. It minimizes the need for general anesthesia and allows the procedure to be repeated on a weekly basis, if dosing regimens so demand. The device may also be configured for use with an endoscope, allowing for a transgastric approach. While this may be attractive for disease located in the left-hand portion of the liver proximal to the stomach, disease distal from the stomach would require a percutaneous or laparoscopic approach. The versatility of the applicators discussed herein would facilitate broad usage potential with surgical, radiologic, and endoscopic applications using a single generator and delivery system.

Embodiments of the inventions described herein provide key potential advantages over conventional liver-directed therapy. Microwave ablation and RFA are limited in that they are only useful for relatively small tumors. Furthermore, some lesions cannot safely be treated with ablation due to proximity to critical structures such as major vascular structures and central bile ducts. The heat associated with ablation is an inherent limitation to microwave and radiofrequency ablation. Chemoembolization and radioembolization require adequate liver function. So many patients with inoperable HCC cannot be treated with ablation, or other liver directed therapies due to anatomic or liver function concerns. Percutaneous treatment options for primary liver tumors, including ablation with radiofrequency currents (RFA), microwaves, or freezing (cryoablation), are typically limited to early disease stages, with the hope of ameliorating the disease before it metastasizes. For example, microwave ablation uses a probe to deliver thermal pulses to the malignant tissue, resulting in an ablation zone. Microwave ablation is seen as an improvement over RFA in its ability to target larger-sized lesions. However, a recent study found that although there was a very low rate of recurrence of the treated lesions, new liver lesions developed in 72% of patients with liver lesions smaller than 4 cm treated with microwave ablation. Therefore, microwave ablation effects appear limited to the treated lesion. This has also been demonstrated for other localized approaches, such as embolizing radiotherapy microspheres. In contrast, rather than directly ablating tumor cells, the TAVO technology has the potential to transiently turn these lesions into cellular factories for immunostimulating cytokines, which can work in concert with other immunotherapies such as checkpoint inhibitors. As illustrated in patients with anti-PD-1-refractory melanoma being treated with TAVO and pembrolizumab in KEYNOTE-695, tumor responses can occur not only in the treated lesion, but also in distant sites. 14 Therefore, TAVO is a localized therapy that can mediate systemic anticancer effects.

In some embodiments, methods of treating non-responder patients who have progressed on or do not respond to checkpoint therapy are described. The methods comprise injecting a cancerous tumor in the non-responder with an effective dose of a plasmid encoding one or more immunomodulatory peptides; administering electroporation therapy to the cancerous tumor; and administering an effective dose of a checkpoint inhibitor to the subject. The one or more immunomodulatory peptides can be, but are not limited to, IL-12, CD3 half-BiTE, CXCL9, CTLA-4 scFv, IL12 and CD3 half-BiTE, IL-12 and CXCL9, and IL-12 and CTLA-4 scFv. The checkpoint inhibitor can be, but is not limited to nivolumab (ONO-4538/BMS-936558, MDX1 106, OPDIVO), pembrolizumab (MK-3475, KEYTRUDA), pidilizumab (CT-011), and MPDL3280A (Roche).

A “non-responder” or “non-responsive” refers to a patient who has a cancer and who: a) is progressing, has progressed on, or has not responded to a cancer therapy, b) does not exhibit a beneficial clinical response following treatment with the cancer therapy, c) is unable to achieve clinical remission or clinical response to the cancer therapy, and/or d) has filed to reach a target response to the cancer therapy. In some embodiments, the non-responder has not cleared a cancer in response to a cancer therapy. In some embodiments, the non-responder has had a relapse, recurrence or metastasis of a cancer following treatment with a cancer therapy. In some embodiments, the non-responder has a negative cancer prognosis after treatment with cancer therapy. The cancer therapy can be, but is not limited to, checkpoint therapy. Checkpoint therapy can be, but is not limited to, anti-PD-1 or anti-PD-L1 antibody therapy.

There is a strong unmet medical need for novel immunotherapy approaches such as TAVO. Both nivolumab (Opdivo®) and pembrolizumab received accelerated approval from the FDA for treating liver cancer, on the basis of efficacy and safety results from early phase trials that had overall response rates of only 14% to 17%. The majority of patients did not respond to these new modalities. Checkpoint inhibitor-refractory disease represents a growing population and an emerging therapeutic challenge. In an ongoing clinical study in patients with metastatic melanoma (KEYNOTE-695) the combination of TAVO and an anti-PD-1 antibody produced an observed preliminary response rate of 24% in patients (anti-PD-1 antibody therapy non-responders) whose disease was truly refractory to anti-PD-1 antibody monotherapy. Combining the anti-PD-1 agent, such as pembrolizumab, with an agent capable of driving an effective T cell response, such as IL-12, may increase the immunogenicity in the non-responder phenotype and enhance response to checkpoint therapy.

In some embodiments, subjects non-responsive or predicted to be non-responsive to checkpoint therapy are treated with a combination of intratumoral electroporation of IL-12 and systemic administration of anti-PD-1 therapy. Non-responders are administered a plasmid (e.g., TAVO) encoded immunostimulatory cytokine and a checkpoint inhibitor using a dosing schedule, wherein the dosing schedule comprises: a) a first cycle of treatment on week 1, wherein: i) the plasmid encoded immunostimulatory cytokine is delivered to a tumor by electroporation on days 1 (±2 days), 5 (±2 days), and 8 (±3 days); and ii) a checkpoint inhibitor delivered systemically to the patient on day 1 (±2 days); b) a second cycle of treatment, wherein the checkpoint inhibitor is delivered systemically to the patient three weeks after the first cycle; and c) continued subsequent treatment cycles wherein the first and second cycles are repeated alternatively every three weeks. In some embodiments, the plasmid encoded immune stimulatory cytokine is administered at every cycle. In some embodiments, the plasmid encoded immune stimulatory cytokine is administered at alternate cycles. In some embodiments, the plasmid encoded immunostimulatory cytokine and the checkpoint inhibitor are delivered concurrently on day 1 of each cycle. In some embodiments the two therapies are administered concurrently on odd numbered cycles and the checkpoint inhibitor is administered alone on even numbered cycles. In some embodiments, the plasmid encoded immunostimulatory cytokine is delivered by electroporation at least one, two, or three days of each cycle or alternating cycles. The intervening period between each cycle can be from about 1 week to about 6 weeks, from about 2 weeks to about 5 weeks. In some embodiments, the intervening period between cycles is about 3 weeks.

In combination with the tumor-agnostic power of IL-12 (e.g., TAVO), the visceral lesion applicator system described herein may be applicable to most internal tumor indications that can be accessed with an endoscope, bronchoscope, catheter, trocar, or the like. TAVO has already proven to show robust efficacy in difficult-to-treat patient populations, including metastatic melanoma refractory to checkpoint inhibitor therapy, as well as TNBC. Notably, TAVO demonstrated and continues to demonstrate a powerful abscopal effect. In its earlier phase 1 monotherapy trial, TAVO demonstrated a 46% response rate in untreated lesions in metastatic melanoma. After cancer has spread, curative resection is typically not possible. Consequently, there are approximately 23,500 new cases of unresectable liver cancer and 49,900 new cases of unresectable pancreatic cancer each year, a diagnosis typically associated with poor prognosis.

Local treatment options are largely limited to ablative procedures, which do not seem to provide a significant benefit over standard of care and exhibit little to no meaningful abscopal effect. Local therapies for liver cancer are typically cytoreductive, not curative, in nature and typically do not have a major impact on the disease course overall. For example, a study comparing radioembolization with yttrium 90 (Y90) vs treatment with a targeted therapy, sorafenib, found that although Y90 treatment significantly delayed disease progression in the liver vs treatment with sorafenib, there was no survival advantage. Indeed, the rate of progression outside the liver was significantly greater with Y90 treatment vs sorafenib, and survival was shorter, although the difference did not reach statistical significance.

The ability to directly inject these tumors with a potent cytokine and concurrently deliver that therapeutic via EP could result in meaningful treatment options for these patients. TAVO may be able to deliver a similar abscopal response in HCC as it does in metastatic melanoma and TNBC.

The systems and methods disclosed herein may be applicable to any nucleic acid-based therapeutic or chemotherapeutic intended for intratumoral delivery (e.g., bleomycin).

The subject matter described herein includes, but is not limited to, the following specific embodiments:

1. A method of treating a lesion at a lung of a subject who is non-responsive or predicted to be non-responsive to anti-PD-1 or anti-PD-L1 therapy, the method comprising:

administering to the lesion an effective dose of at least one plasmid coding for IL-12;

administering electroporation therapy to the lesion; and

administering to the subject an effective dose of at least one checkpoint inhibitor;

wherein administering the electroporation therapy comprises administering an electric pulse to the lesion using an electroporation system comprising:

an applicator comprising:
a plurality of electrodes comprising a first electrode having a first tip and a second electrode having a second tip, wherein the plurality electrodes are configured to move between a retracted position and a deployed position;
wherein a distance between the first tip of the first electrode and the second tip of the second electrode is greater in the deployed position than in the retracted position; and

a generator electrically connected to the plurality of electrodes,

wherein administering the electric pulse to the lesion comprises disposing the first electrode and the second electrode into or adjacent to the lesion, and delivering the electric pulse from the generator to the first electrode and the second electrode.

2. The method of embodiment 1, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

3. The method of embodiment 1 or embodiment 2, wherein the electroporation system further comprises an insertion device comprising one of a rigid trocar or flexible endoscope defining at least one working channel, wherein at least a portion of the applicator is configured to pass through the at least one working channel to access the lesion.

4. The method of any one of embodiments 1-3, wherein the electroporation system further comprises a drug delivery device configured to deliver at least one of the at least one plasmid or the at least one checkpoint inhibitor through the at least one working channel of the insertion device.

5. The method of any one of embodiments 1-4, wherein the applicator further defines a drug delivery channel configured to deliver at least one of the at least one plasmid or the at least one checkpoint inhibitor to the lesion.

6. The method of any one of embodiments 1-5, wherein the electroporation system further comprises at least one robotic arm engaged with the applicator to control a position of the applicator during administration of at least one of the at least one plasmid, the at least one checkpoint inhibitor, or the electroporation therapy.

7. The method of any one of embodiments 1-6, wherein the electroporation system further comprises at least one visualization device configured to generate imagery of the lesion before or during administration of at least one of the at least one plasmid, the at least one checkpoint inhibitor, or the electroporation therapy.

8. The method of embodiment 7, wherein the at least one visualization device comprises a computed tomography scanner.

9. The method of any one of embodiments 1-8, wherein the generator is configured to output low-voltage electric pulses.

10. The method of any one of embodiments 1-9, wherein the electric pulses have a field strength of 700V/cm or less.

11. The method of any one of embodiments 1-8, wherein the generator is configured to output high-voltage electric pulses.

12. The method of any one of embodiments 1-11, wherein the at least one plasmid comprises tavokinogene telseplasmid.

13. The method of any one of embodiments 1-12, wherein the checkpoint inhibitor is administered systemically.

14. The method of any one of embodiments 1-13, wherein the checkpoint inhibitor is an anti-PD-1 antibody or an anti-PD-L1 antibody.

15. The method of any one of embodiments 1-14, wherein the checkpoint inhibitor comprises: nivolumab, pembrolizumab, pidilizumab, or MPDL3280A.

16. A system for treating a lesion at a lung of a subject who is non-responsive or predicted to be non-responsive to anti-PD-1 or anti-PDL1 therapy, the system comprising:

an applicator comprising a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position;

a generator electrically connected to the plurality of electrodes, wherein the generator is configured to deliver an electric pulse to the first electrode and second electrode to administer the electric pulse to the lesion; and

at least one drug delivery device configured to deliver to the subject an effective dose of at least one plasmid coding for IL-12 and an effective dose of at least one checkpoint inhibitor.

17. The system of embodiment 16, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

18. The system of embodiment 16 or embodiment 17 further comprising an insertion device comprising one of a rigid trocar or flexible endoscope defining at least one working channel, wherein at least a portion of the applicator is configured to pass through the at least one working channel to access the lesion.

19. The system of any one of embodiments 16-18 further comprising a drug delivery device configured to deliver the at least one plasmid through the at least one working channel of the insertion device.

20. The system of any one of embodiments 16-19, wherein the applicator further defines a drug delivery channel configured to deliver the at least one plasmid to the lesion.

21. The system of any one of embodiments 16-20 further comprising at least one robotic arm engaged with the applicator to control a position of the applicator during administration of at least one of the at least one plasmid or the electroporation therapy.

22. The system of any one of embodiments 16-21 further comprising at least one visualization device configured to generate imagery of the lesion before or during administration of at least one of the at least one plasmid or the electroporation therapy.

23. The system of embodiment 22, wherein the at least one visualization device comprises a computed tomography scanner.

24. The system of any one of embodiments 16-23, wherein the generator is configured to output low-voltage electric pulses.

25. The system of any one of embodiments 16-24, wherein the electric pulses have a field strength of 700V/cm or less.

26. The system of any one of embodiments 16-23, wherein the generator is configured to output high-voltage electric pulses.

27. The system of any one of embodiments 16-26, wherein the at least one plasmid comprises tavokinogene telseplasmid.

28. A method of treating a lesion at a lung of a subject, the method comprising:

administering to the lesion an effective dose of at least one treatment agent;

administering electroporation therapy to the lesion, the electroporation therapy comprising administering an electric pulse to the lesion using an electroporation system comprising:

an applicator comprising:
a plurality of electrodes comprising a first electrode having a first tip and a second electrode having a second tip, wherein the plurality electrodes are configured to move between a retracted position and a deployed position;
wherein a distance between the first tip of the first electrode and the second tip of the second electrode is greater in the deployed position than in the retracted position; and

a generator electrically connected to the plurality of electrodes,

wherein administering the electric pulse to the lesion comprises disposing the first electrode and the second electrode into or adjacent to the lesion, and delivering the electric pulse from the generator to the first electrode and the second electrode.

29. The method of embodiment 28, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

30. The method of embodiment 28 or embodiment 29, wherein the electroporation system further comprises an insertion device defining at least one working channel, wherein at least a portion of the applicator is configured to pass through the at least one working channel to access the visceral lesion.

31. The method of any one of embodiments 28-30, wherein the electroporation system further comprises a drug delivery device configured to deliver the at least one treatment agent through the at least one working channel of the insertion device.

32. The method of any one of embodiments 28-31, wherein the applicator further defines a drug delivery channel configured to deliver the at least one treatment agent to the visceral lesion.

33. The method of any one of embodiments 28-32, wherein the electroporation system further comprises at least one robotic arm engaged with the applicator to control a position of the applicator during administration of at least one of the at least one treatment agent or the electroporation therapy.

34. The method of any one of embodiments 28-33, wherein the electroporation system further comprises at least one visualization device configured to generate imagery of the visceral lesion before or during administration of at least one of the at least one treatment agent or the electroporation therapy.

35. The method of any one of embodiments 28-34, wherein the generator is configured to output low-voltage electric pulses.

36. The method of any one of embodiments 28-35, wherein the electric pulses have a field strength of 700V/cm or less.

37. The method of any one of embodiments 28-34, wherein the generator is configured to output high-voltage electric pulses.

38. The method of any one of embodiments 28-37, wherein administering to the subject the effective dose of the at least one treatment agent comprises administering an effective dose of at least one plasmid coding for a cytokine.

39. The method of embodiment 38, wherein the at least one plasmid comprises tavokinogene telseplasmid.

40. The method of any one of embodiments 28-39, wherein administering to the subject the effective dose of the at least one treatment agent further comprises administering to the subject an effective dose of at least one checkpoint inhibitor.

41. The method of any one of embodiments 28-40 further comprising inserting a portion of the applicator into the lung of the subject via an esophagus of the subject.

42. A system for treating a lesion at a lung of a subject, the system comprising:

an applicator comprising a plurality of electrodes comprising a first electrode having a first tip and a second electrode having a second tip, wherein the plurality electrodes are configured to move between a retracted position and a deployed position; wherein a distance between the first tip of the first electrode and the second tip of the second electrode is greater in the deployed position than in the retracted position; and

a generator electrically connected to the plurality of electrodes, wherein the generator is configured to deliver an electric pulse to the first electrode and second electrode to administer the electric pulse to the lesion; and

at least one drug delivery channel configured to deliver to the subject an effective dose of at least one treatment agent.

43. The system of embodiment 42, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

44. The system of embodiment 42 or embodiment 43 further comprising an insertion device defining at least one working channel, wherein at least a portion of the applicator is configured to pass through the at least one working channel to access the lesion.

45. The system of any one of embodiments 42-44 further comprising a drug delivery device configured to deliver the at least one treatment agent through the at least one working channel of the insertion device.

46. The system of any one of embodiments 42-45, wherein the insertion device comprises a bronchoscope, and wherein the applicator is at least partially flexible.

47. The system of any one of embodiments 42-46, wherein the applicator further defines a drug delivery channel configured to deliver the at least one treatment agent to the lesion.

48. The system of any one of embodiments 42-47 further comprising at least one robotic arm engaged with the applicator to control a position of the applicator during delivery of at least one of the at least one treatment agent or the electroporation therapy.

49. The system of any one of embodiments 42-48 further comprising at least one visualization device configured to generate imagery of the lesion before or during delivery of at least one of the at least one treatment agent or the electroporation therapy.

50. The system of any one of embodiments 42-49, wherein the generator is configured to output low-voltage electric pulses.

51. The system of any one of embodiments 42-50, wherein the electric pulses have a field strength of 700V/cm or less.

52. The system of any one of embodiments 42-49, wherein the generator is configured to output high-voltage electric pulses.

53. A method of treating a visceral lesion at a pancreas of a subject, the method comprising:

administering to the subject an effective dose of at least one treatment agent;

administering electroporation therapy to the visceral lesion, the electroporation therapy comprising administering an electric pulse to the visceral lesion using an electroporation system comprising:

an applicator comprising:
a plurality of electrodes comprising a first electrode having a first tip and a second electrode having a second tip, wherein the plurality electrodes are configured to move between a retracted position and a deployed position;
wherein a distance between the first tip of the first electrode and the second tip of the second electrode is greater in the deployed position than in the retracted position; and

a generator electrically connected to the plurality of electrodes,

wherein administering the electric pulse to the visceral lesion comprises disposing the first electrode and the second electrode into or adjacent to the visceral lesion, and delivering the electric pulse from the generator to the first electrode and the second electrode.

54. The method of embodiment 53, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

55. The method of embodiment 53 or embodiment 54, wherein the electroporation system further comprises an insertion device defining at least one working channel, wherein at least a portion of the applicator is configured to pass through the at least one working channel to access the visceral lesion.

56. The method of any one of embodiments 53-55, wherein the electroporation system further comprises a drug delivery device configured to deliver the at least one treatment agent through the at least one working channel of the insertion device.

57. The method of any one of embodiments 53-56, wherein the insertion device comprises an endoscope, and wherein the applicator is at least partially flexible.

58. The method of any one of embodiments 53-57, wherein the applicator further defines a drug delivery channel configured to deliver the at least one treatment agent to the visceral lesion.

59. The method of any one of embodiments 53-58, wherein the electroporation system further comprises at least one robotic arm engaged with the applicator to control a position of the applicator during administration of at least one of the at least one treatment agent or the electroporation therapy.

60. The method of any one of embodiments 53-59, wherein the electroporation system further comprises at least one visualization device configured to generate imagery of the visceral lesion before or during administration of at least one of the at least one treatment agent or the electroporation therapy.

61. The method of embodiment 60, wherein the at least one visualization device comprises a computed tomography scanner.

62. The method of any one of embodiments 53-61, wherein the generator is configured to output low-voltage electric pulses.

63. The method of any one of embodiments 53-62, wherein the electric pulses have a field strength of 700V/cm or less.

64. The method of any one of embodiments 53-61, wherein the generator is configured to output high-voltage electric pulses.

65. The method of any one of embodiments 53-64, wherein administering to the subject the effective dose of the at least one treatment agent comprises administering an effective dose of at least one plasmid coding for a cytokine.

66. The method of embodiment 65, wherein the at least one plasmid comprises tavokinogene telseplasmid.

67. The method of any one of embodiments 53-66, wherein administering to the subject the effective dose of the at least one treatment agent further comprises administering to the subject an effective dose of at least one checkpoint inhibitor.

68. The method of any one of embodiments 53-67, wherein the applicator further comprises a piercing tip, the method further comprising:

inserting a portion of the applicator into a stomach of the subject;

piercing a stomach wall with the piercing tip; and

moving the plurality of electrodes from the retracted position to the deployed position.

69. A system for treating a visceral lesion at a pancreas of a subject, the system comprising:

an applicator comprising a plurality of electrodes comprising a first electrode having a first tip and a second electrode having a second tip, wherein the plurality electrodes are configured to move between a retracted position and a deployed position in response to actuation by the actuator; wherein a distance between the first tip of the first electrode and the second tip of the second electrode is greater in the deployed position than in the retracted position; and

a generator electrically connected to the plurality of electrodes, wherein the generator is configured to deliver an electric pulse to the first electrode and second electrode to administer the electric pulse to the visceral lesion; and

at least one drug delivery channel configured to deliver to the subject an effective dose of at least one treatment agent.

70. The system of embodiment 69, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

71. The system of embodiment 69 or embodiment 70 further comprising an insertion device defining at least one working channel, wherein at least a portion of the applicator is configured to pass through the at least one working channel to access the visceral lesion.

72. The system of any one of embodiments 69-71 further comprising a drug delivery device configured to deliver the at least one treatment agent through the at least one working channel of the insertion device.

73. The system of any one of embodiments 69-72, wherein the insertion device comprises a bronchoscope, and wherein the applicator is at least partially flexible.

74. The system of any one of embodiments 69-73, wherein the applicator further defines a drug delivery channel configured to deliver the at least one treatment agent to the visceral lesion.

75. The system of any one of embodiments 69-74 further comprising at least one robotic arm engaged with the applicator to control a position of the applicator during delivery of at least one of the at least one treatment agent or the electroporation therapy.

76. The system of any one of embodiments 69-75 further comprising at least one visualization device configured to generate imagery of the visceral lesion before or during delivery of at least one of the at least one treatment agent or the electroporation therapy.

77. The system of embodiment 76, wherein the at least one visualization device comprises a computed tomography scanner.

78. The system of any one of embodiments 69-77, wherein the generator is configured to output low-voltage electric pulses.

79. The system of any one of embodiments 69-78, wherein the electric pulses have a field strength of 700V/cm or less.

80. The system of any one of embodiments 69-77, wherein the generator is configured to output high-voltage electric pulses.

81. The system of any one of embodiments 69-80, wherein the applicator further comprises a piercing tip configured to pierce a stomach wall of the subject to administer at least one of the at least one treatment agent or the electric pulse to or proximate the visceral lesion on the pancreas.

82. A method of treating a lesion of a subject, the method comprising:

administering to the subject an effective dose of at least one treatment agent;

administering electroporation therapy to the lesion, the electroporation therapy comprising administering an electric pulse to the lesion using an electroporation system comprising:

an applicator comprising a plurality of electrodes comprising a first electrode having a first tip and a second electrode having a second tip; and

a generator electrically connected to the plurality of electrodes,

wherein administering the electric pulse to the lesion comprises disposing the first electrode and the second electrode into or adjacent to the lesion, and delivering the electric pulse from the generator to the first electrode and the second electrode.

83. The method of embodiment 82, wherein the plurality electrodes are configured to move between a retracted position and a deployed position, and wherein a distance between the first tip of the first electrode and the second tip of the second electrode is greater in the deployed position than in the retracted position.

84. The method of embodiment 82 or embodiment 83, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

85. The method of any one of embodiments 82-84, wherein the electroporation system further comprises an insertion device defining at least one working channel, wherein at least a portion of the applicator is configured to pass through the at least one working channel to access the lesion.

86. The method of any one of embodiments 82-85, wherein the electroporation system further comprises a drug delivery device configured to deliver the at least one treatment agent through the at least one working channel of the insertion device.

87. The method of any one of embodiments 82-86, wherein the insertion device comprises an endoscope, and wherein the applicator is at least partially flexible.

88. The method of any one of embodiments 82-87, wherein the insertion device comprises a trocar, and wherein the applicator is substantially rigid.

89. The method of any one of embodiments 82-88, wherein the applicator further defines a drug delivery channel configured to deliver the at least one treatment agent to the lesion.

90. The method of any one of embodiments 82-89, wherein the electroporation system further comprises at least one robotic arm engaged with the applicator to control a position of the applicator during administration of at least one of the at least one treatment agent or the electroporation therapy.

91. The method of any one of embodiments 82-90, wherein the electroporation system further comprises at least one visualization device configured to generate imagery of the lesion before or during administration of at least one of the at least one treatment agent or the electroporation therapy.

92. The method of any one of embodiments 82-91, wherein the generator is configured to output low-voltage electric pulses.

93. The method of any one of embodiments 82-92, wherein the electric pulses have a field strength of 700V/cm or less.

94. The method of any one of embodiments 82-91, wherein the generator is configured to output high-voltage electric pulses.

95. The method of any one of embodiments 82-94, wherein treating the lesion comprises administering an effective dose of at least one plasmid coding for a cytokine.

96. The method of embodiment 95, wherein the cytokine comprises IL-12.

97. The method of embodiment 95, wherein the at least one plasmid comprises tavokinogene telseplasmid.

98. The method of any one of embodiments 82-97, wherein treating the lesion further comprises administering to the subject an effective dose of at least one checkpoint inhibitor.

99. The method of any one of embodiments 82-98, wherein the treatment agent comprises at least one plasmid encoding an immunomodulatory polypeptide.

100. The method of embodiment 99, wherein the immunomodulatory polypeptide comprises: a cytokine, a costimulatory molecule, a genetic adjuvant, an antigen, a genetic adjuvant-antigen fusion polypeptide, a chemokine, or an antigen binding polypeptide.

101. The method of embodiment 100, wherein the immunomodulatory polypeptide comprises: CXCL9, anti-CD3 scFy, or anti-CTLA scFy.

102. A system for treating a lesion of a subject, the system comprising:

an applicator comprising a plurality of electrodes comprising a first electrode having a first tip and a second electrode having a second tip; and

a generator electrically connected to the plurality of electrodes, wherein the generator is configured to deliver an electric pulse to the first electrode and second electrode to administer the electric pulse to the lesion; and

at least one drug delivery channel configured to deliver to the subject an effective dose of at least one treatment agent.

103. The system of embodiment 102, wherein the plurality electrodes are configured to move between a retracted position and a deployed position, and wherein a distance between the first tip of the first electrode and the second tip of the second electrode is greater in the deployed position than in the retracted position.

104. The system of embodiment 102 or embodiment 103, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

105. The system of any one of embodiments 102-104 further comprising an insertion device defining at least one working channel, wherein at least a portion of the applicator is configured to pass through the at least one working channel to access the lesion.

106. The system of any one of embodiments 102-105 further comprising a drug delivery device configured to deliver the at least one treatment agent through the at least one working channel of the insertion device.

107. The system of any one of embodiments 102-106, wherein the insertion device comprises an endoscope, and wherein the applicator is at least partially flexible.

108. The system of any one of embodiments 102-107, wherein the insertion device comprises a trocar, and wherein the applicator is substantially rigid.

109. The system of any one of embodiments 102-108, wherein the applicator further defines a drug delivery channel configured to deliver the at least one treatment agent to the lesion.

110. The system of any one of embodiments 102-109 further comprising at least one robotic arm engaged with the applicator to control a position of the applicator during delivery of at least one of the at least one treatment agent or the electric pulse.

111. The system of any one of embodiments 102-110 further comprising at least one visualization device configured to generate imagery of the lesion before or during delivery of at least one of the at least one treatment agent or the electric pulse.

112. The system of embodiment 111, wherein the at least one visualization device comprises a computed tomography scanner.

113. The system of any one of embodiments 102-112, wherein the generator is configured to output low-voltage electric pulses.

114. The system of any one of embodiments 102-113, wherein the electric pulses have a field strength of 700V/cm or less.

115. The system of any one of embodiments 102-112, wherein the generator is configured to output high-voltage electric pulses.

116. The system of any one of embodiments 102-115, wherein treating the lesion comprises delivering an effective dose of at least one plasmid coding for a cytokine.

117. The system of embodiment 116, wherein the at least one plasmid comprises tavokinogene telseplasmid.

118. The system of any one of embodiments 102-117, wherein delivering to the subject the effective dose of the at least one treatment agent further comprises delivering to the subject an effective dose of at least one checkpoint inhibitor.

119. The system of any one of embodiments 102-118, wherein the treatment agent comprises at least one plasmid encoding an immunomodulatory polypeptide.

120. The system of embodiment 119, wherein the immunomodulatory polypeptide comprises: a cytokine, a costimulatory molecule, a genetic adjuvant, an antigen, a genetic adjuvant-antigen fusion polypeptide, a chemokine, or an antigen binding polypeptide.

121. The system of embodiment 119 or embodiment 120, wherein the immunomodulatory polypeptide comprises: CXCL9, anti-CD3 scFv, or anti-CTLA-4 scFv

122. An applicator for electroporation comprising:

a control portion;

an insertion tube connected to the control portion;

an actuator engaged with the control portion, wherein at least a portion of the actuator is movable relative to the control portion and the insertion tube; and

a plurality of electrodes comprising a first electrode having a first tip and a second electrode having a second tip, wherein the plurality electrodes are configured to move between a retracted position and a deployed position in response to actuation by the actuator;

wherein a distance between the first tip of the first electrode and the second tip of the second electrode is greater in the deployed position than in the retracted position.

123. The applicator of embodiment 122, wherein the first tip and the second tip are recessed entirely within the insertion tube in the retracted position, and wherein at least the first tip and the second tip are configured to extend from the insertion tube into adjacent tissue in the deployed position.

124. The applicator of embodiment 122 or embodiment 123, wherein in the deployed position, the distance between the first tip of the first electrode and the second tip of the second electrode is greater than an external diameter of a distal end of the insertion tube.

125. The applicator of any one of embodiments 122-124, wherein the insertion tube comprises a first angled channel and a second angled channel defined at a distal end of the insertion tube,

wherein the first angled channel and the second angled channel are each oriented at acute angles to a longitudinal axis of the insertion tube,

wherein the first electrode is configured to extend at least partially through the first angled channel in the deployed position,

wherein the second electrode is configured to extend at least partially through the second angled channel in the deployed position,

wherein in the retracted position, the first electrode and the second electrode are disposed parallel to each other within the insertion tube, and

wherein in the deployed position, at least a portion of the first electrode and at least a portion of the second electrode are disposed at the respective acute angles of the first angled channel and the second angled channel.

126. The applicator of any one of embodiments 122-124, further comprising a bladder engaged with the first electrode and the second electrode, wherein the bladder is disposed entirely within the insertion tube in the retracted position, and wherein the bladder is disposed at least partially outside the insertion tube in the deployed position.

127. The applicator of any one of embodiments 122-126, wherein at least a portion of the first electrode and the second electrode comprises nitinol, wherein the nitinol is configured to change shape in an instance in which the plurality of electrodes are in the deployed position, and wherein the nitinol is configured to change shape above human body temperature.

128. The applicator of any one of embodiments 122-127, further comprising a nitinol sleeve attached to each of the first electrode and a second electrode, wherein the nitinol is configured to change shape in an instance in which the plurality of electrodes are in the deployed position, and wherein the nitinol is configured to change shape above human body temperature.

129. The applicator of any one of embodiments 122-128, wherein the first electrode and the second electrode are non-linear.

130. The applicator of any one of embodiments 122-124 or 127-129, further comprising a carrier movably disposed at least partially within the insertion tube, wherein the first electrode and the second electrode are each disposed at least partially within the carrier, wherein the carrier defines a first portion associated with the first electrode and a second portion associated with the second electrode, and wherein the first portion and the second portion are configured to expand radially away from each other when moving from the retracted position to the expanded position.

131. The applicator of embodiment 130, further comprising an inner member configured to receive a force from the actuator to expand the first portion and the second portion of the carrier radially outwardly.

132. The applicator of embodiment 130 or embodiment 131, further comprising a spring disposed between the first portion and the second portion, wherein the spring is configured to expand the first portion and the second portion of the carrier radially outwardly.

133. The applicator of any one of embodiments 122-132, further comprising a drug delivery channel configured to fluidly connect a drug delivery device with a target site via the insertion tube of the applicator.

134. The applicator of embodiment 133, wherein the actuator is configured to displace the drug delivery channel towards the target site.

135. The applicator of embodiment 134, wherein the drug delivery channel is configured to move between a retracted position of the drug delivery channel and the deployed position of the drug delivery channel simultaneously with the plurality of electrodes in response to actuation by the actuator.

136. The applicator of any one of embodiments 122-135, wherein the insertion tube defines a piercing tip at a distal end.

137. The applicator of any one of embodiments 122-136, wherein the insertion tube comprises a flexible portion, wherein the flexible portion is configured to steer a distal end of the insertion tube.

138. The applicator of any one of embodiments 122-137, wherein the insertion tube comprises a rigid portion, wherein the rigid portion is disposed between the distal end of the insertion tube and the control portion of the applicator, wherein the applicator comprises at least one cable disposed within the insertion tube, and wherein the at least one cable is attached to the applicator between the distal end of the insertion tube and the rigid portion to steer the distal end of the insertion tube.

139. A system for electroporation comprising:

an applicator comprising:
a control portion;
an insertion tube connected to the control portion;
an actuator engaged with the control portion, wherein at least a portion of the actuator is movable relative to the control portion and the insertion tube; and
a plurality of electrodes comprising a first electrode having a first tip and a second electrode having a second tip, wherein the plurality electrodes are configured to move between a retracted position and a deployed position in response to actuation by the actuator;
wherein a distance between the first tip of the first electrode and the second tip of the second electrode is greater in the deployed position than in the retracted position;

an insertion device defining a working channel, wherein at least a portion of the insertion tube of the applicator is configured to pass through the working channel;

a generator electrically connected to the plurality of electrodes, wherein the generator is configured to deliver electrical signals to the plurality of electrodes; and

a drug delivery device configured to deliver one or more treatment agents through the working channel of the insertion device.

140. The system of embodiment 139, wherein in the deployed position, the distance between the first tip of the first electrode and the second tip of the second electrode is greater than an internal diameter of the working channel.

141. The system of embodiment 139 or embodiment 140, wherein in the retracted position, the portion of the insertion tube and plurality of electrodes are configured to pass through the working channel of the insertion device.

142. The system of any one of embodiments 139-141, further comprising a processor configured to cause the generator to transmit electrical signals to the first electrode and the second electrode and receive electrical signals indicative of an impedance of a tissue disposed between the first electrode and the second electrode.

143. The system of any one of embodiments 139-142, wherein the insertion device comprises an endoscope.

144. The system of embodiment 143, wherein the endoscope comprises a bronchoscope.

145. The system of any one of embodiments 139-144, wherein the applicator further comprises a drug delivery channel configured to fluidly connect a drug delivery device with a target site via the insertion tube of the applicator.

146. The system of embodiment 145, wherein the actuator is configured to displace the drug delivery channel towards the target site.

147. The system of any one of embodiments 139-146, wherein the drug delivery channel is configured to move between a retracted position of the drug delivery channel and the deployed position of the drug delivery channel simultaneously with the plurality of electrodes in response to actuation by the actuator.

148. The system of any one of embodiments 139-147, wherein the insertion tube comprises a flexible portion, wherein the flexible portion is configured to steer a distal end of the insertion tube.

149. The system of any one of embodiments 139-148, wherein the insertion tube comprises a rigid portion, wherein the rigid portion is disposed between the distal end of the insertion tube and the control portion of the applicator, wherein the applicator comprises at least one cable disposed within the insertion tube, and wherein the at least one cable is attached to the applicator between the distal end of the insertion tube and the rigid portion to steer the distal end of the insertion tube.

150. The system of any one of embodiments 139-149, wherein the applicator comprises a drug delivery channel, and wherein the drug delivery device is configured to deliver the one or more treatment agents via the drug delivery channel.

151. A method of treating a tumor, the method comprising:

inserting an insertion device into a patient until a distal end of the insertion device is disposed adjacent to a target site;

inserting an insertion tube of an applicator into the working channel of the insertion device, such that a distal end of the insertion tube, including a plurality of electrodes, is positioned adjacent to the target site;

administering a treatment agent from a drug delivery device to the target site via a working channel of the insertion device;

delivering one or more electrical pulses from a generator to the electrodes to electroporate the tissue at the target site; and

removing the applicator and insertion device from the patient.

152. The method of embodiment 151, wherein administering a treatment agent comprises inserting a portion of the drug delivery device into the working channel of the insertion device, such that the portion of the drug delivery device is positioned adjacent to the target site; and

wherein the method further comprises removing the portion of the drug delivery device from the insertion device

153. The method of embodiment 151 or embodiment 152, wherein inserting the insertion tube of the applicator into the working channel further comprises positioning a drug delivery channel adjacent to the target site.

154. The method of any one of embodiments 151-153, further comprising actuating the applicator to move the plurality of electrodes and the drug delivery channel into a deployed position after inserting the insertion tube and before administering the treatment agent or delivering the one or more electrical pulses.

155. A method of treating a subject having a tumor comprising:

a) administering to the subject an effective dose of a treatment agent; and

b) administering electroporation therapy to the tumor, the electroporation therapy comprises administering an electric pulse to the tumor using the system of any one of embodiments 102-121 or 139-150.

156. The method of embodiment 155, wherein the treatment agent is administered via a drug delivery device of the applicator.

157. The method of embodiment 155 or embodiment 156, wherein the treatment agent comprises an expression vector encoding a therapeutic polypeptide.

158. The method of embodiment 157, wherein the expression vector encodes one or more of: co-stimulatory polypeptide, immunomodulatory polypeptide, immunostimulatory cytokine, checkpoint inhibitor, adjuvant, antigen, genetic adjuvant-antigen fusion polypeptide, chemokine, or antigen binding polypeptides.

159. The method of embodiment 158, wherein the co-stimulatory molecule is selected from the group consisting of: GITR, CD137, CD134, CD40L, and CD27 agonists.

160. The method of embodiment 158 or embodiment 159, wherein the expression vector encodes a polypeptide comprising CXCL9, anti-CD3 scFv, or anti-CTLA-4 scFv.

161. The method of any one of embodiments 158-160, wherein the immunostimulatory cytokine is selected from the group consisting of: TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ, and a combination of any two of TNFα, IL-1, IL-10, IL-12, IL-12 p35, IL-12 p40, IL-15, IL-15Ra, IL-23, IL-27, IFNα, IFNβ, IFNγ, IL-2, IL-4, IL-5, IL-7, IL-9, IL-21, TGFβ.

162. The method of any one of embodiments 155-161, wherein the method further comprises administering an effective dose of a checkpoint inhibitor to the subject.

163. The method of embodiment 162, wherein the checkpoint inhibitor is administered systemically.

164. The method of embodiment 162 or embodiment 163, wherein the checkpoint inhibitor is encoded on the expression vector encoding an immunostimulatory cytokine or on a second expression vector and delivered to the cancerous tumor by the electroporation therapy.

165. The method of any one of embodiments 162-164, wherein the checkpoint inhibitor is administered prior to, concurrent with, and/or subsequent to electroporation of the immunostimulatory cytokine.

166. The method of any one of embodiments 157-165, wherein the expression vector comprises:

a) P-A-T-C,

b) P-A-T-B-T-C, or

c) P-C-T-A-T-B

wherein P is a promoter, T is a translation modification element, A encodes an immunomodulatory molecule, a chain of an immunomodulatory molecule or a co-stimulatory molecule, B encodes an immunomodulatory molecule, a chain of an immunomodulatory molecule or a co-stimulatory molecule, and C encodes a immunomodulatory molecule, chain of an immunomodulatory molecule a costimulatory molecule, genetic adjuvant, antigen, a genetic adjuvant-antigen fusion polypeptide, chemokine, or antigen binding polypeptide.

167. The method of any one of embodiments 157-166, wherein the expression vector encodes a polypeptide comprising CXCL9, anti-CD3 scFv, or anti-CTLA-4 scFv.

168. The method of any one of embodiments 155-167, further comprising piercing a tissue with a distal end of the applicator to access the tumor.

169. A method of reducing recurrence of tumor cell growth in a mammalian tissue, the method comprising:

a) administering a treatment agent to the tumor and/or a tumor margin tissue;

b) administering electroporation therapy to the tumor and/or the tumor margin tissue using a generator and the applicator of any one of embodiments 102-150.

170. The method of embodiment 169, wherein administering a treatment agent comprises injecting an expression vector encoding the treatment agent into the tumor and/or a tumor margin tissue.

171. The method of embodiment 169 or embodiment 170, wherein the electroporation therapy is administered prior to or after surgical resection or ablation of the tumor.

172. The method of any one of embodiments 169-171, wherein the generator comprises a low-voltage generator.

173. The method of any one of embodiments 169-172, wherein the electroporation therapy is administered using the low-voltage generator producing an electric field of 400V/cm or less.

174. The method of any one of embodiments 169-171, wherein the generator comprises a high-voltage generator.

175. A method of treating a subject having a tumor comprising:

administering to the subject an effective dose of at least one DNA-based treatment agent;

transfecting the at least one DNA-based treatment agent into a plurality of cells of the tumor using an electroporation applicator and generator;

wherein the generator is configured to apply low voltage electroporation pulses to the tumor via the electroporation applicator; and

wherein 8-10% of the at least one DNA-based treatment agent is transfected into cells of the tumor.

176. The method of embodiment 175, wherein the applicator comprises:

a control portion;

an insertion tube connected to the control portion;

an actuator engaged with the control portion, wherein at least a portion of the actuator is movable relative to the control portion and the insertion tube; and

a plurality of electrodes comprising a first electrode having a first tip and a second electrode having a second tip, wherein the plurality electrodes are configured to move between a retracted position and a deployed position in response to actuation by the actuator;

wherein a distance between the first tip of the first electrode and the second tip of the second electrode is greater in the deployed position than in the retracted position.

177. The method of embodiment 176, wherein the generator is electrically connected to the plurality of electrodes, and the generator is configured to deliver electrical signals to the plurality of electrodes.

178. The method of any one of embodiments 175-177, wherein each low voltage electroporation pulse defines a duration of 1 ms or greater.

179. The method of embodiment 178, wherein each low voltage electroporation pulse defines a duration from 0.5 ms to is.

180. The method of any one of embodiments 175-179, wherein the low voltage electroporation pulses comprise a voltage of 600V or less.

181. The method of any one of embodiments 175-180, wherein the low voltage electroporation pulses comprise a voltage from 600V to 5V.

182. The method of any one of embodiments 175-181, wherein the low voltage electroporation pulses comprise a field of 700V/cm or less.

183. A method of treating a subject having a tumor comprising:

administering to the subject an effective dose of at least one DNA-based treatment agent;

transfecting the at least one DNA-based treatment agent into a plurality of cells of the tumor using an electroporation applicator and generator;

wherein the generator is configured to apply high voltage electroporation pulses to the tumor via the electroporation applicator; and

wherein 8-10% of the at least one DNA-based treatment agent is transfected into cells of the tumor.

184. A method of increasing responsiveness to checkpoint inhibitor therapy in a subject comprising:

injecting a tumor in the subject with an effective dose of at least one plasmid coding for a cytokine; and

administering electroporation therapy to the tumor.

185. The method of embodiment 184, wherein the tumor is in the liver.

186. The method of embodiment 184 or embodiment 185, wherein the tumor is hepatocellular carcinoma.

187. The method of any one of embodiments 184-186, wherein the cytokine is selected from the group consisting of: TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ, and a combination of any two of TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, TGFβ.

188. The method of any one of embodiments 184-187, wherein the cytokine is IL-12.

189. The method of any one of embodiments 184-188, wherein the subject has had, is having, or is predicted to have low responsiveness or non-responsiveness to checkpoint inhibitor therapy.

190. The method of any one of embodiments 184-189, wherein modulating checkpoint inhibitor therapy further comprised administering to the subject an effective dose of at least one checkpoint inhibitor.

191. A trocar-based system for electroporation comprising:

an applicator comprising:
a control portion;
an insertion tube connected to the control portion;
an actuator engaged with the control portion, wherein at least a portion of the actuator is movable relative to the control portion and the insertion tube; and
a plurality of electrodes comprising a first electrode having a first tip and a second electrode having a second tip, wherein the plurality electrodes are configured to move between a retracted position and a deployed position in response to actuation by the actuator;
wherein a distance between the first tip of the first electrode and the second tip of the second electrode is greater in the deployed position than in the retracted position

a trocar defining a working channel, wherein at least a portion of the insertion tube of the applicator is configured to pass through the working channel;

a generator electrically connected to the plurality of electrodes, wherein the generator is configured to deliver electrical signals to the plurality of electrodes; and

a drug delivery device configured to deliver one or more treatment agents through the working channel of the trocar.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A method of treating a lesion at a lung of a subject who is non-responsive or predicted to be non-responsive to anti-PD-1 or anti-PD-L1 therapy, the method comprising:
administering to the lesion an effective dose of at least one plasmid coding for IL-12;
administering electroporation therapy to the lesion; and
administering to the subject an effective dose of at least one checkpoint inhibitor;
wherein administering the electroporation therapy comprises administering an electric pulse to the lesion using an electroporation system comprising:
an applicator comprising:
a plurality of electrodes comprising a first electrode having a first tip and a second electrode having a second tip, wherein the plurality electrodes are configured to move between a retracted position and a deployed position;
wherein a distance between the first tip of the first electrode and the second tip of the second electrode is greater in the deployed position than in the retracted position; and
a generator electrically connected to the plurality of electrodes,
wherein administering the electric pulse to the lesion comprises disposing the first electrode and the second electrode into or adjacent to the lesion, and delivering the electric pulse from the generator to the first electrode and the second electrode.

2. The method of claim 1, wherein the applicator further comprises a control portion; an insertion tube connected to the control portion; and an actuator engaged with the control portion, wherein at least a portion of the actuator is movable relative to the control portion and the insertion tube to cause the plurality of electrodes to move between the retracted position and the deployed position.

3. The method of claim 1, wherein the electroporation system further comprises an insertion device comprising one of a rigid trocar or flexible endoscope defining at least one working channel, wherein at least a portion of the applicator is configured to pass through the at least one working channel to access the lesion.

4. The method of claim 3, wherein the electroporation system further comprises a drug delivery device configured to deliver at least one of the at least one plasmid or the at least one checkpoint inhibitor through the at least one working channel of the insertion device.

5. The method of claim 1, wherein the applicator further defines a drug delivery channel configured to deliver at least one of the at least one plasmid or the at least one checkpoint inhibitor to the lesion.

6. The method of claim 1, wherein the electroporation system further comprises at least one robotic arm engaged with the applicator to control a position of the applicator during administration of at least one of the at least one plasmid, the at least one checkpoint inhibitor, or the electroporation therapy.

7. The method of claim 1, wherein the electroporation system further comprises at least one visualization device configured to generate imagery of the lesion before or during administration of at least one of the at least one plasmid, the at least one checkpoint inhibitor, or the electroporation therapy.

8. The method of claim 7, wherein the at least one visualization device comprises a computed tomography scanner.

9. The method of claim 1, wherein the generator is configured to output low-voltage electric pulses.

10. The method of claim 9, wherein the electric pulses have a field strength of 700V/cm or less.

11. The method of claim 1, wherein the generator is configured to output high-voltage electric pulses.

12. The method of claim 1, wherein the at least one plasmid comprises tavokinogene telseplasmid.

13. The method of claim 1, wherein the checkpoint inhibitor is administered systemically.

14. The method of claim 13, wherein the checkpoint inhibitor is an anti-PD-1 antibody or an anti-PD-L1 antibody.

15. The method of claim 13, wherein the checkpoint inhibitor comprises: nivolumab, pembrolizumab, pidilizumab, or MPDL3280A.

16.-191. (canceled)
